(19) United States
(12) Patent Application Publication
(10) Pub. No.: US 2022/0290196 A1
(43) Pub. Date: Sep. 15, 2022

(54) GENETICALLY ENGINEERED MICROBES AND BIOSYNTHETIC METHODS

(71) Applicant: The Regents of the University of California, Oakland, CA (US)

(72) Inventors: Bradley S. MOORE, La Jolla, CA (US); Hanna LUHAVAYA, Oakland, CA (US)

(21) Appl. No.: 17/638,596

(22) PCT Filed: Aug. 28, 2020
(86) PCT No.: PCT/US2020/048570

§ 371 Date: Feb. 25, 2022

Related U.S. Application Data
(60) Provisional application No. 62/892,880, filed on Aug. 28, 2019.

(51) Int. Cl. C12P 13/22

(52) U.S. Cl. CPC C12P 13/22

(57) ABSTRACT

Provided herein, inter alia, are genetically engineered microbes and methods of use thereof for producing L-4-chlorokynrenine from L-Tryptophan. The genetically engineered microbes include, inter alia, one or more exogenous nucleic acids or enzymes for producing L-4-chlorokynrenine.

### CROSS-REFERENCES TO RELATED APPLICATIONS

This application claims the benefit of U.S. Provisional Application No. 62/892,880, filed Aug. 28, 2019, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant no. R01-GM085770 awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 048537-627001WO_SequenceListing_ST25.txt, created Aug. 27, 2020, 94,208 bytes, machine format IBM-PC, MS Windows operating system, is hereby incorporated by reference.

### BACKGROUND

Suicide is 2-7× higher in Veterans than non-veterans, and may be related to brain kynurenine pathway (KP) dysregulation and NMDA receptor (NMDAR) hyperactivation. L-4-Chlorokynurenine (L-4-Cl-Kyn) is a neuropharmaceutical drug candidate that is in development for the treatment of major depressive disorder (Double-Blind, Placebo-Controlled, Phase 2 Trial to Test Efficacy and Safety of AV-101 (L-4-chlorokynurenine) as Adjunct to Current Antidepressant Therapy in Patients With Major Depressive Disorder (the ELEVATE Study)). L-4-Chlorokynurenine has also entered Phase 2 clinical trials as a potential treatment to reduce levodopa-induced dyskinesia in patients with Parkinson's disease (ClinicalTrials.gov Identifier: NCT04147949).

L-4-chlorokynurenine (L-4-Cl-Kyn), a non-proteinogenic amino acid, is a next-generation, fast-acting oral prodrug [1, 2]. Studies report that this drug candidate is effective in animal models for the treatment of neuropathic pain, epilepsy, and Huntington's disease [2]. After active transport across the blood-brain barrier, L-4-Cl-Kyn is enzymatically converted into the active agent 7-chlorokynurenic acid, which is a highly selective competitive antagonist of the N-methyl-D-aspartic acid (NMDA) receptor [3].

To date, only synthetic routes to L-4-Cl-Kyn have been described [3, 4]. The synthetic methods are not applicable for scale-up due to reagents involved, produce racemic mixtures of 4-chlorokynurenine where separation of the enantiomers were not success, or involve multiple chemical steps relying on environmentally toxic chemicals. Disclosed herein, inter alia, are solutions to these and other problems in the art.

### BRIEF SUMMARY

In an aspect is provided a genetically engineered microbe, wherein the genetically engineered microbe includes an exogenous Tar14 encoding nucleic acid, an exogenous Tar13 encoding nucleic acid, or an exogenous Tar16 encoding nucleic acid.

In an aspect is provided a genetically engineered microbe, wherein the genetically engineered microbe includes one or more of an exogenous Tar14 encoding nucleic acid, an exogenous Tar13 encoding nucleic acid, or an exogenous Tar16 encoding nucleic acid.

In an aspect is provided a genetically engineered microbe, wherein the genetically engineered microbe includes a nucleic acid encoding for an exogenous tryptophan halogenase.

In another aspect is provided a genetically engineered microbe, wherein the microbe expresses one or more of Tar14, Tar13, or Tar16.

In an aspect is provided a method of producing L-4-chlorokynurenine (L-4-Cl-Kyn), including contacting a genetically engineered microbe provided herein, including embodiments thereof with L-tryptophan (L-Trp).

In an aspect a method of synthesizing L-4-Cl-Kyn is provided, the method including contacting L-Trp with a Tar14 enzyme, a Tar13 enzyme, and a Tar16 enzyme.

In another aspect is provided a method of making L-4-Cl-Kyn, the method including contacting a microbe with L-Trp, wherein the microbe expresses one or more of Tar14, Tar13, or Tar16, and allowing the microbe to produce L-4-Cl-Kyn from L-Trp.

In an aspect, an isolated nucleic acid is provided, the isolated nucleic acid including a Tar14 encoding nucleic acid, a Tar13 encoding nucleic acid, a Tar16 encoding nucleic acid, or a Tar15 nucleic acid.

In an aspect is provided an isolated nucleic acid, the isolated nucleic acid including one or more of a Tar14 encoding nucleic acid, a Tar13 encoding nucleic acid, a Tar16 encoding nucleic acid, or a Tar5 nucleic acid.

In an aspect, an isolated enzyme is provided, the isolated enzyme including Tar14, Tar13, Tar16, or Tar15, or enzymatically active fragment or variant thereof.

In an aspect is a genetically engineered microbe including an exogenous nucleic acid provided herein, including embodiments thereof.

In another aspect is provided a method of treating a subject having a neurological disorder, the method including administering an effective amount of L-4-Cl-Kyn to the subject, thereby treating the neurological disorder.

### BRIEF DESCRIPTION OF THE DRAWINGS

FIG. 1A shows chemical structures of taromycins A (2) and B (3).

FIG. 1B shows the proposed enzymatic route from L-Trp (4) to L-4-Cl-Kyn (1).

FIG. 1C shows the gene organization of the taromycin BGC; tar13,14,15,16 gene numbers are labelled 13, 14, 15, and 16.

FIGS. 2A-2C are LCMS ion chromatograms. FIG. 2A shows an extracted ion chromatogram (EIC) recovery of taromycin production by S. coelicolor M1146-tarM1Δtar14 mutant fed with 6-Cl-Trp; m/z [M+2H]²⁺ 820.8 corresponds to taromycin A (2), m z [M+2H]²⁺ 827.8—taromycin B (3). FIG. 2B shows total ion chromatograms (TIC) of Tar14-catalyzed halogenation of L-Trp. FIG. 2C are TIC and EIC confirming activity of Tar13 and Tar16, and one-pot conversion of L-Trp to L-4-Cl-Kyn. * not relevant to the reaction product; m/z [M+H]⁺ 271.0 corresponds to N-formyl-L-4-Cl-Kyn, 243.0—L-4-Cl-Kyn.

FIG. 3A is a cartoon representation of the Tar14 structure. Monomers show the secondary structure, including α-helices, β-sheets, and flexible loops. Monomer on the left is labelled box-shaped domain and pyramid domain. Flavin molecules and catalytic K88 and E373 residues are labelled.

FIG. 3B shows superimposition of putative active site residues of Tar14, Th-Hal, and SttH.

FIG. 3C shows protein sequence alignment of Tar14 and selected Trp FDHs. Residues that form the active site in ThaI are highlighted by lines on top of the sequence alignment, sequences surrounding the putative active site in Tar14 are highlighted by lines below the sequence alignment.

FIG. 3D shows superimposition of Tar14 and ThaI structures illustrating difference in arrangement of the putative active site. Greek letters correspond to the respective sequence regions from the sequence alignment in FIG. 3D.

FIG. 4A shows retention time of mono- (2-F, middle panel) and di-fluorinated (2-F₂, bottom panel) analogues of taromycin A (2) and their comparison to 2 (top panel).

FIG. 4B shows LC/ESI-Q-TOF MS² analysis of mono- (2-F, middle panel) and di-fluorinated (2-F₂, bottom panel) analogues of taromycin A (2) and their comparison to 2 (top panel).

FIG. 5 is a schematic showing evaluation of L-4-Cl-Kyn biosynthesis enzymes against a panel of non-native substrate analogues.

FIG. 6 is a Neighbor-Joining tree of characterized flavin dependent halogenases. Support for each clade is indicated by bootstrap percentage values. The name of each terminal branch corresponds to the name of the halogenase. Tree is based on substrate specificity: top—halogenases that act on free L-tryptophan (regiospecificity of halogenation indicated in parentheses), bottom—on carrier protein tethered substrates; left—on peptide substrates. Flavin dependent oxidoreductase FzmM XY332 was used as outgroup (right). Protein sequences were aligned using Geneious 9.1.8 software, MUSCLE algorithm with default parameters. The phylogenetic tree was constructed using Geneious Tree Builder with Jukes-Cantor genetic distance model and Neighbor-Joining method with default parameters.

FIG. 7 shows multiple protein sequence alignment of selected tryptophan flavin-dependent chlorinases. The catalytic lysine (K) and glutamate (E) are marked with stars, residues proposed to interact with tryptophan in SttH and Th-Hal C6 halogenases are marked with triangles, residues proposed to form active site in C6 halogenase ThaI are indicated by diamonds. Lines highlight two flavin-binding conserved consensus motifs (GxGxxG and WxWxIP). The alignment is generated using Geneious 9.1.8, MUSCLE software with default parameters. Residues are shaded by similarity.

FIGS. 8A-8C shows analysis of the purified recombinant flavin dependent halogenase Tar14. FIG. 8A shows SDS-PAGE analysis of the purified Tar14 with expected protein size indicated. EZ-Run™ Rec protein Ladder (Fisher Scientific). Protein fractions did not have obvious yellow color associated with binding flavin (FAD) cofactor. FIG. 8B is a gel-filtration chromatogram of the Tar14 protein confirming its dimeric form in solution. FIG. 8C is a picture of Tar14 crystals under the microscope. The color of the crystal suggests co-crystallization of the protein with cofactor FAD.

FIGS. 9A and 9B show overlay of crystal structures of two sub-types of C6 FDHs: FIG. 9A Tar14, SttH, and Th-Hal; FIG. 9B. Tar14 and ThaI. Tar14, SttH, and Th-Hal proteins show conservation of secondary structure and alignment of putative active site residues (numbering corresponds to Tar14 sequence), while, secondary structure elements (6 and γ loops) that surround the putative active site of Tar14 are formed by different sequence regions compared to ThaI (loops a and 3. Catalytic lysine and glutamate are shown as sticks, cofactor FAD is shown as green sticks, substrate L-tryptophan—as yellow sticks. Residues 160-174 from loop γ in Tar14 structure lacked electron density (dotted line) which can be explained by poor ordering in the absence of substrate.

FIG. 9C is ConSurf software^{[21]} generated cartoon representation of monomeric unit of Tar14 at two different angles. Sequence alignment of Tar14 with other characterized 6-Cl-Trp FDH (KtzR, SttH, Th-Hal) was superimposed onto the Tar14 structure. The K88/E373 catalytic pair is shown as red sticks, flavin cofactor (FAD) is shown as grey sticks. Conserved residues colored in purple with darker color corresponding to higher similarity, shades of blue color highlight variable regions.

FIGS. 10A and 10B are a HPLC chromatogram illustrating separation of substrate/product/internal standard peaks using HPLC method 4 for studing kinetics of Tar14-catalyzed chlorination of L-tryptophan (FIG. 10A), and a calibration curve showing dependence of the ratio of area of the L-6-chlorotryptophan peak to the area of the phenol peak from ratio of concentration of the L-6-chlorotryptophan peak to the concentration of phenol (FIG. 10B). Error bars indicate one standard deviation for triplicate measurements.

FIG. 11 is a product (L-6-chlorotryptophan) formation over time plot for each substrate (L-tryptophan) concentration as indicated. Error bars indicate one standard deviation for triplicate measurements.

FIGS. 12A and 12B are the Michaelis-Menten curve (FIG. 12A) and Lineweaver-Burk plot (FIG. 12B) to determine kinetic parameters for Tar14. Error bars indicate one standard deviation for triplicate measurements.

FIG. 13 is a schematic illustration of in vitro CRISPR/Cas9-mediated in-frame deletion of the tar14 gene in the taromycin BGC captured in pCAP01-tarM1 cosmid. Agarose gels to confirm size of DNA fragments are labelled, Thermo Scientific GeneRuler 1 kb Plus DNA ladder was run alongside with the samples.

FIG. 14 shows LC-HRMS analysis of the metabolite profile of S. coelicolor M1146-pCAP01-tarM1Δtar14 mutant showing production of a non-halogenated taromycin analogue (m/z [M+2H]²⁺ 786.8, RT˜6.3 min) and its comparison to the wild type (WT) S. coelicolor M1146-pCAP01-tarM1 which produces only dihalogenated taromycin (m z [M+2H]²⁺ 820.8). EIC—extracted ion chromatogram, TIC—total ion chromatogram, RT—retention time.

FIG. 15 is LC-HRMS chromatograms of Tar14-catalyzed bromination of L-tryptophan and commercial standards of brominated tryptophan at C4, C5, C6, and C7 of the indole ring. L-5-Bromotryptophan (minor product) and L-6-bromotryptophan were formed by Tar14. Molecular ions of the reaction products showed characteristic isotope distribution pattern for monobrominated molecules. BPC—base peak chromatogram.

FIGS. 16A-16C show analysis of the purified recombinant proteins Tar13, 15, 16, and PtdH. FIG. 16A are images of SDS-PAGE gels; gels are labelled and expected protein sizes are indicated. EZ-Run™ Rec protein Ladder (Fisher Scientific) was run alongside with the samples. Fractions containing TarT3 protein had dark red/brown color corresponding to the holo- (heme-bound) form of the protein. FIG. 16B. is a gel-filtration chromatogram of the Tar13 protein. Tar13 exists in solution as a tetramer, consistent with other characterized family members.^{[22]} FIG. 16C. is a gel-filtration chromatogram of the Tar16 protein. Tar16 exists in solution as a monomer, consistent with other characterized family member.^{[23]}

FIG. 17 shows UV/Vis spectra of Tar13 color coded as per key. Purified Tar13 has a typical Soret band at 405 nm characteristic to a ferric (Fe³⁺) state of heme-containing protein.^{[24]} Presence of the substrate (L-6-chlorotryptophan) results in slight shift of the absorbance (410 nm).

FIGS. 18A and 18B shows FIG. 18A. Calibration curve showing dependence of the ratio of area of the L-6-chlorotryptophan peak to the area of the L-tryptophan peak from ratio of concentration of the L-6-chlorotryptophan peak to the concentration of L-tryptophan. Error bars (indicate one standard deviation for triplicate measurements) are covered by marker labels. FIG. 18B. Fitted Michaelis-Menten curve to determine kinetic parameters for Tar13. Error bars indicate one standard deviation for triplicate measurements. K_{M}=112.3±23.7 μM; k_{cat}=0.031±0.003 s⁻¹; Vₘₐₓ0.061±0.006 μM/s.

FIG. 19 is a graph showing relative Tar13 substrate consumption of L-6-Cl-Trp and L-Trp and their respective accumulation of products N-formyl-L-4-Cl-Kyn and N-formyl-L-4-Kyn over time. Error bars indicate one standard deviation for triplicate measurements.

FIG. 20 shows chemical structures of substrate analogues tested in in vitro assays with Tar14, Tar13, and Tar13/16. Structures 22, 23, 24 were not converted by any of the enzymes.

FIG. 21 shows LC-HRMS analysis of Tar14-catalyzed chlorination and bromination of L-tryptophan, D-tryptophan, L-kynurenine, and L-4-chlorokynurenine substrates as labelled. Peaks corresponding to the reaction product are marked with star. EIC—extracted ion chromatogram, TIC—total ion chromatogram. TICs of reactions are unmarked, EIC of brominated products—marked with triangles, EIC of chlorinated products—marked with circles.

FIG. 22 is LC-HRMS analysis of Tar14-catalyzed chlorination and bromination of L-4-bromotryptophan and D/L-5-bromotryptophan substrates as labelled. Peaks corresponding to the reaction product are marked with star. EIC—extracted ion chromatogram, TIC—total ion chromatogram. TICs of reactions are unmarked, EIC of brominated products—triangle marker, EIC of chlorinated products—circle marker.

FIG. 23 is LC-HRMS analysis of Tar14-catalyzed chlorination and bromination of L-6-bromotryptophan, D/L-5-chlorotryptophan, and L-6-chlorotryptophan substrates as labelled. Peaks corresponding to the reaction product are marked with star. EIC—extracted ion chromatogram, TIC—total ion chromatogram. TICs of reactions are unmarked, EIC of brominated products—triangle, EIC of chlorinated products—circle.

FIG. 24 is LC-HRMS analysis of Tar14-catalyzed chlorination and bromination of D/L-5-methoxytryptophan, D/L-5-hydroxytryptophan, D/L-7-bromotryptophan, and D/L-6-fluorotryptophan substrates as labelled. Peaks corresponding to the reaction product are marked with star. EIC—extracted ion chromatogram, TIC—total ion chromatogram. TICs of reactions are unmarked, EIC of brominated products—triangle marker, EIC of chlorinated products—circle marker.

FIG. 25 is LC-HRMS analysis of Tar14-catalyzed chlorination and bromination of D/L-4-fluorotryptophan, D/L-4-methyltryptophan, and D/L-5-methyltryptophan substrates as labelled. Peaks corresponding to the reaction product are marked with star. EIC—extracted ion chromatogram, TIC—total ion chromatogram. TICs of reactions are unmarked, EIC of brominated products—triangle marker, EIC of chlorinated products—circle marker.

FIG. 26 is LC-HRMS analysis of Tar13- and Tar13/Tar16-catalyzed convertion of L-tryptophan (left) and D-tryptophan (right). Peaks corresponding to the corresponding reaction product are marked with star, S indicates peak of the non-converted substrate. Additional peaks in EIC are isotope ions or ions with high-resolution mass not matching the mass of the reaction product. EIC—extracted ion chromatogram, TIC—total ion chromatogram. TICs of reactions are unmarked, EIC of TarT3 reaction product—circle marker, EIC of Tar13/Tar16 reaction product—triangle marker.

FIG. 27 shows LC-HRMS analysis of Tar13- and Tar13/Tar16-catalyzed convertion of D/L-5-bromotryptophan (left) and D/L-5-chlorotryptophan (right). Peaks corresponding to the corresponding reaction product are marked with a star, S indicates peak of the non-converted substrate. Additional peaks in EIC are isotope ions or ions with high-resolution mass not matching the mass of the reaction product. EIC—extracted ion chromatogram, TIC—total ion chromatogram. TICs of reactions are unmarked, EIC of Tar13 reaction product—circle, EIC of Tar13/Tar16 reaction product—triangle.

FIG. 28 shows LC-HRMS analysis of Tar13- and Tar13/Tar16-catalyzed convertion of D/L-5-methyltryptophan (left) and D/L-5-methoxytryptophan (right). Peaks corresponding to the corresponding reaction product are marked with a star, S indicates peak of the non-converted substrate. Additional peaks in EIC are isotope ions or ions with high-resolution mass not matching the mass of the reaction product. EIC—extracted ion chromatogram, TIC—total ion chromatogram. TICs of reactions are unmarked, EIC of Tar13 reaction product—circle, EIC of Tar13/Tar16 reaction product—triangle.

FIG. 29 shows LC-HRMS analysis of Tar13- and Tar13/Tar16-catalyzed convertion of L-6-bromotryptophan (left) and D/L-6-fluorotryptophan (right). Peaks corresponding to the corresponding reaction product are marked with a star, S indicates peak of the non-converted substrate. EIC—extracted ion chromatogram, TIC—total ion chromatogram. TICs of reactions are unmarked, EIC of Tar13 reaction product—circle, EIC of Tar13/Tar16 reaction product—triangle.

FIG. 30 is LC-HRMS analysis of the metabolite profile of S. coelicolor M1146-pCAP01-tarM1Δtar14 mutant supplemented with 4-bromotryptophan, 5-bromotryptophan, 6-bromotryptophan, and 7-bromotryptophan as labelled. EIC—extracted ion chromatogram, TIC—total ion chromatogram. TICs of extracts are unmarked, EIC of monoincorporated analogues (position of residue-1)—triangle, EIC of biincorporated analogues—circle. Peaks corresponding to new taromycin analogues, identity of which was confirmed by HRMS data, are highlighted in dashed boxes.

FIG. 31 LC-HRMS analysis of the metabolite profile of S. coelicolor M1146-pCAP01-tarM1Δtar14 mutant supplemented with 4-fluorotryptophan, 6-fluorotryptophan, 5-chlorotryptophan, and 6-chlorotryptophan as labelled. EIC—extracted ion chromatogram, TIC—total ion chromatogram. TICs of extracts are unmarked, EIC of monoincorporated analogues (position of residue-1)—triangle, EIC of biincorporated analogues—circle. Peaks corresponding to new taromycin analogues, identity of which was confirmed by HRMS data, are highlighted in dashed boxes.

FIG. 32 LC-HRMS analysis of the metabolite profile of S. coelicolor M1146-pCAP01-tarM1Δtar14 mutant supplemented with 4-methyltryptophan, 5-methyltryptophan, 6-methoxytryptophan, and 5-hydroxytryptophan as labelled. EIC—extracted ion chromatogram, TIC—total ion chromatogram. TICs of extracts are unmarked, EIC of monoincorporated analogues (position of residue-1)—triangle, EIC of biincorporated analogues—circle. Peaks corresponding to new taromycin analogues, identity of which was confirmed by HRMS data, are highlighted in dashed boxes. * peaks correspond to taromycin B series rather than biincorporated analogues of taromycin A series.

FIG. 33 LC-HRMS analysis of the metabolite profile of S. coelicolor M1146-pCAP01-tarM1Δtar14 mutant supplemented with 5-nitrotryptophan and wild type (WT) M1146-pCAP01-tarM1 as a control. EIC—extracted ion chromatogram, TIC—total ion chromatogram. TICs of extracts are unmarked, EIC of monoincorporated analogues (position of residue-1)—triangle, EIC of biincorporated analogues—circle. Peaks corresponding to new taromycin analogues, identity of which was confirmed by HRMS data, are highlighted in dashed boxes.

FIG. 34 is a neighbor-Joining tree of 58 sequences of putative tryptophan 2,3-dioxigenase (TDO) homologues. Support for each clade is indicated by bootstrap percentage values. The name (abbreviation) of each terminal branch corresponds to the name of the TDO is provided in Table 10. Proteins selected for analysis included the closest uncharacterized homologs of Tar13 found using protein BLAST analysis, biochemically characterized BGC-associated TDOs, primary metabolic TDOs from eukaryotic and prokaryotic sources. Key: black square—TDOs that are found within putative NRPS BGCs that also contain putative tryptophan halogenase and adenylation domain specific for kynurenine; black circle—putative TDOs that are found within putative BGCs, however, have not been characterized; white circle—biochemically characterized TDOs found within BGCs; triangle—characterized TDOs that have primary metabolic role in catabolism of tryptophan (eukaryotic and prokaryotic); white square—in vitro characterized TDO proteins from Streptomyces bacteria, not associated with BGCs and are involved in catabolizing tryptophan; unmarked—remaining uncharacterized homologues. MarE,^{[25]} non-canonical TDO that catalyzes mono-oxygenation of the substrate in maremycin biosynthesis, was used as outgroup (star). Protein sequences were aligned using Geneious 9.1.8 software, MUSCLE algorithm with default parameters. The phylogenetic tree was constructed using Geneious Tree Builder with Jukes-Cantor genetic distance model and Neighbor-Joining method with default parameters. Accession numbers, organisms of origin of the proteins, and additional comments are provided in Table 10.

FIG. 35 is a Neighbor-Joining tree of 25 sequences of putative kynurenine formamidase (KF) homologues. Support for each clade is indicated by bootstrap percentage values. The name (abbreviation) of each terminal branch corresponds to the name of the KF is provided in Table 10. Proteins selected for analysis included the closest uncharacterized homologs of Tar16 found using protein BLAST analysis, biochemically characterized BGC-associated KFs, primary metabolic KFs from eukaryotic and prokaryotic sources. Key: square—TDOs that are found within putative NRPS BGCs that also contain putative tryptophan halogenase and adenylation domain specific for kynurenine; triangle—characterized KFs that have primary metabolic role in catabolism of tryptophan (eukaryotic and prokaryotic); circle —in vitro characterized KF from Streptomyces bacteria, not associated with BGCs and are involved in catabolizing ryptophan; unmarked—remaining uncharacterized homologues. Protein sequences were aligned using Geneious 9.1.8 software, MUSCLE algorithm with default parameters. The phylogenetic tree was constructed using Geneious Tree Builder with Jukes-Cantor genetic distance model and Neighbor-Joining method with default parameters. Accession numbers, organisms of origin of the proteins, and additional comments are provided in Table 10.

FIGS. 36A-36C shows bioinformatic analysis of the draft genome of Saccharomonospora sp. CNQ-490. FIG. 36A Blast search result for homologs of tryptophan dioxygenase (TDO)-encoding genes. FIG. 36B Gene neighborhoods of the hit with gene ID: 2515970426. This gene encodes for Tar13 within the taromycin biosynthetic gene cluster. Tar13-encoding gene is labeled. FIG. 36C Gene neighborhoods of the hit with gene ID: 2515966685. This gene encodes for tryptophan dioxygenase that shares only 29% identity with Tar13 and is surrounded by primary metabolic genes. TDO-encoding gene is labeled. This analysis was performed using JGI/IMG online portal (https://img.jgi.doe.gov/).

FIG. 37A-37C shows bioinformatic analysis of the draft genome of Saccharomonospora sp. CNQ-490. FIG. 37A Blast search result for homologs of kynurenine formamidase (KF)-encoding genes. FIG. 37B Gene neighbouhoods of the hit with gene ID: 2515970423. This gene encodes for Tar16 protein within the taromycin biosynthetic gene cluster. Tar16-encoding gene is labeled. FIG. 37C Gene neighbouhoods of the hit with gene ID: 2515968274. This gene encodes for KF that shares only 30% identity with Tar16 and is surrounded by primary metabolic genes. KF-encoding gene is labeled. This analysis was performed using JGI/JMG online portal (https://img.jgi.doe.gov/).

FIG. 38 is an HPLC chromatogram (280 nm) of purification of compound 7. The target peak is indicated by a dot.

FIG. 39 is an HPLC chromatogram (310 nm) of purification of compound 10. The target peak is indicated by a dot.

FIG. 40 is an HPLC chromatogram (360 nm) of purification of compound 1. The target peak is indicated by a dot.

FIG. 41 shows ¹H NMR (600 MHz. CD₃OD) of compound 7. Signal δ˜ 8.5 corresponds to sodium formate from HPLC purification, while additional signals at δ˜3.2-3.7 correspond to the residual glycerol from enzymatic reaction.

FIG. 42 shows HSQC NMR (600 MHz, CD₃OD) of compound 7.

FIG. 43 shows COSY NMR (600 MHz, CD₃OD) of compound 7.

FIG. 44 shows HMBC NMR (600 MHz, CD₃OD) of compound 7.

FIG. 45 is 1H NMR (600 MHz, CD₃OD) of compound 10. Due to the deformylation of the molecule, sample is a mixture of N-formyl-L-4-chlorokynurenine and L-4-chlorokynurenine (1) (aromatic proton shifts highlighted in boxes and signal δ˜8.5 corresponds to sodium formate from HPLC purification). Additional signals at δ˜3.2-3.6 correspond to the residual glycerol from enzymatic reaction.

FIG. 46 is HSQC NMR (600 MHz, CD₃OD) of compound 10. Due to the deformylation of the molecule, sample is a mixture of N-formyl-L-4-chlorokynurenine and L-4-chlorokynurenine (1) (sodium formate and aromatic proton shifts of 1 are highlighted grey).

FIG. 47 is COSY NMR (600 MHz, CD₃OD) of compound 10. Due to the deformylation of the molecule, sample is a mixture of N-formyl-L-4-chlorokynurenine and L-4-chlorokynurenine (1) (sodium formate and aromatic proton shifts of 1 are highlighted grey).

FIG. 48 is HMBC NMR (600 MHz, CD₃OD) of compound 10. Due to the deformylation of the molecule, sample is a mixture of N-formyl-L-4-chlorokynurenine and L-4-chlorokynurenine (1) (sodium formate and aromatic proton shifts of 1 are highlighted grey).

FIG. 49 is ¹H NMR (600 MHz, CD₃OD) of compound 1. Signal δ˜8.5 corresponds to sodium formate from HPLC purification.

FIG. 50 is HSQC NMR (600 MHz, CD₃OD) of compound 1.

FIG. 51 is COSY NMR (600 MHz, CD₃OD) of compound 1.

FIG. 52 is HMBC NMR (600 MHz, CD₃OD) of compound 1.

### DETAILED DESCRIPTION

Described herein is the unprecedented conversion of 1-tryptophan into L-4-Cl-Kyn catalyzed by four enzymes in the taromycin biosynthetic pathway from the marine bacterium Saccharomonospora sp. CNQ-490. Applicants used genetic, biochemical, structural, and analytical techniques to establish l-4-Cl-Kyn biosynthesis, which is initiated by the flavin-dependent tryptophan chlorinase Tar14 and its Flavin reductase partner Tar15. This work revealed the first tryptophan 2,3-dioxygenase (Tar13) and kynurenine formamidase (Tar16) enzymes that are selective for chlorinated substrates. The substrate scope of Tar13, Tar14, and Tar16 was examined and revealed intriguing promiscuity, thereby opening doors for the targeted engineering of these enzymes as useful biocatalysts.

### I. Definitions

The abbreviations used herein have their conventional meaning within the chemical and biological arts. The chemical structures and formulae set forth herein are constructed according to the standard rules of chemical valency known in the chemical arts.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Where substituent groups are specified by their conventional chemical formulae, written from left to right, they equally encompass the chemically identical substituents that would result from writing the structure from right to left, e.g., —CH₂O—is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bondss. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., 0, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—, For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from an heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocycloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the [symbol] remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

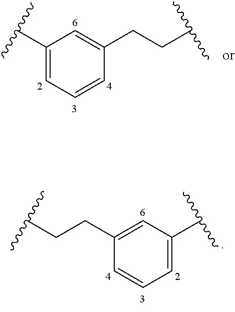 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —Cl₃, —CN, —CHO, —OH, —NH₂, —COOH, —CONH₂, —NO₂, —SH, —SO₂CH₃—SO₃H, —OSO₃H, —SO₂NH₂, □NHNH₂, □ONH₂, □NHC(O)NHNH₂, substituted or unsubstituted C₁-C₅ alkyl or substituted or unsubstituted 2 to 5 membered heteroalkyl). In embodiments, the alkylarylene is unsubstituted.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, halogen, —SiR′R″R″, —OC(O)R′, —C(O)R′. —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, □NR′NR″R′″, □ONR′R″, □NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, □NR′NR″R′″, □ONR′R″, □NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Where a moiety is substituted with an R substituent, the group may be referred to as “R-substituted.” Where a moiety is R-substituted, the moiety is substituted with at least one R substituent and each R substituent is optionally different. For example, where a moiety herein is R^{3A}-substituted or unsubstituted alkyl, a plurality of R^{3A} substituents may be attached to the alkyl moiety wherein each R^{3A} substituent is optionally different. Where an R-substituted moiety is substituted with a plurality of R substituents, each of the R-substituents may be differentiated herein using a prime symbol (′) such as R′, R″, etc. For example, where a moiety is R^{3A}-substituted or unsubstituted alkyl, and the moiety is substituted with a plurality of R^{3A} substituents, the plurality of R^{3A} substituents may be differentiated as R^{3A}′, R^{3A}″, R^{3A}′″, etc. In some embodiments, the plurality of R^{3A} substituents is 3. In some embodiments, the plurality of R^{3A} substituents is 2.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣB—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where variables s and are independently integers of from 0 to 3, and X is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), and silicon (Si).

The reactive functional groups can be chosen such that they do not participate in, or interfere with, the chemical stability of the antigen binding domain and the peptide compound described herein.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

Descriptions of compounds (e.g., peptide compounds, antibodies, antibody-peptide complexes, chemical compounds) of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid”” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acid as used herein also refers to nucleic acids that have the same basic chemical structure as a naturally occurring nucleic acid. Such analogues have modified sugars and/or modified ring substituents, but retain the same basic chemical structure as the naturally occurring nucleic acid. A nucleic acid mimetic refers to chemical compounds that have a structure that is different from the general chemical structure of a nucleic acid, but that functions in a manner similar to a naturally occurring nucleic acid. Examples of such analogues include, without limitation, phosphorothiolates, phosphoramidates, methyl phosphonates, chiral-methyl phosphonates, 2—O-methyl ribonucleotides, and peptide-nucleic acids (PNAs).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids sequences encode any given amino acid residue. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

“Codon optimization” refers to the substitution of certain codons with other codons to increase protein expression levels. Codon optimization may refer to the substitution of less frequent codons with more frequent codons according to genomic codon usage in an organism that serves as the host for protein expression. Because endogeous genes that have coding sequences that comprise frequent codons typically have high protein expression levels, recombinant protein expression may be improved by increasing the codon frequency. Codon optimization may refer to synonomous codon substitution that is predicted to destabilize mRNA secondary structures. The destabilization of mRNA secondary structures may improve recombinant protein expression by enhancing translational efficiency.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The terms “polypeptide.” “peptide,” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5-end). Due to deletions, insertions, truncations, fusions, and the like that may be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The term “Flavin-dependent tryptophan halogenase” or “Flavin-dependent tryptophan halogenase protein” as used herein refers to any of the recombinant or naturally-occurring forms of Flavin-dependent tryptophan halogenase protein (FDH), or variants or homologs thereof that maintain FDH activity (e.g., within at least 50%, 80%, 90%, 95%, 96%, 97%, 98%, 99% or 100% activity compared to FDH). In some aspects, the variants or homologs have at least 90%, 95%, 96%, 97%, 98%, 99%, or 100% amino acid sequence identity across the whole sequence or a portion of the sequence (e.g., a 50, 100, 150 or 200 continuous amino acid portion) compared to a naturally occurring FDH protein. In embodiments, the FDH protein is substantially identical to the protein having the sequence of SEQ ID NO:3, or a variant or homolog having substantial identity thereto. In embodiments, the FDH protein is substantially identical to the protein having the sequence of SEQ ID NO:4, or a variant or homolog having substantial identity thereto. In embodiments, the FDH protein is substantially identical to the protein having the sequence of SEQ ID NO:5, or a variant or homolog having substantial identity thereto. In embodiments, the FDH protein is substantially identical to the protein having the sequence of SEQ ID NO:34, or a variant or homolog having substantial identity thereto. In embodiments, the FDH protein is substantially identical to the protein having the sequence of SEQ ID NO:35, or a variant or homolog having substantial identity thereto. In embodiments, the FDH protein is substantially identical to the protein having the sequence of SEQ ID NO:37, or a variant or homolog having substantial identity thereto. In embodiments, the FDH protein is substantially identical to the protein having the sequence of SEQ ID NO:38, or a variant or homolog having substantial identity thereto. In embodiments, the FDH protein is substantially identical to the protein having the sequence of SEQ ID NO:39, or a variant or homolog having substantial identity thereto. In embodiments, the FDH protein is substantially identical to the protein having the sequence of SEQ ID NO:40, or a variant or homolog having substantial identity thereto. In embodiments, the FDH protein is substantially identical to the protein having the sequence of SEQ ID NO:41, or a variant or homolog having substantial identity thereto. In embodiments, the FDH protein is substantially identical to the protein having the sequence of SEQ ID NO:42, or a variant or homolog having substantial identity thereto. In embodiments, the FDH protein is substantially identical to the protein having the sequence of SEQ ID NO:43, or a variant or homolog having substantial identity thereto. In embodiments, the FDH protein is substantially identical to the protein having the sequence of SEQ ID NO:44, or a variant or homolog having substantial identity thereto.

In embodiments, the FDH protein is substantially identical to the protein identified by the NCBI reference number GI: 1465284460, or a variant or homolog having substantial identity thereto. In embodiments, the FDH protein is substantially identical to the protein identified by the NCBI reference number GI: 1465295985, or a variant or homolog having substantial identity thereto. In embodiments, the FDH protein is substantially identical to the protein identified by the NCBI reference number GI: 1333885101, or a variant or homolog having substantial identity thereto. In embodiments, the FDH protein is substantially identical to the protein identified by the NCBI reference number GI: 1141063773, or a variant or homolog having substantial identity thereto.

The term “Tryptophan 2,3-dioxygenase” or “Tryptophan 2,3-dioxygenase protein” as used herein refers to any of the recombinant or naturally-occurring forms of Tryptophan 2,3-dioxygenase (TDO), or variants or homologs thereof that maintain TDO activity (e.g., within at least 50%, 80%, 90%, 95%, 96%, 97%, 98%. 99%, or 100% activity compared to TDO). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring TDO protein. In embodiments, the TDO protein is substantially identical to the protein having the sequence of SEQ ID NO:1, or a variant or homolog having substantial identity thereto. In embodiments, the TDO protein is substantially identical to the protein having the sequence of SEQ ID NO:2, or a variant or homolog having substantial identity thereto.

In embodiments, the TDO protein is substantially identical to the protein identified by accession number WP_024877504.1, 2NOX, 2NW7, 4PW8, 4HKA, CAJ34362.1, AHF22860.1, NP_627840.1, WP_079163406.1, 2721033010, ADG27362.1, 2656756676, EFE76321.1, AAX31563.1, AET98915, BAE98160.1, BAH04172.1, WP_015786181.1, WP_091369532.1, 2653846435, WP_093406808.1, 2664226347, WP_093154509.1, SEG43548.1, WP_099845516.1, 2741237405, WP_051717236.1, 2768681930, WP_078940749.1, 2768627411, WP_086671565.1, WP_097230801.1, 2718366227, WP_097874054.1, WP_006122811.1, WP_078918332.1, GAX51800.1, WP_023562381.1, 2555809471, WP_027745021.1, 2516109309, WP_027756395.1, 2516102262, WP_035812565.1, WP 080047241.1, WP 020390285.1, WP_020390285.1, WP_027762312.1, WP_084962261.1, WP_091282833.1, SDH75058.1, WP_090933783.1, WP_051264531.1, 2524964422, WP_013017129.1, SCL19875.1, WP_091348456.1, WP_096059116.1, or a variant or homolog having substantial identity thereto.

In embodiments, the TDO protein is substantially identical to the protein identified by the NCBI reference number GI: 5032165, or a variant or homolog having substantial identity thereto. In embodiments, the TDO protein is substantially identical to the protein identified by the NCBI reference number GI: 1266923814, or a variant or homolog having substantial identity thereto. In embodiments, the TDO protein is substantially identical to the protein identified by the NCBI reference number GI: 1158820178, or a variant or homolog having substantial identity thereto.

The term “Kynurenine formamidase” or “Kynurenine formamidase protein” as used herein refers to any of the recombinant or naturally-occurring forms of Kynurenine formamidase (KF), or variants or homologs thereof that maintain KF activity (e.g., within at least 50%, 80%, 90%, 95%, 96%, 97%, 98%, 99%, or 100% activity compared to KF). In some aspects, the variants or homologs have at least 90%, 95%, 96%, 97%, 98%, 99%, or 100% amino acid sequence identity across the whole sequence or a portion of the sequence (e.g., a 50, 100, 150 or 200 continuous amino acid portion) compared to a naturally occurring KF protein. In embodiments, the KF protein is substantially identical to the protein having the sequence of SEQ ID NO:8, or a variant or homolog having substantial identity thereto. In embodiments, the KF protein is substantially identical to the protein having the sequence of SEQ ID NO:9, or a variant or homolog having substantial identity thereto.

In embodiments, the KF protein is substantially identical to the protein identified by accession number WP 037335967.1, WP_003114853.1, 4COG, WP_000858067.1, 4E11, Q63HM1, WP_099845518.1, SFD16583.1, WP_091369528.1, WP_037312927.1, WP_043220233.1, WP 049717738.1, WP_106962696.1, SBU91169.1, WP_033222207.1, WP_035864171.1, WP 099900824.1, WP 093160261.1, WP_024885731.1, WP_037640790.1, WP 104636119.1, WP 059301759.1, WP_087808139.1, WP_030932029.1, WP_003975294.1, or a variant or homolog having substantial identity thereto.

In embodiments, the KF protein is substantially identical to the protein identified by the UniProt reference number Q63HM1, or a variant or homolog having substantial identity thereto. In embodiments, the KF protein is substantially identical to the protein identified by the UniProt reference number K7EMI4, or a variant or homolog having substantial identity thereto.

The term “Flavin reductase” or “Flavin reductase protein” as used herein refers to any of the recombinant or naturally-occurring forms of Flavin reductase, or variants or homologs thereof that maintain Flavin reductase activity (e.g., within at least 50%, 80%, 90%, 95%, 96%, 97%, 98%, 99%, or 100% activity compared to Flavin reductase). In some aspects, the variants or homologs have at least 90%, 95%, 96%, 97%, 98%, 99%, or 100% amino acid sequence identity across the whole sequence or a portion of the sequence (e.g., a 50, 100, 150 or 200 continuous amino acid portion) compared to a naturally occurring Flavin reductase protein. In embodiments, the Flavin reductase protein is substantially identical to the protein having the sequence of SEQ ID NO:6, or a variant or homolog having substantial identity thereto. In embodiments, the Flavin reductase protein is substantially identical to the protein having the sequence of SEQ ID NO:7, or a variant or homolog having substantial identity thereto.

In embodiments, the Flavin reductase protein is substantially identical to the protein identified by the UniProt reference number P30043, or a variant or homolog having substantial identity thereto. In embodiments, the Flavin reductase protein is substantially identical to the protein identified by the UniProt reference number P94424, or a variant or homolog having substantial identity thereto

The term “microbe” is used in accordance with its well understood meaning in Biochemistry and refers generally to a microorganism. For example, a microbe may be a bacterium, fungus, archaea, protest, or virus. In embodiments, the microbe is a gram positive bacteria. In embodiments, the microbe is a gram negative bacteria. In embodiments, the microbe is Escherichia coli. In embodiments, the microbe is Pseudomonas putida. In an embodiment, the microbe is Streptomyces coelicolor M1146. In embodiments, the microbe is Saccharomonospora sp. CNQ-490. In embodiments, the microbe is Corynebacterium glutamicum.

The term “gram negative bacterium” is used in accordance with its well understood meaning in Biochemistry and refers generally to a prokaryotic microorganism (i.e. a bacterium) that includes a cell envelope. Gram negative bacteria include E. coli and P. putida.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 50%, 55%, 60%, 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using a sequence comparison algorithms or by manual alignment and visual inspection. Such sequences that are at least about 80% identical are said to be “substantially identical.” In some embodiments, two sequences are 100% identical. In certain embodiments, two sequences are 100% identical over the entire length of one of the sequences (e.g., the shorter of the two sequences where the sequences have different lengths). In various embodiments, identity may refer to the complement of a test sequence. In some embodiments, the identity exists over a region that is at least about 10 to about 100, about 20 to about 75, about 30 to about 50 amino acids or nucleotides in length. In certain embodiments, the identity exists over a region that is at least about 50 amino acids in length, or more preferably over a region that is 100 to 500, 100 to 200, 150 to 200, 175 to 200, 175 to 225, 175 to 250, 200 to 225, 200 to 250 or more amino acids in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window” refers to a segment of any one of the number of contiguous positions (e.g., at least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In various embodiments, a comparison window is the entire length of one or both of two aligned sequences. In some embodiments, two sequences being compared comprise different lengths, and the comparison window is the entire length of the longer or the shorter of the two sequences. In certain embodiments relating to two sequences of different lengths, the comparison window includes the entire length of the shorter of the two sequences. In some embodiments relating to two sequences of different lengths, the comparison window includes the entire length of the longer of the two sequences.

Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Preferred examples of algorithms that are suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (NCBI), as is known in the art. An exemplary BLAST algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. In certain embodiments, the NCBI BLASTN or BLASTP program is used to align sequences. In certain embodiments, the BLASTN or BLASTP program uses the defaults used by NCBI. In certain embodiments, the BLASTN program (for nucleotide sequences) uses as defaults: a word size (W) of 28; an expectation threshold (E) or 10; max matches in a query range set to 0; match/mismatch scores of 1-2; linear gap costs; the filter for low complexity regions used; and mask for lookup table only used. In certain embodiments, the BLASTP program (for amino acid sequences) uses as defaults a word size (W) of 3; an expectation threshold (E) of 10; max matches in a query range set to 0; the BLOSUM62 matrix (see Henikoff and Henikoff 1992) Proc. Natl. Acad. Sci. USA 89:10915); gap costs of existence: 11 and extension: 1; and conditional compositional score matrix adjustment.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross-reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

A “labeled protein or polypeptide” is one that is bound, either covalently, through a linker or a chemical bond, or noncovalently, through ionic, van der Waals, electrostatic, or hydrogen bonds to a label such that the presence of the labeled protein or polypeptide may be detected by detecting the presence of the label bound to the labeled protein or polypeptide. Alternatively, methods using high affinity interactions may achieve the same results where one of a pair of binding partners binds to the other, e.g., biotin, streptavidin.

The term “fragment,” as used herein, means a portion of a polypeptide or polynucleotide that is less than the entire polypeptide or polynucleotide. As used herein, a “functional fragment” of a protein, e.g., Tar13, Tar14, Tar16, Tar16, is a fragment of the polypeptide that is shorter than the full-length, immature, or mature polypeptide and has at least 25% (e.g., at least 30%, 40%, 50%, 60%, 70%, 80%, 90%, 95%, 98%, 99%, or even 100% or more) of the activity of full-length mature reference protein. Fragments of interest can be made by recombinant, synthetic, or proteolytic digestive methods.

A “ligand” refers to an agent, e.g., a polypeptide or other molecule, capable of binding to a receptor.

The term “recombinant” when used with reference, for example, to a cell, a nucleic acid, a protein, or a vector, indicates that the cell, nucleic acid, protein or vector has been modified by or is the result of laboratory methods. Thus, for example, recombinant proteins include proteins produced by laboratory methods. Recombinant proteins can include amino acid residues not found within the native (non-recombinant) form of the protein or can be include amino acid residues that have been modified, e.g., labeled.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the organism it is expressed by. Conversely, the term “endogenous” or “endogenous promoter” refers to a molecule or substance that is native to, or originates within, a given cell or organism.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell. Expression of a transfected gene can further be accomplished by transposon-mediated insertion into to the host genome. During transposon-mediated insertion, the gene is positioned in a predictable manner between two transposon linker sequences that allow insertion into the host genome as well as subsequent excision. Stable expression of a transfected gene can further be accomplished by infecting a cell with a lentiviral vector, which after infection forms part of (integrates into) the cellular genome thereby resulting in stable expression of the gene.

The terms “plasmid.” “vector,” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The terms “transfection,” “transduction,” “transfecting,” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

“Contacting” is used in accordance with its plain ordinary meaning and refers to the process of allowing at least two distinct species (e.g., chemical compounds including biomolecules or cells) to become sufficiently proximal to react, interact or physically touch. It should be appreciated, that the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a substrate and an enzyme or a ligand and a receptor. In embodiments, contacting includes, for example, allowing an enzyme (e.g., Tar14) to bind a substrate (e.g., L-Trp). In embodiments, contacting includes allowing a substrate (e.g., L-Trp) to contact or enter a cell (e.g., E. coli).

The term “modulation,” “modulate,” or “modulator” are used in accordance with their plain ordinary meaning and refer to the act of changing or varying one or more properties. “Modulator” refers to a composition that increases or decreases the level of a target molecule or the function of a target molecule or the physical state of the target of the molecule. “Modulation” refers to the process of changing or varying one or more properties. For example, as applied to the effects of a modulator on a biological target, to modulate means to change by increasing or decreasing a property or function of the biological target or the amount of the biological target.

As defined herein, the term “inhibition,” “inhibit,” “inhibiting” and the like in reference to a protein-inhibitor (e.g., antagonist) interaction means negatively affecting (e.g., decreasing) the activity or function of the protein relative to the activity or function of the protein in the absence of the inhibitor. In embodiments inhibition refers to reduction of a disease or symptoms of disease. Thus, in embodiments, inhibition includes, at least in part, partially or totally blocking stimulation, decreasing, preventing, or delaying activation, or inactivating, desensitizing, or down-regulating signal transduction or enzymatic activity or the amount of a protein. The amount of inhibition may be 10%, 20%, 30%, 40%, 50%, 60%, 70%, 80%, 90%, 100% or less in comparison to a control in the absence of the antagonist. In embodiments, the inhibition is 1.5-fold, 2-fold, 3-fold, 4-fold, 5-fold, 10-fold, or more than the expression or activity in the absence of the antagonist.

As defined herein, the term “activation,” “activate,” “activating” and the like in reference to a protein-activator (e.g., agonist) interaction means positively affecting (e.g., increasing) the activity or function of the relative to the activity or function of the protein in the absence of the activator. Thus, in embodiments, activation may include, at least in part, partially or totally increasing stimulation, increasing or enabling activation, or activating, sensitizing, or up-regulating signal transduction or enzymatic activity or the amount of a protein decreased in a disease. The amount of activation may be 10%, 20%, 30%, 40%, 50%, 60%, 70%, 80%, 90%, 100% or more in comparison to a control in the absence of the agonist. In embodiments, the activation is 1.5-fold, 2-fold, 3-fold, 4-fold, 5-fold, 10-fold, or more than the expression or activity in the absence of the agonist.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated activity (e.g., by using a method as described herein), results in reduction of the disease or one or more disease symptoms.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The term “genetically engineered cell” as used herein refers to altering the DNA in a cell. For example, a transfer of genes into the cell is used to produce a genetically engineered cell. For example, one base pair modification is used to produce a genetically engineered cell. For example, extracting DNA from another organism's genome and combining it with DNA of that cell is used to produce a genetically engineered cell. The DNA may be either isolated and copied or artificially synthesized. A construct is usually created and used to insert this DNA into the cell. The construct may include a promoter and terminator region, which initiate and end transcription. The gene may also be modified for better expression or effectiveness. Modifications of the gene may be carried out using recombinant DNA techniques, such as restriction digests, ligations and molecular cloning. A number of techniques known in the art may be used to insert genetic material into the host genome of the immune cell.

A “therapeutic agent” or “therapeutic moiety” as referred to herein, is a composition (e.g., small molecule, peptide, nucleic acid, protein, fragment) useful in treating or preventing a disease.

“Patient” or “subject in need thereof” refers to a living member of the animal kingdom suffering from or that may suffer from the indicated disorder. In embodiments, the subject is a member of a species that includes individuals who naturally suffer from the disease. In embodiments, a subject is a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with a compound, pharmaceutical composition, or method provided herein. In embodiments, the disease is an autoimmune disease (e.g., Type I Diabetes).

As used herein, the term “neurodegenerative disorder” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, chronic fatigue syndrome, Cockayne syndrome, Corticobasal degeneration, Creutzfeldt-Jakob disease, frontotemporal dementia, Depression, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Major Depressive Disorder, Multiple sclerosis, Multiple System Atrophy, myalgic encephalomyelitis, Narcolepsy, Neuroborreliosis, Parkinson's disease, Pelizaeus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, progressive supranuclear palsy, or Tabes dorsalis.

The term “associated” or “associated with” in the context of a substance or substance activity or function associated with a disease (e.g., Type I Diabetes) means that the disease (e.g., Type I Diabetes) is caused by (in whole or in part), or a symptom of the disease is caused by (in whole or in part) the substance or substance activity or function.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, include prevention of an injury, pathology, condition, or disease. In embodiments, “treating” refers to treatment of an autoimmune disease.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment

A “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

Formulations suitable for oral administration can consist of (a) liquid solutions, such as an effective amount of the antibodies provided herein suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

“Co-administer” is meant that a composition described herein is administered at the same time, just prior to, or just after the administration of one or more additional therapies. The compounds provided herein can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

Dosages may be varied depending upon the requirements of the patient and the compound being employed. The dose administered to a patient, in the context of the present disclosure, should be sufficient to affect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

Pharmaceutical compositions may include compositions wherein the active ingredient (e.g., compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule, and/or reducing, eliminating, or slowing the progression of disease symptoms.

### II. Methods

L-4-Chlorokynurenine (L-4-Cl-Kyn) is a neuropharmaceutical drug candidate in development for the treatment of major depressive disorder and levodopa-induced dyskinesia in patients with Parkinson's disease. Recently, this amino acid prodrug was naturally found as a residue in the lipopeptide antibiotic taromycin. Provided herein are methods for the conversion of L-tryptophan (L-Trp) to L-4-Cl-Kyn catalyzed by one or more enzymes in the taromycin biosynthetic pathway from the marine bacterium Saccharomonospora sp. CNQ-490. Provided herein are genetic, biochemical, structural, and analytical techniques to establish L-4-Cl-Kyn biosynthesis, which may be initiated by the Tar14 flavin-dependent tryptophan chlorinase and its flavin reductase partner Tar15.

The methods may utilize the first tryptophan 2,3-dioxygenase (Tar13) and kynurenine formamidase (Tar16) enzymes that are selective for chlorinated substrates. The substrate scope of Tar13, Tar14, and Tar16 was examined revealing intriguing promiscuity, thereby opening doors for the targeted engineering of these enzymes as useful biocatalysts.

Thus, in an aspect is provided a method of synthesizing L-4-Cl-Kyn, the method including contacting L-Trp with a Tar14 enzyme, a Tar13 enzyme, and a Tar16 enzyme. In embodiments, the method further includes a Flavin reductase. In embodiments, the Flavin reductase is a Tar15 enzyme. In embodiments, the method occurs within a microbe (e.g. E. coli or Corynebacterium glutamicum).

In embodiments, the microbe overproduces L-Tryptophan (L-Trp). In embodiments, overproduction of L-Trp is at least about 0.25 gram L-Trp per 1 liter (g/L) of microbe culture. In embodiments, overproduction of L-Trp is at least about 0.5 gram L-Trp per 1 liter (g/L) of microbe culture. In embodiments, overproduction of L-Trp is at least about 1 gram L-Trp per 1 liter (g/L) of microbe culture. In embodiments, overproduction of L-Trp is at least about 3 g L-Trp per L of microbe culture. In embodiments, overproduction of L-Trp is at least about 5 g L-Trp per L of microbe culture. In embodiments, overproduction of L-Trp is at least about 7 g L-Trp per L of microbe culture. In embodiments, overproduction of L-Trp is at least about 9 g L-Trp per L of microbe culture. In embodiments, overproduction of L-Trp is at least about 11 g L-Trp per L of microbe culture. In embodiments, overproduction of L-Trp is at least about 13 g L-Trp per L of microbe culture. In embodiments, overproduction of L-Trp is at least about 15 g L-Trp per L of microbe culture. In embodiments, overproduction of L-Trp is at least about 17 g L-Trp per L of microbe culture. In embodiments, overproduction of L-Trp is at least about 19 g L-Trp per L of microbe culture. In embodiments, overproduction of L-Trp is at least about 21 g L-Trp per L of microbe culture. In embodiments, overproduction of L-Trp is at least about 23 g L-Trp per L of microbe culture. In embodiments, overproduction of L-Trp is at least about 25 g L-Trp per L of microbe culture. In embodiments, overproduction of L-Trp is at least about 27 g L-Trp per L of microbe culture. In embodiments, overproduction of L-Trp is at least about 29 g L-Trp per L of microbe culture. In embodiments, overproduction of L-Trp is at least about 31 g L-Trp per L of microbe culture. In embodiments, overproduction of L-Trp is at least about 33 g L-Trp per L of microbe culture. In embodiments, overproduction of L-Trp is at least about 35 g L-Trp per L of microbe culture. In embodiments, overproduction of L-Trp is at least about 37 g L-Trp per L of microbe culture. In embodiments, overproduction of L-Trp is at least about 39 g L-Trp per L of microbe culture. In embodiments, overproduction of L-Trp is at least about 41 g L-Trp per L of microbe culture. In embodiments, overproduction of L-Trp is at least about 43 g L-Trp per L of microbe culture.

In embodiments, overproduction of L-Trp is about 0.25 gram L-Trp per 1 liter (g/L) of microbe culture. In embodiments, overproduction of L-Trp is about 0.5 g L-Trp per 1 L (g/L) of microbe culture. In embodiments, overproduction of L-Trp is about 1 gram L-Trp per 1 liter (g/L) of microbe culture. In embodiments, overproduction of L-Trp is about 3 g L-Trp per L of microbe culture. In embodiments, overproduction of L-Trp is about 5 g L-Trp per L of microbe culture. In embodiments, overproduction of L-Trp is about 7 g L-Trp per L of microbe culture. In embodiments, overproduction of L-Trp is about 9 g L-Trp per L of microbe culture. In embodiments, overproduction of L-Trp is about 11 g L-Trp per L of microbe culture. In embodiments, overproduction of L-Trp is about 13 g L-Trp per L of microbe culture. In embodiments, overproduction of L-Trp is about 15 g L-Trp per L of microbe culture. In embodiments, overproduction of L-Trp is about 17 g L-Trp per L of microbe culture. In embodiments, overproduction of L-Trp is about 19 g L-Trp per L of microbe culture. In embodiments, overproduction of L-Trp is about 21 g L-Trp per L of microbe culture. In embodiments, overproduction of L-Trp is about 23 g L-Trp per L of microbe culture. In embodiments, overproduction of L-Trp is about 25 g L-Trp per L of microbe culture. In embodiments, overproduction of L-Trp is about 27 g L-Trp per L of microbe culture. In embodiments, overproduction of L-Trp is about 29 g L-Trp per L of microbe culture. In embodiments, overproduction of L-Trp is about 31 g L-Trp per L of microbe culture. In embodiments, overproduction of L-Trp is about 33 g L-Trp per L of microbe culture. In embodiments, overproduction of L-Trp is about 35 g L-Trp per L of microbe culture. In embodiments, overproduction of L-Trp is about 37 g L-Trp per L of microbe culture. In embodiments, overproduction of L-Trp is about 39 g L-Trp per L of microbe culture. In embodiments, overproduction of L-Trp is about 41 g L-Trp per L of microbe culture. In embodiments, overproduction of L-Trp is about 43 g L-Trp per L of microbe culture.

In an aspect is provided a method of making L-4-chlorokynurenine (L-4-Cl-Kyn). The method includes converting L-tryptophan (L-Trp) to L-4-Cl-Kyn using one or more of Tar14, Tar13, or Tar16. In embodiment, L-Trp is converted to L-4-Cl-Kyn using Tar14. In embodiments, L-Trp is converted to L-4-Cl-Kyn using Tar13. In embodiments, L-Trp is converted to L-4-Cl-Kyn using Tar16. In embodiments, L-Trp is converted to L-4-Cl-Kyn using Tar14 and Tar 13. In embodiments, L-Trp is converted to L-4-Cl-Kyn using Tar14 and Tar 16. In embodiments, L-Trp is converted to L-4-Cl-Kyn using Tar13 and Tar 16. In embodiments, L-Trp is converted to L-4-Cl-Kyn using Tar13, Tar14 and Tar 16.

In another aspect is provided a method of producing L-4-Cl-Kyn. The method includes contacting a genetically engineered microbe provided herein, including embodiments thereof with L-Trp.

In embodiments, the method includes recombinantly expressing one or more of Tar14, Tar13, Tar15 or Tar16 in a microbe. In embodiments, the microbe is E. coli. In embodiments, the microbe is Corynebacterium glutamicum. In embodiments, the microbe is any microbe known to overproduce L-Trp.

For the methods provided herein, in embodiments, L-4-Cl-Kyn is produced in vivo. In embodiments, L-4-Cl-Kyn is produced in a genetically engineered microbe that overproduces L-Tryptophan. In embodiments, the method includes microbial fermentation. In embodiments, the method includes microbial fermentation of a genetically engineered microbe provided herein.

For the methods, provided here, in embodiments, L-4-Cl-Kyn is produced in vitro. In embodiments, the method includes enzymatic synthesis of of L-4-Cl-Kyn from L-Trp using one or more of recombinantly expressed Tar13, Tar14, Tar15 or Tar16. In embodiments, the method includes enzymatic synthesis of of L-4-Cl-Kyn from L-Trp using recombinantly expressed Tar13, Tar14. TarT5 and Tar16.

For the methods provided herein, in embodiments, the genetically engineered microbe is a human gastrointestinal microbe.

In embodiments, L-4-Cl-Kyn is secreted from the microbe. In embodiments, L-4-Cl-Kyn is purified by separating said L-4-Cl-Kyn from the microbe. In embodiments, separating L-4-Cl-Kyn from the microbe includes a centrifugation step. In embodiments, separating L-4-Cl-Kyn from the microbe includes a filtration step. In embodiments, separating L-4-Cl-Kyn from the microbe further includes activated carbon absorption and chromatographic purification of L-4-Cl-Kyn.

In embodiments, L-4-Cl-Kyn remains within the microbe. In embodiments, L-4-Cl-Kyn is purified by lysing the microbe. In embodiments, L-4-Cl-Kyn is further purified by chromatographic methods known in the art.

In embodiments, the microbe expresses one or more of Tar14, Tar13, or Tar16, and produces L-4-chlorokynurenine from L-Trp. In embodiments, the microbe expresses Tar14. In an embodiment, the microbe expresses Tar13. In embodiments, the microbe expresses Tar16. In embodiments, the microbe expresses Tar14 and Tar13. In embodiments, the microbe expresses Tar14 and Tar16. In embodiments, the microbe expresses Tar13 and Tar16. In embodiments, the microbe expresses Tar14, Tar13 and Tar16. In embodiments, the microbe further expresses Tar15. In embodiments, the microbe is the microbe is a human gastrointestinal microbe.

In an aspect, of method of treating a subject having a neurological disorder is provided. The method includes administering an effective amount of L-4-Cl-Kyn to the subject, thereby treating the neurological disorder. In embodiments, the neurological disorder is major depressive disorder.

### III. Genetically Engineered Microbes

In an aspect is provided a genetically engineered microbe, wherein the genetically engineered microbe includes an exogenous Tar14 encoding nucleic acid, an exogenous Tar13 encoding nucleic acid, or an exogenous Tar16 encoding nucleic acid. In embodiments, the genetically engineered microbe includes an exogenous Tar14 encoding nucleic acid. In embodiments, the genetically engineered microbe includes an exogenous Tar13 encoding nucleic acid. In embodiments, the genetically engineered microbe includes an exogenous Tar16 encoding nucleic acid.

In embodiments, the genetically engineered microbe includes an exogenous Tar14 enzyme, an exogenous Tar13 enzyme, or an exogenous Tar16 enzyme. In embodiments, the genetically engineered microbe includes an exogenous Tar14 enzyme. In embodiments, the genetically engineered microbe includes an exogenous Tar13 enzyme. In embodiments, the genetically engineered microbe includes an exogenous Tar16 enzyme.

In an aspect is provided a genetically engineered microbe, wherein the genetically engineered microbe includes one or more of an exogenous Tar14 encoding nucleic acid, an exogenous Tar13 encoding nucleic acid, or an exogenous Tar16 encoding nucleic acid. In embodiments, the genetically engineered microbe includes an exogenous Tar14 encoding nucleic acid and an exogenous Tar13 encoding nucleic acid. In embodiments, the genetically engineered microbe includes an exogenous Tar14 encoding nucleic acid and an exogenous Tar16 encoding nucleic acid. In embodiments, the genetically engineered microbe includes an exogenous Tar13 encoding nucleic acid and an exogenous Tar16 encoding nucleic acid. In embodiments, the genetically engineered microbe includes an exogenous Tar13 encoding nucleic acid, an exogenous Tar14 encoding nucleic acid, and an exogenous Tar16 encoding nucleic acid.

In embodiments, the genetically engineered microbe includes one or more of an exogenous Tar 14 enzyme, an exogenous Tar13 enzyme, or an exogenous Tar16 enzyme. In embodiments, the genetically engineered microbe includes an exogenous Tar13 enzyme and an exogenous Tar6 enzyme. In embodiments, the genetically engineered microbe includes an exogenous Tar13 enzyme and an exogenous Tar14 enzyme. In embodiments, the genetically engineered microbe includes an exogenous Tar14 enzyme and an exogenous Tar16 enzyme. In embodiments, the genetically engineered microbe includes an exogenous Tar14 enzyme, an exogenous Tar15 enzyme, and an exogenous Tar16 enzyme.

In embodiments, the genetically engineered microbe provided herein does not include an endogenous Tar14 encoding nucleic acid, an endogenous Tar13 encoding nucleic acid, or an endogenous Tar16 encoding nucleic acid. In embodiments, the genetically engineered microbe provided herein does not include one or more of an endogenous Tar14 encoding nucleic acid, an endogenous Tar13 encoding nucleic acid, or an endogenous Tar16 encoding nucleic acid. In embodiments, the genetically engineered microbe does not include an endogenous Tar14 encoding nucleic acid. In embodiments, the genetically engineered microbe does not include an endogenous Tar13 encoding nucleic acid. In embodiments, the genetically engineered microbe does not include an endogenous Tar16 encoding nucleic acid. In embodiments, the genetically engineered microbe does not include an endogenous Tar13 encoding nucleic acid or an endogenous Tar14 encoding nucleic acid. In embodiments, the genetically engineered microbe does not include an endogenous Tar13 encoding nucleic acid or an endogenous TarT6 encoding nucleic acid. In embodiments, the genetically engineered microbe does not include an endogenous Tar14 encoding nucleic acid or an endogenous Tar16 encoding nucleic acid.

In embodiments, the genetically engineered microbe includes an exogenous nucleic acid that has at least 80% nucleotide identity to SEQ ID NO:11, SEQ ID NO:13, or SEQ ID NO:17.

In embodiments, the genetically engineered microbe includes an exogenous nucleic acid that has at least 80% nucleotide identity to SEQ ID NO:11. In embodiments, the genetically engineered microbe includes an exogenous nucleic acid that has at least 81% nucleotide identity to SEQ ID NO:11. In embodiments, the genetically engineered microbe includes an exogenous nucleic acid that has at least 82% nucleotide identity to SEQ ID NO:11. In embodiments, the genetically engineered microbe includes an exogenous nucleic acid that has at least 83% nucleotide identity to SEQ ID NO:11. In embodiments, the genetically engineered microbe includes an exogenous nucleic acid that has at least 84% nucleotide identity to SEQ ID NO:11. In embodiments, the genetically engineered microbe includes an exogenous nucleic acid that has at least 85% nucleotide identity to SEQ ID NO:11. In embodiments, the genetically engineered microbe includes an exogenous nucleic acid that has at least 86% nucleotide identity to SEQ ID NO:11. In embodiments, the genetically engineered microbe includes an exogenous nucleic acid that has at least 87% nucleotide identity to SEQ ID NO:11. In embodiments, the genetically engineered microbe includes an exogenous nucleic acid that has at least 88% nucleotide identity to SEQ ID NO:11. In embodiments, the genetically engineered microbe includes an exogenous nucleic acid that has at least 89% nucleotide identity to SEQ ID NO:11. In embodiments, the genetically engineered microbe includes an exogenous nucleic acid that has at least 90% nucleotide identity to SEQ ID NO:11. In embodiments, the genetically engineered microbe includes an exogenous nucleic acid that has at least 91% nucleotide identity to SEQ ID NO:11. In embodiments, the genetically engineered microbe includes an exogenous nucleic acid that has at least 92% nucleotide identity to SEQ ID NO:11. In embodiments, the genetically engineered microbe includes an exogenous nucleic acid that has at least 93% nucleotide identity to SEQ ID NO:11. In embodiments, the genetically engineered microbe includes an exogenous nucleic acid that has at least 94% nucleotide identity to SEQ ID NO:11. In embodiments, the genetically engineered microbe includes an exogenous nucleic acid that has at least 95% nucleotide identity to SEQ ID NO:11. In embodiments, the genetically engineered microbe includes an exogenous nucleic acid that has at least 96% nucleotide identity to SEQ ID NO:11. In embodiments, the genetically engineered microbe includes an exogenous nucleic acid that has at least 97% nucleotide identity to SEQ ID NO:11. In embodiments, the genetically engineered microbe includes an exogenous nucleic acid that has at least 98% nucleotide identity to SEQ ID NO:11. In embodiments, the genetically engineered microbe includes an exogenous nucleic acid that has at least 99% nucleotide identity to SEQ ID NO:11.

In embodiments, the exogenous nucleic acid has a sequence identity of 81% to SEQ ID NO:11. In embodiments, the exogenous nucleic acid has a sequence identity of 82% to SEQ ID NO:11. In embodiments, the exogenous nucleic acid has a sequence identity of 83% to SEQ ID NO: 11. In embodiments, the exogenous nucleic acid has a sequence identity of 84% to SEQ ID NO: 11. In embodiments, the exogenous nucleic acid has a sequence identity of 85% to SEQ ID NO: 11. In embodiments, the exogenous nucleic acid has a sequence identity of 86% to SEQ ID NO: 11. In embodiments, the exogenous nucleic acid has a sequence identity of 87% to SEQ ID NO: 11. In embodiments, the exogenous nucleic acid has a sequence identity of 88% to SEQ ID NO:11. In embodiments, the exogenous nucleic acid has a sequence identity of 89% to SEQ ID NO:11. In embodiments, the exogenous nucleic acid has a sequence identity of 90% to SEQ ID NO:11. In embodiments, the exogenous nucleic acid has a sequence identity of 91% to SEQ ID NO:11. In embodiments, the exogenous nucleic acid has a sequence identity of 92% to SEQ ID NO: 11. In embodiments, the exogenous nucleic acid has a sequence identity of 93% to SEQ ID NO:11. In embodiments, the exogenous nucleic acid has a sequence identity of 94% to SEQ ID NO:11. In embodiments, the exogenous nucleic acid has a sequence identity of 95% to SEQ ID NO: 11. In embodiments, the exogenous nucleic acid has a sequence identity of 96% to SEQ ID NO:11. In embodiments, the exogenous nucleic acid has a sequence identity of 97% to SEQ ID NO: 11. In embodiments, the exogenous nucleic acid has a sequence identity of 98% to SEQ ID NO:11. In embodiments, the exogenous nucleic acid has a sequence identity of 99% to SEQ ID NO: 11. In embodiments, the exogenous nucleic acid is the sequence of SEQ ID NO:11.

In embodiments, the genetically engineered microbe includes an exogenous nucleic acid that has at least 80% nucleotide identity to SEQ ID NO:13. In embodiments, the genetically engineered microbe includes an exogenous nucleic acid that has at least 810% nucleotide identity to SEQ ID NO:13. In embodiments, the genetically engineered microbe includes an exogenous nucleic acid that has at least 82% nucleotide identity to SEQ ID NO:13. In embodiments, the genetically engineered microbe includes an exogenous nucleic acid that has at least 83% nucleotide identity to SEQ ID NO:13. In embodiments, the genetically engineered microbe includes an exogenous nucleic acid that has at least 84% nucleotide identity to SEQ ID NO:13. In embodiments, the genetically engineered microbe includes an exogenous nucleic acid that has at least 85% nucleotide identity to SEQ ID NO:13. In embodiments, the genetically engineered microbe includes an exogenous nucleic acid that has at least 86% nucleotide identity to SEQ ID NO:13. In embodiments, the genetically engineered microbe includes an exogenous nucleic acid that has at least 87% nucleotide identity to SEQ ID NO:13. In embodiments, the genetically engineered microbe includes an exogenous nucleic acid that has at least 88% nucleotide identity to SEQ ID NO:13. In embodiments, the genetically engineered microbe includes an exogenous nucleic acid that has at least 89% nucleotide identity to SEQ ID NO:13. In embodiments, the genetically engineered microbe includes an exogenous nucleic acid that has at least 90% nucleotide identity to SEQ ID NO:13. In embodiments, the genetically engineered microbe includes an exogenous nucleic acid that has at least 91% nucleotide identity to SEQ ID NO:13. In embodiments, the genetically engineered microbe includes an exogenous nucleic acid that has at least 92% nucleotide identity to SEQ ID NO:13. In embodiments, the genetically engineered microbe includes an exogenous nucleic acid that has at least 93% nucleotide identity to SEQ ID NO:13. In embodiments, the genetically engineered microbe includes an exogenous nucleic acid that has at least 94% nucleotide identity to SEQ ID NO:13. In embodiments, the genetically engineered microbe includes an exogenous nucleic acid that has at least 95% nucleotide identity to SEQ ID NO:13. In embodiments, the genetically engineered microbe includes an exogenous nucleic acid that has at least 96% nucleotide identity to SEQ ID NO:13. In embodiments, the genetically engineered microbe includes an exogenous nucleic acid that has at least 97% nucleotide identity to SEQ ID NO:13. In embodiments, the genetically engineered microbe includes an exogenous nucleic acid that has at least 98% nucleotide identity to SEQ ID NO:13. In embodiments, the genetically engineered microbe includes an exogenous nucleic acid that has at least 99% nucleotide identity to SEQ ID NO:13.

In embodiments, the exogenous nucleic acid has a sequence identity of 81% to SEQ ID NO:13. In embodiments, the exogenous nucleic acid has a sequence identity of 82% to SEQ ID NO:13. In embodiments, the exogenous nucleic acid has a sequence identity of 83% to SEQ ID NO: 13. In embodiments, the exogenous nucleic acid has a sequence identity of 84% to SEQ ID NO:13. In embodiments, the exogenous nucleic acid has a sequence identity of 85% to SEQ ID NO:13. In embodiments, the exogenous nucleic acid has a sequence identity of 86% to SEQ ID NO:13. In embodiments, the exogenous nucleic acid has a sequence identity of 87% to SEQ ID NO:13. In embodiments, the exogenous nucleic acid has a sequence identity of 88% to SEQ ID NO:13. In embodiments, the exogenous nucleic acid has a sequence identity of 89% to SEQ ID NO:13. In embodiments, the exogenous nucleic acid has a sequence identity of 90% to SEQ ID NO:13. In embodiments, the exogenous nucleic acid has a sequence identity of 91% to SEQ ID NO:13. In embodiments, the exogenous nucleic acid has a sequence identity of 92% to SEQ ID NO: 13. In embodiments, the exogenous nucleic acid has a sequence identity of 93% to SEQ ID NO:13. In embodiments, the exogenous nucleic acid has a sequence identity of 94% to SEQ ID NO:13. In embodiments, the exogenous nucleic acid has a sequence identity of 95% to SEQ ID NO:13. In embodiments, the exogenous nucleic acid has a sequence identity of 96% to SEQ ID NO:13. In embodiments, the exogenous nucleic acid has a sequence identity of 97% to SEQ ID NO:13. In embodiments, the exogenous nucleic acid has a sequence identity of 98% to SEQ ID NO: 13. In embodiments, the exogenous nucleic acid has a sequence identity of 99% to SEQ ID NO: 13. In embodiments, the exogenous nucleic acid is the sequence of SEQ ID NO: 13.

In embodiments, the genetically engineered microbe includes an exogenous nucleic acid that has at least 80% nucleotide identity to SEQ ID NO:17. In embodiments, the genetically engineered microbe includes an exogenous nucleic acid that has at least 81% nucleotide identity to SEQ ID NO:17. In embodiments, the genetically engineered microbe includes an exogenous nucleic acid that has at least 82% nucleotide identity to SEQ ID NO:17. In embodiments, the genetically engineered microbe includes an exogenous nucleic acid that has at least 83% nucleotide identity to SEQ ID NO:17. In embodiments, the genetically engineered microbe includes an exogenous nucleic acid that has at least 84% nucleotide identity to SEQ ID NO:17. In embodiments, the genetically engineered microbe includes an exogenous nucleic acid that has at least 85% nucleotide identity to SEQ ID NO:17. In embodiments, the genetically engineered microbe includes an exogenous nucleic acid that has at least 86% nucleotide identity to SEQ ID NO:17. In embodiments, the genetically engineered microbe includes an exogenous nucleic acid that has at least 87% nucleotide identity to SEQ ID NO:17. In embodiments, the genetically engineered microbe includes an exogenous nucleic acid that has at least 88% nucleotide identity to SEQ ID NO:17. In embodiments, the genetically engineered microbe includes an exogenous nucleic acid that has at least 89% nucleotide identity to SEQ ID NO:17. In embodiments, the genetically engineered microbe includes an exogenous nucleic acid that has at least 90% nucleotide identity to SEQ ID NO:17. In embodiments, the genetically engineered microbe includes an exogenous nucleic acid that has at least 91% nucleotide identity to SEQ ID NO:17. In embodiments, the genetically engineered microbe includes an exogenous nucleic acid that has at least 92% nucleotide identity to SEQ ID NO:17. In embodiments, the genetically engineered microbe includes an exogenous nucleic acid that has at least 93% nucleotide identity to SEQ ID NO:17. In embodiments, the genetically engineered microbe includes an exogenous nucleic acid that has at least 94% nucleotide identity to SEQ ID NO:17. In embodiments, the genetically engineered microbe includes an exogenous nucleic acid that has at least 95% nucleotide identity to SEQ ID NO:17. In embodiments, the genetically engineered microbe includes an exogenous nucleic acid that has at least 96% nucleotide identity to SEQ ID NO:17. In embodiments, the genetically engineered microbe includes an exogenous nucleic acid that has at least 97% nucleotide identity to SEQ ID NO:17. In embodiments, the genetically engineered microbe includes an exogenous nucleic acid that has at least 98% nucleotide identity to SEQ ID NO:17. In embodiments, the genetically engineered microbe includes an exogenous nucleic acid that has at least 99% nucleotide identity to SEQ ID NO:17.

In embodiments, the exogenous nucleic acid has a sequence identity of 81% to SEQ ID NO:17. In embodiments, the exogenous nucleic acid has a sequence identity of 82% to SEQ ID NO:17. In embodiments, the exogenous nucleic acid has a sequence identity of 83% to SEQ ID NO: 17. In embodiments, the exogenous nucleic acid has a sequence identity of 84% to SEQ ID NO: 17. In embodiments, the exogenous nucleic acid has a sequence identity of 85% to SEQ ID NO:17. In embodiments, the exogenous nucleic acid has a sequence identity of 86% to SEQ ID NO:17. In embodiments, the exogenous nucleic acid has a sequence identity of 87% to SEQ ID NO:17. In embodiments, the exogenous nucleic acid has a sequence identity of 88% to SEQ ID NO: 17. In embodiments, the exogenous nucleic acid has a sequence identity of 89% to SEQ ID NO: 17. In embodiments, the exogenous nucleic acid has a sequence identity of 90% to SEQ ID NO:17. In embodiments, the exogenous nucleic acid has a sequence identity of 91% to SEQ ID NO:17. In embodiments, the exogenous nucleic acid has a sequence identity of 92% to SEQ ID NO: 17. In embodiments, the exogenous nucleic acid has a sequence identity of 93% to SEQ ID NO:17. In embodiments, the exogenous nucleic acid has a sequence identity of 94% to SEQ ID NO:17. In embodiments, the exogenous nucleic acid has a sequence identity of 95% to SEQ ID NO:17. In embodiments, the exogenous nucleic acid has a sequence identity of 96% to SEQ ID NO:17. In embodiments, the exogenous nucleic acid has a sequence identity of 97% to SEQ ID NO: 17. In embodiments, the exogenous nucleic acid has a sequence identity of 98% to SEQ ID NO: 17. In embodiments, the exogenous nucleic acid has a sequence identity of 99% to SEQ ID NO: 17. In embodiments, the exogenous nucleic acid is the sequence of SEQ ID NO:17.

For the genetically engineered microbe provided herein, in embodiments, the exogenous nucleic acid provided herein has at least 80% nucleotide identity to SEQ ID NO:10, SEQ ID NO:12, or SEQ ID NO:16.

In embodiments, the genetically engineered microbe includes an exogenous nucleic acid that has at least 80% nucleotide identity to SEQ ID NO:10. In embodiments, the genetically engineered microbe includes an exogenous nucleic acid that has at least 81% nucleotide identity to SEQ ID NO:10. In embodiments, the genetically engineered microbe includes an exogenous nucleic acid that has at least 82% nucleotide identity to SEQ ID NO:10. In embodiments, the genetically engineered microbe includes an exogenous nucleic acid that has at least 83% nucleotide identity to SEQ ID NO:10. In embodiments, the genetically engineered microbe includes an exogenous nucleic acid that has at least 84% nucleotide identity to SEQ ID NO:10. In embodiments, the genetically engineered microbe includes an exogenous nucleic acid that has at least 85% nucleotide identity to SEQ ID NO:10. In embodiments, the genetically engineered microbe includes an exogenous nucleic acid that has at least 86% nucleotide identity to SEQ ID NO:10. In embodiments, the genetically engineered microbe includes an exogenous nucleic acid that has at least 87% nucleotide identity to SEQ ID NO:10. In embodiments, the genetically engineered microbe includes an exogenous nucleic acid that has at least 88% nucleotide identity to SEQ ID NO:10. In embodiments, the genetically engineered microbe includes an exogenous nucleic acid that has at least 89% nucleotide identity to SEQ ID NO:10. In embodiments, the genetically engineered microbe includes an exogenous nucleic acid that has at least 90% nucleotide identity to SEQ ID NO:10. In embodiments, the genetically engineered microbe includes an exogenous nucleic acid that has at least 91% nucleotide identity to SEQ ID NO:10. In embodiments, the genetically engineered microbe includes an exogenous nucleic acid that has at least 92% nucleotide identity to SEQ ID NO:10. In embodiments, the genetically engineered microbe includes an exogenous nucleic acid that has at least 93% nucleotide identity to SEQ ID NO:10. In embodiments, the genetically engineered microbe includes an exogenous nucleic acid that has at least 94% nucleotide identity to SEQ ID NO:10. In embodiments, the genetically engineered microbe includes an exogenous nucleic acid that has at least 95% nucleotide identity to SEQ ID NO:10. In embodiments, the genetically engineered microbe includes an exogenous nucleic acid that has at least 96% nucleotide identity to SEQ ID NO:10. In embodiments, the genetically engineered microbe includes an exogenous nucleic acid that has at least 97% nucleotide identity to SEQ ID NO:10. In embodiments, the genetically engineered microbe includes an exogenous nucleic acid that has at least 98% nucleotide identity to SEQ ID NO:10. In embodiments, the genetically engineered microbe includes an exogenous nucleic acid that has at least 99% nucleotide identity to SEQ ID NO:10.

In embodiments, the exogenous nucleic acid has a sequence identity of 81% to SEQ ID NO:10. In embodiments, the exogenous nucleic acid has a sequence identity of 82% to SEQ ID NO:10. In embodiments, the exogenous nucleic acid has a sequence identity of 83% to SEQ ID NO: 10. In embodiments, the exogenous nucleic acid has a sequence identity of 84% to SEQ ID NO: 10. In embodiments, the exogenous nucleic acid has a sequence identity of 85% to SEQ ID NO:10. In embodiments, the exogenous nucleic acid has a sequence identity of 86% to SEQ ID NO: 10. In embodiments, the exogenous nucleic acid has a sequence identity of 87% to SEQ ID NO:10. In embodiments, the exogenous nucleic acid has a sequence identity of 88% to SEQ ID NO:10. In embodiments, the exogenous nucleic acid has a sequence identity of 89% to SEQ ID NO:10. In embodiments, the exogenous nucleic acid has a sequence identity of 90% to SEQ ID NO:10. In embodiments, the exogenous nucleic acid has a sequence identity of 91% to SEQ ID NO:10. In embodiments, the exogenous nucleic acid has a sequence identity of 92% to SEQ ID NO: 10. In embodiments, the exogenous nucleic acid has a sequence identity of 93% to SEQ ID NO:10. In embodiments, the exogenous nucleic acid has a sequence identity of 94% to SEQ ID NO: 10. In embodiments, the exogenous nucleic acid has a sequence identity of 95% to SEQ ID NO: 10. In embodiments, the exogenous nucleic acid has a sequence identity of 96% to SEQ ID NO:10. In embodiments, the exogenous nucleic acid has a sequence identity of 97% to SEQ ID NO: 10. In embodiments, the exogenous nucleic acid has a sequence identity of 98% to SEQ ID NO: 10. In embodiments, the exogenous nucleic acid has a sequence identity of 99% to SEQ ID NO:10. In embodiments, the exogenous nucleic acid is the sequence of SEQ ID NO:10.

In embodiments, the genetically engineered microbe includes an exogenous nucleic acid that has at least 80% nucleotide identity to SEQ ID NO:12. In embodiments, the genetically engineered microbe includes an exogenous nucleic acid that has at least 810% nucleotide identity to SEQ ID NO:12. In embodiments, the genetically engineered microbe includes an exogenous nucleic acid that has at least 82% nucleotide identity to SEQ ID NO:12. In embodiments, the genetically engineered microbe includes an exogenous nucleic acid that has at least 83% nucleotide identity to SEQ ID NO:12. In embodiments, the genetically engineered microbe includes an exogenous nucleic acid that has at least 84% nucleotide identity to SEQ ID NO:12. In embodiments, the genetically engineered microbe includes an exogenous nucleic acid that has at least 85% nucleotide identity to SEQ ID NO:12. In embodiments, the genetically engineered microbe includes an exogenous nucleic acid that has at least 86% nucleotide identity to SEQ ID NO:12. In embodiments, the genetically engineered microbe includes an exogenous nucleic acid that has at least 87% nucleotide identity to SEQ ID NO:12. In embodiments, the genetically engineered microbe includes an exogenous nucleic acid that has at least 88% nucleotide identity to SEQ ID NO:12. In embodiments, the genetically engineered microbe includes an exogenous nucleic acid that has at least 89% nucleotide identity to SEQ ID NO:12. In embodiments, the genetically engineered microbe includes an exogenous nucleic acid that has at least 90% nucleotide identity to SEQ ID NO:12. In embodiments, the genetically engineered microbe includes an exogenous nucleic acid that has at least 91% nucleotide identity to SEQ ID NO:12. In embodiments, the genetically engineered microbe includes an exogenous nucleic acid that has at least 92% nucleotide identity to SEQ ID NO:12. In embodiments, the genetically engineered microbe includes an exogenous nucleic acid that has at least 93% nucleotide identity to SEQ ID NO:12. In embodiments, the genetically engineered microbe includes an exogenous nucleic acid that has at least 94% nucleotide identity to SEQ ID NO:12. In embodiments, the genetically engineered microbe includes an exogenous nucleic acid that has at least 95% nucleotide identity to SEQ ID NO:10. In embodiments, the genetically engineered microbe includes an exogenous nucleic acid that has at least 96% nucleotide identity to SEQ ID NO:12. In embodiments, the genetically engineered microbe includes an exogenous nucleic acid that has at least 97% nucleotide identity to SEQ ID NO:12. In embodiments, the genetically engineered microbe includes an exogenous nucleic acid that has at least 98% nucleotide identity to SEQ ID NO:12. In embodiments, the genetically engineered microbe includes an exogenous nucleic acid that has at least 99% nucleotide identity to SEQ ID NO:12.

In embodiments, the exogenous nucleic acid has a sequence identity of 81% to SEQ ID NO:12. In embodiments, the exogenous nucleic acid has a sequence identity of 82% to SEQ ID NO:12. In embodiments, the exogenous nucleic acid has a sequence identity of 83% to SEQ ID NO: 12. In embodiments, the exogenous nucleic acid has a sequence identity of 84% to SEQ ID NO: 12. In embodiments, the exogenous nucleic acid has a sequence identity of 85% to SEQ ID NO:12. In embodiments, the exogenous nucleic acid has a sequence identity of 86% to SEQ ID NO:12. In embodiments, the exogenous nucleic acid has a sequence identity of 87% to SEQ ID NO: 12. In embodiments, the exogenous nucleic acid has a sequence identity of 88% to SEQ ID NO:12. In embodiments, the exogenous nucleic acid has a sequence identity of 89% to SEQ ID NO:12. In embodiments, the exogenous nucleic acid has a sequence identity of 90% to SEQ ID NO:12. In embodiments, the exogenous nucleic acid has a sequence identity of 91% to SEQ ID NO:12. In embodiments, the exogenous nucleic acid has a sequence identity of 92% to SEQ ID NO: 12. In embodiments, the exogenous nucleic acid has a sequence identity of 93% to SEQ ID NO:12. In embodiments, the exogenous nucleic acid has a sequence identity of 94% to SEQ ID NO: 12. In embodiments, the exogenous nucleic acid has a sequence identity of 95% to SEQ ID NO: 12. In embodiments, the exogenous nucleic acid has a sequence identity of 96% to SEQ ID NO:12. In embodiments, the exogenous nucleic acid has a sequence identity of 97% to SEQ ID NO:12. In embodiments, the exogenous nucleic acid has a sequence identity of 98% to SEQ ID NO: 12. In embodiments, the exogenous nucleic acid has a sequence identity of 99% to SEQ ID NO: 12. In embodiments, the exogenous nucleic acid is the sequence of SEQ ID NO:12.

In embodiments, the genetically engineered microbe includes an exogenous nucleic acid that has at least 80% nucleotide identity to SEQ ID NO:16. In embodiments, the genetically engineered microbe includes an exogenous nucleic acid that has at least 810% nucleotide identity to SEQ ID NO:16. In embodiments, the genetically engineered microbe includes an exogenous nucleic acid that has at least 82% nucleotide identity to SEQ ID NO:16. In embodiments, the genetically engineered microbe includes an exogenous nucleic acid that has at least 83% nucleotide identity to SEQ ID NO:16. In embodiments, the genetically engineered microbe includes an exogenous nucleic acid that has at least 84% nucleotide identity to SEQ ID NO:16. In embodiments, the genetically engineered microbe includes an exogenous nucleic acid that has at least 85% nucleotide identity to SEQ ID NO:16. In embodiments, the genetically engineered microbe includes an exogenous nucleic acid that has at least 86% nucleotide identity to SEQ ID NO:16. In embodiments, the genetically engineered microbe includes an exogenous nucleic acid that has at least 87% nucleotide identity to SEQ ID NO:16. In embodiments, the genetically engineered microbe includes an exogenous nucleic acid that has at least 88% nucleotide identity to SEQ ID NO:16. In embodiments, the genetically engineered microbe includes an exogenous nucleic acid that has at least 89% nucleotide identity to SEQ ID NO:16. In embodiments, the genetically engineered microbe includes an exogenous nucleic acid that has at least 90% nucleotide identity to SEQ ID NO:16. In embodiments, the genetically engineered microbe includes an exogenous nucleic acid that has at least 91% nucleotide identity to SEQ ID NO:16. In embodiments, the genetically engineered microbe includes an exogenous nucleic acid that has at least 92% nucleotide identity to SEQ ID NO:16. In embodiments, the genetically engineered microbe includes an exogenous nucleic acid that has at least 93% nucleotide identity to SEQ ID NO:16. In embodiments, the genetically engineered microbe includes an exogenous nucleic acid that has at least 94% nucleotide identity to SEQ ID NO:16. In embodiments, the genetically engineered microbe includes an exogenous nucleic acid that has at least 95% nucleotide identity to SEQ ID NO:16. In embodiments, the genetically engineered microbe includes an exogenous nucleic acid that has at least 96% nucleotide identity to SEQ ID NO:16. In embodiments, the genetically engineered microbe includes an exogenous nucleic acid that has at least 97% nucleotide identity to SEQ ID NO:16. In embodiments, the genetically engineered microbe includes an exogenous nucleic acid that has at least 98% nucleotide identity to SEQ ID NO:16. In embodiments, the genetically engineered microbe includes an exogenous nucleic acid that has at least 99% nucleotide identity to SEQ ID NO:16.

In embodiments, the exogenous nucleic acid has a sequence identity of 81% to SEQ ID NO:16. In embodiments, the exogenous nucleic acid has a sequence identity of 82% to SEQ ID NO:16. In embodiments, the exogenous nucleic acid has a sequence identity of 83% to SEQ ID NO: 16. In embodiments, the exogenous nucleic acid has a sequence identity of 84% to SEQ ID NO: 16. In embodiments, the exogenous nucleic acid has a sequence identity of 85% to SEQ ID NO:16. In embodiments, the exogenous nucleic acid has a sequence identity of 86% to SEQ ID NO:16. In embodiments, the exogenous nucleic acid has a sequence identity of 87% to SEQ ID NO:16. In embodiments, the exogenous nucleic acid has a sequence identity of 88% to SEQ ID NO:16. In embodiments, the exogenous nucleic acid has a sequence identity of 89% to SEQ ID NO: 16. In embodiments, the exogenous nucleic acid has a sequence identity of 90% to SEQ ID NO:16. In embodiments, the exogenous nucleic acid has a sequence identity of 91% to SEQ ID NO:16. In embodiments, the exogenous nucleic acid has a sequence identity of 92% to SEQ ID NO: 16. In embodiments, the exogenous nucleic acid has a sequence identity of 93% to SEQ ID NO:16. In embodiments, the exogenous nucleic acid has a sequence identity of 94% to SEQ ID NO: 16 In embodiments, the exogenous nucleic acid has a sequence identity of 95% to SEQ ID NO:16. In embodiments, the exogenous nucleic acid has a sequence identity of 96% to SEQ ID NO:16. In embodiments, the exogenous nucleic acid has a sequence identity of 97% to SEQ ID NO: 16. In embodiments, the exogenous nucleic acid has a sequence identity of 98% to SEQ ID NO: 16. In embodiments, the exogenous nucleic acid has a sequence identity of 99% to SEQ ID NO: 16. In embodiments, the exogenous nucleic acid is the sequence of SEQ ID NO: 16.

In embodiments, the genetically engineered microbe provided herein includes an exogenous Flavin reductase encoding nucleic acid. In embodiments, the genetically engineered microbe provided herein includes an exogenous Flavin reductase.

In embodiments, the genetically engineered microbe includes an exogenous Tar15 encoding nucleic acid. In embodiments, the exogenous Tar15 encoding nucleic acid has at least 80% nucleotide identity to SEQ ID NO:15. In embodiments, the exogenous Tar15 encoding nucleic acid has at least 81% nucleotide identity to SEQ ID NO:15. In embodiments, the exogenous Tar15 encoding nucleic acid has at least 82% nucleotide identity to SEQ ID NO:15. In embodiments, the exogenous TarT5 encoding nucleic acid has at least 83% nucleotide identity to SEQ ID NO:15. In embodiments, the exogenous Tar15 encoding nucleic acid has at least 84% nucleotide identity to SEQ ID NO:15. In embodiments, the exogenous Tar15 encoding nucleic acid has at least 85% nucleotide identity to SEQ ID NO:15. In embodiments, the exogenous Tar15 encoding nucleic acid has at least 86% nucleotide identity to SEQ ID NO:15. In embodiments, the exogenous Tar15 encoding nucleic acid has at least 87% nucleotide identity to SEQ ID NO:15. In embodiments, the exogenous Tar15 encoding nucleic acid has at least 88% nucleotide identity to SEQ ID NO:15. In embodiments, the exogenous Tar15 encoding nucleic acid has at least 89% nucleotide identity to SEQ ID NO:15. In embodiments, the exogenous Tar15 encoding nucleic acid has at least 90% nucleotide identity to SEQ ID NO:15. In embodiments, the exogenous Tar15 encoding nucleic acid has at least 91% nucleotide identity to SEQ ID NO:15. In embodiments, the exogenous Tar15 encoding nucleic acid has at least 92% nucleotide identity to SEQ ID NO:15. In embodiments, the exogenous Tar15 encoding nucleic acid has at least 93% nucleotide identity to SEQ ID NO:15. In embodiments, the exogenous Tar15 encoding nucleic acid has at least 94% nucleotide identity to SEQ ID NO:15, In embodiments, the exogenous Tar15 encoding nucleic acid has at least 95% nucleotide identity to SEQ ID NO:15. In embodiments, the exogenous Tar15 encoding nucleic acid has at least 96% nucleotide identity to SEQ ID NO:15. In embodiments, the exogenous Tar15 encoding nucleic acid has at least 97% nucleotide identity to SEQ ID NO:15. In embodiments, the exogenous Tar15 encoding nucleic acid has at least 98% nucleotide identity to SEQ ID NO:15. In embodiments, the exogenous Tar15 encoding nucleic acid has at least 99% nucleotide identity to SEQ ID NO:15.

In embodiments, the exogenous Tar15 nucleic acid has a sequence identity of 81% to SEQ ID NO:15. In embodiments, the exogenous TarT5 nucleic acid has a sequence identity of 82% to SEQ ID NO: 15. In embodiments, the exogenous Tar15 nucleic acid has a sequence identity of 83% to SEQ ID NO: 15. In embodiments, the exogenous Tar15 nucleic acid has a sequence identity of 84% to SEQ ID NO:15. In embodiments, the exogenous Tar15 nucleic acid has a sequence identity of 85% to SEQ ID NO:15. In embodiments, the exogenous Tar15 nucleic acid has a sequence identity of 86% to SEQ ID NO:15. In embodiments, the exogenous Tar15 nucleic acid has a sequence identity of 87% to SEQ ID NO:15. In embodiments, the exogenous Tar15 nucleic acid has a sequence identity of 88% to SEQ ID NO:15. In embodiments, the exogenous Tar15 nucleic acid has a sequence identity of 89% to SEQ ID NO:15. In embodiments, the exogenous Tar15 nucleic acid has a sequence identity of 90% to SEQ ID NO:15. In embodiments, the exogenous TarT5 nucleic acid has a sequence identity of 91% to SEQ ID NO:15. In embodiments, the exogenous Tar15 nucleic acid has a sequence identity of 92% to SEQ ID NO:15. In embodiments, the exogenous Tar15 nucleic acid has a sequence identity of 93% to SEQ ID NO:15. In embodiments, the exogenous Tar5 nucleic acid has a sequence identity of 94% to SEQ ID NO:15. In embodiments, the exogenous Tar15 nucleic acid has a sequence identity of 95% to SEQ ID NO:15. In embodiments, the exogenous Tar15 nucleic acid has a sequence identity of 96% to SEQ ID NO: 15. In embodiments, the exogenous Tar5 nucleic acid has a sequence identity of 97% to SEQ ID NO:15. In embodiments, the exogenous TarT5 nucleic acid has a sequence identity of 98% to SEQ ID NO:15. In embodiments, the exogenous Tar15 nucleic acid has a sequence identity of 99% to SEQ ID NO:15. In embodiments, the exogenous Tar15 nucleic acid is the sequence of SEQ ID NO: 15.

In embodiments, the genetically engineered microbe includes an exogenous Tar15 enzyme.

In embodiments, the exogenous nucleic acid provided herein includes at least 1 optimized codon. In embodiments, the exogenous nucleic acid provided herein includes at least 2 optimized codons. In embodiments, the exogenous nucleic acid provided herein includes at least 4 optimized codons. In embodiments, the exogenous nucleic acid provided herein includes at least 6 optimized codons. In embodiments, the exogenous nucleic acid provided herein includes at least 8 optimized codons. In embodiments, the exogenous nucleic acid provided herein includes at least 10 optimized codons. In embodiments, the exogenous nucleic acid provided herein includes at least 12 optimized codons. In embodiments, the exogenous nucleic acid provided herein includes at least 14 optimized codons. In embodiments, the exogenous nucleic acid provided herein includes at least 16 optimized codons. In embodiments, the exogenous nucleic acid provided herein includes at least 18 optimized codons. In embodiments, the exogenous nucleic acid provided herein includes at least 20 optimized codons. In embodiments, the exogenous nucleic acid provided herein includes at least 22 optimized codons. In embodiments, the exogenous nucleic acid provided herein includes at least 24 optimized codons. In embodiments, the exogenous nucleic acid provided herein includes at least 26 optimized codons. In embodiments, the exogenous nucleic acid provided herein includes at least 28 optimized codons. In embodiments, the exogenous nucleic acid provided herein includes at least 30 optimized codons. In embodiments, the exogenous nucleic acid provided herein includes at least 32 optimized codons. In embodiments, the exogenous nucleic acid provided herein includes at least 34 optimized codons. In embodiments, the exogenous nucleic acid provided herein includes at least 36 optimized codons. In embodiments, the exogenous nucleic acid provided herein includes at least 38 optimized codons. In embodiments, the exogenous nucleic acid provided herein includes at least 40 optimized codons. In embodiments, the exogenous nucleic acid provided herein includes at least 42 optimized codons. In embodiments, the exogenous nucleic acid provided herein includes at least 44 optimized codons. In embodiments, the exogenous nucleic acid provided herein includes at least 46 optimized codons. In embodiments, the exogenous nucleic acid provided herein includes at least 48 optimized codons. In embodiments, the exogenous nucleic acid provided herein includes at least 50 optimized codons. In embodiments, the exogenous nucleic acid provided herein includes at least 52 optimized codons. In embodiments, the exogenous nucleic acid provided herein includes at least 54 optimized codons. In embodiments, the exogenous nucleic acid provided herein includes at least 56 optimized codons. In embodiments, the exogenous nucleic acid provided herein includes at least 58 optimized codons. In embodiments, the exogenous nucleic acid provided herein includes at least 60 optimized codons. In embodiments, the exogenous nucleic acid provided herein includes at least 62 optimized codons. In embodiments, the exogenous nucleic acid provided herein includes at least 64 optimized codons. In embodiments, the exogenous nucleic acid provided herein includes at least 66 optimized codons. In embodiments, the exogenous nucleic acid provided herein includes at least 68 optimized codons. In embodiments, the exogenous nucleic acid provided herein includes at least 70 optimized codons. In embodiments, the exogenous nucleic acid provided herein includes at least 72 optimized codons. In embodiments, the exogenous nucleic acid provided herein includes at least 74 optimized codons. In embodiments, the exogenous nucleic acid provided herein includes at least 76 optimized codons. In embodiments, the exogenous nucleic acid provided herein includes at least 78 optimized codons. In embodiments, the exogenous nucleic acid provided herein includes at least 80 optimized codons.

For the genetically engineered microbe provided herein, in embodiments, the exogenous Tar14 encoding nucleic acid, exogenous Tar13 encoding nucleic acid, or exogenous Tar16 encoding nucleic acid further comprises an exogenous promoter. In embodiments, one or more of the exogenous Tar14 encoding nucleic acid, exogenous Tar13 encoding nucleic acid, or exogenous Tar16 encoding nucleic acid further comprises an exogenous promoter.

In embodiments, the exogenous promoter is BG51 (SEQ ID NO:18), Pfer (SEQ ID NO:19), Ptac (SEQ ID NO:20), Pem7 (SEQ ID NO:21), arcB (SEQ ID NO:26), aroF (SEQ ID NO: 27), glk (SEQ ID NO:28), mqsR (SEQ ID NO:29), recA (SEQ ID NO:30), rpoS (SEQ ID NO:31), rpsU (SEQ ID NO:32), or sigX (SEQ ID NO:33). In embodiments, the exogenous promoter is BG51 (SEQ ID NO:18). In embodiments, the exogenous promoter is Pfer (SEQ ID NO:19). In embodiments, the exogenous promoter is Ptac (SEQ ID NO:20). In embodiments, the exogenous promoter is Pem7 (SEQ ID NO:21). In embodiments, the exogenous promoter is arcB (SEQ ID NO:26). In embodiments, the exogenous promoter is aroF (SEQ ID NO:27). In embodiments, the exogenous promoter is glk (SEQ ID NO:28). In embodiments, the exogenous promoter is mqsR (SEQ ID NO:29). In embodiments, the exogenous promoter is recA (SEQ ID NO:30). In embodiments, the exogenous promoter is rpoS (SEQ ID NO:31). In embodiments, the exogenous promoter is rpoS (SEQ ID NO:31). In embodiments, the exogenous promoter is rpsU (SEQ ID NO:32). In embodiments, the exogenous promoter is sigX (SEQ ID NO:33).

For the genetically engineered microbe provided herein, in embodiments, the exogenous Tar14 encoding nucleic acid, exogenous Tar13 encoding nucleic acid, or exogenous Tar16 encoding nucleic acid further comprises an exogenous terminator. In embodiments, one or more of the exogenous Tar14 encoding nucleic acid, exogenous Tar13 encoding nucleic acid, or exogenous Tar16 encoding nucleic acid further comprises an exogenous terminator.

In embodiments, the genetically engineered microbe is a gram negative bacterium. In embodiments, the gram negative bacterium is E. coli or P. putida. In embodiments, the gram negative bacterium is E. coli. In embodiments, the gram negative bacterium is P. putida.

In embodiments, the genetically engineered microbe is a gram positive bacterium. In embodiments, the gram positive bacterium is Corynebacterium glutanicum.

In embodiments, the genetically engineered microbe is a human gastrointestinal microbe.

In an aspect is provided a genetically engineered microbe. The microbe expresses one or more of Tar 14, Tar13, or Tar16. In an embodiment, the genetically engineered microbe expresses Tar14. In an embodiment, the genetically engineered microbe expresses Tar13. In an embodiment, the genetically engineered microbe expresses Tar16. In an embodiment, the genetically engineered microbe expresses Tar14 and Tar13. In an embodiment, the genetically engineered microbe expresses Tar14 and Tar16. In an embodiment, the genetically engineered microbe expresses Tar13 and Tar16. In an embodiment, the genetically engineered microbe expresses Tar14, Tar13 and Tar16. In an embodiment, the microbe is a human gastrointestinal microbe. In embodiments, the genetically engineered microbe further expresses Tar15.

### IV. Nucleic Acids

In an aspect is provided an isolated nucleic acid, the isolated nucleic acid including a Tar14 encoding nucleic acid, a Tar13 encoding nucleic acid, a Tar16 encoding nucleic acid, or a Tar15 nucleic acid. In embodiments, the isolated nucleic acid includes a Tar14 encoding nucleic acid provided herein, including embodiments thereof. In embodiments, the isolated nucleic acid includes a Tar13 encoding nucleic acid provided herein, including embodiments thereof. In embodiments, the isolated nucleic acid includes a Tar14 encoding nucleic acid provided herein, including embodiments thereof. In embodiments, the isolated nucleic acid includes a Tar16 encoding nucleic acid provided herein, including embodiments thereof. In embodiments, the isolated nucleic acid includes a Tar15 encoding nucleic acid provided herein, including embodiments thereof.

In embodiments, the isolated nucleic acid has at least 85% nucleotide identity to SEQ ID NO:11, SEQ ID NO:13, SEQ ID NO: 15, or SEQ ID NO: 17. In embodiments, the isolated nucleic acid has at least 85% nucleotide identity to SEQ ID NO: 11. In embodiments, the isolated nucleic acid has at least 85% nucleotide identity to SEQ ID NO: 13. In embodiments, the isolated nucleic acid has at least 85% nucleotide identity to SEQ ID SEQ ID NO:15. In embodiments, the isolated nucleic acid has at least 85% nucleotide identity to SEQ ID NO:17.

In an aspect is provided an isolated nucleic acid, the isolated nucleic acid including one or more of a Tar14 encoding nucleic acid, a Tar13 encoding nucleic acid, a Tar16 encoding nucleic acid, or a Tar5 nucleic acid. In embodiments, the isolated nucleic acid includes a Tar14 encoding nucleic acid, a Tar13 encoding nucleic acid, a Tar16 encoding nucleic acid, and a Tar15 nucleic acid. In embodiments, the isolated nucleic acid includes a Tar14 encoding nucleic acid and a Tar13 encoding nucleic acid. In embodiments, the isolated nucleic acid includes a Tar14 encoding nucleic acid and a Tar16 encoding nucleic acid. In embodiments, the isolated nucleic acid includes a Tar16 encoding nucleic acid and a Tar13 encoding nucleic acid. In embodiments, the isolated nucleic acid further includes a Tar15 encoding nucleic acid. In embodiments, the isolated nucleic acid is a plasmid.

In embodiments, the isolated nucleic acid comprises one or more sequences having at least 85% nucleotide identity to SEQ ID NO:11, SEQ ID NO:13, SEQ ID NO:15, or SEQ ID NO:17. In embodiments, the isolated nucleic acid includes one or more of any combination of the nucleic acids provided herein, including embodiments thereof.

In embodiments, the isolated nucleic acid provided herein includes at least one optimized codon.

In an aspect is provided a plasmid including one or more nucleic acid sequences encoding for Tar13, Tar14, Tar15 or Tar16.

### V. Enzymes

In an aspect, an isolated enzyme is provided, the isolated enzyme including Tar14, Tar13, Tar16, or Tar15, or an enzymatically active fragment or variant thereof. In embodiments, the isolated enzyme includes Tar14, or an enzymatically active fragment or variant thereof. In embodiments, the isolated enzyme includes Tar13, or an enzymatically active fragment or variant thereof. In embodiments, the isolated enzyme includes Tar16, or an enzymatically active fragment or variant thereof. In embodiments, the isolated enzyme includes Tar15, or an enzymatically active fragment or variant thereof.

In embodiments, the Tar13 enzyme provided herein has at least 80% sequence identity to SEQ ID NO: 1. In embodiments, the Tar13 enzyme provided herein has at least 81% sequence identity to SEQ ID NO:1. In embodiments, the Tar13 enzyme provided herein has at least 82% sequence identity to SEQ ID NO:1. In embodiments, the Tar13 enzyme provided herein has at least 83% sequence identity to SEQ ID NO: 1. In embodiments, the Tar13 enzyme provided herein has at least 84% sequence identity to SEQ ID NO: 1. In embodiments, the Tar13 enzyme provided herein has at least 85% sequence identity to SEQ ID NO: 1. In embodiments, the Tar13 enzyme provided herein has at least 86% sequence identity to SEQ ID NO:1. In embodiments, the Tar13 enzyme provided herein has at least 87% sequence identity to SEQ ID NO:1. In embodiments, the Tar13 enzyme provided herein has at least 88% sequence identity to SEQ ID NO:1. In embodiments, the Tar13 enzyme provided herein has at least 89% sequence identity to SEQ ID NO: 1. In embodiments, the Tar13 enzyme provided herein has at least 90% sequence identity to SEQ ID NO:1. In embodiments, the Tar13 enzyme provided herein has at least 91% sequence identity to SEQ ID NO:1. In embodiments, the Tar13 enzyme provided herein has at least 92% sequence identity to SEQ ID NO:1. In embodiments, the Tar13 enzyme provided herein has at least 93% sequence identity to SEQ ID NO:1. In embodiments, the Tar13 enzyme provided herein has at least 94% sequence identity to SEQ ID NO:1. In embodiments, the Tar13 enzyme provided herein has at least 95% sequence identity to SEQ ID NO: 1. In embodiments, the Tar13 enzyme provided herein has at least 96% sequence identity to SEQ ID NO:1. In embodiments, the Tar13 enzyme provided herein has at least 97% sequence identity to SEQ ID NO:1. In embodiments, the Tar13 enzyme provided herein has at least 98% sequence identity to SEQ ID NO:1. In embodiments, the Tar13 enzyme provided herein has at least 99% sequence identity to SEQ ID NO:1.

In embodiments, the Tar13 enzyme has a sequence identity of 81% to SEQ ID NO:1. In embodiments, the Tar13 enzyme has a sequence identity of 82% to SEQ ID NO:1. In embodiments, the Tar13 enzyme has a sequence identity of 83% to SEQ ID NO: 1. In embodiments, the Tar13 enzyme has a sequence identity of 84% to SEQ ID NO:1. In embodiments, the Tar13 enzyme has a sequence identity of 85% to SEQ ID NO: 1. In embodiments, the Tar13 enzyme has a sequence identity of 86% to SEQ ID NO:1. In embodiments, the Tar13 enzyme has a sequence identity of 87% to SEQ ID NO: 1. In embodiments, the Tar13 enzyme has a sequence identity of 88% to SEQ ID NO: 1. In embodiments, the Tar13 enzyme has a sequence identity of 89% to SEQ ID NO: 1. In embodiments, the Tar13 enzyme has a sequence identity of 90% to SEQ ID NO: 1. In embodiments, the Tar13 enzyme has a sequence identity of 91% to SEQ ID NO: 1. In embodiments, the Tar13 enzyme has a sequence identity of 92% to SEQ ID NO: 1. In embodiments, the Tar13 enzyme has a sequence identity of 93% to SEQ ID NO: 1. In embodiments, the Tar13 enzyme has a sequence identity of 94% to SEQ ID NO:1. In embodiments, the Tar13 enzyme has a sequence identity of 95% to SEQ ID NO: 1. In embodiments, the Tar13 enzyme has a sequence identity of 96% to SEQ ID NO: 1. In embodiments, the Tar13 enzyme has a sequence identity of 97% to SEQ ID NO: 1. In embodiments, the Tar13 enzyme has a sequence identity of 98% to SEQ ID NO: 1. In embodiments, the Tar13 enzyme has a sequence identity of 99% to SEQ ID NO:1. In embodiments, the Tar13 enzyme is the sequence of SEQ ID NO: 1.

In embodiments, the TarT3 enzyme provided herein has at least 80% sequence identity to SEQ ID NO:2. In embodiments, the Tar13 enzyme provided herein has at least 81% sequence identity to SEQ ID NO:2. In embodiments, the Tar13 enzyme provided herein has at least 82% sequence identity to SEQ ID NO:2. In embodiments, the Tar13 enzyme provided herein has at least 83% sequence identity to SEQ ID NO:2. In embodiments, the Tar13 enzyme provided herein has at least 84% sequence identity to SEQ ID NO:2. In embodiments, the Tar13 enzyme provided herein has at least 85% sequence identity to SEQ ID NO:2. In embodiments, the Tar13 enzyme provided herein has at least 86% sequence identity to SEQ ID NO:2. In embodiments, the Tar13 enzyme provided herein has at least 87% sequence identity to SEQ ID NO:2. In embodiments, the Tar13 enzyme provided herein has at least 88% sequence identity to SEQ ID NO:2. In embodiments, the Tar13 enzyme provided herein has at least 89% sequence identity to SEQ ID NO:2. In embodiments, the Tar13 enzyme provided herein has at least 90% sequence identity to SEQ ID NO:2. In embodiments, the Tar13 enzyme provided herein has at least 91% sequence identity to SEQ ID NO:2. In embodiments, the Tar13 enzyme provided herein has at least 92% sequence identity to SEQ ID NO:2. In embodiments, the Tar13 enzyme provided herein has at least 93% sequence identity to SEQ ID NO:2. In embodiments, the Tar13 enzyme provided herein has at least 94% sequence identity to SEQ ID NO:2. In embodiments, the Tar13 enzyme provided herein has at least 95% sequence identity to SEQ ID NO:2. In embodiments, the Tar13 enzyme provided herein has at least 96% sequence identity to SEQ ID NO:2. In embodiments, the Tar13 enzyme provided herein has at least 97% sequence identity to SEQ ID NO:2. In embodiments, the Tar13 enzyme provided herein has at least 98% sequence identity to SEQ ID NO:2. In embodiments, the Tar13 enzyme provided herein has at least 99% sequence identity to SEQ ID NO:2.

In embodiments, the Tar13 enzyme has a sequence identity of 81% to SEQ ID NO:2. In embodiments, the Tar13 enzyme has a sequence identity of 82% to SEQ ID NO:2. In embodiments, the Tar13 enzyme has a sequence identity of 83% to SEQ ID NO:2. In embodiments, the Tar13 enzyme has a sequence identity of 84% to SEQ ID NO:2. In embodiments, the Tar13 enzyme has a sequence identity of 85% to SEQ ID NO:2. In embodiments, the Tar13 enzyme has a sequence identity of 86% to SEQ ID NO:2. In embodiments, the Tar13 enzyme has a sequence identity of 87% to SEQ ID NO:2. In embodiments, the Tar13 enzyme has a sequence identity of 88% to SEQ ID NO:2. In embodiments, the Tar13 enzyme has a sequence identity of 89% to SEQ ID NO:2. In embodiments, the Tar13 enzyme has a sequence identity of 90% to SEQ ID NO:2. In embodiments, the Tar13 enzyme has a sequence identity of 91% to SEQ ID NO:2. In embodiments, the Tar13 enzyme has a sequence identity of 92% to SEQ ID NO:2. In embodiments, the Tar13 enzyme has a sequence identity of 93% to SEQ ID NO:2. In embodiments, the Tar13 enzyme has a sequence identity of 94% to SEQ ID NO:2. In embodiments, the Tar13 enzyme has a sequence identity of 95% to SEQ ID NO:2. In embodiments, the Tar13 enzyme has a sequence identity of 96% to SEQ ID NO:2. In embodiments, the Tar13 enzyme has a sequence identity of 97% to SEQ ID NO:2. In embodiments, the Tar13 enzyme has a sequence identity of 98% to SEQ ID NO:2. In embodiments, the Tar13 enzyme has a sequence identity of 99% to SEQ ID NO:2. In embodiments, the Tar13 enzyme is the sequence of SEQ ID NO:2.

In embodiments, the Tar14 enzyme provided herein has at least 80% sequence identity to SEQ ID NO:3. In embodiments, the Tar14 enzyme provided herein has at least 81% sequence identity to SEQ ID NO:3. In embodiments, the Tar14 enzyme provided herein has at least 82% sequence identity to SEQ ID NO:3. In embodiments, the Tar14 enzyme provided herein has at least 83% sequence identity to SEQ ID NO:3. In embodiments, the Tar14 enzyme provided herein has at least 84% sequence identity to SEQ ID NO:3. In embodiments, the Tar14 enzyme provided herein has at least 85% sequence identity to SEQ ID NO:3. In embodiments, the Tar14 enzyme provided herein has at least 86% sequence identity to SEQ ID NO:3. In embodiments, the Tar14 enzyme provided herein has at least 87% sequence identity to SEQ ID NO:3. In embodiments, the Tar14 enzyme provided herein has at least 88% sequence identity to SEQ ID NO:3. In embodiments, the Tar14 enzyme provided herein has at least 89% sequence identity to SEQ ID NO:3. In embodiments, the Tar14 enzyme provided herein has at least 90% sequence identity to SEQ ID NO:3. In embodiments, the Tar14 enzyme provided herein has at least 91% sequence identity to SEQ ID NO:3. In embodiments, the Tar14 enzyme provided herein has at least 92% sequence identity to SEQ ID NO:3 In embodiments, the Tar14 enzyme provided herein has at least 93% sequence identity to SEQ ID NO:3. In embodiments, the Tar14 enzyme provided herein has at least 94% sequence identity to SEQ ID NO:3. In embodiments, the Tar14 enzyme provided herein has at least 95% sequence identity to SEQ ID NO:3. In embodiments, the Tar14 enzyme provided herein has at least 96% sequence identity to SEQ ID NO:3. In embodiments, the Tar14 enzyme provided herein has at least 97% sequence identity to SEQ ID NO:3. In embodiments, the Tar14 enzyme provided herein has at least 98% sequence identity to SEQ ID NO:3. In embodiments, the Tar14 enzyme provided herein has at least 99% sequence identity to SEQ ID NO:3.

In embodiments, the Tar14 enzyme has a sequence identity of 81% to SEQ ID NO:3. In embodiments, the Tar14 enzyme has a sequence identity of 82% to SEQ ID NO:3. In embodiments, the Tar14 enzyme has a sequence identity of 83% to SEQ ID NO:3. In embodiments, the Tar14 enzyme has a sequence identity of 84% to SEQ ID NO:3. In embodiments, the Tar14 enzyme has a sequence identity of 85% to SEQ ID NO:3. In embodiments, the Tar14 enzyme has a sequence identity of 86% to SEQ ID NO:3. In embodiments, the Tar14 enzyme has a sequence identity of 87% to SEQ ID NO:3. In embodiments, the Tar14 enzyme has a sequence identity of 88% to SEQ ID NO:3. In embodiments, the Tar14 enzyme has a sequence identity of 89% to SEQ ID NO:3. In embodiments, the Tar14 enzyme has a sequence identity of 90% to SEQ ID NO:3. In embodiments, the Tar14 enzyme has a sequence identity of 91% to SEQ ID NO:3. In embodiments, the Tar14 enzyme has a sequence identity of 92% to SEQ ID NO:3. In embodiments, the Tar14 enzyme has a sequence identity of 93% to SEQ ID NO:3. In embodiments, the Tar14 enzyme has a sequence identity of 94% to SEQ ID NO:3. In embodiments, the Tar14 enzyme has a sequence identity of 95% to SEQ ID NO:3. In embodiments, the Tar14 enzyme has a sequence identity of 96% to SEQ ID NO:3. In embodiments, the Tar14 enzyme has a sequence identity of 97% to SEQ ID NO:3. In embodiments, the Tar14 enzyme has a sequence identity of 98% to SEQ ID NO:3. In embodiments, the Tar14 enzyme has a sequence identity of 99% to SEQ ID NO:3. In embodiments, the Tar14 enzyme is the sequence of SEQ ID NO:3.

In embodiments, the Tar14 enzyme provided herein has at least 80% sequence identity to SEQ ID NO:4. In embodiments, the Tar14 enzyme provided herein has at least 81% sequence identity to SEQ ID NO:4. In embodiments, the Tar14 enzyme provided herein has at least 82% sequence identity to SEQ ID NO:4. In embodiments, the Tar14 enzyme provided herein has at least 83% sequence identity to SEQ ID NO:4. In embodiments, the Tar14 enzyme provided herein has at least 84% sequence identity to SEQ ID NO:4. In embodiments, the Tar14 enzyme provided herein has at least 85% sequence identity to SEQ ID NO:4. In embodiments, the Tar14 enzyme provided herein has at least 86% sequence identity to SEQ ID NO:4. In embodiments, the Tar14 enzyme provided herein has at least 87% sequence identity to SEQ ID NO:4. In embodiments, the Tar14 enzyme provided herein has at least 88% sequence identity to SEQ ID NO:4. In embodiments, the Tar14 enzyme provided herein has at least 89% sequence identity to SEQ ID NO:4. In embodiments, the Tar14 enzyme provided herein has at least 90% sequence identity to SEQ ID NO:4. In embodiments, the Tar14 enzyme provided herein has at least 91% sequence identity to SEQ ID NO:4. In embodiments, the Tar14 enzyme provided herein has at least 92% sequence identity to SEQ ID NO:4. In embodiments, the Tar14 enzyme provided herein has at least 93% sequence identity to SEQ ID NO:4. In embodiments, the Tar14 enzyme provided herein has at least 94% sequence identity to SEQ ID NO:4. In embodiments, the Tar14 enzyme provided herein has at least 95% sequence identity to SEQ ID NO:4 In embodiments, the Tar14 enzyme provided herein has at least 96% sequence identity to SEQ ID NO:4. In embodiments, the Tar14 enzyme provided herein has at least 97% sequence identity to SEQ ID NO:4 In embodiments, the Tar14 enzyme provided herein has at least 98% sequence identity to SEQ ID NO:4. In embodiments, the Tar14 enzyme provided herein has at least 99% sequence identity to SEQ ID NO:4.

In embodiments, the Tar14 enzyme has a sequence identity of 81% to SEQ ID NO:4. In embodiments, the Tar14 enzyme has a sequence identity of 82% to SEQ ID NO:4. In embodiments, the Tar14 enzyme has a sequence identity of 83% to SEQ ID NO:4 In embodiments, the Tar14 enzyme has a sequence identity of 84% to SEQ ID NO:4. In embodiments, the Tar14 enzyme has a sequence identity of 85% to SEQ ID NO:4. In embodiments, the Tar14 enzyme has a sequence identity of 86% to SEQ ID NO:4. In embodiments, the Tar14 enzyme has a sequence identity of 87% to SEQ ID NO:4. In embodiments, the Tar14 enzyme has a sequence identity of 88% to SEQ ID NO:4. In embodiments, the Tar14 enzyme has a sequence identity of 89% to SEQ ID NO:4. In embodiments, the Tar14 enzyme has a sequence identity of 90% to SEQ ID NO:4. In embodiments, the Tar14 enzyme has a sequence identity of 91% to SEQ ID NO:4. In embodiments, the Tar14 enzyme has a sequence identity of 92% to SEQ ID NO:4. In embodiments, the Tar14 enzyme has a sequence identity of 93% to SEQ ID NO:4 In embodiments, the Tar14 enzyme has a sequence identity of 94% to SEQ ID NO:4. In embodiments, the Tar14 enzyme has a sequence identity of 95% to SEQ ID NO:4. In embodiments, the Tar14 enzyme has a sequence identity of 96% to SEQ ID NO:4. In embodiments, the Tar14 enzyme has a sequence identity of 97% to SEQ ID NO:4. In embodiments, the Tar14 enzyme has a sequence identity of 98% to SEQ ID NO:4. In embodiments, the Tar14 enzyme has a sequence identity of 99% to SEQ ID NO:4. In embodiments, the Tar14 enzyme is the sequence of SEQ ID NO:4.

In embodiments, the Tar15 enzyme provided herein has at least 80% sequence identity to SEQ ID NO:6. In embodiments, the Tar15 enzyme provided herein has at least 81% sequence identity to SEQ ID NO:6. In embodiments, the Tar15 enzyme provided herein has at least 82% sequence identity to SEQ ID NO:6. In embodiments, the Tar15 enzyme provided herein has at least 83% sequence identity to SEQ ID NO:6. In embodiments, the Tar15 enzyme provided herein has at least 84% sequence identity to SEQ ID NO:6. In embodiments, the Tar15 enzyme provided herein has at least 85% sequence identity to SEQ ID NO:6. In embodiments, the Tar15 enzyme provided herein has at least 86% sequence identity to SEQ ID NO:6. In embodiments, the Tar15 enzyme provided herein has at least 87% sequence identity to SEQ ID NO:6. In embodiments, the Tar15 enzyme provided herein has at least 88% sequence identity to SEQ ID NO:6. In embodiments, the Tar15 enzyme provided herein has at least 89% sequence identity to SEQ ID NO:6. In embodiments, the Tar15 enzyme provided herein has at least 90% sequence identity to SEQ ID NO:6. In embodiments, the Tar15 enzyme provided herein has at least 91% sequence identity to SEQ ID NO:6. In embodiments, the Tar15 enzyme provided herein has at least 92% sequence identity to SEQ ID NO:6. In embodiments, the Tar15 enzyme provided herein has at least 93% sequence identity to SEQ ID NO:6. In embodiments, the Tar15 enzyme provided herein has at least 94% sequence identity to SEQ ID NO:6. In embodiments, the Tar15 enzyme provided herein has at least 95% sequence identity to SEQ ID NO:6. In embodiments, the Tar15 enzyme provided herein has at least 96% sequence identity to SEQ ID NO:6. In embodiments, the Tar15 enzyme provided herein has at least 97% sequence identity to SEQ ID NO:6. In embodiments, the Tar15 enzyme provided herein has at least 98% sequence identity to SEQ ID NO:6. In embodiments, the Tar15 enzyme provided herein has at least 99% sequence identity to SEQ ID NO:6.

In embodiments, the Tar15 enzyme has a sequence identity of 81% to SEQ ID NO:6. In embodiments, the Tar15 enzyme has a sequence identity of 82% to SEQ ID NO:6. In embodiments, the Tar15 enzyme has a sequence identity of 83% to SEQ ID NO:6. In embodiments, the Tar15 enzyme has a sequence identity of 84% to SEQ ID NO:6. In embodiments, the Tar15 enzyme has a sequence identity of 85% to SEQ ID NO:6. In embodiments, the Tar15 enzyme has a sequence identity of 86% to SEQ ID NO: 6. In embodiments, the Tar15 enzyme has a sequence identity of 87% to SEQ ID NO: 6. In embodiments, the Tar15 enzyme has a sequence identity of 88% to SEQ ID NO: 6. In embodiments, the Tar15 enzyme has a sequence identity of 89% to SEQ ID NO: 6. In embodiments, the Tar15 enzyme has a sequence identity of 90% to SEQ ID NO: 6. In embodiments, the Tar15 enzyme has a sequence identity of 91% to SEQ ID NO: 6. In embodiments, the Tar15 enzyme has a sequence identity of 92% to SEQ ID NO:6. In embodiments, the Tar15 enzyme has a sequence identity of 93% to SEQ ID NO:6. In embodiments, the Tar15 enzyme has a sequence identity of 94% to SEQ ID NO:6. In embodiments, the Tar15 enzyme has a sequence identity of 95% to SEQ ID NO: 6. In embodiments, the Tar15 enzyme has a sequence identity of 96% to SEQ ID NO: 6. In embodiments, the Tar15 enzyme has a sequence identity of 97% to SEQ ID NO: 6. In embodiments, the Tar15 enzyme has a sequence identity of 98% to SEQ ID NO: 6. In embodiments, the Tar15 enzyme has a sequence identity of 99% to SEQ ID NO: 6. In embodiments, the Tar15 enzyme is the sequence of SEQ ID NO:6.

In embodiments, the Tar15 enzyme provided herein has at least 80% sequence identity to SEQ ID NO:7. In embodiments, the Tar15 enzyme provided herein has at least 81% sequence identity to SEQ ID NO:7. In embodiments, the Tar15 enzyme provided herein has at least 82% sequence identity to SEQ ID NO:7. In embodiments, the Tar15 enzyme provided herein has at least 83% sequence identity to SEQ ID NO:7. In embodiments, the Tar15 enzyme provided herein has at least 84% sequence identity to SEQ ID NO:7. In embodiments, the Tar15 enzyme provided herein has at least 85% sequence identity to SEQ ID NO:7. In embodiments, the Tar15 enzyme provided herein has at least 86% sequence identity to SEQ ID NO:7. In embodiments, the Tar15 enzyme provided herein has at least 87% sequence identity to SEQ ID NO:7. In embodiments, the Tar15 enzyme provided herein has at least 88% sequence identity to SEQ ID NO:7. In embodiments, the Tar15 enzyme provided herein has at least 89% sequence identity to SEQ ID NO:7. In embodiments, the Tar15 enzyme provided herein has at least 90% sequence identity to SEQ ID NO:7. In embodiments, the Tar15 enzyme provided herein has at least 91% sequence identity to SEQ ID NO:7. In embodiments, the Tar15 enzyme provided herein has at least 92% sequence identity to SEQ ID NO:7. In embodiments, the Tar15 enzyme provided herein has at least 93% sequence identity to SEQ ID NO:7. In embodiments, the Tar15 enzyme provided herein has at least 94% sequence identity to SEQ ID NO:7. In embodiments, the TarT5 enzyme provided herein has at least 95% sequence identity to SEQ ID NO:7. In embodiments, the Tar15 enzyme provided herein has at least 96% sequence identity to SEQ ID NO:7. In embodiments, the Tar15 enzyme provided herein has at least 97% sequence identity to SEQ ID NO:7. In embodiments, the Tar15 enzyme provided herein has at least 98% sequence identity to SEQ ID NO:7. In embodiments, the Tar15 enzyme provided herein has at least 99% sequence identity to SEQ ID NO:7.

In embodiments, the Tar15 enzyme has a sequence identity of 81% to SEQ ID NO:7. In embodiments, the Tar15 enzyme has a sequence identity of 82% to SEQ ID NO:7. In embodiments, the Tar15 enzyme has a sequence identity of 83% to SEQ ID NO:7. In embodiments, the Tar15 enzyme has a sequence identity of 84% to SEQ ID NO:7. In embodiments, the Tar15 enzyme has a sequence identity of 85% to SEQ ID NO: 7. In embodiments, the Tar15 enzyme has a sequence identity of 86% to SEQ ID NO: 7. In embodiments, the Tar15 enzyme has a sequence identity of 87% to SEQ ID NO: 7. In embodiments, the Tar15 enzyme has a sequence identity of 88% to SEQ ID NO: 7. In embodiments, the Tar15 enzyme has a sequence identity of 89% to SEQ ID NO: 7. In embodiments, the Tar15 enzyme has a sequence identity of 90% to SEQ ID NO: 7. In embodiments, the Tar15 enzyme has a sequence identity of 91% to SEQ ID NO: 7. In embodiments, the Tar15 enzyme has a sequence identity of 92% to SEQ ID NO:7. In embodiments, the Tar15 enzyme has a sequence identity of 93% to SEQ ID NO:7. In embodiments, the Tar15 enzyme has a sequence identity of 94% to SEQ ID NO:7. In embodiments, the Tar15 enzyme has a sequence identity of 95% to SEQ ID NO: 7. In embodiments, the Tar15 enzyme has a sequence identity of 96% to SEQ ID NO: 7. In embodiments, the Tar15 enzyme has a sequence identity of 97% to SEQ ID NO: 7. In embodiments, the Tar15 enzyme has a sequence identity of 98% to SEQ ID NO: 7. In embodiments, the Tar15 enzyme has a sequence identity of 99% to SEQ ID NO: 7. In embodiments, the Tar15 enzyme is the sequence of SEQ ID NO:7.

In embodiments, the TarT6 enzyme provided herein has at least 80% sequence identity to SEQ ID NO:8. In embodiments, the Tar16 enzyme provided herein has at least 81% sequence identity to SEQ ID NO:8. In embodiments, the Tar16 enzyme provided herein has at least 82% sequence identity to SEQ ID NO:8. In embodiments, the TarT6 enzyme provided herein has at least 83% sequence identity to SEQ ID NO:8. In embodiments, the Tar16 enzyme provided herein has at least 84% sequence identity to SEQ ID NO:8. In embodiments, the Tar16 enzyme provided herein has at least 85% sequence identity to SEQ ID NO:8. In embodiments, the Tar16 enzyme provided herein has at least 86% sequence identity to SEQ ID NO:8. In embodiments, the Tar16 enzyme provided herein has at least 87% sequence identity to SEQ ID NO:8. In embodiments, the Tar16 enzyme provided herein has at least 88% sequence identity to SEQ ID NO:8. In embodiments, the Tar16 enzyme provided herein has at least 89% sequence identity to SEQ ID NO:8. In embodiments, the Tar16 enzyme provided herein has at least 90% sequence identity to SEQ ID NO:8. In embodiments, the Tar16 enzyme provided herein has at least 91% sequence identity to SEQ ID NO:8. In embodiments, the Tar16 enzyme provided herein has at least 92% sequence identity to SEQ ID NO:8. In embodiments, the Tar16 enzyme provided herein has at least 93% sequence identity to SEQ ID NO:8. In embodiments, the Tar16 enzyme provided herein has at least 94% sequence identity to SEQ ID NO:8. In embodiments, the Tar16 enzyme provided herein has at least 95% sequence identity to SEQ ID NO:8. In embodiments, the Tar16 enzyme provided herein has at least 96% sequence identity to SEQ ID NO:8. In embodiments, the Tar16 enzyme provided herein has at least 97% sequence identity to SEQ ID NO:8. In embodiments, the Tar16 enzyme provided herein has at least 98% sequence identity to SEQ ID NO:8. In embodiments, the Tar16 enzyme provided herein has at least 99% sequence identity to SEQ ID NO:8.

In embodiments, the Tar16 enzyme has a sequence identity of 81% to SEQ ID NO:8. In embodiments, the Tar16 enzyme has a sequence identity of 82% to SEQ ID NO:8. In embodiments, the Tar16 enzyme has a sequence identity of 83% to SEQ ID NO:8. In embodiments, the Tar16 enzyme has a sequence identity of 84% to SEQ ID NO: 8. In embodiments, the Tar16 enzyme has a sequence identity of 85% to SEQ ID NO: 8. In embodiments, the Tar16 enzyme has a sequence identity of 86% to SEQ ID NO: 8. In embodiments, the Tar16 enzyme has a sequence identity of 87% to SEQ ID NO: 8. In embodiments, the Tar16 enzyme has a sequence identity of 88% to SEQ ID NO: 8. In embodiments, the Tar16 enzyme has a sequence identity of 89% to SEQ ID NO: 8. In embodiments, the Tar16 enzyme has a sequence identity of 90% to SEQ ID NO:8. In embodiments, the Tar16 enzyme has a sequence identity of 91% to SEQ ID NO: 8. In embodiments, the Tar16 enzyme has a sequence identity of 92% to SEQ ID NO:8. In embodiments, the Tar16 enzyme has a sequence identity of 93% to SEQ ID NO:8. In embodiments, the Tar16 enzyme has a sequence identity of 94% to SEQ ID NO: 8. In embodiments, the Tar16 enzyme has a sequence identity of 95% to SEQ ID NO:8. In embodiments, the Tar16 enzyme has a sequence identity of 96% to SEQ ID NO: 8. In embodiments, the Tar16 enzyme has a sequence identity of 97% to SEQ ID NO: 8. In embodiments, the Tar16 enzyme has a sequence identity of 98% to SEQ ID NO: 8. In embodiments, the Tar16 enzyme has a sequence identity of 99% to SEQ ID NO: 8. In embodiments, the Tar16 enzyme is the sequence of SEQ ID NO: 8.

In embodiments, the Tar16 enzyme provided herein has at least 80% sequence identity to SEQ ID NO:9. In embodiments, the Tar16 enzyme provided herein has at least 81% sequence identity to SEQ ID NO:9. In embodiments, the Tar16 enzyme provided herein has at least 82% sequence identity to SEQ ID NO:9. In embodiments, the Tar16 enzyme provided herein has at least 83% sequence identity to SEQ ID NO:9. In embodiments, the Tar16 enzyme provided herein has at least 84% sequence identity to SEQ ID NO:9. In embodiments, the Tar16 enzyme provided herein has at least 85% sequence identity to SEQ ID NO:9. In embodiments, the Tar16 enzyme provided herein has at least 86% sequence identity to SEQ ID NO:9. In embodiments, the Tar16 enzyme provided herein has at least 87% sequence identity to SEQ ID NO:9. In embodiments, the Tar16 enzyme provided herein has at least 88% sequence identity to SEQ ID NO:9. In embodiments, the Tar16 enzyme provided herein has at least 89% sequence identity to SEQ ID NO:9. In embodiments, the Tar16 enzyme provided herein has at least 90% sequence identity to SEQ ID NO:9. In embodiments, the Tar16 enzyme provided herein has at least 91% sequence identity to SEQ ID NO:9. In embodiments, the Tar16 enzyme provided herein has at least 92% sequence identity to SEQ ID NO:9. In embodiments, the Tar16 enzyme provided herein has at least 93% sequence identity to SEQ ID NO:9. In embodiments, the Tar16 enzyme provided herein has at least 94% sequence identity to SEQ ID NO:9. In embodiments, the Tar16 enzyme provided herein has at least 95% sequence identity to SEQ ID NO:9. In embodiments, the Tar16 enzyme provided herein has at least 96% sequence identity to SEQ ID NO:9. In embodiments, the Tar16 enzyme provided herein has at least 97% sequence identity to SEQ ID NO:9. In embodiments, the Tar16 enzyme provided herein has at least 98% sequence identity to SEQ ID NO:9. In embodiments, the Tar16 enzyme provided herein has at least 99% sequence identity to SEQ ID NO:9.

In embodiments, the Tar16 enzyme has a sequence identity of 81% to SEQ ID NO:9. In embodiments, the Tar16 enzyme has a sequence identity of 82% to SEQ ID NO:9. In embodiments, the Tar16 enzyme has a sequence identity of 83% to SEQ ID NO:9. In embodiments, the Tar16 enzyme has a sequence identity of 84% to SEQ ID NO:9. In embodiments, the Tar16 enzyme has a sequence identity of 85% to SEQ ID NO: 9. In embodiments, the Tar16 enzyme has a sequence identity of 86% to SEQ ID NO: 9. In embodiments, the Tar16 enzyme has a sequence identity of 87% to SEQ ID NO: 9. In embodiments, the Tar16 enzyme has a sequence identity of 88% to SEQ ID NO: 9. In embodiments, the Tar16 enzyme has a sequence identity of 89% to SEQ ID NO: 9 In embodiments, the Tar16 enzyme has a sequence identity of 90% to SEQ ID NO:9. In embodiments, the Tar16 enzyme has a sequence identity of 91% to SEQ ID NO:9. In embodiments, the Tar16 enzyme has a sequence identity of 92% to SEQ ID NO:9. In embodiments, the Tar16 enzyme has a sequence identity of 93% to SEQ ID NO: 9. In embodiments, the Tar16 enzyme has a sequence identity of 94% to SEQ ID NO: 9. In embodiments, the Tar16 enzyme has a sequence identity of 95% to SEQ ID NO: 9. In embodiments, the Tar16 enzyme has a sequence identity of 96% to SEQ ID NO: 9. In embodiments, the Tar16 enzyme has a sequence identity of 97% to SEQ ID NO: 9. In embodiments, the Tar16 enzyme has a sequence identity of 98% to SEQ ID NO: 9. In embodiments, the Tar16 enzyme has a sequence identity of 99% to SEQ ID NO: 9 In embodiments, the Tar16 enzyme is the sequence of SEQ ID NO:9.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### VI. Embodiments

### P Embodiments

Embodiment P1. A method of making L-4-chlorokynurenine (L-4-Cl-Kyn), the method comprising converting L-tryptophan (L-Trp) to L-4-Cl-Kyn using one or more of Tar14, Tar13, or Tar16.

Embodiment P2. A method of making L-4-Cl-Kyn, the method comprising contacting a microbe with L-Trp, wherein the microbe expresses one or more of Tar14, Tar13, or Tar16, and allowing said microbe to produce L-4-Cl-Kyn from L-Trp.

Embodiment P3. A genetically engineered microbe, wherein the microbe expresses one or more of Tar 14, Tar13, or Tar16.

Embodiment P4. The microbe of any one of embodiments P1-P3, wherein the microbe is a human gastrointestinal microbe.

Embodiment P5. A method of treating a subject having a neurological disorder, the method comprising administering an effective amount of L-4-Cl-Kyn to the subject, thereby treating said neurological disorder.

### Embodiments

Embodiment 1. A genetically engineered microbe, wherein the genetically engineered microbe comprises an exogenous Tar14 encoding nucleic acid, an exogenous Tar13 encoding nucleic acid, or an exogenous Tar16 encoding nucleic acid.

Embodiment 2. The genetically engineered microbe of embodiment 1, wherein the genetically engineered microbe comprises an exogenous Tar 14 enzyme, an exogenous Tar13 enzyme, or an exogenous TarT6 enzyme.

Embodiment 3. A genetically engineered microbe, wherein the genetically engineered microbe comprises one or more of an exogenous Tar14 encoding nucleic acid, an exogenous Tar13 encoding nucleic acid, or an exogenous Tar16 encoding nucleic acid.

Embodiment 4. The genetically engineered microbe of embodiment 3, wherein the genetically engineered microbe comprises one or more of an exogenous Tar 14 enzyme, an exogenous Tar13 enzyme, or an exogenous Tar16 enzyme.

Embodiment 5. The genetically engineered microbe of any one of embodiments 1 to 4, wherein the genetically engineered microbe does not comprise an endogenous Tar14 encoding nucleic acid, an endogenous Tar13 encoding nucleic acid, or an endogenous Tar16 encoding nucleic acid.

Embodiment 6. The genetically engineered microbe of any one of embodiments 1 to 4, wherein the genetically engineered microbe does not comprise one or more of an endogenous Tar14 encoding nucleic acid, an endogenous TarT3 encoding nucleic acid, or an endogenous TarT6 encoding nucleic acid.

Embodiment 7. The genetically engineered microbe of embodiment 6, wherein the genetically engineered microbe does not comprise an endogenous Tar14 encoding nucleic acid.

Embodiment 8. The genetically engineered microbe of embodiment 6, wherein the genetically engineered microbe does not comprise an endogenous Tar13 encoding nucleic acid.

Embodiment 9. The genetically engineered microbe of embodiment 6, wherein the genetically engineered microbe does not comprise an endogenous Tar16 encoding nucleic acid.

Embodiment 10. The genetically engineered microbe of embodiment 6, wherein the genetically engineered microbe does not comprise an endogenous Tar13 encoding nucleic acid or an endogenous Tar14 encoding nucleic acid.

Embodiment 11. The genetically engineered microbe of embodiment 6, wherein the genetically engineered microbe does not comprise an endogenous Tar13 encoding nucleic acid or an endogenous Tar16 encoding nucleic acid.

Embodiment 12. The genetically engineered microbe of embodiment 6, wherein the genetically engineered microbe does not comprise an endogenous Tar14 encoding nucleic acid or an endogenous Tar16 encoding nucleic acid.

Embodiment 13. The genetically engineered microbe of any one of embodiments 1 to 4, wherein the genetically engineered microbe comprises an exogenous nucleic acid that has at least 85% nucleotide identity to SEQ ID NO:11, SEQ ID NO:13, or SEQ ID NO:17.

Embodiment 14. The genetically engineered microbe of embodiment 3 or 4, wherein the genetically engineered microbe comprises one or more of an exogenous nucleic acid having at least 85% nucleotide identity to SEQ ID NO:11, SEQ ID NO:13, or SEQ ID NO:17.

Embodiment 15. The genetically engineered microbe of embodiment 13 or 14, wherein the exogenous nucleic acid has at least 85% nucleotide identity to SEQ ID NO:11.

Embodiment 16. The genetically engineered microbe of embodiment 13 or 14, wherein the exogenous nucleic acid has at least 85% nucleotide identity to SEQ ID NO:13.

Embodiment 17. The genetically engineered microbe of embodiment 13 or 14, wherein the exogenous nucleic acid has at least 85% nucleotide identity to SEQ ID NO:17.

Embodiment 18. The genetically engineered microbe of any one of embodiments 1 to 17, wherein the genetically engineered microbe comprises an exogenous Flavin reductase encoding nucleic acid.

Embodiment 19. The genetically engineered microbe of any one of embodiments 1 to 18, wherein the genetically engineered microbe comprises an exogenous Flavin reductase.

Embodiment 20. The genetically engineered microbe of any one of embodiments 1 to 19, wherein the microbe comprises an exogenous Tar15 encoding nucleic acid.

Embodiment 21. The genetically engineered microbe of any one of embodiments 1 to 20, wherein the genetically engineered microbe comprises an exogenous Tar15 enzyme.

Embodiment 22. The genetically engineered microbe of embodiment 20, wherein the exogenous Tar15 encoding nucleic acid has at least 85% nucleotide identity to SEQ ID NO:15.

Embodiment 23. The genetically engineered microbe of any one of embodiments 1 to 12, wherein the encoding nucleic acid has at least 85% nucleotide identity to SEQ ID NO:10, SEQ ID NO:12, or SEQ ID NO:16.

Embodiment 24. The genetically engineered microbe of any of embodiments 1 to 23, wherein the exogenous Tar14 encoding nucleic acid, exogenous Tar13 encoding nucleic acid, or exogenous Tar16 encoding nucleic acid further comprises an exogenous promoter.

Embodiment 25. The genetically engineered microbe of embodiment 24, wherein the exogenous promoter is BG51, Pfer, Ptac, Pem7, arcB, aroF, glk, mqsR, recA, rpoS, rpsU, or sigX.

Embodiment 26. The genetically engineered microbe of any one of embodiments to 22, wherein the exogenous Tar15 encoding nucleic acid further comprises an exogenous promoter.

Embodiment 27. The genetically engineered microbe of embodiment 26, wherein the exogenous promoter is BG51, Pfer, Ptac, Pem7, arcB, aroF, glk, mqsR, recA, rpoS, rpsU, or sigX.

Embodiment 28. The genetically engineered microbe of any one of embodiments 1 to 27, wherein the microbe is a gram negative bacterium.

Embodiment 29. The genetically engineered microbe of embodiment 28, wherein the gram negative bacterium is E. coli or P. putida.

Embodiment 30. The genetically engineered microbe of any one of embodiments 1 to 28, wherein the microbe is a human gastrointestinal microbe.

Embodiment 31. A method of producing L-4-Cl-Kyn comprising contacting the genetically engineered microbe of any one of embodiments 1 to 30 with L-tryptophan.

Embodiment 32. The method of embodiment 31, comprising isolating L-4-Cl-Kyn from cells.

Embodiment 33. A genetically engineered microbe, wherein the genetically engineered microbe comprises a nucleic acid encoding for an exogenous tryptophan halogenase.

Embodiment 34. The genetically engineered microbe of embodiment 33, wherein the genetically engineered microbe comprises an exogenous tryptophan halogenase.

Embodiment 35. The genetically engineered microbe of embodiment 34, wherein the exogenous tryptophan halogenase is ClaH, AbeH, PyrH, ThdH, Th-Hal, SttH, KtzR, BorH, KtzQ, PmA, RebH, or AtmH.

Embodiment 36. The genetically engineered microbe of any one of embodiments 33 to 34, wherein the exogenous tryptophan halogenase has at least 85% identity to SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44.

Embodiment 37. The genetically engineered microbe of any one of embodiments 33 to 34, wherein the encoded nucleic acid comprises at least on optimized codon.

Embodiment 38. The genetically engineered microbe of any one of embodiments 33 to 37, wherein the nucleic acid encoding the exogenous tryptophan halogenase further comprises an exogenous promoter.

Embodiment 39. The genetically engineered microbe of embodiment 38, wherein the exogenous promoter is BG51, Pfer, Ptac, Pem7, arcB, aroF, glk, mqsR, recA, rpoS, rpsU, or sigX.

Embodiment 40. The genetically engineered microbe of any one of embodiments 33 to 39, wherein the microbe is a gram negative bacterium.

Embodiment 41. The genetically engineered microbe of embodiment 40, wherein the gram negative bacterium is E. coli or P. putida.

Embodiment 42. The genetically engineered microbe of any one of embodiments 33 to 40, wherein the microbe is a human gastrointestinal microbe.

Embodiment 43. A method of synthesizing L-4-Cl-Kyn, said method comprising contacting L-Trp with a Tar14 enzyme, a Tar13 enzyme, and a Tar16 enzyme.

Embodiment 44. The method of embodiment 43, further comprising a Flavin reductase.

Embodiment 45. The method of embodiment 44, wherein the Flavin reductase is Tar15 enzyme.

Embodiment 46. An isolated nucleic acid, said isolated nucleic acid comprising a Tar14 encoding nucleic acid, a Tar13 encoding nucleic acid, a Tar16 encoding nucleic acid, or a Tar15 nucleic acid.

Embodiment 47. An isolated nucleic acid, said isolated nucleic acid comprising one or more of a Tar14 encoding nucleic acid, a Tar13 encoding nucleic acid, a Tar6 encoding nucleic acid, or a Tar15 nucleic acid.

Embodiment 48. The isolated nucleic acid of embodiment 46, wherein said isolated nucleic acid has at least 85% nucleotide identity to SEQ ID NO:11, SEQ ID NO:13, SEQ ID NO:15, or SEQ ID NO:17.

Embodiment 49. The isolated nucleic acid of embodiment 47, comprising one or more sequences having at least 85% nucleotide identity to SEQ ID NO: 11, SEQ ID NO:13, SEQ ID NO:15, or SEQ ID NO:17.

Embodiment 50. The isolated nucleic acid of any one of embodiments 46 to 48, wherein the isolated nucleic acid comprises at least one optimized codon.

Embodiment 51. An isolated enzyme, said isolated enzyme comprising Tar 14, Tar13, Tar16, or TarT5, or enzymatically active fragment or variant thereof.

Embodiment 52. The isolated enzyme of embodiment 51, wherein said enzyme has at least 85% identity to SEQ ID NO:1, SEQ ID NO:3, SEQ ID NO:6, or SEQ ID NO:8.

### EXAMPLES

### Example 1: Biosynthesis of L-4-Chrlorokynurenine, a Lipopeptide Antibiotic Non-Proteinogenic Amino Acid and Antidepressant Prodrug

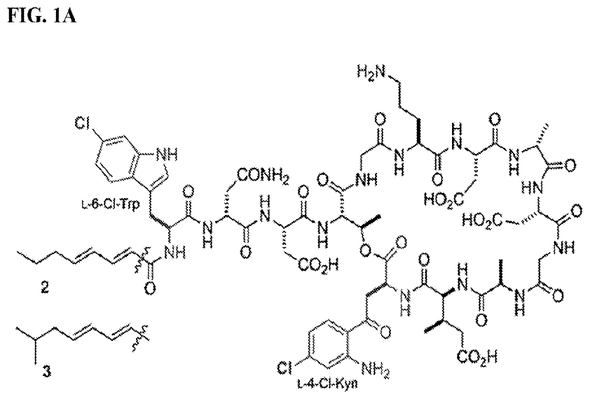

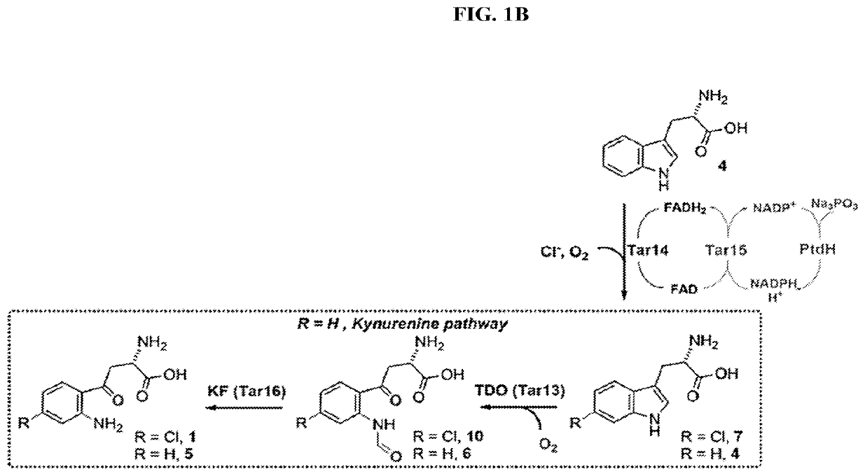

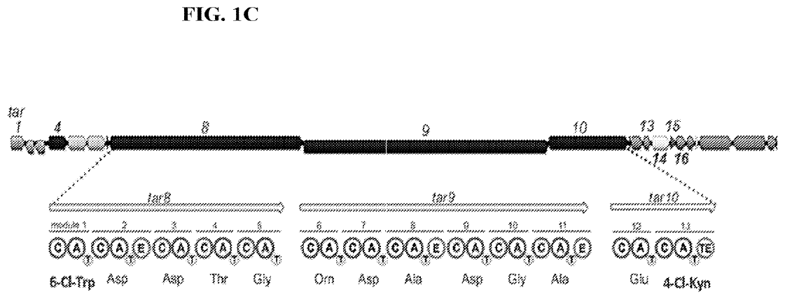

Biologically, L-4-chlorokynurenine (L-4-Cl-Kyn, 1) was recently identified as an amino acid building block in the lipopeptide antibiotics taromycin A (2) and B (3) (FIG. 1A)^{[5,6]} and in the putative glycopeptide antibiotic complex INA-5812.^{[7]} With the concurrent discovery of the taromycin biosynthetic gene cluster (BGC) from the marine actinomycete Saccharomonospora sp. CNQ-490, applicants sought to establish the biosynthetic logic for the bacterial synthesis of L-4-Cl-Kyn. Herein applicants report the concise three-step L-4-Cl-Kyn pathway originating from L-tryptophan (L-Trp, 4) and present it as an orthogonal approach to produce this promising drug candidate.

Enzymatic conversion of L-Trp to L-kynurenine (L-Kyn, 5) is part of the kynurenine pathway (FIG. 1B), the major Trp catabolic pathway in eukaryotes, which leads to vital biochemicals such as the neurotransmitter serotonin and the cofactor NADH.^{[8]} The initial and rate-limiting step of the pathway is catalyzed by the Fe²⁺/heme-dependent enzyme tryptophan-2,3-dioxygenase (TDO) leading to formation of N-formyl-L-Kyn (6), which is hydrolyzed by kynurenine formamidase (KF) to give L-Kyn. Considering the functional importance of the products of this pathway, TDOs and KFs show specificity for L-Trp and N-formyl-L-Kyn, respectively. Homologous enzymes have been identified in some prokaryotes,^{[9]} however, they are not essential for bacterial survival, as bacteria catabolize Trp mainly through non-oxidative degradation.^{[11]} Recently, BGCs with co-clustered TDO-encoding genes have been discovered, but in vitro studies of these enzymes remain limited. Cluster-specific TDOs have been shown to have broader substrate specificities (e.g., the actinomycin TDO AcmG accepts L-Trp, D/L-α-CH₃-Trp, D/L-5-CH₃-Trp, and D/L-5-F-Trp).^{[11-14]} Only one representative of a BGC-associated KF, from actinomycin biosynthesis, has been tested in vitro.^{[15]} This enzyme predominantly worked on N-formyl-L-Kyn, but was also able to deformylate at reduced rates N′,N-α-diformyl-L-Kyn and O-formamino acetophenone. Bioinformatic analysis of the taromycin BGC (tar) identified an unprecedented quartet of enzymes—Tar13, 14, 15, and 16—that show close homology to TDO, flavin-dependent halogenase (FDH), flavin reductase, and KF, respectively, and are encoded by adjacent genes (FIG. 1C).

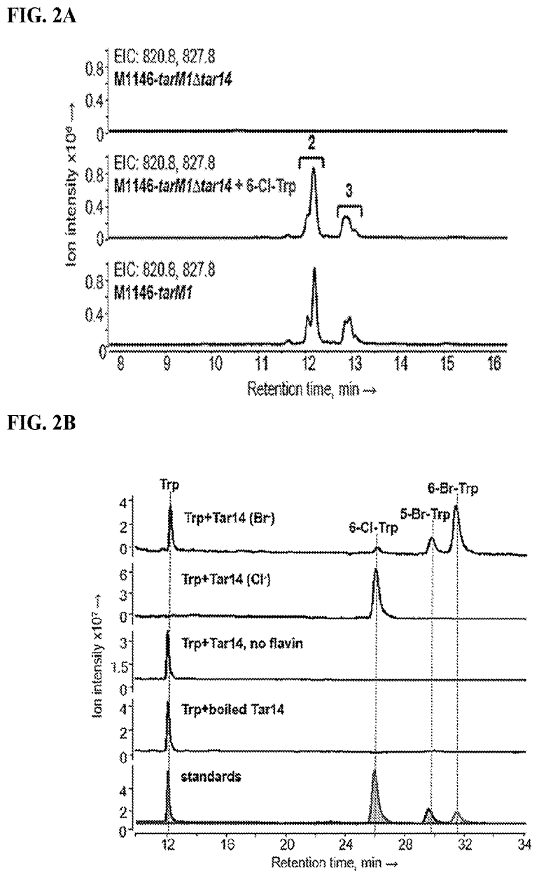

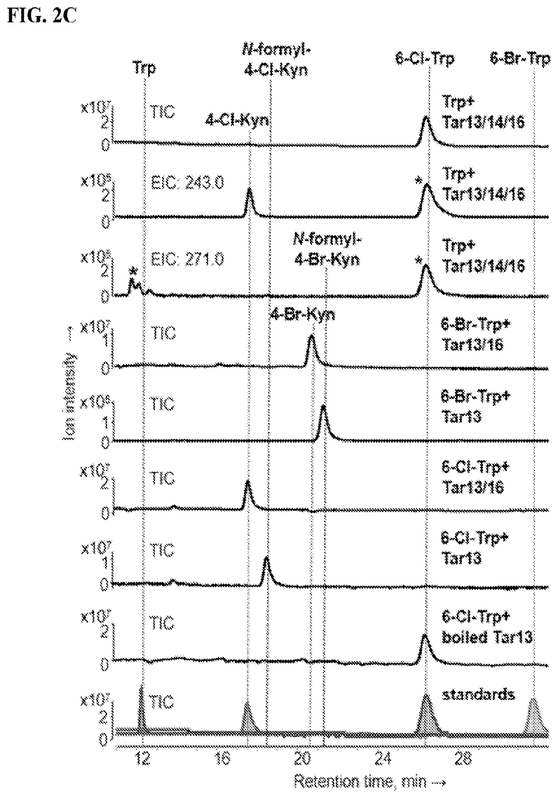

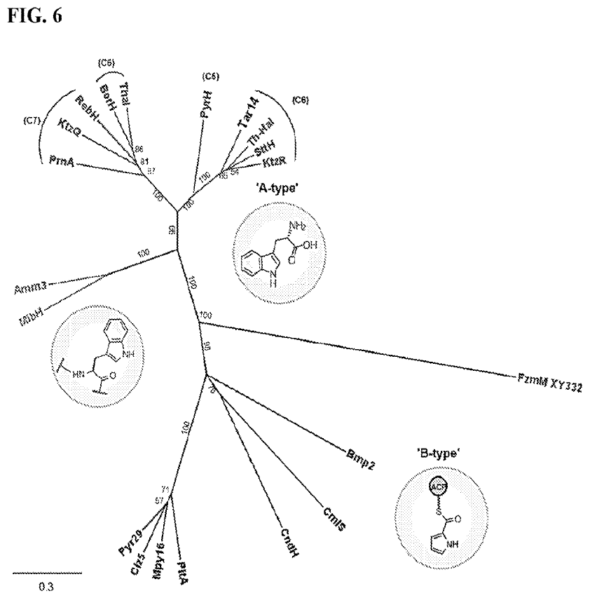

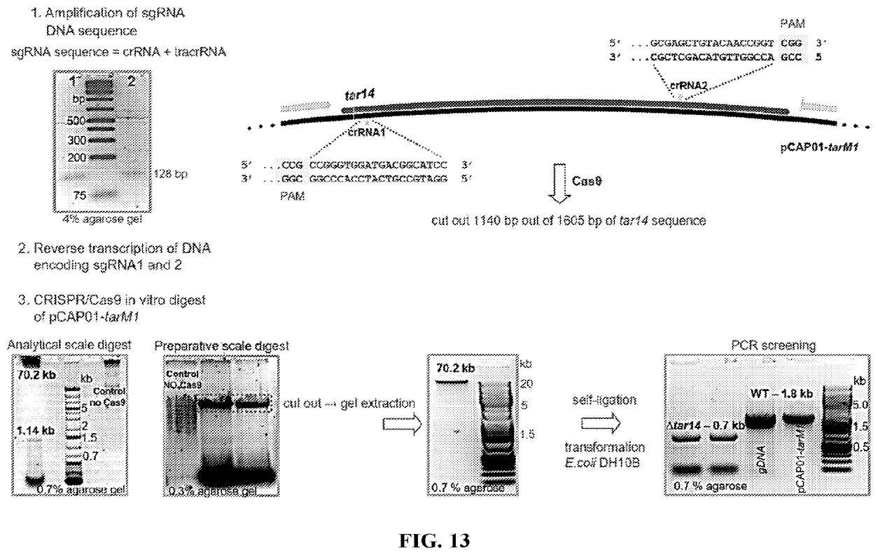

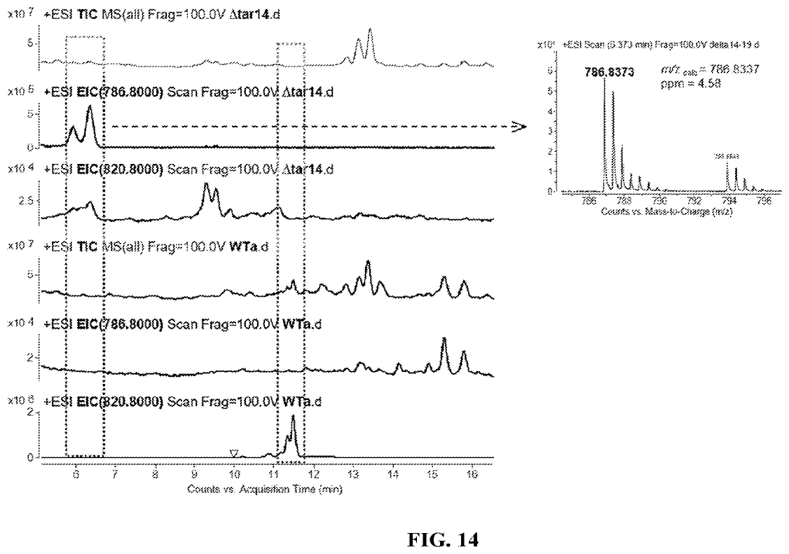

Considering that taromycin contains a second chlorinated amino acid residue, L-6-Cl-Trp (7), and that its BGC encodes only one halogenase, applicants hypothesized that the chlorination reaction takes place directly on L-Trp versus on a carrier protein bound substrate^{[16]} or post-assembly on a released peptide substrate.^{[17]} Phylogenetic analysis of Tar14 showed that it clades with FDHs acting on free L-Trp (FIG. 6). To validate this, applicants performed an in-frame gene deletion of tar14. The availability of the 62.4 kb tar BGC cloned in pCAP01-tarM1^{[6]} allowed rapid genetic interrogations of the pathway. The tar cluster showed instability upon use of recombineering techniques, therefore, applicants chose an in vitro CRISPR/Cas9 approach (FIG. 13).^{[18]} The deletion construct, pCAP01-tarM1Δtar14, was integrated into the genome of Streptomyces coelicolor M1146 for heterologous expression.^{[19]} Liquid chromatography mass spectrometry (LCMS) analysis of the mutant culture extracts revealed abolished production of 2 and 3 (FIG. 2A). Chemical complementation of the mutant strain with 6-Cl-Trp restored taromycin production. These results validated that free L-Trp is the substrate for Tar14, and strongly suggested that L-4-Cl-Kyn is formed by conversion of L-6-Cl-Trp rather than direct halogenation of L-Kyn.

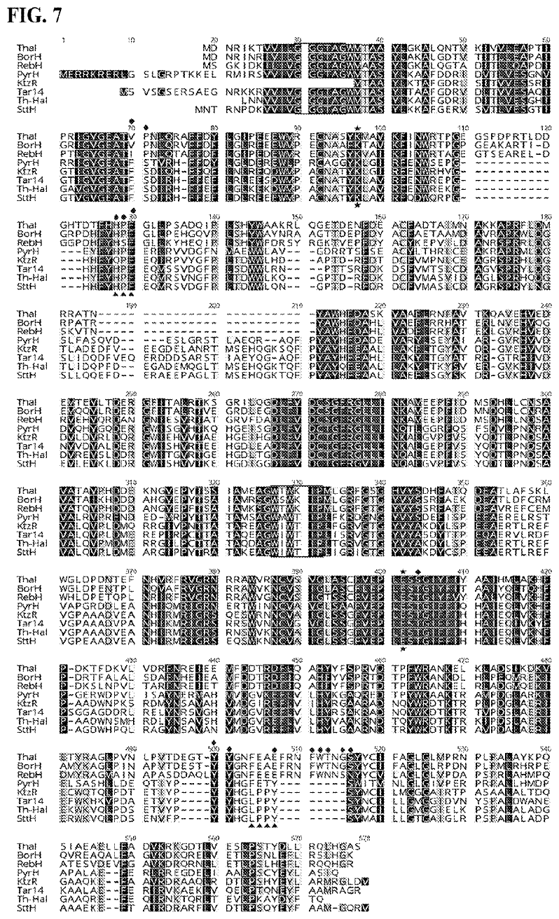

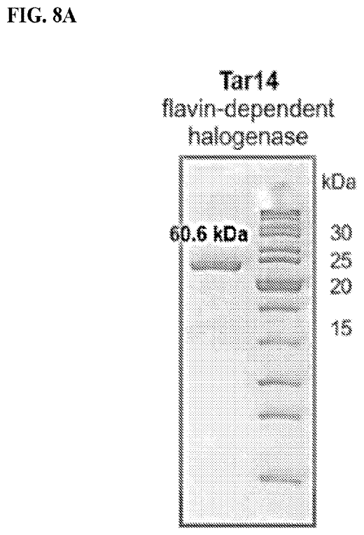

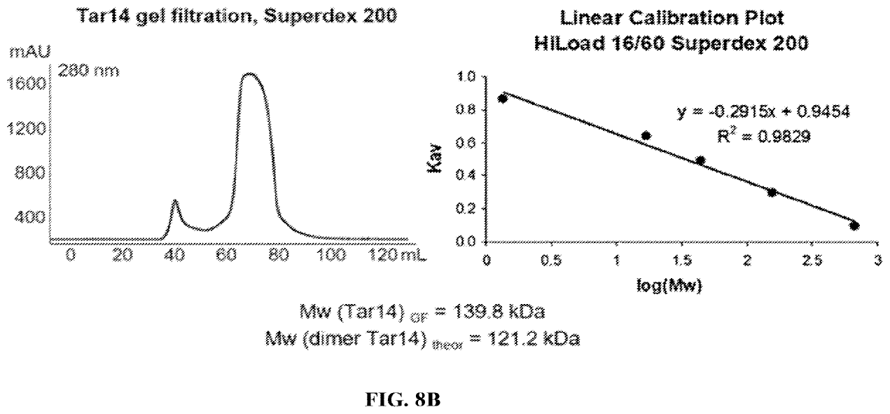

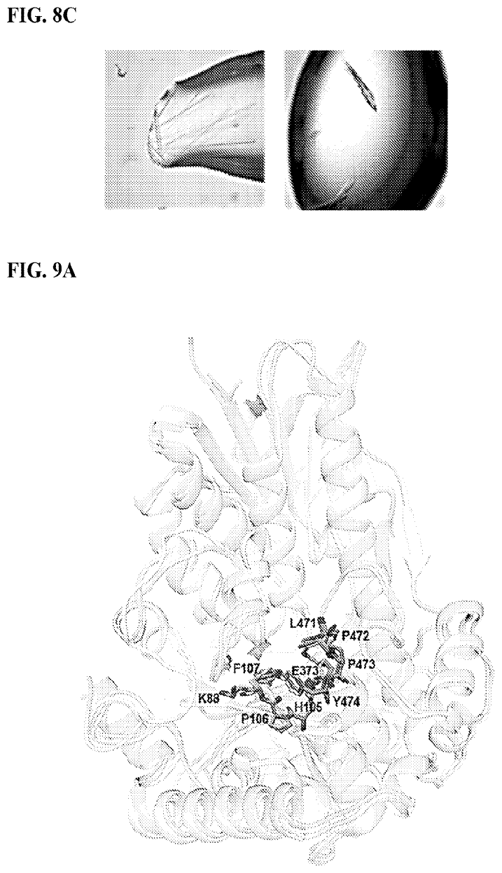

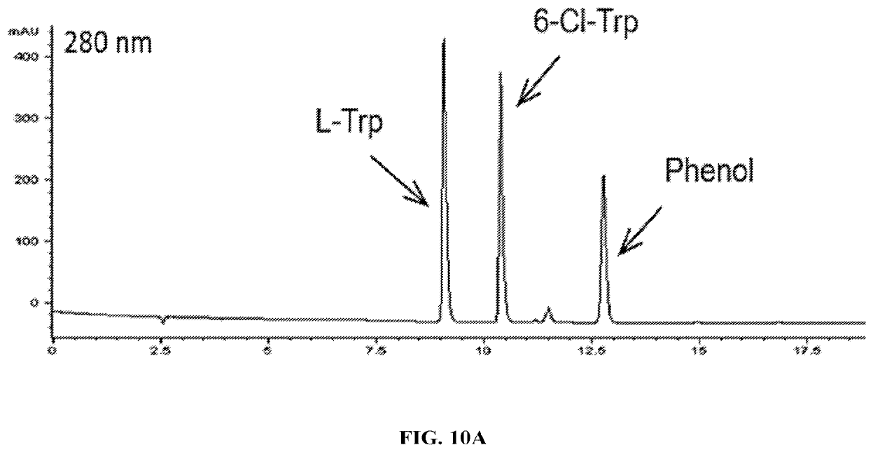

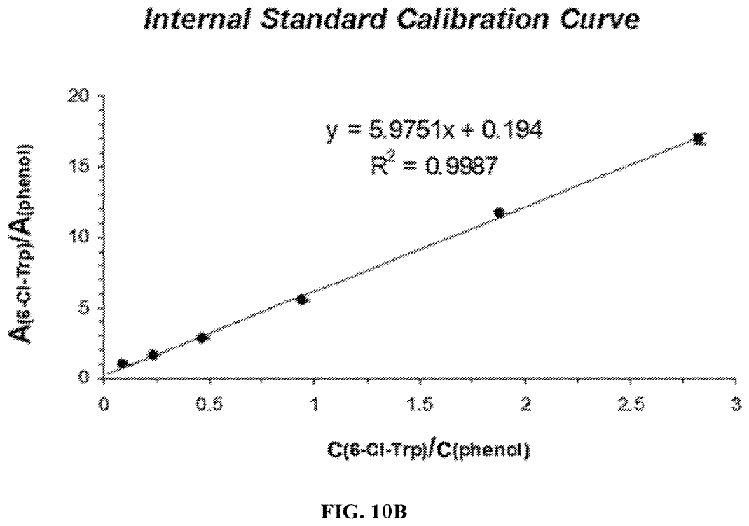

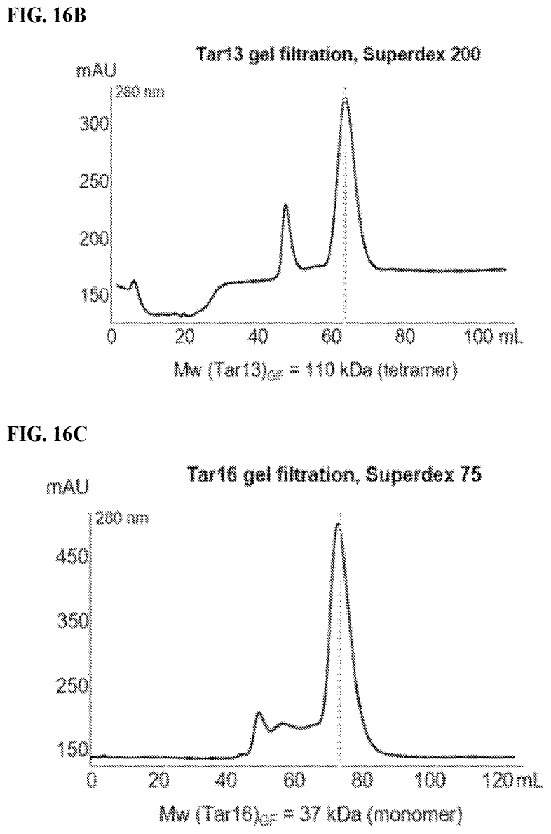

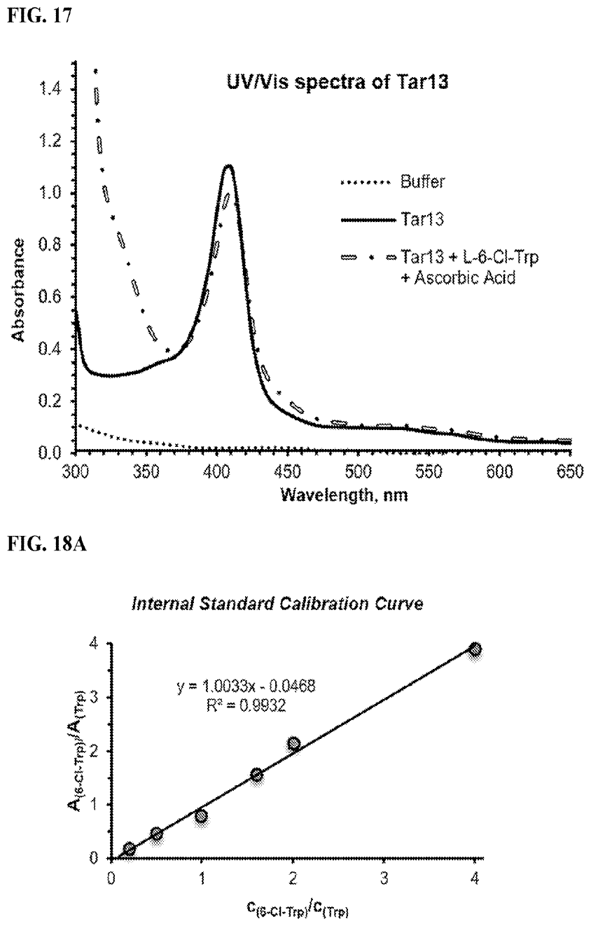

Next, applicants proceeded with the in vitro reconstitution of L-Trp to L-4-Cl-Kyn. Genes encoding Tar13, 15, and 16 were individually cloned and expressed in Escherichia coil (FIGS. 16A-16C), while Tar14 was expressed in S. coelicolor CH999 (FIGS. 8A-8C).^{[20]} However, no yellow color associated with flavin binding was observed for Tar14, suggestive of either enzyme misfolding or a weak binding affinity.^{[17,21]} Upon incubation of Tar14 with flavin and a flavin reductase system (FIG. 2B),^{[22]} applicants observed conversion of L-Trp to L-6-Cl-Trp as confirmed by high resolution mass spectrometry (HRMS) and NMR characterization of the purified product (Supplementary Information). Kinetic parameters of Tar14 with L-Trp were determined: k_{cat}=0.4 min⁻¹, K_{M}=12 μM, k_{cat}/K_{M}=0.03 min⁻¹ M⁻¹, and are consistent with other characterized FDHs (FIGS. 10A, 10B, 11, 12A and 12B, Table 1).^{[23]}

TABLE 1
Kinetic parameters for Tar14 determined
in present study and for other FDHs.^{[16]}

| Enzyme | k_{cat}, min⁻¹ | K_{M}, μM | k_{cat}/K_{M}, min⁻¹ μM⁻¹ |
|---|---|---|---|
| Tar14 | 0.42 ± 0.05 | 12.0 ± 1.9 | 0.04 ± 0.01 |
| Th-Hal (30° C.) | 4.3 ± 0.5 | 12.2 ± 1.8 | 0.35 ± 0.07 |
| Th-Hal (45° C.) | 5.1 ± 0.4 | 20.4 ± 1.3 | 0.25 ± 0.03 |
| SttH | 1.7 ± 0.1 | 25.3 ± 3.2 | 0.07 ± 0.01 |
| RebH | 0.6 ± 0.1 | 28.7 ± 1.3 | 0.02 ± 0.004 |
| PyrH | 2.4 ± 0.4 | 15.2 ± 4.2 | 0.16 ± 0.05 |
| PrnA | 1.1 ± 0.1 | 20.7 ± 0.1 | 0.05 ± 0.005 |
| KtzR | 0.4 ± 0.1 | 34.1 ± 2.1 | 0.01 ± 0.003 |

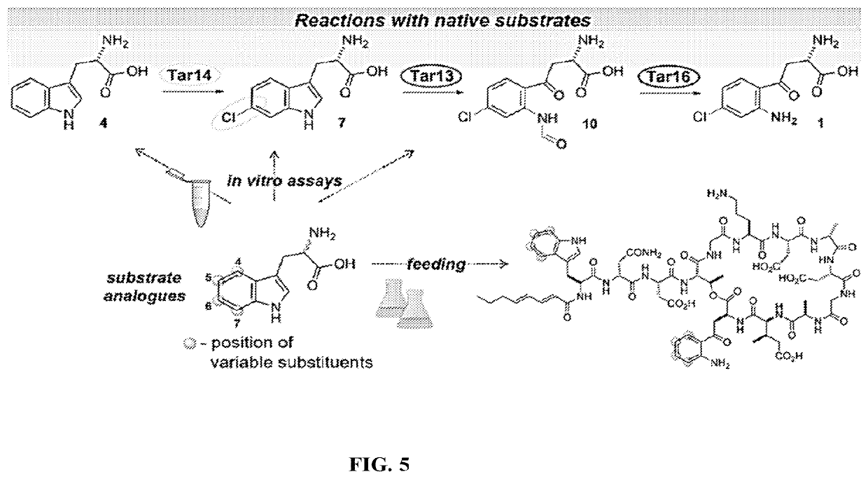

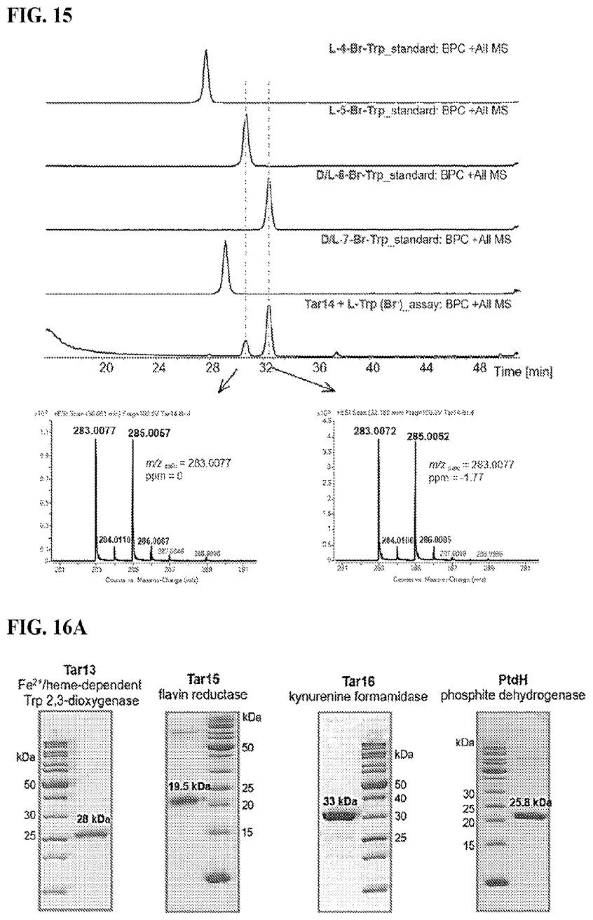

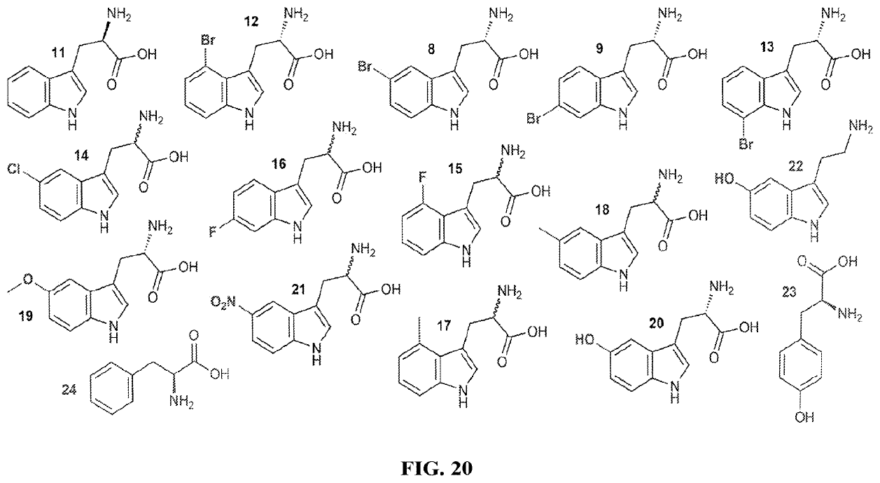

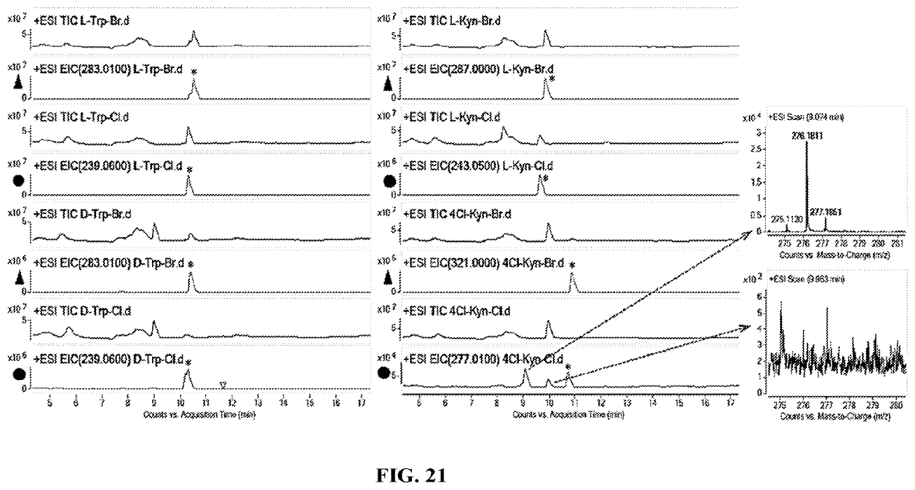

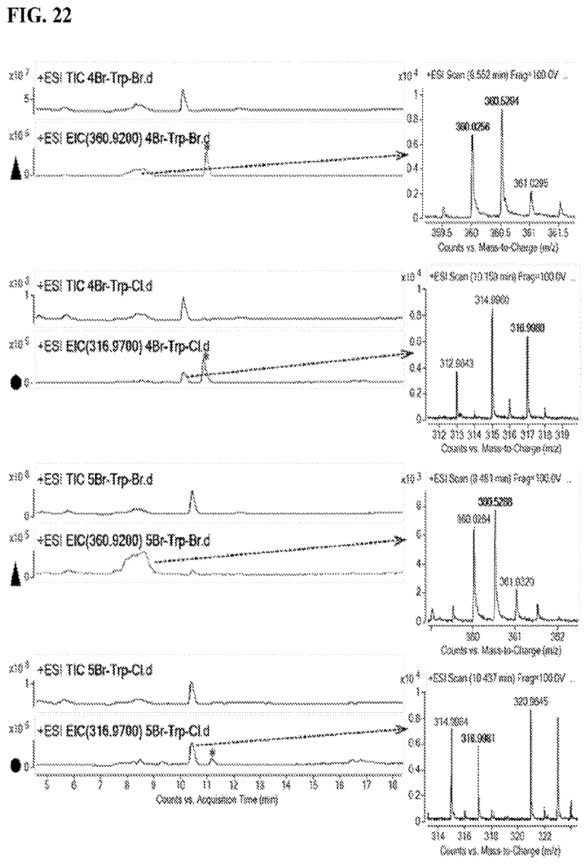

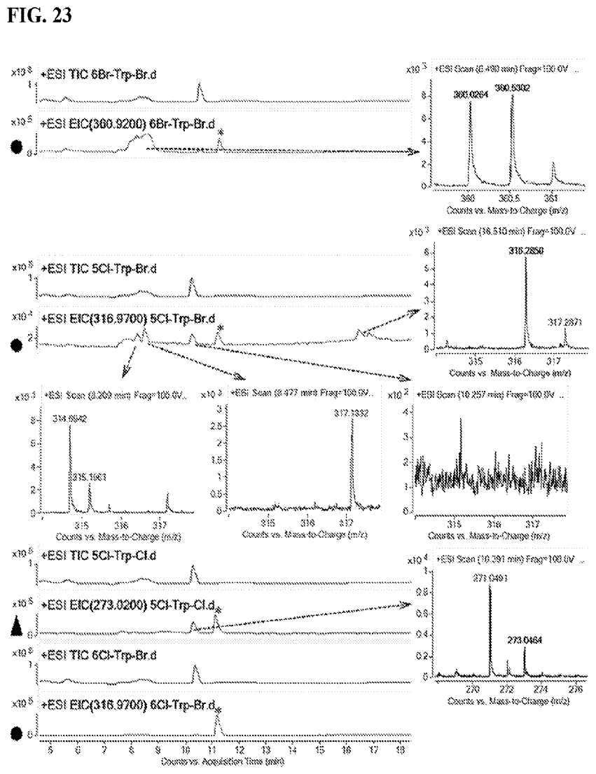

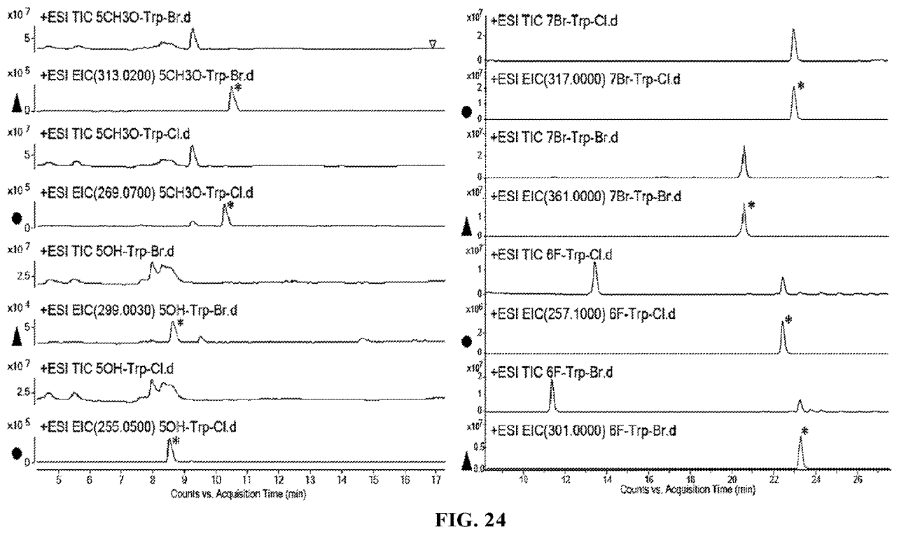

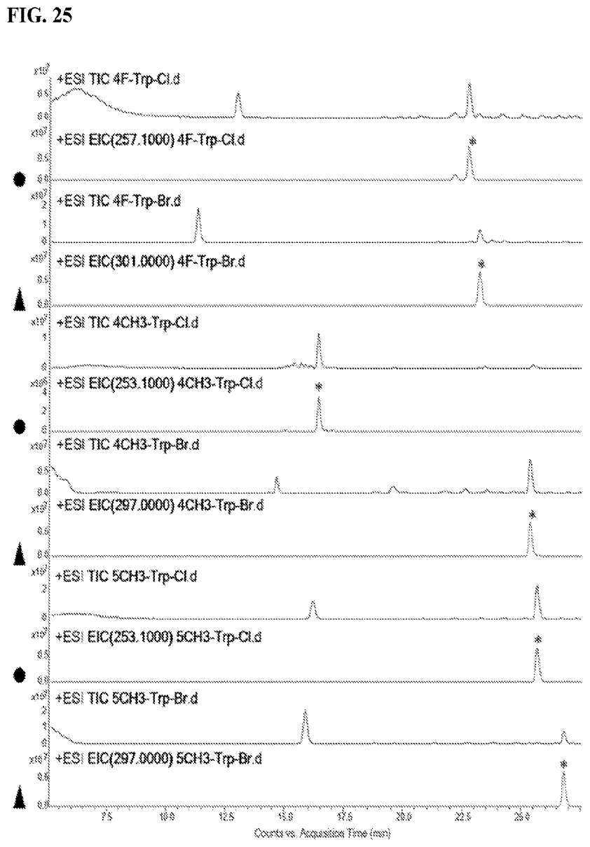

Applicants next probed the substrate specificity of Tar14 towards different halide partners (FIG. 2B, FIG. 5, Table 2). Tar14 readily brominated L-Trp, however, it exhibited an erosion of regiospecificity, producing a 1:5 mixture of L-5-Br-Trp (8) and L-6-Br-Trp (9) as determined by HRMS and retention time comparison with commercial standards (FIG. 2B, FIG. 15). No iodinated tryptophan products were detected when incubated with iodide. To further investigate its biocatalytic halogenation potential, Tar14 was interrogated with a library of Trp derivatives (FIG. 5, Table 2, FIGS. 20-25, Table 8). Tar14 was capable of generating a wide variety of dihalogenated Trp species by accepting monohalogenated substrates. Previously, only the kutzneride FDH KtzR was shown to have L-7-Cl-Trp as its preferred substrate yielding L-6,7-diCl-Trp,^{[24]} while FDH KrmI from a sponge metagenome was able to chlorinate L-7-F-Trp,^{[25]} albeit at trace level.

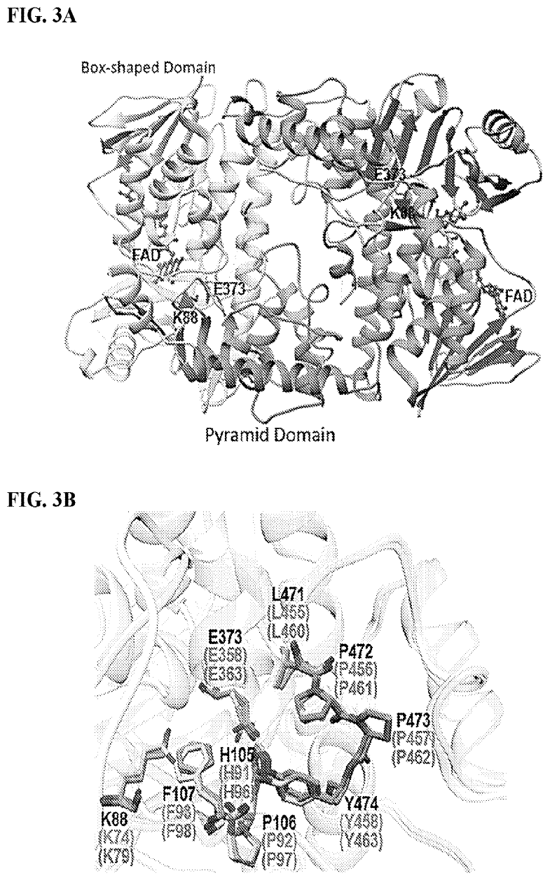

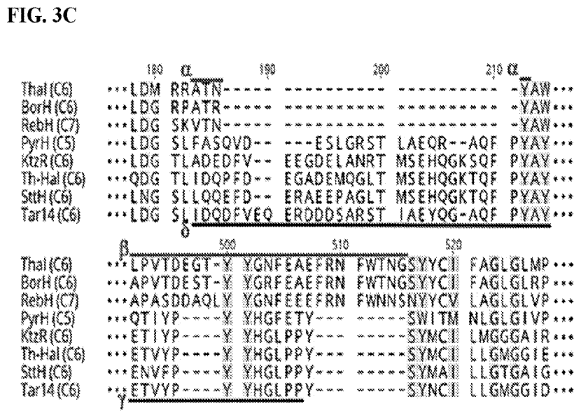

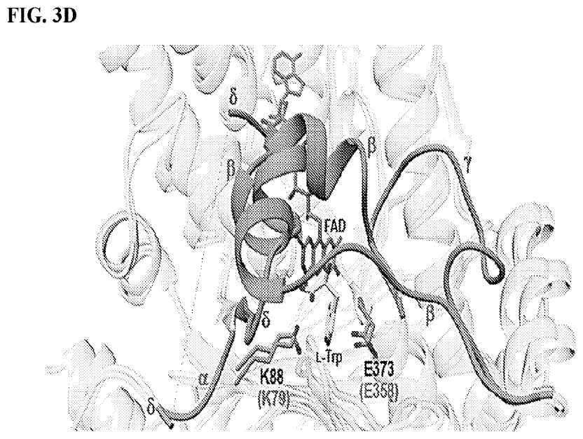

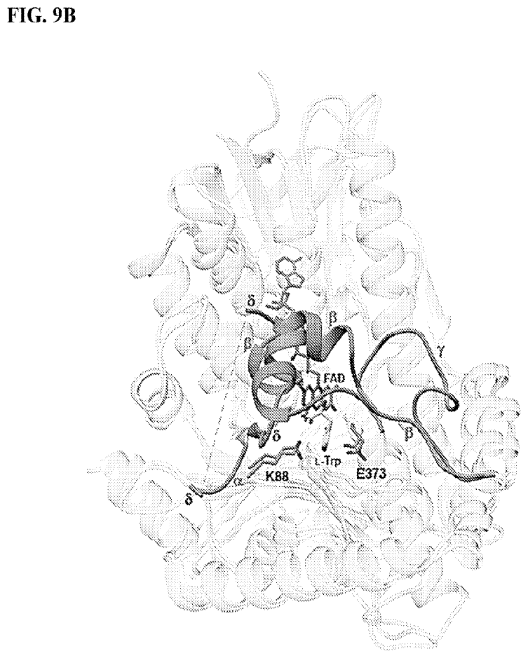

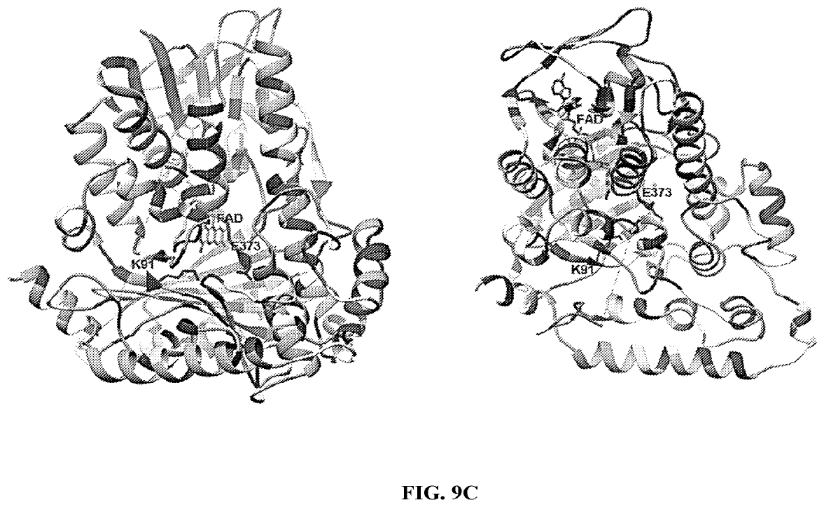

Applicants solved the X-ray crystal structure of Tar14 with bound flavin cofactor to a resolution of 1.74 Å (PDB: 6NSD) using the structure of C6 Trp halogenase Th-Hal (PDB: 5LV9, 73% identity)^{[23]} as a molecular replacement model (Table 7). Two protomers were observed in the asymmetric unit, consistent with the homodimeric form of Tar14 in solution (FIGS. 8A-8C). Each monomer adopts the classical FDH fold comprised of pyramid-like and box-shaped domains (FIG. 3A)^{[21]} Catalytic lysine and glutamate residues identified for all characterized FDHs, remain conserved in Tar14. Bioinformatic analysis of Tar14 and other characterized Trp FDHs clearly shows existence of two sub-types of C6 halogenases: ThaI^{[26]} and BorH^{[27]} are closely related to C7 Trp halogenases, while Tar14, together with SttH,^{[28]} Th-Hal,^{[23]} and KtzR,^{[24]} forms a separate clade on the phylogenetic tree, and shows more sequence and structural similarity to C5 Trp halogenase PyrH^{[29]} (FIGS. 6, 7, 9A and 9B, Table 3). Superimposition of Tar14 with related Th-Hal and SttH structures shows conservation of proposed active site residues (FIG. 3B). However, structural superimposition with the phylogenetically distinct ThaI reveals that active site of these two enzymes are formed by different protein regions (FIG. 3): residues of L-Trp interacting region β of ThaI do not have equivalents in Ta 4, while Tar4 residues of regions γ and δ, positioned in the vicinity of the proposed active site, are missing in ThaI. In light of the observed substrate flexibility of Tar14, applicants looked for distinct structural and sequence features of Tar14 in comparison to SttH. Th-Hal, and KtzR. Sequence alignment superimposed onto the Tar14 structure showed that the putative substrate binding region remains conserved among these enzymes, and amino acid variations in Tar14 are distantly located from the putative active site (FIG. 9C).

TABLE 2
Evaluation of _{L}-4-Cl-Kyn biosynthesis enzymes against
a panel of non-native substrate analogues.

| | Enzyme assays, % conversion | | | | Feeding, % incorporation * | |
|---|---|---|---|---|---|---|
| | Tar14, | Tar14, | | | A₁ | A₁ + A₁₃ |
| Substrate | Cl⁻ | Br⁻ | Tar13 | Tar16^{#} | domain | domain |
| 1, L-4-Cl-Kyn | <1 | 5 | nt | nt | nt | nt |
| 4, L-Trp | 100 | 100 | 15 | 100 | — | <5 |
| 5, L-Kyn | 30 | 30 | nt | nt | nt | nt |
| 7, L-6-Cl-Trp | — | <5 | 100 | 100 | — | 100 |
| 8, L-5-Br-Trp | <1 | — | <1 | 100 | 50 | — |
| 9, L-6-Br-Trp | — | <1 | 100 | 100 | — | 20 |
| 11, D-Trp | 15 | 20 | <1 | 40 | — | — |
| 12, L-4-Br-Trp | <1 | <5 | — | nt | — | — |
| 13, L-7-Br- Trp | 100 | 100 | — | nt | <5 | — |
| 14, D/L-5-Cl-Trp | <1 | <1 | <1 | 100 | 30 | — |
| 15, D/L-4-F-Trp | 10 | 15 | — | nt | 15 | 15 |
| 16, D/L-6-F-Trp | 25 | 25 | 5 | 100 | <5 | 90 |
| 17, D/L-4-CH₃-Trp | 100 | 75 | — | nt | 40 | — |
| 18, D/L-5-CH₃-Trp | 50 | 25 | <1 | 70 | 35 | — |
| 19, L-5-CH₃O-Trp | <1 | <5 | <1 | 100 | 15 | — |
| 20, L-5-OH-Trp | 5 | <1 | — | | 10 | — |
| 21, D/L-5-NO₂-Trp | — | — | — | | 60 | — |
| 22, serotonine | — | — | — | nt | | |
| 23, L-tyrosine | — | — | nt | | nt | nt |
| 24, L-phenylalanine | — | — | nt | | | |

nt—not tested;
— no activity;
^{#}substrates generated in situ by Tar13;
*judged by comparison to taromycin yields by S. coelicolor M1146-tarM1.

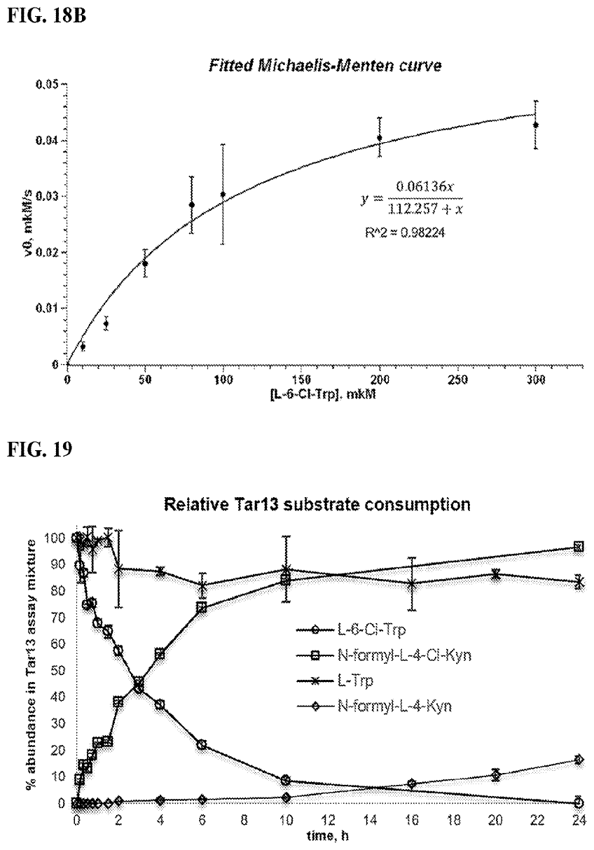

Applicants next examined the activity of TDO Tar113. Incubation of Tar13 with L-6-Cl-Trp (7) resulted in its complete conversion into a new early eluting compound (FIG. 2C). HRMS and NMR data confirmed that the new peak corresponded to N-formyl-L-4-Cl-Kyn (10) (Supplementary Information). Kinetic parameters of Tar13 with L-6-Cl-Trp were determined: k_{cat}=0.03 s⁻¹, K_{M}=12.3 μM, k_{cat}/K_{M}=0.27 min⁻¹ mM⁻¹ (FIGS. 18A and 18B). Upon mildly acidic purification conditions, 10 showed partial deformylation to L-4-Cl-Kyn (1). Therefore, to test the activity of KF Tar16, applicants performed a coupled Tar13/Tar16 assay in which the substrate for Tar16 was generated in situ, as described herein. Addition of Tar6 to the reaction resulted in full conversion of 7 to 1, thus confirming the role of Tar16 in the biosynthesis of 1. Applicants additionally explored the possibility of directly converting L-Trp to L-4-Cl-Kyn in a one-pot reaction. The expected product was detected. The production of L-4-Cl-Kyn was below 1% largely due to the incompability of optimal assay conditions for each individual enzyme in vitro (FIG. 2C, Supplementary Information). The optimal assay conditions for each individual enzyme is optimized.

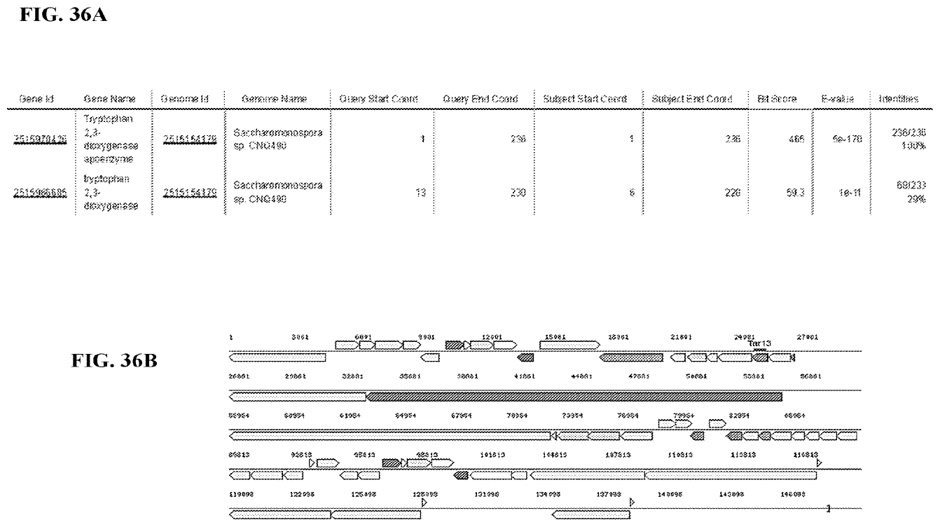

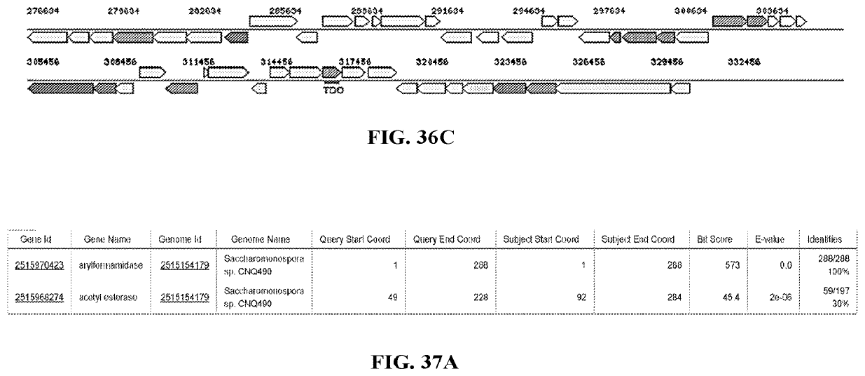

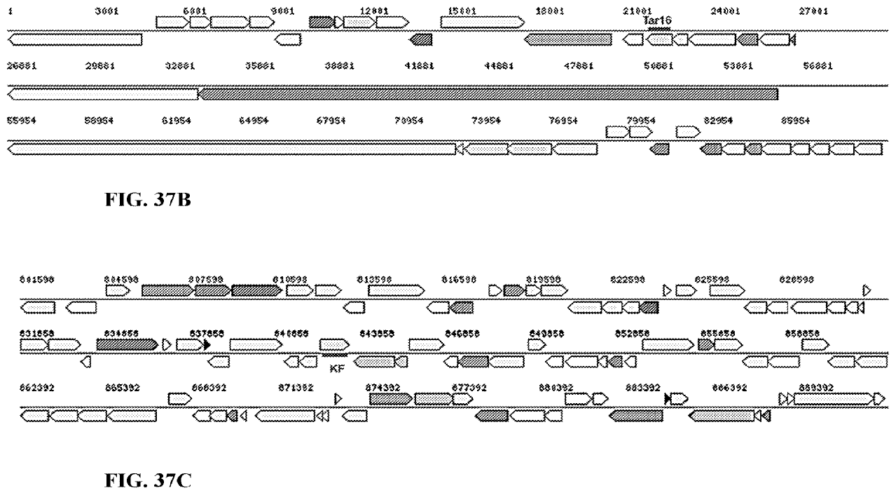

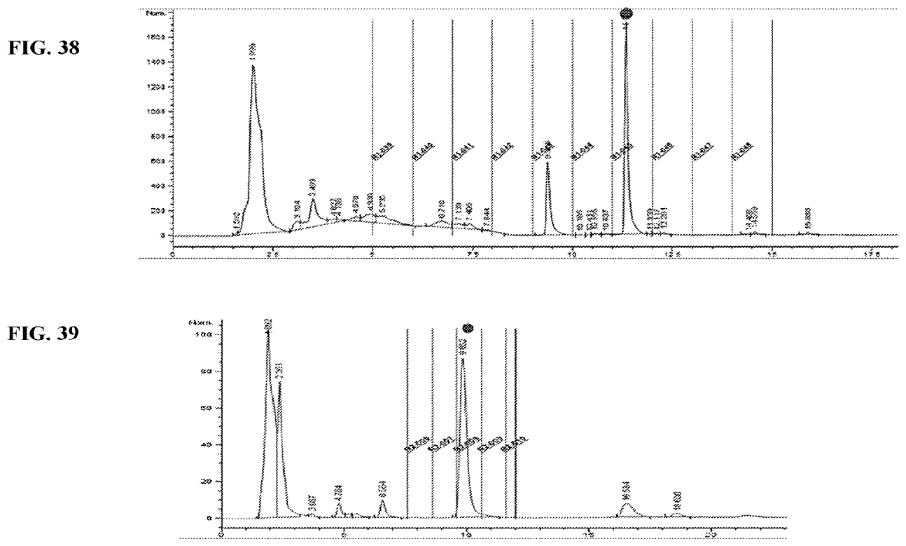

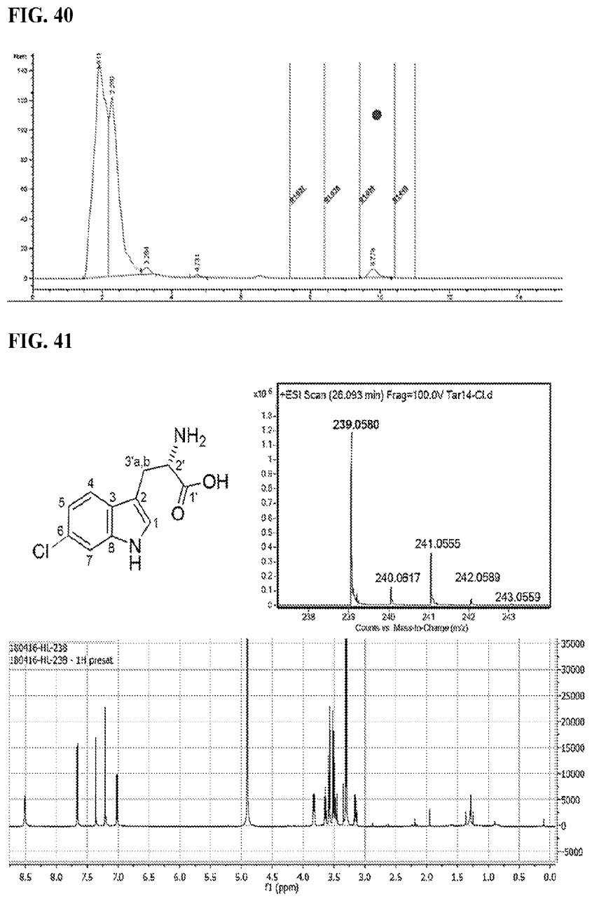

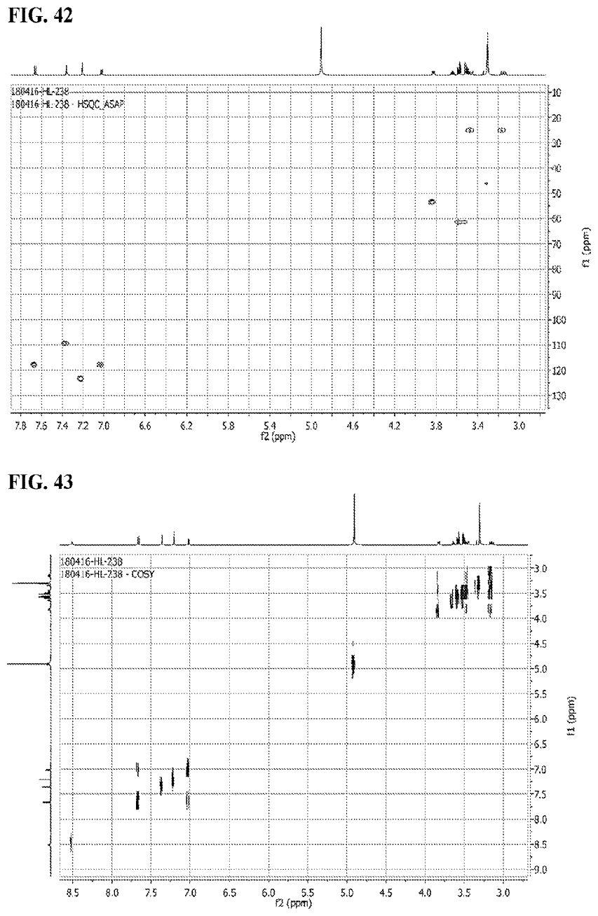

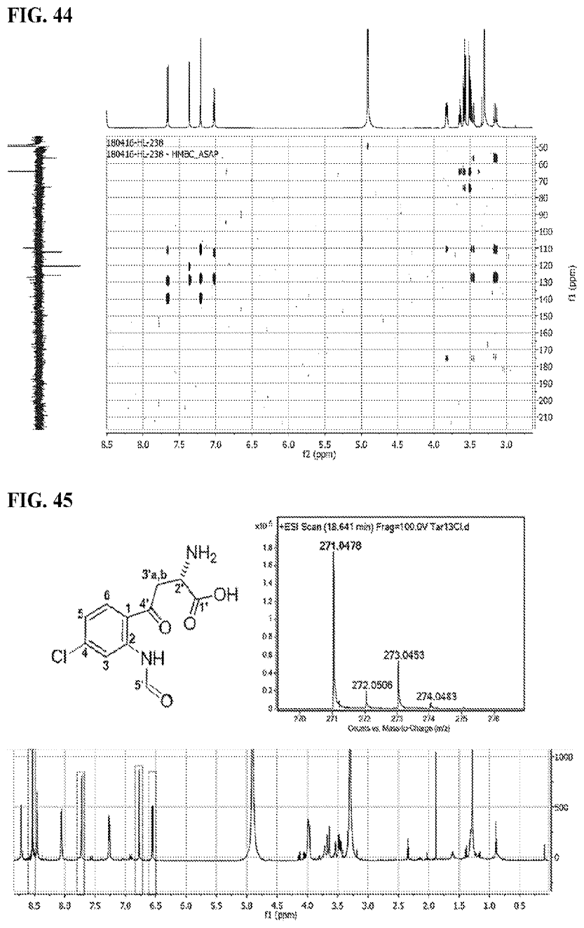

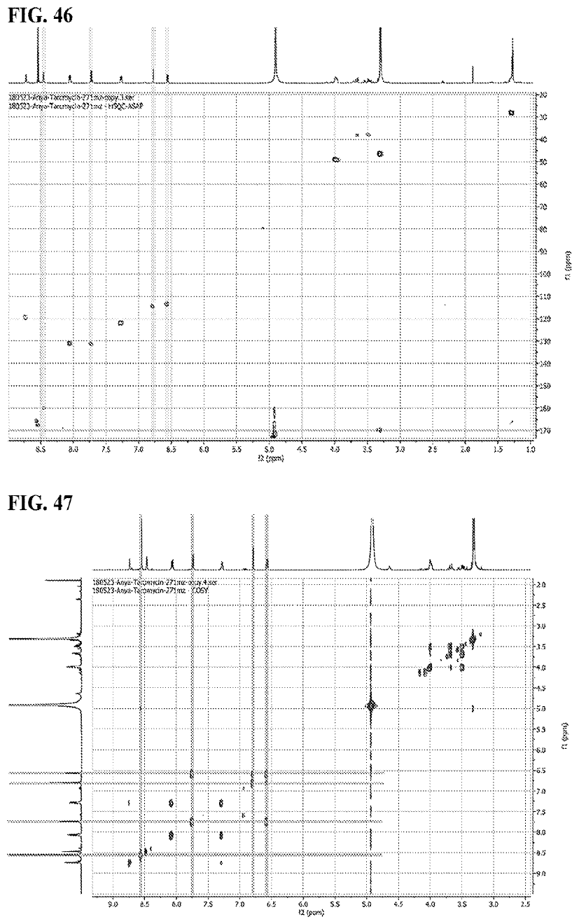

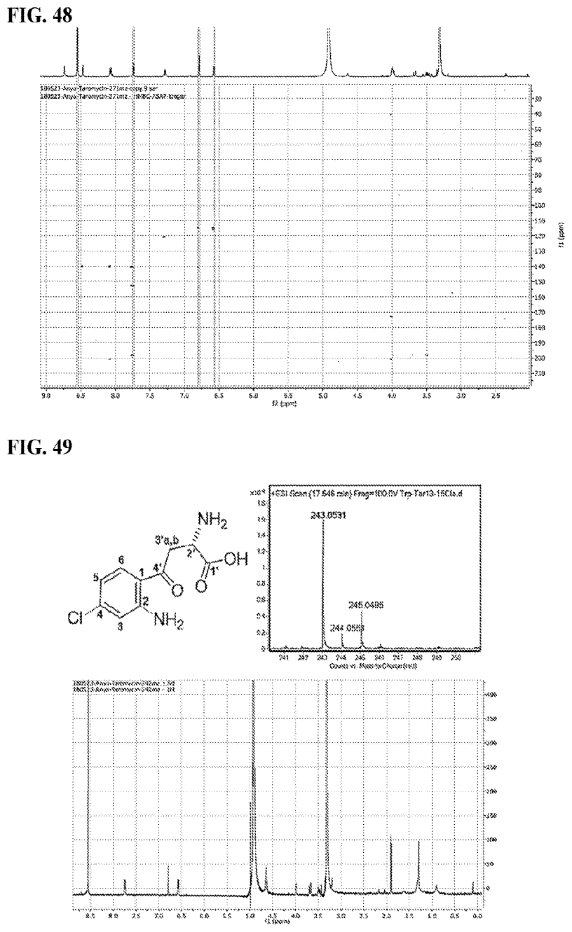

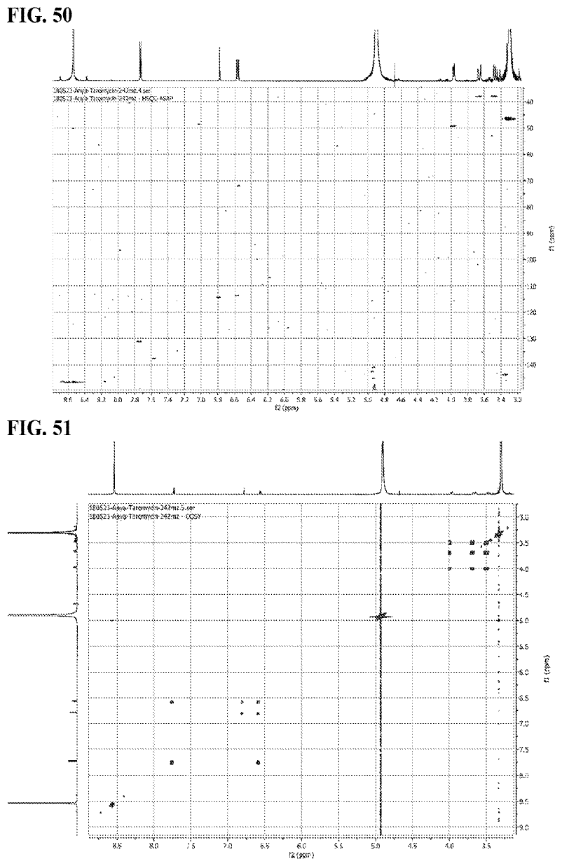

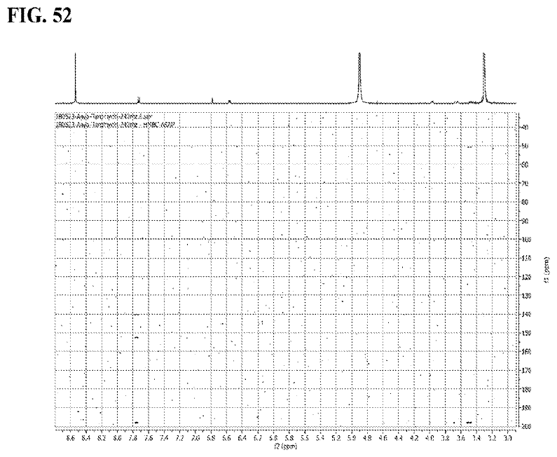

When applicants tested L-Trp as a substrate for Tar13, only trace activity was measured. This stark difference in the substrate preference of Tar13 was illuminated in a comparative in vitro substrate consumption assay of L-Trp versus L-6-Cl-Trp (FIG. 19). Tar13's preference for the halogenated substrate was clearly evident by the slow rate of L-Trp consumption and failure to achieve its complete conversion. Bioinformatic analysis of the draft genome sequence of S. sp. CNQ-490 revealed a second pair of TDO/KF enzymes with less than 30% identity to Tar13/Tar16 and that are likely associated with L-Trp metabolism (FIGS. 36A-36C and 37A-37C). Applicants hypothesize that Tar13 and Tar16 diverged from catabolic TDOs and KFs, respectively, and coevolved to serve a specialized role in L-4-Cl-Kyn biosynthesis after gene duplication. Collectively, results indicate that Tar13 and Tar16 are the first representatives of their enzyme families to prefer chlorinated substrates.

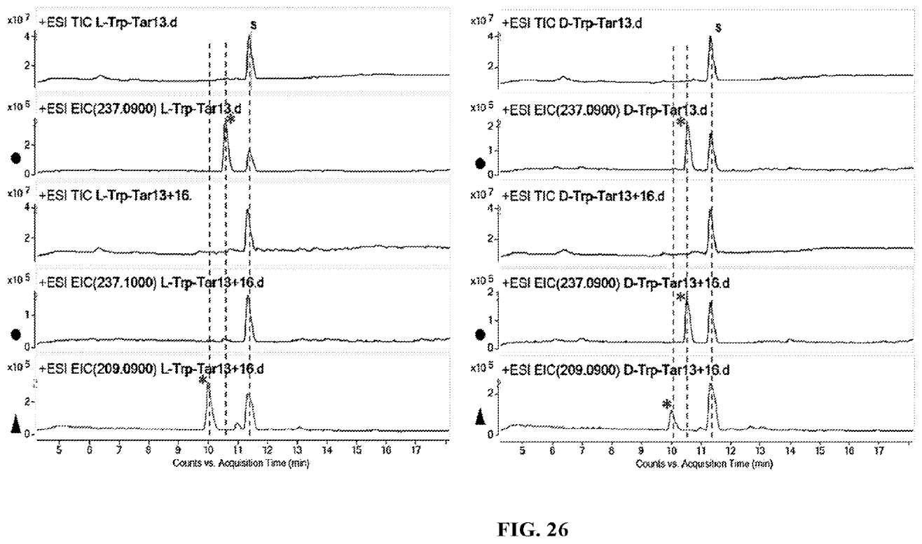

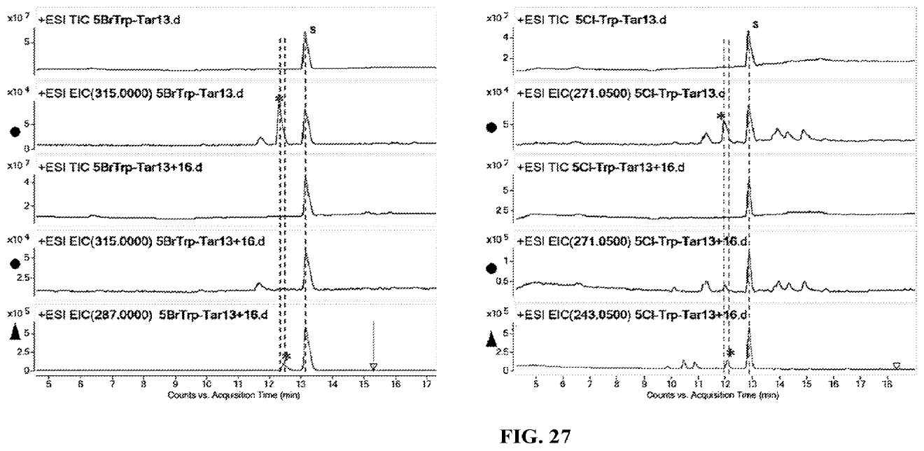

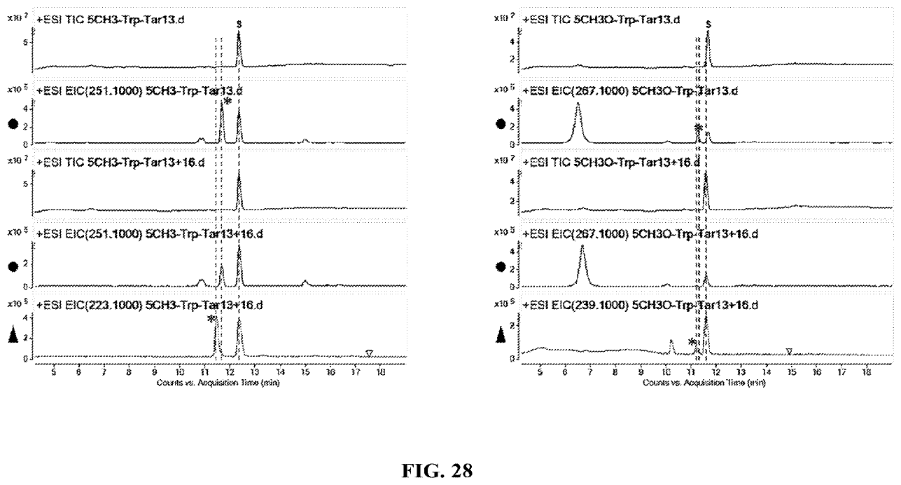

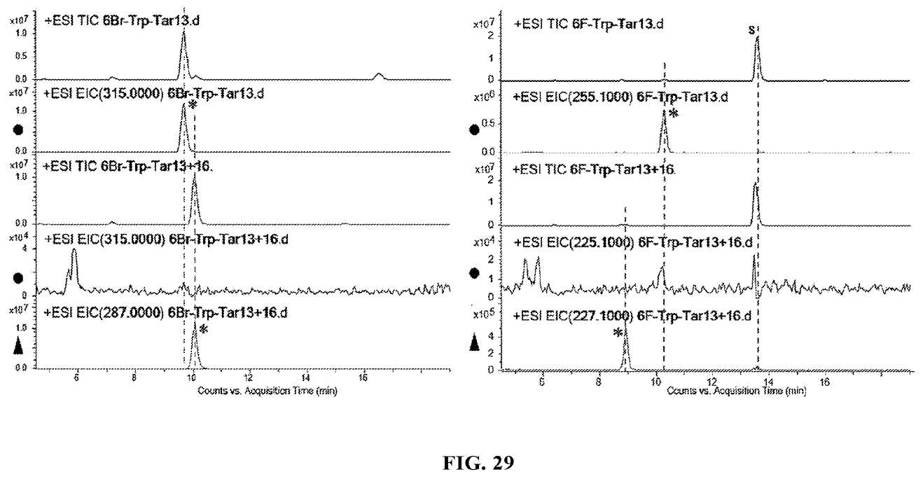

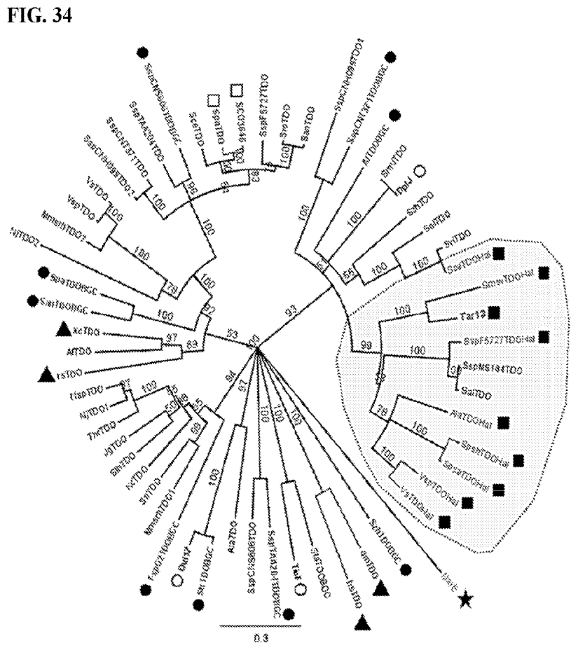

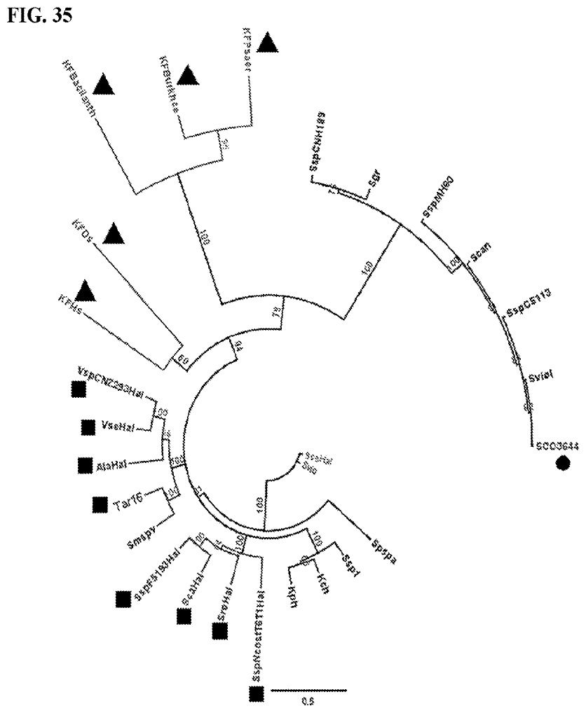

Applicants further probed Tar13 and Tar16 activity with a variety of substrate analogues (FIG. 5, Table 2) and identified a preference of these enzymes towards C6 halogenated Trp derivatives (FIGS. 26-29, Table 8). Phylogenetic analysis revealed that Tar13 and Tar16 are evolutionarily distinct from catabolic TDO and KFs, respectively (FIGS. 34 and 35). Interestingly, they also branch out from other BGC-associated enzymes and form a separate clade with uncharacterized homologs. Inspection of gene neighborhoods of these homologs revealed nonribosomal peptide synthetase (NRPS) BGCs that also contain putative Trp FDH, KF, and adenylation (A) domain with predicted specificity towards Kyn (Table 10). This observation suggests halogenated kynurenine residues may be more widespread than previously thought in peptide natural products, and that Tar13/Tar16 sequences may be convenient “search hooks” for their discovery.

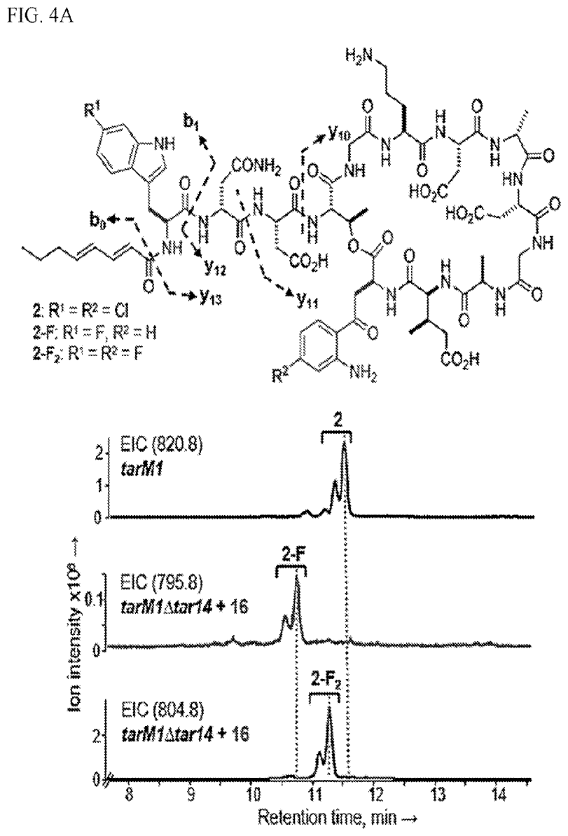

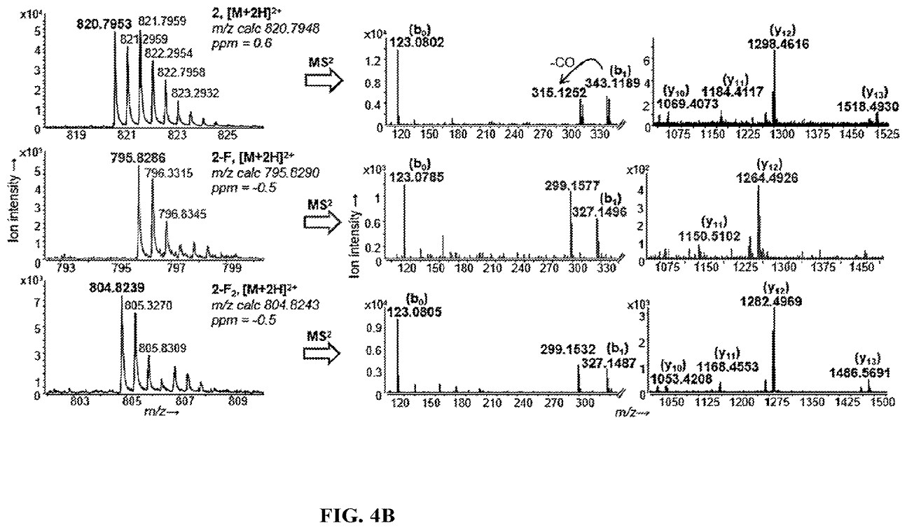

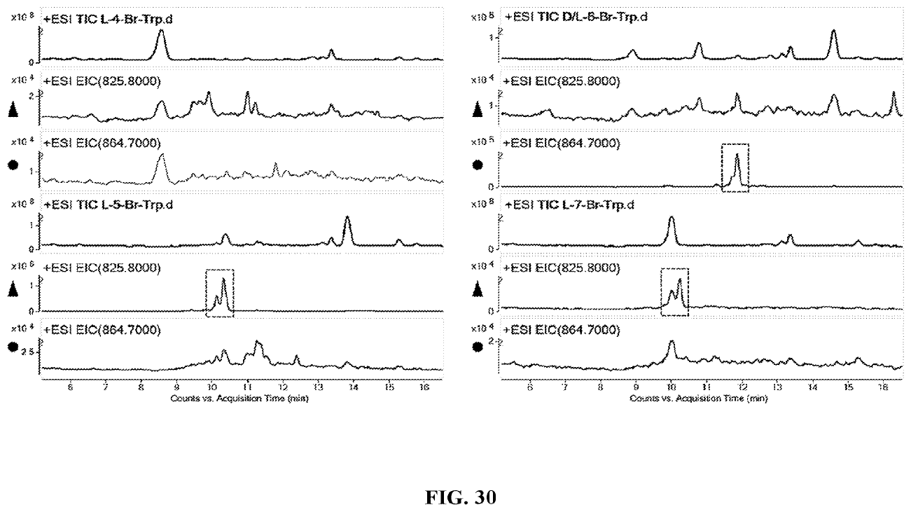

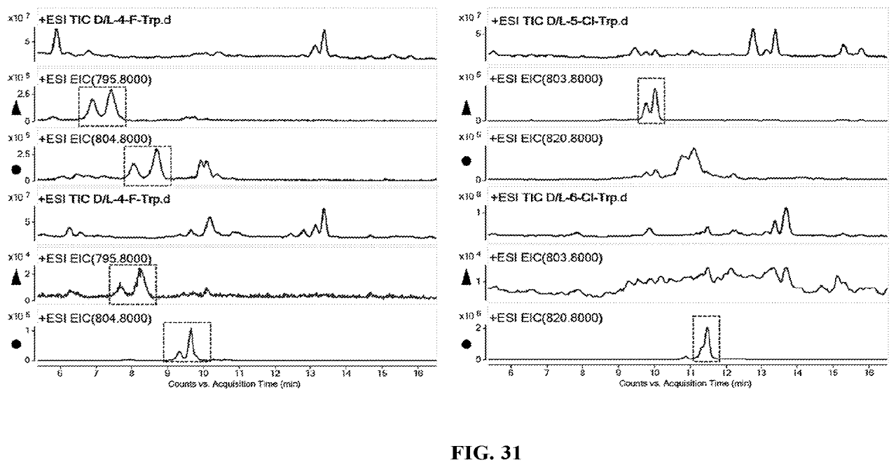

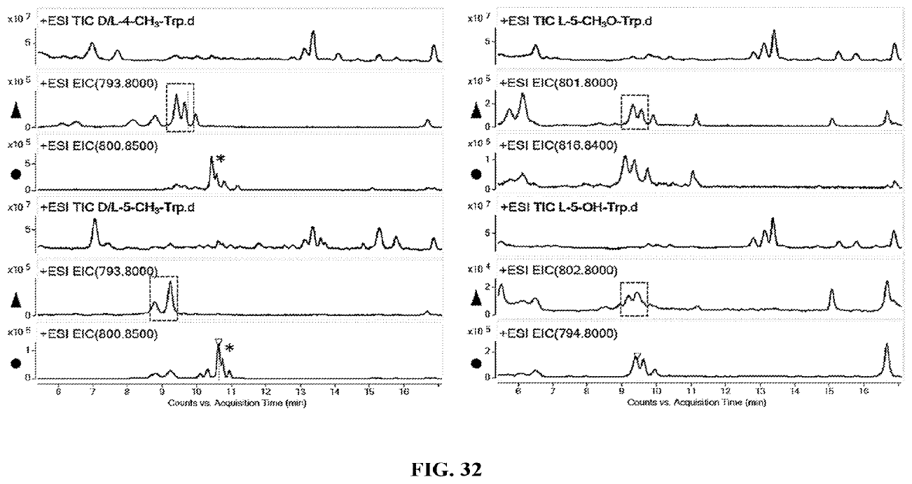

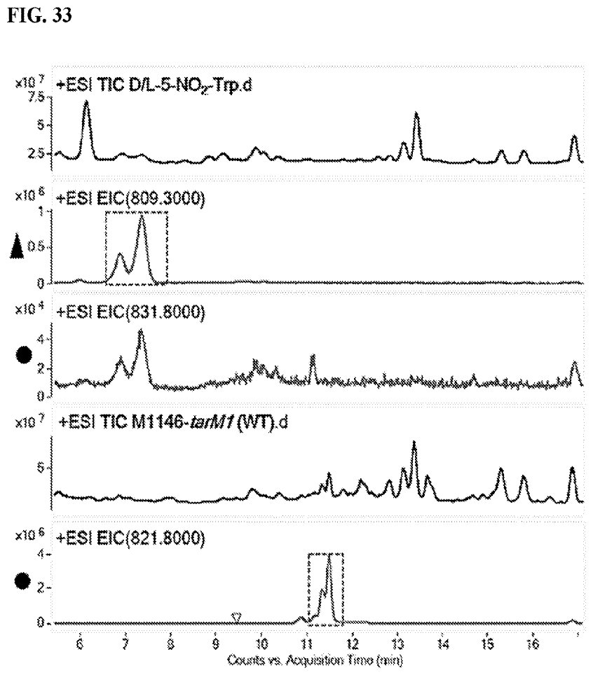

The in vitro substrate flexibilities of Tar13 and Tar16 encouraged applicants to probe whether analogous promiscuity is retained in vivo. The incorporation of Trp and Kyn derivatives additionally relies on the relaxed specificity of the corresponding NRPS A domains (A₁ and A₁₃). Applicants fed a variety of Trp derivatives (FIG. 5, Table 2) to S. coelicolor M1146-tarM1Δtar14. Analysis of LC-HRMSⁿ data showed that all analogues, apart from 12, were incorporated into residue-1 by A₁ (FIG. 5, FIGS. 26-33, Table 9). However, applicants observed limited incorporation of non-native Kyn derivatives at residue-13 by A₁₃. This mirrors the in vitro activities of Tar13/Tar16, suggesting that the inability to generate corresponding Kyn derivatives in vivo likely precludes formation of disubstituted taromycins. Especially pleasing was the incorporation of fluorinated amino acids with yields close to taromycin production level (FIG. 4, FIG. 5, Table 2). Addition of fluorine is an important modification in medicinal chemistry that often results in improved selectively, stability, and cell permeability of the therapeutically relevant compounds.^{[30]} Applicants generated eight analogues of taromycin (four each of the 2 and 3 series) that contain either one or two fluorine substitutions and are presently exploring yield optimization and bioactivity testing.

In summary, applicants genetically and biochemically validated the three-step enzymatic route from L-Trp to L-4-Cl-Kyn. Applicants anticipate that these enzymes will find utility as biocatalysts, especially when combined with enzyme engineering, and are a valuable addition to the toolkit for potential chemoenzymatic synthesis of halogenated aromatic molecules. Further, engineered biosynthetic enzymes can be applied for chemoenzymatic synthesis of kynurenine analogues, for example isotope-labeled or analogs with substituents on the aromatic portion of the molecule. Importantly, given the valuable therapeutic properties of L-4-Cl-Kyn and its ability to readily cross the blood-brain barrier, Tar13-16 enzymes represent an exciting opportunity for development as a microbiome-based therapy^{[31]} for the treatment of neurological disorders.^{[32]}

### Example 2: Materials and Methods

1. DNA and RNA Materials, Isolation, and Manipulation

Plasmids and oligonucleotides (Integrated DNA Technologies) used in this work are summarized in Tables 4 and 5, respectively.

Plasmid DNA was isolated from an overnight culture using the QIAprep Spin miniprep Kit (Qiagen) according to the manufacturer's protocol. DNA clean-up after PCR or agarose gel electrophoresis was performed with QIAquick PCR & Gel Cleanup Kit according to the manufacturer's protocol. High molecular weight DNA was recovered from agarose gels using Zymoclean Large Fragment DNA Recovery Kit (Zymo Research) according to the manufacturer's protocol. High molecular weight DNA was concentrated using isopropanol precipitation procedure^{[1]} DNA sequencing was carried out by the Genewiz Sequencing Facility, San Diego, Calif., USA.

Gene cloning and DNA assembly was done using HiFi DNA Assembly Master Mix (NEB).

In vitro transcription of DNA templates to generate gRNA for in vitro CRISPR/Cas9 gene deletion was performed using the TranscriptAid 17 high yield transcription kit (ThermoFisher Scientific) following the instructions.

TABLE 4
List and description of vectors and DNA constructs used.
Antibiotic resistance markers are highlighted in bold.

| Name | Description | Application | Reference |
|---|---|---|---|
| pET-28a(+) | neo, P_{T7}, ori^{pBR322}, lacI, N- and C- | Overexpression of genes | Novagen |
| | terminal His₆-tag. ori^{F1} | in E. coli | |
| pCJW93 | aac(3)IV, tsr, Hcn replicon, ori^{pIJ101}, | Heterologous protein | [2] |
| | tipAp, ori^{pUC}, oriT | production in | |
| | | Streptomyces | |
| pCRISPomyces-2 | aac(3)IV, oriT, rep^{pSG5(ts)}, ori^{ColE}, | Template to amplify trans- | [3] |
| | cas9, synthetic gRNA cassette | activating crRNA | |
| pUB307 | Self-transmissible plasmid: RP4, neo | Helper plasmid to transfer | [4] |
| | | in trans plasmids with | |
| | | oriT into heterologous | |
| | | host via conjugation | |
| pCAP01-tarM1 | Derivative of TAR cloning and | Heterologous expression | [5] |
| | broad-host-range heterologous | of tar BGC in | |
| | expression vector pCAP01 containing | Streptomyces host | |
| | captured tar BGC; neo, CEN6/ARS4, | | |
| | oriT, traJ, ori^{pUC}, URA3, ADH1p, | | |
| | TRP1, φC31 int-attP, aph(3)II, | | |
| | Δtar19-20, tar1-18 | | |
| pET28-Tar13 | pET-28a(+)-derived, tar13 gene | Overexpression of tar13 | this study |
| | cloned with N-terminal His₆-tag | | |
| pET28-Tar15 | pET-28a(+)-derived, tar15 gene | Overexpression of tar15 | this study |
| | cloned with N-terminal His₆-tag | | |
| pET28-Tar16 | pET-28a(+)-derived, tar16 gene | Overexpression of tar16 | this study |
| | cloned with N-terminal His₆-tag | | |
| pCJW93-Tar14 | pCJW93-derived, tar14 gene cloned | Overexpression of tar14 | this study |
| | with N-terminal His₆-tag | | |
| pET28-PtdH | pET-28a(+)-derived, ptdH gene | Overexpression of ptdH | [6] |
| | cloned with N-terminal His₆-tag | | |
| pET28-SsuE | pET-28a(+)-derived, ssuE gene | Overexpression of ssuE | [7] |
| | cloned with N-terminal His₆-tag | | |
| pCAP01- | Derivative of TAR cloning and | In vivo examine effect of | this study |
| tarM1Δtar14 | broad-host-range heterologous | deletion of the tar14 gene | |
| | expression vector pCAP01 containing | on taromycin production | |
| | captured tar BGC; neo, CEN6/ARS4, | | |
| | oriT, traJ, ori^{pUC}, URA3, ADH1p, | | |
| | TRP1, φC31 int-attP, aph(3)II, | | |
| | Δtar14, 19-20, tar1-18 | | |

TABLE 5
Oligonucleotides used. *-temperature went down 0.2 °C. each consecutive
cycle.

| | | |
|---|---|---|
| Name | Sequence, 5′→3′ | Taneal, °C. |

Primers used for in vitro in-frame deletion of the tar14 gene

| | | |
|---|---|---|
| Tar14_KO- | GACTGACACTGATAATACGACTCACTATAGGATGCCGTC | 69 (-0.2)* |
| gRNA1-F | ATCCACCCGGGTTTTAGAGCTAGAAATAGCAAGTT | |
| | (SEQ ID NO: 45) | |
| Tar14_KO- | GACTGACACTGATAATACGACTCACTATAGGCGAGCTGT | 69 (-0.2)* |
| gRNA2-F | ACAACCGGTGTTTTAGAGCTAGAAATAGCAAGTT | |
| | (SEQ ID NO: 46) | |
| Tar14_KO- | AAAAGCACCGACTCGGTGC | 69 (-0.2)* |
| gRNA-R | (SEQ ID NO: 47) | |
| Tar14_KO- | GATAGCGCTGTACGAATACTG | 62 |
| conf-F | (SEQ ID NO: 48) | |
| Tar14_KO- | CATACTCACGCTGCACAATGC | 62 |
| conf-R | (SEQ ID NO: 49) | |

Primers used for cloning genes for heterologous expression

| | | |
|---|---|---|
| Tar14_ | GGCCTGGTGCCGCGCGGCAGCCATATGTCTGTCAGTGGC | 72 |
| pCJW93_F | TCCGAAAGATCGGCC | |
| | (SEQ ID NO: 50) | |
| Tar14_ | GATCTGGGGAATTCGGATCCAAGCTTTTAGCGTCCGGCC | 72 |
| pCJW93_R | CGCATGGCCGCGAAGTA | |
| | (SEQ ID NO: 51) | |
| Tar13_F | CCTGGTGCCGCGCGGCAGCCATATGACCGAGCGGACCGC | 72 |
| | CACCCGAACGG | |
| | (SEQ ID NO: 52) | |
| Tar13_R | GTGGTGGTGGTGCTCGAGTGCGGCCGCCTATCCGCCTGTC | 72 |
| | CCGGATGCCCTCCG | |
| | (SEQ ID NO: 53) | |
| Tar15_F | CCTGGTGCCGCGCGGCAGCCATATGTCGATCGGTCGAAG | 72 |
| | CACTGCCGAG | |
| | (SEQ ID NO: 54) | |
| Tar15_R | GTGGTGGTGGTGCTCGAGTGCGGCCGCTCACCTCGCTTCG | 72 |
| | TCGAGCAGGCTC | |
| | (SEQ ID NO: 55) | |
| Tar16_F | CCTGGTGCCGCGCGGCAGCCATATGGCTGCGGCGGTGTT | 72 |
| | CCGGTCGTAC | |
| | (SEQ ID NO: 56) | |
| Tar16_R | GTGGTGGTGGTGCTCGAGTGCGGCCGCTCATTCGACAAG | 72 |
| | GCGGGCAACCGCCGC | |
| | (SEQ ID NO: 57) | |

2. Chemical and Biological Reagents

All chemicals used were purchased from commercial suppliers (Acros, Sigma Aldrich, Fluka, Chem-Impex International, or Toronto Research Chemicals Inc.). All organic solvents used were HPLC grade, for high resolution mass spectrometry MS grade solvents were used.

Restriction endonucleases, Phusion high-fidelity polymerase with GC buffer, T4 DNA ligase, Cas9 nuclease from S. pyogenes, and HiFi DNA Assembly Master Mix were purchased from New England Biolabs (NEB). Media components were purchased from BD (Difco and Bacto).

3. Bacterial Strains and Growth Conditions

All bacterial strains used or generated in the current study are summarized in Table 6.

Streptomyces coelicolor strains were grown on SFM solid medium (2% mannitol, 2% soya flour, 2% agar) for conjugation and strain maintenance, or in TSBY liquid medium (3% tryptone soy broth, 10.3% sucrose, 0.5% yeast extract). For liquid cultures, the strains were grown at 30° C. with shaking at 220 rpm in a rotary incubator for 36-44 h. For solid culture, the strains were grown at 30° C. for 4-7 days.

For taromycin production, seed culture in TSBY medium was inoculated with corresponding S. coelicolor spore suspension (collected from fresh SFM plates) and was cultured for 36-48 h till a dense creamy consistency was reached. Kanamycin at 10 μg mL⁻¹ final concentration was added to the seed cultures of S. coelicolor M1146-pCAP01-tarM1 and S. coelicolor M1146-pCAP01-tarM1Δtar14. Inocula of seed culture (5%, v/v) were used for fermentation in MP medium (1% glucose, 1% soluble starch, 0.4% peptone, 0.3% yeast extract, 0.3% soytone, 0.2% meat extract, 0.2% CaCO₃, and 0.5% NaCl, pH 7.2). Fermentation was carried out at 30° C. and 220 rpm in a rotary incubator for 6 days. A maximum of 40 mL of production medium was added into 250 mL flask with metal springs.

For protein production in S. coelicolor CH999 host, the strain was first activated by growing preculture in TSBY medium for 36-48 h. TSBY preculture (100 DL) was used to inoculate 30 mL of Super-YEME (0.3% yeast extract, 0.5% peptone, 1% glucose, 0.3% malt extract, 34% sucrose, 0.5% glycine, 0.235% MgCl₂.6H₂O, 7.5·10⁻³% L-proline, 7.5·10⁻³% L-arginine, 7.5·10⁻³% L-cysteine, 0.01% L-histidine, 1.5·10⁻³% uracil, pH 7.2) “primary culture” containing antibiotic apramycin at 50 μg mL⁻¹ final concentration in a 250 mL flask with a metal spring. The “primary culture” was grown at 30° C., 220 rpm for 4 days. Next, 10% (v/v) inocula of “primary culture” were used to inoculate a “secondary culture” of Super-YEME medium (30 mL supplemented with 50 μg mL⁻¹ apramycin). The “secondary culture” was incubated at 30° C., 220 rpm for 2 days. Finally, the “expression culture” (Super-YEME with 50 μg mL⁻¹ apramycin) was inoculated with 5% (v/v) of “secondary culture”, and incubated at 30° C., 220 rpm for 2 days. After 2 days antibiotic thiostrepton was added to a final concentration of 10 μg mL⁻¹ to induce gene expression from tipA promotor; also 50 mg of riboflavin per 600 mL of culture was added as a precursor of flavin cofactor. The cultures were further incubated at 30° C., 220 rpm for 2 days. “Expression cultures” were grown in 2.8 L flasks with metal springs containing 600 mL of culture maximum.

The standard spore conjugation protocol and general procedures with Streptomyces bacteria from Practical Streptomyces Genetics were followed.^{[8]}

E. coli strains were grown on solid (2% agar) or liquid LB (1% tryptone, 0.5% yeast extract, 1% NaCl) or 2TY (1.6% tryptone, 1% yeast extract, 0.5% NaCl) media supplemented with appropriate antibiotics (apramycin 50 μg mL⁻¹, chloramphenicol 25 μg mL⁻¹, kanamycin 50 μg mL⁻¹). Liquid cultures were shaken at 220 rpm at 37° C. unless otherwise stated. For protein production TB medium (1.2% tryptone, 2.4% yeast extract, 0.4% (v/v) glycerol, 2.31% KH₂PO₄. 12.54% K₂HPO₄) supplemented with kanamycin at 50 μg mL⁻¹ was used.

TABLE 6
List and description of the strains used.

| Strain | Genotype/Description | Reference |
|---|---|---|
| E. coli DH10B | F⁻, endA1, recA1, galE15, galK16, nupG, rpsL, ΔlacX74, | Invitrogen |
| | Φ80, lacZΔM15, araD139, | |
| | Δ(ara, leu)7697, mcrA, Δ(mrr-hsdRMS-mcrBC), λ⁻; Host | |
| | for DNA manipulations | |
| E. coli BL21(DE3) Gold | F⁻, ompT, gal, dcm, lon, hsdS_{B}(r_{B}⁻ m_{B}⁻), λ(DE3 [lacI | Novagen |
| | lacUV5-T7gene1, ind1, sam7, nin5]); | |
| | Host for gene expression | |
| E. coli ET12567 | F⁻, dam-13::Tn9, dcm-6, hsdM, hsdR, recF143, zjj- | [9] |
| | 202::Tn10, galK2, galT22, ara14, pacY1, | |
| | xyl-5, leuB6, thi-1) Donor strain for conjugation | |
| Streptomyces coelicolor | Heterologous host strain derived from S. coelicolor | [10] |
| M1146 | M145: Δact, Δred, Δcpk, Δcda | |
| S. coelicolor M1146- | Heterologous host with integrated into the genome tar | [5] |
| pCAP01-tarM1 | BGC | |
| S. coelicolor M1146- | Heterologous host with integrated into the genome tar | this study |
| pCAP01-tarM1Δtar14 | BGC with the deleted tar14 gene | |
| S. coelicolor CH999 | Heterologous host for protein overproduction, Δact, redE⁻ | [11] |
| | pro, arg | |
| S. coelicolor CH999- | Heterologous production of the Tar14 protein; S. | this study |
| pCJW93-Tar14 | coelicolor CH999 with replicative plasmid | |
| | pCJW93-Tar14, aac(3)IV, tsr. | |

4. Protein Overproduction in E. coli and Purification

E. coli BL21(DE3) cells transformed with the expression plasmids for Tar13, Tar15, Tar16, and PtdH proteins (Table 4) were inoculated into 10 mL of LB medium containing 50 μg mL⁻¹ kanamycin and grown overnight at 37° C., 220 rpm. Typically, 1 L of TB medium containing 50 μg mL⁻¹ kanamycin in a 2.8 L flask was inoculated with 10 mL of overnight culture (when more protein was required the culture volume was scaled up to 5 L). The cultures used to express Tar15, Tar16, and PtdH were incubated at 37° C., 220 rpm until the A₆₀₀ reached 0.6-0.8, at which point gene expression was induced by adding 1 M isopropyl-β-D-thiogalactopyranoside (IPTG) aqueous solution to a final concentration of 0.15 mM. In the case of the Tar13 protein (heme-dependent tryptophan 2,3-dioxygenase), the culture was incubated at 37° C., 220 rpm until the A₆₀₀ reached 0.4-0.5, then hemin and S-aminolevulinic acid stock solutions were added to final concentrations 7 μM and 1 mM, respectively, followed by further incubation at 37° C., 220 rpm. When A₆₀₀ reached 0.6-0.8, tar13 gene expression was induced by adding IPTG to a final concentration of 0.15 mM. After induction, the cultures were incubated at 18° C., 120 rpm for 18 h. Cells were harvested by centrifugation at 12,000×g, 5 min, 4° C. The cell pellet was resuspended in Binding buffer (40 mM Tris, 0.1 M NaCl, 20 mM imidazole, pH 7.4) and lysed by sonication on ice (Qsonica sonicator, CL-334 at 40% amplitude, 15 s pulse on/45 s pulse off, for 5 total min “on” time). The sonicate was centrifuged at 35,000×g, 45 min, 4° C., after which the soluble fraction was removed, filter sterilized, and subjected to column chromatography.

Protein purification was performed on an AKTApurifier instrument (GE Healthcare) with the modules Box-900, UPC-900, R-900 and Frac-900 with all buffers filtered through a nylon membrane 0.2 μm GDWP (Merck) prior to use. FPLC data was analyzed with UNICORN 5.31 (Built 743) software.

All proteins were purified by Ni²⁺-affinity chromatography using 1 mL or 5 mL HisTrap HP (GE Healthcare) columns. Buffers used were as follows: Ni buffer A (50 mM Tris, 500 mM NaCl, 10% glycerol, pH 8.0) and Ni buffer B (50 mM Tris, 500 mM NaCl, 300 mM imidazole, 10% glycerol, pH 8.0). The proteins were eluted in a gradient of Ni buffer B from 10 to 100% over 40 min at a flow rate of 2.5 mL/min. All steps were carried out at 4° C. with chilled buffers. SDS-PAGE (12.5% acrylamide) was used to determine protein-containing fractions. Following treatments and purification steps varied for different target proteins as described below.

Tar13. The protein started to elute from Ni²⁺ column at 90 mM imidazole. The fractions containing Tar 13 (dark red/brow color) were pooled together (FIGS. 16A-16C). To perform buffer exchange, we tried PD10 desalting columns (GE Healthcare), dialysis, protein concentration followed by dilution in storage buffer (various compositions of Tris, phosphate, HEPES buffers were tested). However, all attempts to exchange buffer to one that contained less salt resulted in immediate protein precipitation. The Tar13 protein remained stable only when left in Ni buffers (˜60% buffer B). Therefore, we next concentrated the protein fractions to a final volume of 2 mL using Amicon Ultra centrifugal filters with molecular weight cut-off (MWCO) 10 kDa (Millipore). Concentrated protein was subjected to size exclusion chromatography using Supedex 200 column (16 cm×60 cm, GE Healthcare) and buffer: 50 mM Tris, 500 mM NaCl, 180 mM imidazole, 10% glycerol, pH 8.0 (FIGS. 16A-16C). Fractions containing Tar13 (tetramer in solution) were combined and concentrated to 42 mg/mL. The protein was aliquoted and flash frozen in a dry ice/ethanol/water bath.

Tar15. After the protein eluted from Ni²⁺ column (100 mM imidazole), the fractions containing Tar15 were combined and concentrated to 2.5 mL using Amicon Ultra filter with MWCO 10 kDa (Millipore). Buffer exchange to a storage buffer A (40 mM Tris, 100 mM NaCl, 10% glycerol, pH 8.0) was performed using PD10 desalting column (GE Healthcare). Tar15 was obtained at 9 mg/mL concentration and was aliquoted and flash frozen in a dry ice/ethanol/water bath for storage.

Tar16. After Ni²⁺ chromatography, the fractions containing Tar16 were pooled and concentrated to 5 mL. This was diluted to a volume of 50 mL with IEx buffer A (20 mM Tris, 10% glycerol, pH 8.0). The protein was next applied on an ion exchange column HiTrap Q HP 5 mL (GE Healthcare) and eluted in a gradient 5-100% of IEx buffer B (20 mM Tris, 1 M KCl, 10% glycerol, pH 8.0) over 40 min at a flow rate of 2.5 mL/min. Fractions with Tar16 were concentrated to 2 mL with Amicon Ultra filter with MWCO 10 kDa (Millipore) and further purified by size exclusion chromatography using Supedex 75 column (16 cm×60 cm, GE Healthcare) and buffer: 40 mM Tris, 100 mM NaCl, 10% glycerol, pH 8.0 (FIGS. 16A-16C). After concentration, ice-cold glycerol was added to the protein to a final glycerol concentration 30% (v/v). Tar16 at 4.7 mg/mL was aliquoted and flash frozen in a dry ice/ethanol/water bath. All purification steps of Tar16 were done in one day as leaving protein overnight at 4° C. resulted in protein degradation.

PtdH. After elution from Ni²⁺ column, the protein fractions were combined and concentrated using Amicon filter with MWCO 10 kDa (Millipore). While concentrating, the protein was washed four times with 5 mL of Storage buffer B (50 mM MOPS, 200 mM NaCl, 1 mM DTT, pH 7.2). The protein at final concentration 18 mg/mL was aliquoted and flash frozen in a dry ice/ethanol/water bath.

The purified proteins were examined by SDS-PAGE (12.5% acrylamide) (FIGS. 16A-16C). Protein concentrations were determined using NanoDrop 1000 spectrophotometer V3.8 (Thermo Scientific).

Expression and purification of E. coli SsuE flavin reductase was performed according to a published protocol.^{[7]}

5. Purification of Flavin Dependent Halogenase Tar14 from S. coelicolor CH999

Attempts to produce soluble Tar14 in E. coli failed (N-, C-terminal His6-tag, N-terminal MBP-tag, coexpression with chaperones GroES-GroEL and DnaK-DnaJ-GrpE-GroES-GroEL (TAKARA), Rosetta (DE3) expression cells (Novagen)), therefore, we proceeded with expression in Streptomyces host. 2.4 L of S. coelicolor CH999-pCJW93-Tar14 culture was grown as described in section 3. Due to a high viscosity of the culture, it was diluted 1:1 with MilliQ water prior spinning cells down at 16,000×g, 30 min, 4° C. The cell pellet was resuspended in Binding buffer (40 mM Tris, 0.1 M NaCl, 20 mM imidazole, pH 7.4) and lysed by sonication on ice (Qsonica sonicator, CL-334 at 70% amplitude, 15 s pulse on/45 s pulse oft for 10 total min “on” time). The sonicate was centrifuged at 35,000×g, 60 min, 4° C., after which the soluble fraction was removed, filter sterilized, and subjected to column chromatography.

First, Tar14 was purified by Ni-affinity chromatography using 5 mL HisTrap HP (GE Healthcare) column. Buffers used: Ni buffer A (50 mM Tris, 500 mM NaCl, 10% glycerol, pH 8.0) and Ni buffer B (50 mM Tris, 500 mM NaCl, 300 mM imidazole, 10% glycerol, pH 8.0). The protein was eluted in a gradient of Ni buffer B from 10 to 100% over 40 min at a flow rate of 2.5 mL/min. All steps were carried out at 4° C. with chilled buffers. The protein started to elute from Ni²⁺ column at 60 mM imidazole. SDS-PAGE (12.5% acrylamide) was used to determine protein-containing fractions. Fractions with the target protein were combined and thrombin was added to a final concentration of 1 unit/mg recombinant protein. The protein was next dialyzed overnight against 20 mM Tris-HCl, 50 mM KCL, 10% glycerol, pH 8.9 buffer at 4° C. The dialyzed protein was purified by ion exchange chromatography using HiTrap Q HP 5 mL column (GE Healthcare) and was eluted in a gradient 5-100% of IEx buffers A (20 mM Tris, 10% glycerol, pH 8.0) and B (20 mM Tris, 1 M KCl, 10% glycerol, pH 8.0) over 40 min at flow rate 2.5 mL/min. Fractions with Tar14 were concentrated to 2 mL with Amicon Ultra filter with MWCO 30 kDa (Millipore) and further purified by size exclusion chromatography using Supedex 200 column (16 cm×60 cm, GE Healthcare) and buffer: 40 mM Tris, 100 mM NaCl, 10% glycerol, pH 8.0 (FIGS. 8A-8C). After concentration to 12 mg/mL, Tar14 was aliquoted and flash frozen in dry ice/ethanol/water bath. For crystallization purposes, freshly purified protein was used every time.

6. In Vitro Biochemical Assays

Tar14. Tar14 reaction mixture of total volume 100 μL in 1.5 mL Eppendorf tube was composed of the following components:

Buffer (10 mM Tris, 10% glycerol, pH 7.6): up to 100 μL

Sodium phosphite: 10 mM

PtdH: 2.5 μM

Tar15: 5 μM

FAD: 1 μM

NaCl (or KBr): 100 mM

NADP⁺: 2.4 mM

Substrate: 0.25 mM

Tar14: 5 μM

The reactions were incubated at 30° C. for 4 h, after which the assays were quenched by addition of equal volume of HPLC grade methanol and stored at −70° C. till further analysis by LCMS. * Exogeneous flavin reductase protein SsuE^{[7]} was also tried instead of Tar15, however, that did not affect activity of Tar14 and substrate conversion.

Tar13. Upon testing different reaction compositions (buffers, concentration of ascorbic acids and hemin, pH), the following conditions were found as optimal:

Buffer: up to 50 μL (50 mM Tris, 500 mM NaCl 10% glycerol, pH 8.0)

Substrate: 0.25 mM

In a separate Eppendorf tube premix:

Buffer: up to 50 μL (50 mM Tris, 500 mM NaCl 10% glycerol, pH 8.0)

L-ascorbic acid: 0.1 mM

Hemin: 6 [M

Tar13: 20 μM

Add substrate solution to the enzyme mixture to start reaction

When Tar13 was directly added to the enzyme reaction, immediate protein precipitation occurred. The same was observed if the enzyme was not pre-mixed with hemin and ascorbic acid. Full substrate conversion was achieved after overnight incubation at 30° C. in 1.5 mL Eppendorf tube with shaking. The reactions were quenched by addition of equal volume of HPLC grade methanol.

Coupled Tar13 Tar]6. The reaction mixture of total volume 100 μL in 1.5 mL Eppendorf tube was composed of the following components:

Buffer: up to 50 μL (50 mM Tris, 500 mM NaCl 10% glycerol, pH 8.0

Substrate (for Tar13 enzyme): 0.25 μM

In a separate Eppendorf tube premix:

Buffer: up to 50 μM (50 mM Tris, 500 mM NaCl 10% glycerol, pH 8.0)

L-ascorbic acid: 0.1 mM

hemin: 6 μM

Tar13: 20 μM

Tar16: 10 μM

Add substrate solution to the enzyme mixture to start reaction.

No difference in conversion was observed when Tar16 was added into the assay mixture either together with Tar13, or after 2 h of incubation at 30° C., or after overnight incubation at 30° C., followed by another 4 h of incubation after addition of Tar16. Therefore, for convenience, all components were mixed at the same time point, the reactions were incubated at 30° C. overnight, after which the assays were quenched by addition of equal volume of HPLC grade methanol.

One-pot Tar13 Tar14 Tar16 The following reaction composition was found to give best yields for conversion of L-tryptophan to L-4-chlorolynurenine (or L-4-bromokynurenine):

Buffer: up to 100 μL (50 mM Tris, 500 mM NaCl (or KBr) 10% glycerol, pH 8.0)

Sodium phosphite: 10 mM

PtdH: 2.5 μM

Tar15: 5 μM

FAD: 1 μM

NADP⁺: 2.4 mM

L-tryptophan: 0.25 mM

Tar14 (added last): 2.5 μM

Incubate 45 min at 30° C., followed by addition of premixed:

Buffer: up to 50 μL (50 mM Tris, 500 mM NaCl (or KBr) 10% glycerol, pH 8.0)

L-ascorbic acid: 0.1 mM

Hemin: 6 μM

Tar13: 20 μM

Tar16: 10 μM

The reactions were incubated at 30° C. overnight, after which the assays were quenched by addition of equal volume of HPLC grade methanol and frozen at −70° C. till further analysis by LCMS.

7. Kinetic Characterization of Tar14

To determine kinetic parameters for Tar14-catalyzed chlorination of L-tryptophan, phenol was first tested as an internal standard for quantification of the product (L-6-chlorotryptophan) formation. An HPLC method that gives separation of substrate/product/internal standard peaks (method 4) was developed (FIGS. 10A and 108). It was also verified that activity of Tar14 was not affected by the presence of phenol in the reaction mixture. Using commercial standard of L-6-chlorotryptophan and phenol mixed at known concentrations, a calibration curve was built (FIGS. 10A and 10B).

Tar14 reaction mixture of total volume 100 μL in 1.5 mL Eppendorf tube was composed of the following components:

Buffer (10 mM Tris, 10% glycerol, pH 7.6) up to 100 μL

Sodium phosphite: 10 mM

PtdH: 2.5 μM

Tar15: 5 μM

FAD: 1 μM

NaCl: 100 mM

NADP⁺: 2.4 mM

L-tryptophan (various concentrations): 0.01; 0.025; 0.05; 0.1; 0.2; 0.3 mM

Tar14 (added last): 2.5 μM

Phenol: 0.1 mM

The reactions were incubated at 30° C. for 5, 10, 25, 45, 60, 120, and 240 min, after which the assays were quenched by addition of equal volume of HPLC grade methanol and frozen at −70° C. till further analysis by HPLC. All assays were performed in triplicates for each substrate concentration and every time point. In parallel, for each time point and substrate concentration, assays with no added phenol and with no added enzyme were used as controls.

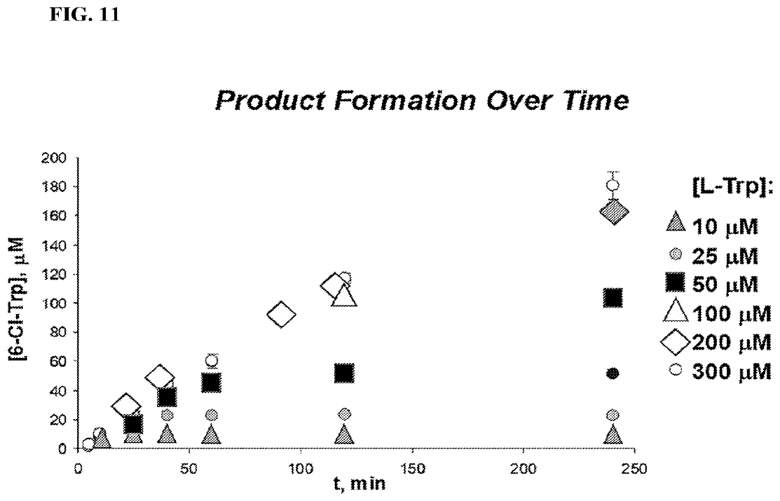

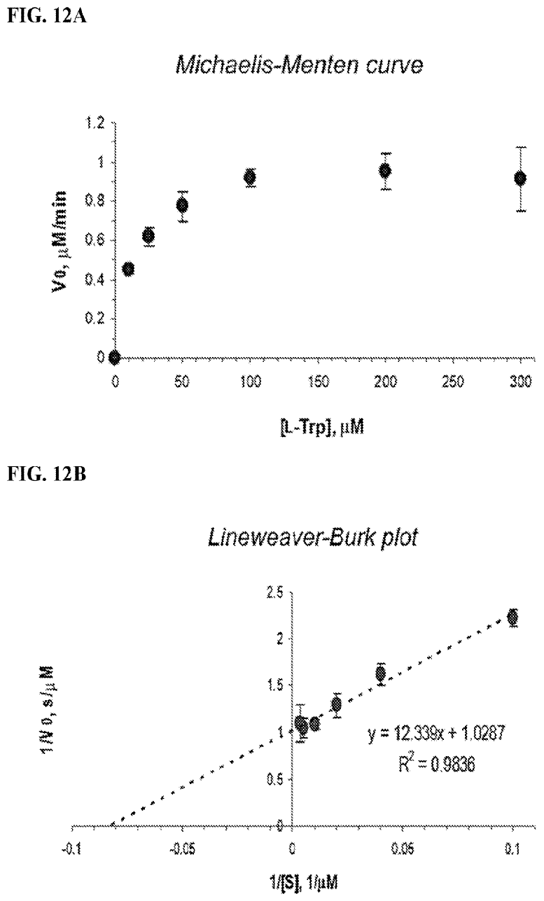

Product formation was quantitated by calculating the ratio of peak areas of product to internal standard and fitting that value to a calibration curve. These values were used to build product formation over time plot to determine observed initial rates for each substrate concentration (FIG. 11). These data were used to build a Michaelis-Menten curve and the kinetic parameters (K_{M} and k_{cat}) were determined using the Lineweaver-Burk plot (see FIGS. 12A and 12B).

8. Kinetic Characterization of Tar13

To determine kinetic parameters for Tar13-catalyzed oxidation of L-6-Cl-tryptophan, the following reaction conditions were used:

Buffer: up to 50 μL (50 mM Tris, 500 mM NaCl 10% glycerol, pH 8.0)

L-6-Cl-tryptophan (various concentrations): 0.01; 0.025; 0.05; 0.08; 0.1; 0.2; 0.3 mM

In a separate Eppendorf tube premix:

Buffer: up to 50 μL (50 mM Tris, 500 mM NaCl 10% glycerol, pH 8.0)

L-ascorbic acid: 0.1 mM

Hemin: 6 μM

Tar13: 2 μM

The enzyme mixture was added to the substrate solution to start the reactions. The reactions were incubated at 30° C. for 5, 10, 15, 30 min, after which the assays were quenched by addition of equal volume of HPLC grade methanol and frozen at −70° C. till further analysis by HPLC. All assays were performed in triplicates for each substrate concentration and every time point. In parallel, for each time point and substrate concentration, assays with no added enzyme were used as controls. Prior HPLC analysis (method 5), L-tryptophan was added to the quenched reaction mixtures as an internal standard for quantification.

Product formation was quantitated by calculating decrease of the area of the remaining substrate in comparison to the substrate peak area in the control with no enzyme added, and the ratio of peak areas to internal standard were fitted into a calibration curve to determine product concentrations. These data were used to determine observed initial rates for each substrate concentration and to build a Michaelis-Menten curve to determine the kinetic parameters (K_{M} and k_{cat}) (see FIGS. 18A and 18B).

9. Crystallization and Structure Determination of Tar14

Tar14 was purified in 40 mM Tris-HCl (pH 8.0), 100 mM NaCl, 10% glycerol and was used for crystallization at a concentration of 8 mg mL⁻¹ supplemented with cofactor FAD and substrate L-tryptophan, both at final concentration 1 mM. Approximately 300 conditions from different commercialized crystallization kits (HR2-110, HR2-112, HR2-144, Natrix HR2-116 from Hampton Research, and Wizard 1009533 Rigaku from Emerald Biosystems) were screened in hanging-vapor drop format at 10° C. Crystals were obtained by vapor diffusion by equilibrating 2 μL hanging drops containing a 1:1 mixture of protein solution and crystallization buffer over a 150 μL reservoir of the corresponding crystallization buffer. After optimization, the following condition gave reproducible crystal growth: 0.1 M BisTris methane (pH 6.5), 0.35 M Li₂SO₄, 28% PEG 3350. Crystals generally appeared within 1-3 days. The crystals were harvested and stabilized by soaking briefly in a cryoprotectant solution (25% PEG 3350, 0.25 M Li₂SO₄, 0.1 M BisTris methane (pH 6.5), 10% glycerol) prior to being flash frozen in liquid nitrogen for data collection. X-ray diffraction data were collected on beamline 8.2.1 at the Advanced Light Source (Berkeley, Calif., USA) using a wavelength of 0.99992 Å. Data were indexed, integrated, and scaled using XDS^{[12]} and autoPROC^{[13]} software in the space group P1 and at a resolution of 1.74 Å.

The structure of Tar14 was solved by molecular replacement using PHASER[¹⁴] as implemented in the PHENIX.software suite.^{[15]} The structure of FDH Th-Hal (PDB code: 5LV9) was used as a search model^{[16]} An initial model was built using PHENIX AutoBuild. The structure was manually rebuilt and visualized using the program COOT,^{[17]} followed by rounds of refinement using phenix.refine. The figures were prepared using Chimera.^{[18]} The Tar14 atomic coordinates and structure factor have been deposited in the Protein Data Bank (PDB) with accession code: 6NSD. The statistics of data collection and refinement are detailed in Table 7.

TABLE 7
Data collection and refinement statistics of Tar14. (*) the
values in parentheses refer to the highest resolution shell
^{a}R_{sym} = Σₕ|Iₕ − <I>|/ΣₕIₕ, where Iₕ is the intensity of reflection
h <I> is the mean intensity of all symmetry-related reflections

| | |
|---|---|
| PDB ID (Accession code) | 6NSD |

X-ray data collection

| | |
|---|---|
| Wavelength (Å) | 0.99992 |
| Space group | P1 |
| Subunits in the asymmetric unit | 2 |

Unit cell

| | |
|---|---|
| a, b, c (Å) | 51.35 68.21 85.36 |
| α, β, γ (°) | 104.68 103.77 106.42 |
| Resolution range (Å) | 61.43-1.74 |
| Highest resolution shell (Å) | 1.77-1.74 |
| R_{sym}^{a} (%) (*) | 11.3 (48.5) |
| Completeness (%) (*) | 95.9 (95.6) |
| Number of unique reflections (*) | 100573 (5046) |
| Multiplicity (*) | 3.8 (3.7) |
| Average intensity, <I/σ(I)> (*) | 5.3 (2.2) |
| CC(1/2) | 0.99 (0.79) |

Data refinement

| | |
|---|---|
| Resolution range (Å) | 41.74-1.74 |
| Completeness (%) | 95.88 |
| Number of reflections | 100551 |
| R_{work}/R_{free} | 0.1636/0.1957 |
| Number of all non-hydrogen | 9002 |
| atoms | |
| Number of water molecules | 841 |
| Number of non-hydrogen protein | 8035 |
| atoms | |
| Number of ligand atoms | 126 |

B-factor analysis (Å²)

| | |
|---|---|
| Protein | 21.34 |
| Ligands | 21.81 |
| Water molecules | 31.17 |

Ramachandran plot

| | |
|---|---|
| Outliers (%) | 0.71 |
| Most favored (%) | 97.98 |
| Additional allowed (%) | 2.02 |
| Bond length (Å) | 0.006 |
| Bond angles (°) | 0.872 |

10. Construction of pCAP01-tarM1ΔTar14 Using In Vitro CRISPR/Cas9 Approach

General strategy and protocol described by Liu et al. was followed to delete tar14 gene in cosmid pCAP01-tarM1 and is schematically illustrated on FIG. 13.^{[19]} Primers used to amplify DNA encoding sgRNA sequences are provided in Table 4.

Linearized with Cas9 cosmid DNA was self-ligated in a reaction that contained: 5 L DNA fragment (90 ng or 2 fmol), 1 μL 10×T4 DNA ligase buffer, 1 μL of T4 DNA ligase, 3 μL of water. The mixture was incubated at 16° C. for 24 h followed by transformation into freshly prepared room temperature electrocompetent cells.^{[20]}

11. Feeding/Chemical Complementation Experiment

S. coelicolor M1146-pCAP01-tarM1Δtar14 cultures were grown as described in Section 3 for taromycin production. After 18 and 36 h of cultivation, tryptophan analogues in the form of water suspension were added to the production cultures to a final concentration 2 mM. The cultures were further incubated for 5 days before extraction and further analysis by LC-HRMSⁿ. All experiments were repeated independently twice with three replicates for each compound. In parallel, cultures of S. coelicolor M1146-pCAP01-tarM1 and S. coelicolor M1146-pCAP01-tarM1Δtar14 were grown as controls.

12. Analytical Procedures

NMR spectra were recorded on a Bruker Avance III spectrometer (600 MHz) using a 5 mm inverse detection triple resonance (H-C/N/D) cryoprobe and deuterated methanol (CD₃OD) as a solvent. Chemical shifts were recorded using an internal deuterium lock for ¹³C and residual ¹H in CD₃OD (δH 3.31, δC 49.0) and are given in ppm on a scale relative to δ_{TMS}=0. NMR spectra were recorded using Bruker Topspin (v. 2.1.6) software and NMR data were analyzed with MestReNova V.12.0 software.

LCMS analysis was carried out on an Agilent Technologies 1200 Series system with a diode-array detector coupled to an Agilent Technologies 6530 Accurate-Mass Q-TOF mass spectrometer. The mass spectrometer was run in positive ionization mode. Data was analyzed with Agilent MassHunter software B.05.01. Low resolution data was acquired using a Bruker Amazon Ion Trap MS system coupled to an Agilent 1260 Infinity LC system an Agilent 1260, and data was analyzed with Bruker Compass DataAnalysis 4.2 software. Phenomenex Luna C18 reversed-phase analytical HPLC column (5 μm, 250 mm×4.6 mm) was used for small molecule separation. A solvent system of water (A) and acetonitrile (B) both containing 0.10% formic acid (v/v) and the following methods were used:

Method 1 (for analysis of Tar14, Tar13, Tar13/16, and Tar13/14/16 enzymatic assays): 0.75 mL/min flow rate; 0-5 min 5% B, 5-15 min 5-12% B, 15-30 min 12-15% B, 30-35 min 15-100% B, 35-38 min 100% B, 38-40 min 100-5% B, 40-45 min 5% B.

Method 2 (for analysis of Tar14 assays with tryptophan analogues): 0.75 m/min flow rate; 0-5 min 2% B, 5-26 min 2-100% B, 26-30 min 100% B, 30-31 min 100-2% B, 31-35 min 2% B.

Method 3 (analysis of taromycins): 0.7 mL/min flow rate; 0-20 min 10-32% B, 20-33 min 32-70% B, 33-38 min 70-100% B, 38-41 min 100% B, 41-42 min 100-10% B, 42-45 min 10% B.

Method 4 (kinetics of Tar14): 0.75 mL/min flow rate; 0-3 min 2-5% B, 3-18 min 5-100% B, 18-20 min 100% B, 20-20.5 min 100-2% B, 20.5-23 min 2% B.

Method 5 (kinetics of Tar13): 0.75 mL/min flow rate; 0-3 min 5-10% B, 3-13 min 10-15% B, 13-24 min 15-100% B, 24-25 min 100% B. 25-26 min 100-5% B, 26-29 min 5% B.

Semi-preparative HPLC was carried out on an Agilent Technologies 1200 Series system with a multiple wavelength detector using a Phenomenex C18 Luna column (5 m, 250 mm×10 mm). HPLC data were processed using Agilent OpenLAB CDS C.01.05 ChemStation Edition software. The following method was used:

Method 6 (for purification of compounds 1 and 13): 3 mL/min flow rate; 0-30 min 10-15% B, 30-45 min 15-25% B, 45-55 min 25-100% B, 55-56 min 100% B, 56-60 min 100-10% B, 60-64 min 10% B.

Preparative HPLC purification was performed using a Phenomenex C18 Luna column (5 μm, 100 mm×21.2 mm) connected to the Agilent 1200 series HPLC. Data were processed using Agilent OpenLAB CDS C.01.05 ChemStation Edition software. The following methods were used.

Method 7 (for isolation of L-6-chlorotryptophan): 15 mL/min flow rate; 0-5 min 2-5% B, 5-26 min 5-100% B, 26-30 min 100% B, 30-31 min 100-2% B, 31-35 min 2% B.

Method 8 (for isolation of N-formyl-L-4-chlorokynurenine and L-4-chlorokynurenine): 15 mL/min flow rate; 0-30 min 10-15% B, 30-45 min 15-25% B, 45-55 min 25-100% B, 55-56 min 100% B, 56-60 min 100-10% B.

To analyze production of taromycins and analogues, solid phase extraction of the culture supernatants was performed with Amberlite XAD-16 resin (SIGMA). For analytical purposes around 1 g of washed and equilibrated resin was added to 20 mL of supernatant followed by shaking for 30-60 min. Samples were then spun down and decanted. The resin was washed twice with water (15 mL), before elution with methanol (5 mL). The methanol extracts from the resin were used for LCMS analysis without additional concentration step.

13. Purification of Products of the Enzymatic Reactions

L-6-Chlorotryptophan (7): Tar14-catalyzed chlorination of L-tryptophan was scaled up to 15 mL (50×300 μL reactions in 1.5 mL Eppendorf with ratio of all components as described in section 8 but with concentration of L-tryptophan 1 mM). After 6 h of incubation the reactions were combined, frozen on dry ice, and lyophilized. The dried sample was redissolved in a 500 μL water/methanol (1/1) mixture and subjected to purification by preparative HPLC using method 7. Prior to combining, the fractions were checked by direct injection into the Bruker Ion Trap mass spectrometer. Organic solvent from combined fractions was removed by evaporation at reduced pressure and the aqueous sample was frozen following by lyophilization. The desired product 7 (2 mg) was obtained as a pale beige solid.

N-formyl-L-4-Cl-kynurenine (10): The Tar13 biochemical assay was scaled to 57.6 mL (six 96 well plates with 100 μL reaction mixture as described in section 8 in each well). After overnight incubation, the assays were pooled together, frozen on dry ice, and lyophilized. The dried sample was redissolved in a 4000 μL of water/methanol (1/1) mixture and subjected to purification by preparative HPLC using method 8. Organic solvent from combined fractions (checked by direct injection into the Bruker Ion Trap mass spectrometer) was removed by evaporation at reduced pressure and the aqueous sample was frozen following by lyophilization. Due to the presence of closely eluting impurities, further purification step using semi-preparative HPLC and method 6 was used. The target peak was collected manually. After removing organic solvent under reduced pressure and lyophilization, the desired product 10 (1.8 mg) was obtained. Note: partial deformylation of N-formyl-L-4-Cl-kynurenine (10) to L-4-Cl-kynurenine (1) was observed every time after lyophilization step.

L-4-Cl-kynurenine (1): The Tar13/Tar16 coupled assay was scaled to 57.6 mL (six 96 well plates with 100 μL reaction mixture in each well). After reaction completion, the assays were pooled together, frozen on dry ice, and lyophilized. The dried sample was redissolved in a 4000 μL of water/methanol (1/1) mixture and subjected to purification by preparative HPLC using method 8. Fractions were checked by direct injection into the Bruker Ion Trap mass spectrometer and the combined sample was dried. Further purification step using semi-preparative HPLC and method 6 was required. The target peak was collected manually. After removing solvent and water, the desired product (1.3 mg) was obtained.

TABLE 8
High resolution MS data for products of enzymatic reaction catalyzed by
Tar14, Tar13, and Tar13/Tar16 with a library of substrate analogues.

| | | Tar14 reaction product | | | | Tar13 reaction product | |
|---|---|---|---|---|---|---|---|
| | | | Calculated | Observed | | | Calculated |
| | | | m/z | m/z | Error | | m/z |
| Substrate | | Molecular formula | [M + H]⁺ | [M + H]⁺ | ppm | Molecular formula | [M + H]⁺ |
| 4, L-Trp | Cl⁻ | C₁₁H₁₂ClN₂O₂⁺ | 239.05 | 239.058 | 0.84 | C₁₁H₁₃N₂O₄⁺ | 237.087 |
| | Br⁻ | C₁₁H₁₂BrN₂O₂⁺ | 283.00 | 283.007 | 0.35 | | |
| 1, L-4-Cl-Kyn | Cl⁻ | C₁₀H₁₁Cl₂N₂O₃⁺ | 277.01 | 277.014 | 0 | | |
| | Br⁻ | C₁₀H₁₁BrClN₂O₃⁺ | 320.96 | 320.96 | 0.62 | | |
| 5, L-Kyn | Cl⁻ | C₁₀H₁₂ClN₂O₃+ | 243.05 | 243.05 | 0 | | |
| | Br⁻ | C₁₀H₁₂BrN₂O₃⁺ | 287.00 | 287.002 | 0.7 | | |
| 11, D-Trp | Cl⁻ | C₁₁H₁₂ClN₂O₂⁺ | 239.05 | 239.058 | −0.42 | C₁₁H₁₃N₂O₄⁺ | 237.087 |
| | Br⁻ | C₁₁H₁₂BrN₂O₂⁺ | 283.00 | 283.007 | 0.71 | | |
| 12, L-4-Br-Trp | Cl⁻ | C₁₁H₁₁BrClN₂O₂⁺ | 316.96 | 316.96 | −0.32 | C₁₁H₁₂BrN₂O₄⁺ | 314.99 |
| | Br⁻ | C₁₁H₁₁Br₂N₂O₂⁺ | 360.91 | 360.91 | −0.28 | | |
| 8, L-5-Br-Trp | Cl⁻ | C₁₁H₁₁BrClN₂O₂⁺ | 316.96 | 316.96 | 0.63 | C₁₁H₁₂BrN₂O₄⁺ | 314.99 |
| | Br⁻ | C₁₁H₁₁Br₂N₂O₂⁺ | 360.91 | ND | | | |
| 9, L-6-Br-Trp | Cl⁻ | C₁₁H₁₁BrClN₂O₂⁺ | 316.96 | ND | | C₁₁H₁₂BrN₂O4⁺ | 314.99 |
| | Br⁻ | C₁₁H₁₁Br₂N₂O₂⁺ | 360.91 | 360.91 | 0 | | |
| 13, L-7-Br-Trp | Cl⁻ | C₁₁H₁₁BrClN₂O₂⁺ | 316.96 | 316.96 | 0.63 | C₁₁H₁₂BrN₂O₄⁺ | 314.99 |
| | Br⁻ | C₁₁H₁₁Br₂N₂O₂⁺ | 360.91 | 360.91 | −0.28 | | |
| 14, D/L-5-Cl-Trp | Cl⁻ | C₁₁H₁₁Cl₂N₂O₂⁺ | 273.01 | 273.01 | 0 | C₁₁H₁₂ClN₂O₄⁺ | 271.04 |
| | Br⁻ | C₁₁H₁₁BrClN₂O₂⁺ | 316.96 | 316.96 | 0.32 | | |
| 7, L-6-Cl-Trp | Cl⁻ | C₁₁H₁₁Cl₂N₂O₂⁺ | 273.01 | ND | | C₁₁H₁₂ClN₂O₄ | 271.04 |
| | Br⁻ | C₁₁H₁₁BrClN₂O₂⁺ | 316.96 | 316.96 | 0.63 | | |
| 16, D/L-6-F-Trp | Cl⁻ | C₁₁H₁₁ClFN₂O₂⁺ | 257.048 | 257.04 | 1.17 | C₁₁H₁₂FN₂O₄⁺ | 255.077 |
| | Br⁻ | C₁₁H₁₁BrFN₂O₂⁺ | 300.99 | 300.99 | 0.33 | | |
| 15, D/L-4-F-Trp | Cl⁻ | C₁₁H₁₁ClFN₂O₂⁺ | 257.04 | 257.04 | 0.39 | C₁₁H₁₂FN₂O₄⁺ | 255.077 |
| | Br⁻ | C₁₁H₁₁BrFN₂O₂⁺ | 300.99 | 300.998 | −0.66 | | |
| 18, D/L-5-CH₃-Trp | Cl⁻ | C₁₂H₁₄ClN₂O₂⁺ | 253.07 | 253.07 | −0.79 | C₁₂H₁₅N₂O₄⁺ | |
| | Br⁻ | C₁₂H₁₄BrN₂O2⁺ | 297.02 | 297.02 | 1.01 | | 251.102 |
| 19, L-5-CH₃O-Trp | Cl⁻ | C₁₂H₁₄ClN₂O₃⁺ | 269.06 | 269.06 | −0.74 | C₁₂H₁₅N₂O₅⁺ | |
| | Br⁻ | C₁₂H₁₄BrN₂O₃⁺ | 313.01 | 313.01 | −0.32 | | 267.097 |
| 17, D/L-4-CH₃-Trp | Cl⁻ | C₁₂H₁₄ClN₂O₂⁺ | 253.07 | 253.07 | 0 | C₁₂H₁₅N₂O₄⁺ | |
| | Br⁻ | C₁₂H₁₄BrN₂O₂⁺ | 297.02 | 297.02 | 0.34 | | 251.102 |
| 20, L-5-OH-Trp | Cl⁻ | C₁₁H₁₂ClN₂O₃⁺ | 255.05 | 255.05 | 0.39 | C₁₁H₁₃N₂O₅⁺ | |
| | Br⁻ | C₁₁H₁₂BrN₂O₃⁺ | 299.00 | 299.00 | −0.33 | | 253.081 |

| | | Tar13 reaction product | | Tar13/Tar16 coupled reaction product | | | |
|---|---|---|---|---|---|---|---|
| | | Observed | | | Calculated | Observed | |
| | | m/z | Error | | m/z | m/z | Error, |
| Substrate | | [M + H]⁺ | ppm | Molecular formula | [M + H]⁺ | [M + H]⁺ | ppm |
| 4, L-Trp | Cl⁻ | 237.08 | 0.42 | C₁₀H₁₃N₂O₃⁺ | 209.092 | 209.092 | 0.48 |
| | Br⁻ | | | | | | |
| 1, L-4-Cl-Kyn | Cl⁻ | | | | | | |
| | Br⁻ | | | | | | |
| 5, L-Kyn | Cl⁻ | | | | | | |
| | Br⁻ | | | | | | |
| 11, D-Trp | Cl⁻ | 237.08 | 0 | C₁₀H₁₃N₂O₃⁺ | 209.092 | 209.092 | 0.96 |
| | Br⁻ | | | | | | |
| 12, L-4-Br-Trp | Cl⁻ | ND | | | | | |
| | Br⁻ | | | | | | |
| 8, L-5-Br-Trp | Cl⁻ | 314.98 | 0.95 | C₁₀H₁₂BrN₂O₃⁺ | 287.002 | 287.002 | 0.35 |
| | Br⁻ | | | | | | |
| 9, L-6-Br-Trp | Cl⁻ | 314.99 | 0.32 | C₁₀H₁₂BrN₂O₃⁺ | 287.002 | 287.002 | −1.05 |
| | Br⁻ | | | | | | |
| 13, L-7-Br-Trp | Cl⁻ | ND | | | | | |
| | Br⁻ | | | | | | |
| 14, D/L-5-Cl-Trp | Cl⁻ | 271.04 | −0.74 | C₁₀H₁₂ClN₂O₃⁺ | 243.053 | 243.053 | 0.82 |
| | Br⁻ | | | | | | |
| 7, L-6-Cl-Trp | Cl⁻ | 271.04 | −0.74 | C₁₀H₁₂ClN₂O₃⁺ | 243.053 | 243.053 | 0 |
| | Br⁻ | | | | | | |
| 16, D/L-6-F-Trp | Cl⁻ | 255.07 | 0 | C₁₀H₁₂FN₂O₃⁺ | 227.082 | 227.082 | −0.88 |
| | Br⁻ | | | | | | |
| 15, D/L-4-F-Trp | Cl⁻ | ND | | | | | |
| | Br⁻ | | | | | | |
| 18, D/L-5-CH₃-Trp | Cl⁻ | | | | | | |
| | Br⁻ | 251.10 | −0.4 | C₁₁H₁₅N₂O₃⁺ | 223.107 | 223.107 | 0.45 |
| 19, L-5-CH₃O-Trp | Cl⁻ | | | | | | |
| | Br⁻ | 267.09 | −0.37 | C₁₁H₁₅N₂O₄⁺ | 239.102 | 239.102 | 0 |
| 17, D/L-4-CH₃-Trp | Cl⁻ | | | | | | |
| | Br⁻ | ND | | | | | |
| 20, L-5-OH-Trp | Cl⁻ | | | | | | |
| | Br⁻ | ND | | | | | |

ND—not detected.

TABLE 9
High resolution MS data for taromycin analogues detected in feeding experiments
when tryptophan derivatives (bold) were added to S. coelicolor M1146-tarM1Δtar14
cultures. (A₁) stands for incorporation of the respective analogue only in the position of
residue-1 in taromycin A (instead of L-6-chlorotryptophan), (A₁ + A₁₃)
stands for incorporation in positions of both, L-6-chlorotryptophan and
L-4-chlorokynurenine (residue-1 and residue-14, respectively).

| | | | | |
|---|---|---|---|---|
| | | Calculated m/z | Observed m/z | |
| Compound | Molecular formula | [M + 2H]²⁺ | [M + 2H]²⁺ | Error, ppm |

D/L-4-F-Trp

| | | | | |
|---|---|---|---|---|
| Tar (A₁) | C₇₀H₉₄FN₁₇O₂₅²⁺ | 795.8290 | 795.8292 | 0.25 |
| Tar (A₁ + A₁₃) | C₇₀H₉₃F₂N₁₇O₂₅²⁺ | 804.8243 | 804.8258 | 1.86 |

D/L-6-F-Trp

| | | | | |
|---|---|---|---|---|
| Tar (A₁) | C₇₀H₉₄FN₁₇O₂₅²⁺ | 795.8290 | 795.8286 | −0.50 |
| Tar (A₁ + A₁₃) | C₇₀H₉₃F₂N₁₇O₂₅²⁺ | 804.8243 | 804.8249 | 0.75 |

L-4-Br-Trp

| | | | |
|---|---|---|---|
| Tar (A₁) | C₇₀H₉₄BrN₁₇O₂₅²⁺ | 825.78895 | NO |
| Tar (A₁+ A₁₃) | C₇₀H₉₃Br₂N₁₇O₂₅²⁺ | 864.7442 | NO |

L-5-Br-Trp

| | | | | |
|---|---|---|---|---|
| Tar (A₁) | C₇₀H₉₄BrN₁₇O₂₅²⁺ | 825.78895 | 825.7898 | 1.03 |
| Tar (A₁ + A₁₃) | C₇₀H₉₃Br₂N₁₇O₂₅²⁺ | 864.7442 | NO | |

L-6-Br-Trp

| | | | | |
|---|---|---|---|---|
| Tar (A₁) | C₇₀H₉₄BrN₁₇O₂₅²⁺ | 825.78895 | NO | |
| Tar (A₁ + A₁₃) | C₇₀H₉₃Br₂N₁₇O₂₅²¹ | 864.7442 | 864.7463 | 2.43 |

L-7-Br-Trp

| | | | | |
|---|---|---|---|---|
| Tar (A₁) | C₇₀H₉₄BrN₁₇O₂₅²⁺ | 825.78895 | 825.7902 | 1.51 |
| Tar (A₁ + A₁₃) | C₇₀H₉₃Br₂N₁₇O₂₅²⁺ | 864.7442 | NO | |

D/L-5-Cl-Trp

| | | | | |
|---|---|---|---|---|
| Tar (A₁) | C₇₀H₉₄ClN₁₇O₂₅²⁺ | 803.8142 | 803.8143 | 0.12 |
| Tar (A₁ + A₁₃) | C₇₀H₉₃Cl₂N₁₇O₂₅²⁺ | 820.7948 | NO | |

D/L-6-Cl-Trp

| | | | | |
|---|---|---|---|---|
| Tar (A₁) | C₇₀H₉₄ClN₁₇O₂₅²⁺ | 803.8142 | NO | |
| Tar (A₁ + A₁₃) | C₇₀H₉₃Cl₂N₁₇O₂₅²⁺ | 820.7948 | 820.7962 | 1.71 |

D/L-4-CH₃-Trp

| | | | | |
|---|---|---|---|---|
| Tar (A₁) | C₇₁H₉₇N₁₇O₂₅²⁺ | 793.8416 | 793.8428 | 1.51 |
| Tar (A₁ + A₁₃) | C₇₂H₉₉N₁₇O₂₅²⁺ | 800.8494 | NO | |

D/L-5-CH₃-Trp

| | | | | |
|---|---|---|---|---|
| Tar (A₁) | C₇₁H₉₇N₁₇O₂₅²⁺ | 793.8416 | 793.8415 | −0.13 |
| Tar (A₁ + A₁₃) | C₇₂H₉₉N₁₇O₂₅²⁺ | 800.8494 | NO | |

D/L-5-NO₂-Trp

| | | | | |
|---|---|---|---|---|
| Tar (A₁) | C₇₀H₉₄N₁₈O₂₇²⁺ | 809.3263 | 809.3273 | 1.24 |
| Tar (A₁ + A₁₃) | C₇₀H₉₃N₁₉O₂₉²⁺ | 831.8188 | NO | |

L-5-CH₃O-Trp

| | | | | |
|---|---|---|---|---|
| Tar (A₁) | C₇₁H₉₇N₁₇O₂₆²⁺ | 801.8390 | 801.8398 | 1.00 |
| Tar (A₁ + A₁₃) | C₇₂H₉₉N₁₇O₂₇²⁺ | 816.8443 | NO | |

L-5-OH-Trp

| | | | | |
|---|---|---|---|---|
| Tar (A₁) | C₇₀H₉₅N₁₇O₂₆²⁺ | 794.8312 | 794.8314 | 0.25 |
| Tar (A₁ + A₁₃) | C₇₀H₉₅N₁₇O₂₇²⁺ | 802.8286 | NO | |

TABLE 10
Summary of homologues of tryptophan 2,3-dioxygenase and kynurenine formamidase
proteins used for phylogenetic analysis of Tar13 and Tar16, respectively.

| | | | | | |
|---|---|---|---|---|---|
| | | | Associated with | Halogenase is | |
| | | Accession | putative NRPS | present within the | |
| Abbreviation/name | Organism | number | BGC | putative BGC | Comments, % identity |

Tryptophan 2,3-dioxygenases

| | | | | | |
|---|---|---|---|---|---|
| Tar13 | Saccharomonospora | WP_024877504.1 | YES | YES | Current study |
| | sp. CNQ-490 | | | | |
| rsTDO | Ralstonia | 2NOX | | | 41%, catabolism of |
| | metallidurans | | | | tryptophan, tested in vitro |
| xcTDO | Xanthomonas | 2NW7 | | | 41%, catabolism of |
| | campestris | | | | tryptophan, tested in vitro |
| hsTDO | Homo sapiens | 4PW8 | | | 30%, catabolism of |
| | | | | | tryptophan, tested in vitro |
| dmTDO | Drosophila | 4HKA | | | 30%, catabolism of |
| | melanogaster | | | | tryptophan, tested in vitro |
| TioF | Micromonospora sp. | CAJ34362.1 | YES | | 34%, thiocaroline BGC, |
| | ML1 | | | | tested in vitro |
| MarE | Streptomyces sp. | AHF22860.1 | YES | | 25%, maremycin BGC, |
| | CNQ-617 | | | | inserts only one oxygen |
| | | | | | atom, tested in vitro |
| SCO3646 | Streptomyces | NP_627840.1 | | | 37%, catabolism of |
| | coelicolor A3(2) | | | | tryptophan, tested in vitro |
| SpaTDOBGC | Streptomyces | WP_079163406.1 | YES | | 36%, actinomycin BGC, |
| | parvulus | | | | tested in vitro |
| SpaTDO | Streptomyces | 2721033010 | | | 37%, catabolism of |
| | parvulus | | | | tryptophan, tested in vitro |
| SanTDOBGC | Streptomyces | ADG27362.1 | YES | | 36%, not studied |
| | anulatus | | | | |
| SanTDO | Streptomyces | 2656756676 | | | 39%, not studied |
| | anulatus | | | | |
| SroTDO | Streptomyces | EFE76321.1 | | | 38%, not studied |
| | roseosporus | | | | |
| | NRRL 11379 | | | | |
| DptJ | Streptomyces | AAX31563.1 | YES | | 65%, daptomycin DGC |
| | roseosporus | | | | |
| | NRRL 11379 | | | | |
| Qui17 | Streptomyces | AET98915 | YES | | 42%, echinomycin BGC, |
| | griseovariabilis | | | | tested in vitro |
| SlaTDOBGC | Streptomyces | BAE98160.1 | YES | | 33%, not studied |
| | lasaliensis | | | | |
| StrTDOBGC | Streptomyces | BAH04172.1 | YES | | 49%, not studied |
| | triostinicus | | | | |
| SmsvTDOBGC | Saccharomonospora | WP_015786181.1 | YES | | 82%, not studied, identical |
| | viridis | | | | BGC organization as tar, but |
| | | | | | missing halogenase and |
| | | | | | flavin reductase encoding |
| | | | | | genes |
| AlaTDOHal | Alloactinosynnema | WP_091369532.1 | YES | YES | 69%, not studied |
| | album | | | | |
| AlaTDO | Alloactinosynnema | 2653846435 | | | 43%, not studied |
| | album | | | | |
| VsTDOHal | Verrucosispora | WP_093406808.1 | YES | YES | 68%, not studied |
| | sediminis | | | | |
| VsTDO | Verrucosispora | 2664226347 | | | 35%, not studied |
| | sediminis | | | | |
| SpsaTDOHal | Saccharopolyspora | WP_093154509.1 | YES | YES | 68%, not studied |
| | antimicrobica | | | | |
| SpshTDOHal | Saccharopolyspora | SEG43548.1 | YES | YES | 69%, not studied |
| | hirsuta | | | | |
| VspTDOHal | Verrucosispora sp. | WP_099845516.1 | YES | YES | 70%, not studied |
| | CNZ293 | | | | |
| VspTDO | Verrucosispora sp. | 2741237405 | | | 35%, not studied |
| | CNZ293 | | | | |
| SspF5727TDOHal | Streptomyces sp. | WP_051717236.1 | YES | YES | 69%, not studied |
| | NRRL F-5727 | | | | |
| SspF5727TDO | Streptomyces sp. | 2768681930 | | | 38%, not studied |
| | NRRL F-5727 | | | | |
| SceTDOHal | Streptomyces | WP_078940749.1 | YES | YES | 63%, not studied |
| | cellulosae | | | | |
| SceTDO | Streptomyces | 2768627411 | | | 39%, not studied |
| | cellulosae | | | | |
| SalTDO | Streptomyces | WP_086671565.1 | | | 67%, not studied |
| | albovinaceus | | | | |
| SzhTDOBGC | Streptomyces | WP_097230801.1 | YES | | 62%, not studied |
| | zhaozhouensis | | | | |
| SzhTDO | Streptomyces | 2718366227 | | | 49%, not studied |
| | zhaozhouensis | | | | |
| SspMS184TDO | Streptomyces sp. | WP_097874054.1 | | | 68%, not studied |
| | ms184 | | | | |
| SmuTDO | Streptomyces | WP_006122811.1 | | | 65%, not studied |
| | multispecies | | | | |
| SviTDO | Streptomyces | WP_078918332.1 | | | 62%, not studied |
| | violaceoruber | | | | |
| SolTDO | Streptomyces | GAX51800.1 | | | 66%, not studied |
| | olivochromogenes | | | | |
| AfTDOBGC | Actinoplanes | WP_023562381.1 | YES | | 61%, not studied |
| | friuliensis | | | | |
| AfTDO | Actinoplanes | 2555809471 | | | 41%, not studied |
| | friuliensis | | | | |
| SspCNT371TDOBGC | Streptomyces sp. | WP_027745021.1 | YES | | 58%, not studied |
| | CNT371 | | | | |
| SspCNT371TDO | Streptomyces sp. | 2516109309 | | | 37%, not studied |
| | CNT371 | | | | |
| SspCNH099TDO1 | Streptomyces | WP_027756395.1 | | | 52%, not studied |
| | sp. CNH099 | | | | |
| SspCNH099TDO2 | Streptomyces | 2516102262 | | | 38%, not studied |
| | sp.CNH099 | | | | |
| JgTDO | Jiangella gansuensis | WP_035812565.1 | | | 47%, not studied |
| NspTDO | Nonomuraea sp. | WP_080047241.1 | | | 48%, not studied |
| | ATCC 55076 | | | | |
| KcTDO | Kribbella | WP_020390285.1 | | | 46%, not studied |
| | catacumbae | | | | |
| SspCNS606TDO | Streptomyces sp. | WP_020390285.1 | | | 38%, not studied |
| | CNS606 | | | | |
| SspCNS606TDOBGC | Streptomyces sp. | WP_027762312.1 | YES | | 51%, not studied |
| | CNS606 | | | | |
| ThrTDO | Thermoactinospora | WP_084962261.1 | | | 47%, not studied |
| | rubra | | | | |
| FspG2TDOBGC | Frankia sp. G2 | WP_091282833.1 | YES | | 44%, not studied |
| NjTDO1 | Nonomuraea | SDH75058.1 | | | 47%, not studied |
| | jiangxiensis | | | | |
| NjTDO2 | Nonomuraea | WP_090933783.1 | | | 32%, not studied |
| | jiangxiensis | | | | |
| SspTAA204TDOBGC | Streptomyces sp. | WP_051264531.1 | YES | | 49%, not studied |
| | TAA204 | | | | |
| SspTAA204TDO | Streptomyces sp. | 2524964422 | | | 38%, not studied |
| | TAA204 | | | | |
| SnTDO | Stackebrandtia | WP_013017129.1 | | | 47%, not studied |
| | nassauensis | | | | |
| MmsrhTDO1 | Micromonospora | SCL19875.1 | | | 46%, not studied |
| | rhizosphaerae | | | | |
| MmsrhTDO2 | Micromonospora | WP_091348456.1 | | | 34%, not studied |
| | rhizosphaerae | | | | |
| SthTDO | Streptomyces | WP_096059116.1 | | | 47%, not studied |
| | thermoautotrophicus | | | | |

Kynurenine formamidases

| | | | | | |
|---|---|---|---|---|---|
| Tar16 | Saccharomonospora | WP_037335967.1 | YES | YES | Current study |
| | sp. CNQ490 | | | | |
| KFPsaer | Pseudomonas | WP_003114853.1 | | | 20%, catabolism of |
| | aeruginosa | | | | tryptophan, tested in vitro |
| KFBurkhce | Burkholderia | 4COG | | | 24%, catabolism of |
| | cenocepacia | | | | tryptophan, tested in vitro |
| KFBacilanth | Bacillus | WP_000858067.1 | | | 24%, catabolism of |
| | thuringiensis | | | | tryptophan, tested in vitro |
| KFDs | Drosophila | 4.00E+11 | | | 39%, catabolism of |
| | melanogaster | | | | tryptophan, tested in vitro |
| KFHs | Homo sapiens | Q63HM1 | | | 46%, catabolism of |
| | | | | | tryptophan, tested in vitro |
| VspCNZ293Hal | Verrucosisnora sp. | WP_099845518.1 | YES | YES | 76%, not studied |
| | CNZ293 | | | | |
| VseHal | Verrucosispora | SFD16583.1 | YES | YES | 73%, not studied |
| | sediminis | | | | |
| AlaHal | Alloactinosynnema | WP_091369528.1 | YES | YES | 74%, not studied |
| | album | | | | |
| SmspV | Saccharomonospora | WP_037312927.1 | YES | | 86%, not studied, identical |
| | viridis | | | | BGC organization as tar, but |
| | | | | | missing halogenase and |
| | | | | | flavin reductase encoding |
| | | | | | genes |
| SspF5193Hal | Streptomyces sp. | WP_043220233.1 | YES | YES | 65%, not studied |
| | NRRL F-5193 | | | | |
| ScaHal | Streotomyces | WP_049717738.1 | YES | YES | 62%, not studied |
| | caatingaensis | | | | |
| SroHal | Streptomyces | WP_106962696.1 | YES | YES | 62%, not studied |
| | roseochromogenus | | | | |
| SspNcostT6T1Hal | Streptomyces sp. | SBU91169.1 | YES | YES | 66%, not studied |
| | Ncost-T6T-1 | | | | |
| Kph | Kitasatospora | WP_033222207.1 | YES | YES | 63%, not studied |
| | phosalacinea | | | | |
| Kch | Kitasatospora | WP_035864171.1 | | | 66%, not studied |
| | cheerisanensis | | | | |
| Ssp1 | Streptomyces sp. 1 | WP_099900824.1 | | | 65%, not studied |
| Spspa | Saccharopolyspora | WP_093160261.1 | | | 59%, not studied |
| | antimicrobica | | | | |
| SspCNH189 | Streptomyces sp. | WP_024885731.1 | | | 32%, not studied |
| | CNH189 | | | | |
| Sgr | Streptomyces | WP_037640790.1 | | | 32%, not studied |
| | griseorubens | | | | |
| SspMh60 | Streptomyces sp. | WP_104636119.1 | | | 33%, not studied |
| | MH60 | | | | |
| Scan | Streptomyces | WP_059301759.1 | | | 32%, not studied |
| | canus | | | | |
| SspCS113 | Streptomyces sp. | WP_087808139.1 | | | 31%, not studied |
| | CS113 | | | | |
| Sviol | Streptomyces | WP_030932029.1 | | | 32%, not studied |
| | violaceoruber | | | | |
| SCO3644 | Streptomyces | WP_003975294.1 | | | 32%, catabolism of |
| | coelicolor A3(2) | | | | tryptophan, tested in vitro |

TABLE 11
Tryptophan halogenases that halogenate other positions of the trptophan indole ring at C5-7. These
may be used in addition to Tar14 to halogenate other positions or to increase production titers.

| Enzyme | | | | | | Sequence | |
|---|---|---|---|---|---|---|---|
| *not tested | Reaction | | Biosynthetic | | Accession | Identity/Similarity | |
| in-vitro | Site | Organism | Class | FDH? | Number | to Tar14 (%) | Literature |
| ClaH* | 5 | Streptomyces | Cladoniamides | Yes | AEO12707.1 | | Ryan, K. S. PLoS ONE |
| | | uncialis L72 | | | (GenBank) | | 6 (8), E23694 (2011) |
| AbeH* | 5 | uncultered | BE-54017 | Yes | AEF32095.1 | | Chang, F. Y. and Brady, |
| | | bacterium | (indolotryptoline | | (GenBank) | | S. F. J. Am. Chem. Soc. |
| | | AB1650 | core) | | | | 133 (26), 9996-9999 (2011) |
| PyrH | 5 | Streptomyces | Pyrroindomycin | Yes | AFV71318.1 | 53/69 | He, H.Y., Tang, G.L. |
| | | rugosporus | | | (GenBank) | | et al. Chem. Biol. 19 (10), |
| | | LL-42D005 | | | | | 1313-1323 (2012) |
| ThdH | 6 | Streptomyces | Thienodoline | Yes | ANW12118.1 | 37/54 | Milbredt, D., Patallo, |
| (also known | | albogriseolus | | | (GenBank) | | E. P. and van Pee, K. H. |
| as ThaI) | | MJ286-76F7 | | | | | Chembiochem 15 (7), |
| | | | | | | | 1011-1020 (2014) |
| Th-Hal | 6 | Streptomyces | Unknown? | Yes | 5LV9 (PDB) | 72/83 | Menon, B. R., Micklefield, |
| | | violaceusniger | | | | | J. et al. Org. Biomol. |
| | | SPC6 | | | | | Chem. 14 (39), 9354-9361 |
| | | | | | | | (2016) |
| SttH | 6 | Streptomyces | Unknown? | Yes | ADW94630.1 | 70/80 | Zeng, J. and Zhan, J. |
| | | toxytricini | | | (GenBank) | | Biotechnol. Lett. 33 (8), |
| | | NRRL 15443 | | | | | 1607-1613 (2011) |
| KtzR | 6 | Kutzneria | Kutzneride | Yes | ABV56598.1 | 69/80 | Fujimori, D. G., Walsh, |
| | | sp. 744 | | | (GenBank) | | C. T. cl al. Proc. Natl. |
| | | | | | | | Acad. Sci. U.S.A. 104 |
| | | | | | | | (42), 16498-16503 (2007) |
| BorH* | 6 | uncultered | Borregomycin | Yes | AGI62217.1 | 38/53 | Chang, F. Y. and Brady, |
| | | bacterium | | | (GenBank) | | S. F. Proc. Natl. Acad. |
| | | AB 1091 | | | | | Sci. U.S.A. 110 (7), |
| | | | | | | | 2478-2483 (2013) |
| KtzQ | 7 | Kutzneria | Kutzneride | Yes | ABV56597.1 | 36/53 | Fujimori, D. G., Walsh, |
| | | sp. 744 | | | (GenBank) | | C. T. el al. Proc. Natl. |
| | | | | | | | Acad. Sci. U.S.A. 104 |
| | | | | | | | (42), 16498-16503 (2007) |
| PrnA | 7 | Pseudomonas | Pyrrolnitrin | Yes | 2ARD (PDB) | 38/55 | Dong, C., Naismith J. H. |
| | | fluorescens | | | | | et al. Science. 309 (5744), |
| | | | | | | | 2216-2219 (2005) |
| RebH | 7 | Lechevalieria | Rebeccamycin | Yes | 2E46 (PDB) | 36/53 | E. Yeh, S. Garneau, C. T. |
| | | aerocolonigenes | | | | | Walsh. Proc. Natl. Acad. |
| | | ATCC 39243 | | | | | Sci. USA, 102, 3960-3965 |
| | | | | | | | (2005) |
| AtmH* | 7 | | AT2433 | Yes | | | Gao Q., Thorson J. S. |
| | | | (indolocarbazole) | | | | et al. Chem Biol. 13, |
| | | | | | | | 733-3 (2006) |

### REFERENCES

### Example 1. References

[1] T. L. Yaksh, R. Schwarcz, H. R. Snodgrass, J. Pain 2017, 18, 1184-1196.
[2] L. Vécsei, L. Szalárdy, F. Fülöp, J. Toldi, Nat. Rev. Drug Discov. 2013, 12, 64-82.
[3] F. G. Salituro, R. C. Tomlinson, B. M. Baron, M. G. Palfreyman, I. A. McDonald, W. Schmidt, H. Q. Wu, P. Guidetti, R. Schwarcz, J. Med. Chem. 1994, 37, 334-336.
[4] M. Varasi, A. Della Torre, F. Heidempergher, P. Pevarello, C. Speciale, P. Guidetti, D. Wells, R. Schwarcz, Eur. J. Med. Chem. 1996, 31, 11-21.
[5] K. A. Reynolds, H. Luhavaya, J. Li, S. Dahesh, V. Nizet, K. Yamanaka, B. S. Moore, J. Antibiot. 2018, 71, 333-338.
[6] K. Yamanaka, K. A. Reynolds, R. D. Kersten, K. S. Ryan, D. J. Gonzalez, V. Nizet, P. C. Dorrestein, B. S. Moore, Proc. Natl. Acad. Sci. U.S.A. 2014, 111, 1957-1962.
[7] V. A. Alferova, et al., Amino Acids 2018, 50, 1697-1705.
[8] R. Schwarcz, Curr. Opin. Pharmacol. 2004, 4, 12-17.
[9] O. Kumasov, L. Jablonski, B. Polanuyer, P. Dorrestein, T. Begley, A. Osterman, FEMS Microbiol. Lett. 2003, 227, 219-227.
[10] M. Nozaki, Y. Ishimura, Biochem. J 1972, 128, 24P-25P.
[11] A. Sheoran, A. King, A. Velasco, J. M. Pero, S. Gameau-Tsodikova, Mol. BioSyst. 2008, 4, 622-628.
[12] M. J. M. Hitchcock, E. Katz, Arch. Biochem. Biophys. 1988, 261, 148-160.
[13] U. Keller, M. Lang, I. Cmovcic, F. Pfennig, F. Schauwecker, J. Bacteriol. 2010, 192, 2583-2595.
[14] C. Zhang, L. Kong, Q. Liu, X. Lei, T. Zhu, J. Yin, B. Lin, Z. Deng, D. You, PLoS One 2013, 8, e56772.
[15] D. Brown, M. J. Hitchcock, E. Katz, Can. J. Microbiol. 1986, 32, 465-472.
[16] P. C. Dorrestein, E. Yeh, S. Garneau-Tsodikova, N. L. Kelleher, C. T. Walsh, Proc. Natl. Acad. Sci. U.S.A. 2005, 102, 13843-13848.
[17] M. A. Ortega, et al., ACS Chem. Biol. 2017, 12, 548-557.
[18] Y. Liu, W. Tao, S. Wen, Z. Li, A. Yang, Z. Deng, Y. Sun. mBio 2015, 6, e01714-e01715.
[19] J. P. Gomez-Escribano, M. J. Bibb, Microb. Biotechnol. 2011, 4, 207-215.
[20] C. J. Wilkinson, Z. A. Hughes-Thomas, C. J. M. Rowe, I. Bohm, M. Deacon, M. Wheatcroft, G. Wirtz, J. Staunton, P. Leadlay, J. Mol. Microbiol. Biotechnol. 2002, 4, 417-426.
[21] C. Dong, S. Flecks, S. Unversucht, C. Haupt, K.-H. van Pée, J. H. Naismith, Science 2005, 309, 2216-2219.
[22] A. E. Gamal, et al., Proc. Natl. Acad. Sci. U.S.A. 2016, 113, 3797-3802.
[23] B. R. K. Menon, J. Latham, M. S. Dunstan, E. Brandenburger, U. Klemstein, D. Leys, C. Karthikeyan, M. F. Greaney, S. A. Shepherd, J. Micklefield, Org. Biomol. Chem. 2016, 14, 9354-9361.
[24] J. R. Heemstra, C. T. Walsh, J. Am. Chem. Soc. 2008, 130, 14024-14025.
[25] D. R. M. Smith, A. R. Uria, E. J. N. Helfrich, D. Milbredt, K.-H. van Pée, J. Piel, R. J. M. Goss, ACS Chem. Biol. 2017, 12, 1281-1287.
[26] A.-C. Moritzer. H. Minges, T. Prior, M. Frese, N. Sewald, H. H. Niemann, J. Biol. Chem. 2018. jbc.RA118.005393.
[27] F.-Y. Chang, S. F. Brady, Proc. Natl. Acad. Sci. U.S.A. 2013, 110, 2478-2483.
[28] S. A. Shepherd, B. R. K. Menon. H. Fisk, A.-W. Struck, C. Levy, D. Leys, J. Micklefield. ChemBioChem 2016, 17, 821-824.
[29] X. Zhu, W. De Laurentis, K. Leang, J. Herrmann, K. Ihlefeld, K.-H. van Pee, J. H. Naismith, J Mol. Biol. 2009, 391, 74-85.
[30] K. Miller, C. Faeh, F. Diederich, Science 2007, 317, 1881.
[31] V. M. Isabella, et al., Nature Biotechnol. 2018, 36, 857-864.
[32] P. J. Kennedy, J. F. Cryan, T. G. Dinan, G. Clarke, Neuropharmacology 2017, 112, 399-412.

### Example 2. References

[1] M. R. Green, J. Sambrook, Cold Spring Harb. Protoc. 2017, 2017, pdb.prot093385.
[2] C. J. Wilkinson, Z. A. Hughes-Thomas, C. J. M. Rowe, I. Bohm, M. Deacon. M. Wheatcroft, G. Wirtz, J. Staunton, P. Leadlay, J. Mol. Microbiol. Biotechnol. 2002, 4, 417-426.
[3] R. E. Cobb, Y. Wang, H. Zhao, ACS Synth. Biol. 2015, 4, 723-728.
[4] F. Flett, V. Mersinias, C. P. Smith, FEMS Microbiol. Lett. 1997, 155, 223-229.
[5] K. Yamanaka, K. A. Reynolds, R. D. Kersten, K. S. Ryan, D. J. Gonzalez, V. Nizet, P. C. Dorrestein, B. S. Moore, Proc. Natl. Acad. Sci. U.S.A. 2014, 111, 1957-1962.
[6] A. M. G. Costas, A. K. White, W. W. Metcalf, J. Biol. Chem. 2001, 276, 17429-17436.
[7] E. Eichhom, J. R. van der Ploeg, T. Leisinger, J. Biol. Chem. 1999, 274, 26639-26646.
[8] T. Kieser, M. J. Bibb, M. J. Buttner, K. F. Chater, D. A. Hopwood, Practical Streptomyces Genetics. The John Innes Foundation, Norwich, 2000.
[9] K. A. Datsenko, B. L. Wanner, Proc. Natl. Acad. Sci. U.S.A. 2000, 97, 6640-6645.
[10] J. P. Gomez-Escribano, M. J. Bibb, Microb. Biotechnol. 2011, 4, 207-215.
[11] R. McDaniel, S. Ebert-Khosla, D. Hopwood, C. Khosla, Science 1993, 262, 1546.
[12] W. Kabsch, Acta Crystallogr. D Biol. Crystallogr. 2010, 66, 125-132.
[13] C. Vonrhein, C. Flensburg, P. Keller, A. Sharff, O. Smart, W. Paciorek, T. Womack, G. Bricogne, Acta Cryst. D 2011, 67, 293-302.
[14] A. J. McCoy, R. W. Grosse-Kunstleve, P. D. Adams, M. D. Winn, L. C. Storoni, R. J. Read, J. Appl. Crystallogr. 2007, 40, 658-674.
[15] P. D. Adams, P. V. Afonine, G. Bunkóczi, V. B. Chen, I. W. Davis, N. Echols, J. J. Headd, L.-W. Hung, G. J. Kapral, R. W. Grosse-Kunstleve, et al., Acta Crystallogr. D Biol. Crystallogr. 2010, 66, 213-221.
[16] B. R. K. Menon, J. Latham, M. S. Dunstan, E. Brandenburger, U. Klemstein, D. Leys, C. Karthikeyan, M. F. Greaney, S. A. Shepherd, J. Micklefield, Org. Biomol. Chem. 2016, 14, 9354-9361.
[17] P. Emsley, B. Lohkamp, W. G. Scott, K. Cowtan, Acta Crystallogr. D Biol. Crystallogr. 2010, 66, 486-501.
[18] E. F. Pettersen, T. D. Goddard, C. C. Huang, G. S. Couch, D. M. Greenblatt, E. C. Meng, T. E. Ferrin, J. Comput. Chem. 2004, 25, 1605-1612.
[19] Y. Liu, W. Tao, S. Wen, Z. Li, A. Yang, Z. Deng, Y. Sun, mBio 2015, 6, e01714-e01715.
[20] Q. Tu, J. Yin, J. Fu, J. Herrmann, Y. Li, Y. Yin, A. F. Stewart, R. Müller, Y. Zhang, Sci. Rep. 2016, 6, 24648.
[21] M. Landau, I. Mayrose, Y. Rosenberg, F. Glaser, E. Martz, T. Pupko, N. Ben-Tal, Nucleic Acids Res. 2005, 33, W299-W302.
[22] F. Forouhar, J. L. R. Anderson, C. G. Mowat, S. M. Vorobiev, A. Hussain, M. Abashidze, C. Bruckmann, S. J. Thackray, J. Seetharaman, T. Tucker, R. Xiao, L.-C. Ma, L. Zhao, T. B. Acton, G. T. Montelione, S. K. Chapman, Proc. Natl. Acad. Sci. U.S.A. 2007. 104, 473-478.
[23] E. Katz, D. Brown, M. J. Hitchcock, Meth. Enzymol. 1987, 142, 225-234.
[24] J. Basran, S. A. Rafice, N. Chauhan, I. Efimov, M. R. Cheesman, L. Ghamsari, E. L. Raven, Biochemistry 2008, 47, 4752-4760.
[25] Y. Zhang, Y. Zou, N. L. Brock, T. Huang, Y. Lan, X. Wang, Z. Deng, Y. Tang, S. Lin, J. Am. Chem. Soc. 2017, 139, 11887-11894.

INFORMAL SEQUENCE LISTING
Tar protein sequences and nucleic acid sequences
SEQ ID NO: 1 (Tar13 Wild Type)
MTERTATRTEPAYGEILRLDELLELACVNDEADRALFLSAHQACEIWFAVVLRHLED
VTDALSLDDGATAAELLERLPRIITVIIEHFEVLGTLKPEAFDRIRADLGSSSGFQSVQY
REIEYLCGARDTRFLNTAGFRDRDRRRLRERLAKRSLSNVFLEYRGRAGDRDACRIS
DALHEFDDSVRALRLRHAGIAELFLGSIPGTAGTAGAAYLRRSASRTLFPELFDRRAS
GTGG
SEQ ID NO: 2 (Tar13 Expressed)
MGSSHHHHHHSSGLVPRGSHMTERTATRTEPAYGEILRLDELLELACVNDEADRALF
LSAHQACEIWFAVVLRHLEDVTDALSLDDGATAAELLERLPRIITVIIEHFEVLGTLKP
EAFDRIRADLGSSSGFQSVQYREIEYLCGARDTRFLNTAGFRDRDRRRLRERLAKRSL
SNVFLEYRGRAGDRDACRISDALHEFDDSVRALRLRHAGIAELFLGSIPGTAGTAGA
AYLRRSASRTLFPELFDRRASGTGG
SEQ ID NO: 3 (Tar14 Wild Type)
MSVSGSERSAEGNRKKRVVIVGGGTAGWMTASYLTAAFGDRVDLTVVESAQIGTIG
VGEATFSDIRHFFEFLRLEESDWMPECNATYKLAVRFENWREPGHHFYHPFEQMSSV
DGFPLSDWWLRNATTSRFDKDSFVMTSLCDAGVSPRYLDGSLIDQDFVEQERDDDS
ARSTIAEYQGAQFPYAYHFEAHLLAKYLTGYATRRGTRHIVDNVVDVALDERGWIS
HVRTEEHGDLEADLFVDCTGFRGLLLNKALGEPFVSYQDTLPNDSAVALQVPLDME
REPIRPCTTATAQEAGWIWTIPLISRVGTGYVYASDYTTPEQAERVLRDFVGPAAAD
VPANHIKMRIGRSRRSWVNNCVGVGLSSGFVEPLESTGIFFIHHAIEQIVKYFPSGGAG
DDRLRELYNRSVGHVMDGVREFLVLHYRSAKRADNQYWKDTKTRTVPDSLAERIE
FWKHKVPDAETVYPYYHGLPPYSYNCILLGMGGIDVNYSPALDWANEKAALAEFER
IRVKAEKLVQELPTQNEYFAAMRAGR
SEQ ID NO: 4 (Tar14 Expressed)
MGSSHHHHHHSSGLVPRGSHMSVSGSERSAEGNRKKRVVIVGGGTAGWMTASYLT
AAFGDRVDLTVVESAQIGTIGVGEATFSDIRHFFEFLRLEESDWMPECNATYKLAVRF
ENWREPGHHFYHPFEQMSSVDGFPLSDWWLRNPTTSRFDKDSFVMTSLCDAGVSPR
YLDGSLIDQDFVEQERDDDSARSTIAEYQGAQFPYAYHFEAHLLAKYLTGYATRRGT
RHIVDNVVDVALDERGWISHVRTEEHGDLEADLFVDCTGFRGLLLNKALGEPFVSY
QDTLPNDSAVALQVPLDMEREPIRPCTTATAQEAGWIWTIPLISRVGTGYVYASDYT
TPEQAERVLRDFVGPAAADVPANHIKMRIGRSRRSWVNNCVGVGLSSGFVEPLESTG
IFFIHHAIEQIVKYFPSGGAGDDRLRELYNRSVGHVMDGVREFLVLHYRSAKRADNQ
YWKDTKTRTVPDSLAERIEFWKHKVPDAETVYPYYHGLPPYSYNCILLGMGGIDVN
YSPALDWANEKAALAEFERIRVKAEKLVQELPTQNEYFAAMRAGR
SEQ ID NO: 5 (Tar14 Expressed with cleaved His tag)
GSHMSVSGSERSAEGNRKKRVVIVGGGTAGWMTASYLTAAFGDRVDLTVVESAQI
GTIGVGEATFSDIRHFFEFLRLEESDWMPECNATYKLAVRFENWREPGHHFYHPFEQ
MSSVDGFPLSDWWLRNPTTSRFDKDSFVMTSLCDAGVSPRYLDGSLIDQDFVEQER
DDDSARSTIAEYQGAQFPYAYHFEAHLLAKYLTGYATRRGTRHIVDNVVDVALDER
GWISHVRTEEHGDLEADLFVDCTGFRGLLLNKALGEPFVSYQDTLPNDSAVALQVPL
DMEREPIRPCTTATAQEAGWIWTIPLISRVGTGYVYASDYTTPEQAERVLRDFVGPA
AADVPANHIKMRIGRSRRSWVNNCVGVGLSSGFVEPLESTGIFFIHHAIEQIVKYFPSG
GAGDDRLRELYNRSVGHVMDGVREFLVLHYRSAKRADNQYWKDTKTRTVPDSLA
ERIEFWKHKVPDAETVYPYYHGLPPYSYNCILLGMGGIDVNYSPALDWANEKAALA
EFERIRVKAEKLVQELPTQNEYFAAMRAGR
SEQ ID NO: 6 (Tar15 WT)
MSIGRSTAEAGAMASFRDAMASFPTGVSVVTTMHTDGAPRGMTCSALCSVSMEPPL
LLVCLRTASPTLDAIRVRGGFVVNLLKYQARDTARLFASGDTGRFDQVAWRHHPGT
AGPCLVDDAHAAVDCQVLRRDEAGDHVVVLGEVVGVRTLSGAAPLLYGLRRYAR
WPDASSLLDEAR
SEQ ID NO: 7 (Tar15 Expressed)
MGSSHHHHHHSSGLVPRGSHMSIGRSTAEAGAMASFRDAMASFPTGVSVVTTMHTD
GAPRGMTCSALCSVSMEPPLLLVCLRTASPTLDAIRVRGGFVVNLLKYQARDTARLF
ASGDTGRFDQVAWRHHPGTAGPCLVDDAHAAVDCQVLRRDEAGDHVVVLGEVVG
VRTLSGAAPLLYGLRRYARWPDASSLLDEAR
SEQ ID NO: 8 (Tar16 WT)
MAAAVFRSYDQHELDIQYSPSSRVDDVQSYLREYARLSARARTEIDGFVEIRYGEFPE
QVVDYFPAGTSGGSLLVFVHGGYWQELSRRESAFMAADL1ERGVSVAALGYGLAPR
YTVPEIVTMVSEGVRWLCRNAAGLPGSPRRVVLSGSSAGAHLTTMSLLDEAGWRRD
GWRPAEAVSGAVLLSGVYDLDPVRRTYVNAPLGLDADTALACSPQRRPLAGLPPLV
VARGDNETGEFARQQREFVAAVRRAGGSVNDLVVRGRNHFDLAFDLGDPATSLGA
AVARLVE
SEQ ID NO: 9 (Tar16 Expressed)
MGSSHHHHHHSSGLVPRGSHMAAAVFRSYDQHELDIQYSPSSRVDDVQSYLREYAR
LSARARTEIDGFVEIRYGEFPEQVVDYFPAGTSGGSLLVFVHGGYWQELSRRESAFM
AADLIERGVSVAALGYGLAPRYTVPEIVTMVSEGVRWLCRNAAGLPGSPRRVVLSG
SSAGAHLTTMSLLDEAGWRRDGWRPAEAVSGAVLLSGVYDLDPVRRTYVNAPLGL
DADTALACSPQRRPLAGLPPLVVARGDNETGEFARQQREFVAAVRRAGGSVNDLVV
RGRNHFDLAFDLGDPATSLGAAVARLVE
SEQ ID NO: 10 (tar13_CTHF_tag)
ATGACCGAGCGTACGGCCACCCGCACCGAACCTGCATATGGCGAGATTTTACGTC
TTGATGAGCTCCTTGAGCTCGCGTGTGTAAACGACGAAGCAGATCGCGCATTATT
CTTATCTGCTCACCAGGCGTGTGAGATTTGGTTCGCGGTTGTTTTACGCCACTTAG
AAGACGTGACGGATGCGCTGAGTTTAGACGACGGTGCTACGGCTGCGGAGTTGC
TTGAGCGTCTCCCTCGCATCATCACCGTTATCATTGAGCACTTCGAAGTCCTCGGT
ACCCTTAAGCCCGAGGCTTTTGATCGCATCCGCGCTGATCTGGGCAGCAGCTCCG
GCTTTCAATCGGTTCAATACCGTGAGATCGAGTATCTCTGCGGAGCCCGCGACAC
GCGCTTCCTTAATACGGCGGGCTTCCGTGATCGCGATCGCCGCCGTCTCCGTGAA
CGTTTAGCGAAGCGCTCGCTGAGCAACGTTTTCCTTGAATATCGTGGTCGCGCCG
GTGACCGCGATGCATGCCGTATTTCGGACGCGCTTCATGAATTTGACGACTCAGT
TCGTGCCCTGCGCTTGCGCCATGCTGGTATCGCTGAGCTGTTCTTAGGCAGCATC
CCTGGCACGGCAGGCACGGCGGGAGCGGCGTATCTCCGTCGTTCTGCTTCCCGCA
CCCTGTTTCCAGAATTGTTTGACCGCCGTGCGTCCGGAACAGGTGGCGCGCTCG
TCCCGCGTGGTTCTCACCATCACCATCACCACGACTACAAGGACGACGACG
ACAAATGA
SEQ ID NO: 11 (tar13)
ATGACCGAGCGTACGGCCACCCGCACCGAACCTGCATATGGCGAGATTTTACGTC
TTGATGAGCTCCTTGAGCTCGCGTGTGTAAACGACGAAGCAGATCGCGCATTATT
CTTATCTGCTCACCAGGCGTGTGAGATTTGGTTCGCGGTTGTTTTACGCCACTTAG
AAGACGTGACGGATGCGCTGAGTTTAGACGACGGTGCTACGGCTGCGGAGTTGC
TTGAGCGTCTCCCTCGCATCATCACCGTTATCATTGAGCACTTCGAAGTCCTCGGT
ACCCTTAAGCCCGAGGCTTTTGATCGCATCCGCGCTGATCTGGGCAGCAGCTCCG
GCTTTCAATCGGTTCAATACCGTGAGATCGAGTATCTCTGCGGAGCCCGCGACAC
GCGCTTCCTTAATACGGCGGGCTTCCGTGATCGCGATCGCCGCCGTCTCCGTGAA
CGTTTAGCGAAGCGCTCGCTGAGCAACGTTTTCCTTGAATATCGTGGTCGCGCCG
GTGACCGCGATGCATGCCGTATTTCGGACGCGCTTCATGAATTTGACGACTCAGT
TCGTGCCCTGCGCTTGCGCCATGCTGGTATCGCTGAGCTGTTCTTAGGCAGCATC
CCTGGCACGGCAGGCACGGCGGGAGCGGCGTATCTCCGTCGTTCTGCTTCCCGCA
CCCTGTTTCCAGAATTGTTTGACCGCCGTGCGTCCGGAACAGGTGGC
SEQ ID NO: 12 (tar14_CTHF_tag)
ATGTCCGTGTCTGGTAGCGAGCGCAGCGCCGAAGGAAATCGTAAGAAACGTGTG
GTCATCGTTGGCGGTGGCACCGCCGGGTGGATGACTGCAAGTTATCTTACCGCAG
CGTTTGGAGATCGTGTAGACTTGACCGTCGTAGAATCAGCACAAATTGGAACCAT
CGGTGTTGGAGAGGCGACATTTTCGGACATCCGCCATTTCTTCGAATTTCTGCGC
TTAGAGGAGAGCGACTGGATGCCGGAATGTAATGCGACATACAAACTGGCAGTA
CGTTTTGAGAATTGGCGTGAACCAGGGCACCATTTCTATCATCCTTTTGAGCAGA
TGTCCTCTGTTGACGGCTTCCCTTTAAGTGACTGGTGGTTGCGTAATCCAACAACC
AGCCGCTTCGATAAAGATAGCTTTGTTATGACCTCGTTATGTGATGCGGGAGTAT
CTCCACGCTACTTAGACGGCTCATTAATTGATCAAGATTTCGTCGAACAAGAGCG
CGATGACGACTCGGCGCGCAGTACAATCGCGGAGTATCAAGGCGCGCAATTTCC
GTATGCATATCACTTCGAGGCACACCTCTTGGCGAAGTACTTAACGGGATATGCC
ACCCGTCGTGGTACGCGTCACATCGTGGACAATGTAGTGGACGTGGCACTCGATG
AGCGTGGCTGGATCAGCCATGTACGCACAGAGGAGCACGGGGATTTAGAAGCAG
ACTTGTTCGTTGATTGTACTGGGTTCCGTGGCCTTTTGCTGAATAAGGCCTTAGGC
GAGCCTTTTGTGTCTTATCAAGACACGCTCCCGAATGACAGCGCAGTGGCCCTGC
AAGTTCCTCTGGATATGGAACGTGAGCCAATCCGTCCTTGCACTACTGCCACCGC
CCAAGAGGCCGGCTGGATTTGGACGATTCCACTGATCAGCCGTGTGGGAACGGG
CTATGTTTACGCGTCGGATTACACAACCCCCGAGCAAGCTGAACGTGTGCTTCGT
GATTTTGTAGGTCCAGCAGCTGCAGACGTACCAGCGAACCACATCAAGATGCGT
ATCGGCCGCAGTCGTCGCAGCTGGGTTAATAATTGTGTCGGTGTCGGGTTATCCA
GCGGATTCGTCGAGCCGTTGGAGTCAACGGGCATCTTCTTTATCCATCACGCAAT
TGAACAAATTGTGAAGTATTTTCCGTCGGGCGGCGCGGGAGATGACCGCTTGCGT
GAGCTTTACAATCGCAGCGTTGGGCACGTGATGGACGGAGTTCGTGAATTCTTGG
TTTTACATTATCGTTCAGCAAAGCGTGCGGATAACCAATATTGGAAGGATACCAA
GACACGTACCGTACCTGACTCGTTGGCGGAGCGTATCGAATTCTGGAAACACAA
GGTACCCGATGCTGAGACGGTATATCCGTACTATCACGGCCTTCCGCCCTATAGC
TACAATTGTATTCTTCTCGGAATGGGCGGCATTGATGTTAACTACAGCCCCGCAT
TGGATTGGGCAAATGAGAAGGCCGCGTTGGCCGAGTTCGAACGCATTCGCGTTA
AAGCAGAGAAACTCGTCCAGGAGCTGCCTACACAAAATGAGTACTTCGCGGCCA
TGCGTGCGGGCCGCGCGCTGGTTCCGCGCGGCAGTCATCACCACCATCACCA
TGATTACAAGGACGACGACGATAAATGA
SEQ ID NO: 13 (tar14)
ATGTCCGTGTCTGGTAGCGAGCGCAGCGCCGAAGGAAATCGTAAGAAACGTGTG
GTCATCGTTGGCGGTGGCACCGCCGGGTGGATGACTGCAAGTTATCTTACCGCAG
CGTTTGGAGATCGTGTAGACTTGACCGTCGTAGAATCAGCACAAATTGGAACCAT
CGGTGTTGGAGAGGCGACATTTTCGGACATCCGCCATTTCTTCGAATTTCTGCGC
TTAGAGGAGAGCGACTGGATGCCGGAATGTAATGCGACATACAAACTGGCAGTA
CGTTTTGAGAATTGGCGTGAACCAGGGCACCATTTCTATCATCCTTTTGAGCAGA
TGTCCTCTGTTGACGGCTTCCCTTTAAGTGACTGGTGGTTGCGTAATCCAACAACC
AGCCGCTTCGATAAAGATAGCTTTGTTATGACCTCGTTATGTGATGCGGGAGTAT
CTCCACGCTACTTAGACGGCTCATTAATTGATCAAGATTTCGTCGAACAAGAGCG
CGATGACGACTCGGCGCGCAGTACAATCGCGGAGTATCAAGGCGCGCAATTTCC
GTATGCATATCACTTCGAGGCACACCTCTTGGCGAAGTACTTAACGGGATATGCC
ACCCGTCGTGGTACGCGTCACATCGTGGACAATGTAGTGGACGTGGCACTCGATG
AGCGTGGCTGGATCAGCCATGTACGCACAGAGGAGCACGGGGATTTAGAAGCAG
ACTTGTTCGTTGATTGTACTGGGTTCCGTGGCCTTTTGCTGAATAAGGCCTTAGGC
GAGCCTTTTGTGTCTTATCAAGACACGCTCCCGAATGACAGCGCAGTGGCCCTGC
AAGTTCCTCTGGATATGGAACGTGAGCCAATCCGTCCTTGCACTACTGCCACCGC
CCAAGAGGCCGGCTGGATTTGGACGATTCCACTGATCAGCCGTGTGGGAACGGG
CTATGTTTACGCGTCGGATTACACAACCCCCGAGCAAGCTGAACGTGTGCTTCGT
GATTTTGTAGGTCCAGCAGCTGCAGACGTACCAGCGAACCACATCAAGATGCGT
ATCGGCCGCAGTCGTCGCAGCTGGGTTAATAATTGTGTCGGTGTCGGGTTATCCA
GCGGATTCGTCGAGCCGTTGGAGTCAACGGGCATCTTCTTTATCCATCACGCAAT
TGAACAAATTGTGAAGTATTTTCCGTCGGGCGGCGCGGGAGATGACCGCTTGCGT
GAGCTTTACAATCGCAGCGTTGGGCACGTGATGGACGGAGTTCGTGAATTCTTGG
TTTTACATTATCGTTCAGCAAAGCGTGCGGATAACCAATATTGGAAGGATACCAA
GACACGTACCGTACCTGACTCGTTGGCGGAGCGTATCGAATTCTGGAAACACAA
GGTACCCGATGCTGAGACGGTATATCCGTACTATCACGGCCTTCCGCCCTATAGC
TACAATTGTATTCTTCTCGGAATGGGCGGCATTGATGTTAACTACAGCCCCGCAT
TGGATTGGGCAAATGAGAAGGCCGCGTTGGCCGAGTTCGAACGCATTCGCGTTA
AAGCAGAGAAACTCGTCCAGGAGCTGCCTACACAAAATGAGTACTTCGCGGCCA
TGCGTGCGGGCCGC
SEQ ID NO: 14 (tar15_CTHF_tag)
ATGAGTATTGGGCGTTCGACAGCAGAGGCGGGTGCTATGGCGAGTTTTCGCGAC
GCCATGGCAAGCTTCCCAACTGGCGTAAGTGTTGTGACGACTATGCACACGGAC
GGGGCGCCCCGCGGGATGACTTGTTCCGCGTTGTGTAGCGTTTCGATGGAGCCGC
CGTTACTTCTGGTGTGTCTCCGTACGGCAAGCCCAACATTGGACGCAATCCGCGT
TCGTGGCGGCTTCGTAGTTAACCTGTTAAAGTATCAGGCCCGCGATACGGCGCGT
TTATTCGCCTCGGGTGACACTGGCCGCTTTGACCAAGTAGCATGGCGTCATCATC
CCGGAACTGCAGGCCCATGTCTTGTGGACGACGCACATGCCGCTGTTGACTGTCA
AGTACTGCGTCGCGATGAAGCAGGGGATCACGTGGTGGTTTTAGGCGAGGTCGT
CGGTGTACGCACTCTGAGCGGCGCAGCTCCTCTTCTCTATGGACTCCGTCGCTAC
GCCCGTTGGCCAGATGCCTCAAGTCTTTTGGACGAGGCACGTGCACTTGTTCCA
CGCGGCAGCCATCACCACCATCACCACGACTACAAGGACGATGATGATAAG
TGA
SEQ ID NO: 15 (tar15)
ATGAGTATTGGGCGTTCGACAGCAGAGGCGGGTGCTATGGCGAGTTTTCGCGAC
GCCATGGCAAGCTTCCCAACTGGCGTAAGTGTTGTGACGACTATGCACACGGAC
GGGGCGCCCCGCGGGATGACTTGTTCCGCGTTGTGTAGCGTTTCGATGGAGCCGC
CGTTACTTCTGGTGTGTCTCCGTACGGCAAGCCCAACATTGGACGCAATCCGCGT
TCGTGGCGGCTTCGTAGTTAACCTGTTAAAGTATCAGGCCCGCGATACGGCGCGT
TTATTCGCCTCGGGTGACACTGGCCGCTTTGACCAAGTAGCATGGCGTCATCATC
CCGGAACTGCAGGCCCATGTCTTGTGGACGACGCACATGCCGCTGTTGACTGTCA
AGTACTGCGTCGCGATGAAGCAGGGGATCACGTGGTGGTTTTAGGCGAGGTCGT
CGGTGTACGCACTCTGAGCGGCGCAGCTCCTCTTCTCTATGGACTCCGTCGCTAC
GCCCGTTGGCCAGATGCCTCAAGTCTTTTGGACGAGGCACGT
SEQ ID NO: 16 (NT_6xHis_Thrombim_tar16)
ATGGGGAGTAGCCACCACCATCACCACCATTCCTGTGGCTTGGTCCCGCGC
GGTTCGCACATGGCGGCCGCCGTATTCCGCAGCTACGATCAGCATGAGTTAGAC
ATTCAGTATAGCCCAAGCTCGCGTGTCGACGACGTGCAATCATACCTTCGCGAGT
ACGCTCGTCTTAGTGCACGTGCCCGCACCGAGATCGACGGATTTGTAGAGATTCG
TTACGGTGAATTCCCGGAACAAGTTGTGGATTACTTTCCAGCTGGAACGTCGGGC
GGCTCGCTCTTAGTGTTTGTGCACGGCGGCTACTGGCAAGAATTGTCCCGCCGTG
AGTCCGCGTTCATGGCAGCGGACTTAATCGAGCGCGGTGTTTCAGTGGCAGCTCT
GGGCTATGGTTTAGCACCTCGTTATACTGTGCCGGAAATCGTGACCATGGTGAGC
GAAGGTGTCCGTTGGTTGTGTCGTAATGCGGCCGGCCTGCCGGGGAGCCCACGTC
GTGTTGTACTGTCGGGTAGCTCCGCAGGCGCTCATCTTACCACCATGAGCCTGTT
AGATGAAGCGGGGTGGCGTCGCGACGGTTGGCGTCCTGCAGAGGCGGTGAGCGG
TGCGGTTTTGTTAAGCGGCGTGTACGACTTAGACCCGGTCCGTC GCACATACGTC
AATGCACCATTGGGACTGGACGCTGATACAGCTCTGGCCTGTTCTCCTCAGCGTC
GTCCGTTGGCCGGCCTGCCCCCTCTTGTTGTGGCCCGCGGCGACAATGAAACCGG
TGAATTTGCACGTCAACAACGTGAGTTCGTTGCGGCAGTGCGCCGCGCGGGTGG
AAGTGTGAATGACCTGGTGGTGCGCGGTCGCAACCACTTTGACTTAGCATTCGAC
TTGGGCGACCCAGCCACGTCACTTGGCGCTGCAGTGGCACGTCTCGTTGAATGA
SEQ ID NO: 17 (tar16)
ATGGCGGCCGCCGTATTCCGCAGCTACGATCAGCATGAGTTAGACATTCAGTATA
GCCCAAGCTCGCGTGTCGACGACGTGCAATCATACCTTCGCGAGTACGCTCGTCT
TAGTGCACGTGCCCGCACCGAGATCGACGGATTTGTAGAGATTCGTTACGGTGAA
TTCCCGGAACAAGTTGTGGATTACTTTCCAGCTGGAACGTCGGGCGGCTCGCTCT
TAGTGTTTGTGCACGGCGGCTACTGGCAAGAATTGTCCCGCCGTGAGTCCGCGTT
CATGGCAGCGGACTTAATCGAGCGCGGTGTTTCAGTGGCAGCTCTGGGCTATGGT
TTAGCACCTCGTTATACTGTGCCGGAAATCGTGACCATGGTGAGCGAAGGTGTCC
GTTGGTTGTGTCGTAATGCGGCCGGCCTGCCGGGGAGCCCACGTCGTGTTGTACT
GTCGGGTAGCTCCGCAGGCGCTCATCTTACCACCATGAGCCTGTTAGATGAAGCG
GGGTGGCGTCGCGACGGTTGGCGTCCTGCAGAGGCGGTGAGCGGTGCGGTTTTG
TTAAGCGGCGTGTACGACTTAGACCCGGTCCGTCGCACATACGTCAATGCACCAT
TGGGACTGGACGCTGATACAGCTCTGGCCTGTTCTCCTCAGCGTCGTCCGTTGGC
CGGCCTGCCCCCTCTTGTTGTGGCCCGCGGCGACAATGAAACCGGTGAATTTGCA
CGTCAACAACGTGAGTTCGTTGCGGCAGTGCGCCGCGCGGGTGGAAGTGTGAAT
GACCTGGTGGTGCGCGGTCGCAACCACTTTGACTTAGCATTCGACTTGGGCGACC
CAGCCACGTCACTTGGCGCTGCAGTGGCACGTCTCGTTGAATGA
Sequences for native and synthetic promoters and terminators to be
used in E. coli and P. putido heterologous hosts.
Synthetic promoters to be used in P. putido system. Underlined
sequences may also be used in E. coli.
SEQ ID NO: 18 (BG51)
AGGCCTCGTGGTCTACTTGACATCCGACATTCGCGACTGTATAATAAGTTGAGGG
C
SEQ ID NO: 19 (Pfer)
GGCGAGCGGTAGTAAAAAACTTCAAAATAAACGCTTGACATGTCACGTCGCGTG
ATTATAATTGCGCGTCCGACATGATCATCAGTACAATAGGAGATATCGCC
SEQ ID NO: 20 (Ptac)
GGCGCTATGGAGGTCAGGTATGATTACTATTGACAATTAATCATCGGCTCGTATA
ATGTGATCAGACCTGGAATTGTGAGCGGATAACAATTCTTAAGATTAACTCACAC
ACGAGGGTATCATGAGCG
SEQ ID NO: 21 (Pem7)
TGGGCGTTGTTGACAATTAATCATCGGCATAGTATATCGGCATAGTATAATACGA
CAAGGTGAGGAACTAAACCGG
Synthetic terminators to be used in P. putida system
SEQ ID NO: 22 (T1)
GAGCGCATGCTCGAGTACTTCGCCTGGACCATGCTCGCCGTCGTCTTCGGCTTCC
TGCACTTCGTCAACCTCGCCTACGTCCCCCTCGGCCACTGGGCCGAGACGTTCGC
CGGCTTCTTCAAATTTTCAGGGCTGCCGCACCCCATCGACTGGGGGCTAAG
SEQ ID NO: 23 (T7)
ATTATCGCTCGTGCTCGCCGCGACCTTCCTCTTCATGCACCTCTTCGGCATCGGGC
TGCACAATCTA
SEQ ID NO: 24 (T9)
GGAATCTCCTTCTCGCCTCTTTCGCTGAACACGAAAGCGAATCCGTTCAGCACAC
ACTTTACGATATGGCGCAAAAAGTCCTCGGCTGCGTGCCGGAAGTGAAAGACAT
CCACCTCACCATGCCCAACAAGCATTGCCTGCTCGTGGACCTGTCCCGCTTCGGT
CAGGACAATCCCAACGA
SEQ ID NO: 25 (T12)
CTCGCTCTATCTCCATCACCCGACCGCGACCGACCCAAAAATGGCGCCGCCGGGC
CATTCGACCTTCTACGCGCTCGCGCCGGTCCCCCATCTCGGCAAATTCCCCGTCG
ACTGGGCGCGCGTCGGGCCAATAGTATCTTA
Native promoters to be used in E. coli system
SEQ ID NO: 26 (arcB)
GTCGTTGAGGGGAATTCCGCATTTCTCACACAATTTATAACGTAACTGTCAGAAT
TGGGTATTATTGGGGCAGGTTGTCGTGAAGGAATTCCCTA
SEQ ID NO: 27 (aroF)
AAGCATAGCGGATTGTTTTCAAAGGGAGTGTAAATTTATCTATACAGAGGTAAG
GGTTGAAAGCGCGACTAAATTGCCTGTGTAAATAAAAATGTACGAAATATGGAT
TGAAAACTTTACTTTATGTGTTATCGTTACGTCATCCTCGCTGAGGATCAACTATC
GCAAACGAGCATAAACAGGATCGCCATC
SEQ ID NO: 28 (glk)
GTTCTATTCCTTATGCGGGGTCAGATACTTAGTTTGCCCAGCTTGCAAAAAGGCA
TCGCTGCAATTGGTGCTGAAACGATAAAGTAATTGTGTGACCCAGATCGATATTT
ACAGGGAGCCTGCCTTTCCGGCGTTGTTGTTATGCCCCCAGGTATTTACAGTGTG
AGAAAGAATTATTTTGACTTTAGCGGAGCAGTTGAAGA
SEQ ID NO: 29 (mqsR)
AACCCCCGCCTCCCTGTTACTTTAGTTATAACCTAAAAGGTTAATTACAGCAATG
AAAAAGCACCTAAAAGGTTAGTTAGATGTACGGAGATAGTGACCACACAAAACG
TATTCTTTAAGGAAAGTGATTGACCATATAAGAAAGTGGCGCATTAGTAGCGCCA
GTTTGAAGCAGGAATTTATAAGGGAAGCTGGAGTCAGGCA
Native promoters to be used in P. putida system
SEQ ID NO: 30 (recA)
ATCGACGACAGGGGTTTGCGCGGGCGTCTGCCTGTGGAATAATACTGGCTACTTA
TACAGGTATTCCGGCCGTCAGGGCCAAGTCGAACACGTGAGGATTTCA
SEQ ID NO: 31 (rpoS)
CCCAGCCTGTTCCTGTGATGTAGAGGGGACAGGCTCAAGCGCTGCCAGGGAGAA
AGGTGCCGCTCGAGTCTGAGTTCGAACTCAGCAAAGGATTATAACA
SEQ ID NO: 32 (rpsU)
TTGTGGGGGCTGAATTCGAAGCCGCGCATGATAGTCCCCGTGTCGGGTGCCGACC
AGCGGTTTTCGATCAGAGGCTTTGCATTCCGGCTTGCTAAGGGTTAACATCCGCA
ACCCTTGAAAACCGACGTTCTCCAGCACACCTTTGTTTTGCCAGGAGCACGTCTA
CCCCGGTAATGAATTAAGGTAGCCCTGG
SEQ ID NO: 33 (sigX)
CCGCACAAAAGCTGTTAATGTATGCCGCCGCGAAATTCGACCCACGGGGTCGCG
CGGCGACATTGACCTGACTGTCGGCCAGATCCGTTTTGAATAAAGTTCATTCGCC
GCCC
Tryptophan halogenases which may halogenate positions of the
tryptophan indole ring at C5-7.
SEQ ID NO: 34 (ClaH)
MLESIVVVGG GTSGWMTASY LSAAFGERIS VTVVESARVG TIGVGEATFS
TVRHFFEYLG LSEETWMPAC NATYKLGIRF ENWRAPGHHF YHPFERQRVV
DGFTLPDWWL ADGGATERFD KECFLVGTLC DTMRSPRHMD GALFEGDLTD
RPAGRSTLAE QGTQFPYAYH FDAALLADFL RDYAVARGVL HVVDDVVHVA
RDERGWISHV ATRGSGDLAG DLFVDCTGFR GLLINDALDE PFESYQDTLP
NDSAVALRVP VDMEREGLRP CTTSTAQAAG WIWTIPLFGR VGTGYVYARD
YCTPEEAERT LRRFVGPAAD DLEANHIRMR IGRSRRSWVN NCVAVGLSSG
FVEPLESTGI FFIQHAIEQL VKHFPDADWD PALRSAYNTL VNRCMDGVRE
FLVLHYYGAA RADNEYWRDT KTRKIPDSLA ERVEQWRTKL PHPESVYPHY
HGFEAYSYVC MVLGLGGIPL KPSPALRMLD PSAAQREFRL LATQAEDLRR
TLPSQYAYFA QFR
SEQ ID NO: 35 (AbeH)
MLKNVVVVGG GTAGWMTASY LTAAFGDRIG VTLVESKRVG SIGVGEATFS
TVRHFFEYLG LEEKEWMPAC NATYKLAIRF ENWREPGHHF YHPFERQRVV
DGFPLTDWWL REPRSDRFDK DCFLVGTLCD DLKSPRQLNG ELFEGGLGGR
SAYRTTLAEQ TTQFPYAYHF DATLVANYLR DYAVARGVKH VLDDVQDVAL
DDRGWISHVV TGESGNLTGD LFIDCTGFRS LLLGKALAEP FQSYQDSLPN
DSAVALRVPQ DMENRGLRPC TTATAQEAGW IWTIPLFDRI GTGYVYAGDY
ISPEEAERTL RAFVGPAAEH ADANHIKMRI GRSNRHWVNN CVAVGLSSGF
VEPLESTGIF FIQHAIEQLV KHFPDERWDD GLRTAYNKLV NNVMDGVREF
LVVHYYAAKR QDNQYWKDAK TRPLPDGLAE RLERWQTRLP DNESVFPHYH
GFESYSYVCM LLGLGGLDLK SSPALGLMDA APARHEFKLV GEQAAELART
LPTQYEYFAQ LHRAR
SEQ ID NO: 36 (PyrH)
MERRKRERLG SLGRPTKKEL RMIRSVVIVG GGTAGWMTAS YLKAAFDDRI
DVTLVESGNV RRIGVGEATF STVRHFFDYL GLDEREWLPR CAGGYKLGIR
FENWSEPGEY FYHPFERLRV VDGFNMAEWW LAVGDRRTSF SEACYLTHRL
CEAKRAPRML DGSLFASQVD ESLGRSTLAE QRAQFPYAYH FDADEVARYL
SEYAIARGVR HVVDDVQHVG QDERGWISGV HTKQHGEISG DLFVDCTGFR
GLLINQTLGG RFQSFSDVLP NNRAVALRVP RENDEDMRPY TTATAMSAGW
MWTIPLFKRD GNGYVYSDEF ISPEEAEREL RSTVAPGRDD LEANHIQMRI
GRNERTWINN CVAVGLSAAF VEPLESTGIF FIQHAIEQLV KHFPGERWDP
VLISAYNERM AHMVDGVKEF LVLHYKGAQR EDTPYWKAAK TRAMPDGLAR
KLELSASHLL DEQTIYPYYH GFETYSWITM NLGLGIVPER PRPALLHMDP
APALAEFERL RREGDELIAA LPSCYEYLAS IQ
SEQ ID NO: 37 (ThdH; also known as Thai)
MDNRIKTVVI LGGGTAGWMT AAYLGKALQN TVKIVVLEAP TIPRIGVGEA
TVPNLQRAFF DYLGIPEEEW MRECNASYKM AVKFINWRTP GEGSPDPRTL
DDGHTDTFHH PFGLLPSADQ IPLSHYWAAK RLQGETDENF DEACFADTAI
MNAKKAPRFL DMRRATNYAW HFDASKVAAF LRNFAVTKQA VEHVEDEMTE
VLTDERGFIT ALRTKSGRIL QGDLFVDCSG FRGLLINKAM EEPFIDMSDH
LLCNSAVATA VPHDDEKNGV EPYTSSIAME AGWTWKIPML GRFGSGHVYS
DHFATQDEAT LAFSKLWGLD PDNTEFNHVR FRVGRNRRAW VRNCVSVGLA
SCFVEPLESS GIYFIYAAIH MLAKHFPDKT FDKVLVDRFN REIEEMFDDT
RDFLQAHYYF SPRVDTPFWR ANKELKLADS IKDKVETYRA GLPVNLPVTD
EGTYYGNFEA EFRNFWTNGS YYCIFAGLGL MPRNPLPALA YKPQSIAEAE
LLFADVKRKG DTLVESLPST YDLLRQLHGA S
SEQ ID NO: 38 (Th-Hal)
LNNVVIVGGGTAGWMTASYLKAAFGDRIDITLVESGHIGAVGVGEATFSDIRHFFEF
LGLKEKDWMPACNATYKLAVRFENWREKGHYFYHPFEQMRSVNGFPLTDWWLKQ
GPTDRFDKDCFVMASVIDAGLSPRHQDGTLIDQPFDEGADEMQGLTMSEHQGKTQF
PYAYQFEAALLAKYLTKYSVERGVKHIVDDVREVSLDDRGWITGVRTGEHGDLTGD
LFIDCTGFRGLLLNQALEEPFISYQDTLANDSAVALQVPMDMERRGILACTTATAQDA
GWIWTIPLTGRVGTGYVYAKDYLSPEEAERTLREFVGPAAADVEANHIRMRIGRSRN
SWVKNCVAIGLSSGFVEPLESTGIFFIHHAIEQLVKNFPAADWNSMHRDLYNSAVSH
VMDGVREFLVLHYVAAKRNDTQYWRDTKTRKIPDSLAERIEKWKVQLPDSETVYP
YYHGLPPYSYMCILLGMGGIELKPSPALALADGGAAQREFEQIRNKTQRLTEVLPKA
YDYFTQ
SEQ ID NO: 39 (SttH)
MNTRNPDKVV IVGGGTAGWM TASYLKKAFG ERVSVTLVES GTIGTVGVGE
ATFSDIRHFF EFLDLREEEW MPACNATYKL AVRFQDWQRP GHHFYHPFEQ
MRSVDGFPLT DWWLQNGPTD RFDRDCFVMA SLCDAGRSPR YLNGSLLQQE
FDERAEEPAG LTMSEHQGKT QFPYAYHFEA ALLAEFLSGY SKDRGVKHVV
DEVLEVKLDD RGWISHVVTK EHGDIGGDLF VDCTGFRGVL LNQALGVPFV
SYQDTLPNDS AVALQVPLDM EARGIPPYTR ATAKEAGWIW TIPLIGRIGT
GYVYAKDYCS PEEAERTLRE FVGPEAADVE ANHIRMRIGR SEQSWKNNCV
AIGLSSGFVE PLESTGIFFI HHAIEQLVKH FPAGDWHPQL RAGYNSAVAN
VMDGVREFLV LHYLGAARND TRYWKDTKTR AVPDALAERI ERWKVQLPDS
ENVFPYYHGL PPYSYMAILL GTGAIGLRPS PALALADPAA AEKEFTAIRD
RARFLVDTLP SQYEYFAAMG QRV
SEQ ID NO: 40 (KtzR)
MTAAYLKTAF GDRLSITVVE SSRIGTIGVG EATFSDIQHF FQFLNLREQD
WMPACNATYK LGIRFENWRH VGHHFYQPFE QIRPVYGFPL TDWWLHDAPT
DRFDTDCFVM PNLCEAGRSP RHLDGTLADE DFVEEGDELA NRTMSEHQGK
SQFPYAYHFE AALLAKFLTG YAVDRGVEHV VDDVLDVRLD QRGWIEHVVT
AEHGEIHGDL FVDCTGFRGL LLNKALGVPF VSYQDTLPND SAVALQVPLD
MQRRGIVPNT TATAREAGWI WTIPLFGRVG TGYVYAKDYL SPEEAERTLR
EFVGPAAADV EANHIRMRIG RSQESWRNNC VAIGLSSGFV EPLESTGIFF
IHHAIEQLVK HFPAADWNPK SRDMYNSAVA HVMDGIREFL VIHYRGAARA
DNQYWRDTKT RPLPDGLAER IECWQTQLPD TETIYPYYHG LPPYSYMCIL
MGGGAIRTPA SAALALTDQG AAQKEFAAVR DRAAQLRDTL PSHYEYLARM
RGLDV
SEQ ID NO: 41 (BorH)
MDNRINRIVI LGGGTAGWMT ASYLAKALGD TVTITLLEAP AIGRIGVGEA
TVPNLQRVFF DFLGLREEEW MPECNAAFKT AVKFINWRTP GPGEAKARTI
DGRPDHFYHP FGLLPEHGQV PLSHYWAYNR AAGTTDEPFD YACFAETAAM
DAVRAPKWLD GRPATRYAWH FDAHLVAEFL RRHATERLNV EHVQGEMQQV
LRDERGFITA LRTVEGRDLE GDLFIDCSGF RGLLINKAME EPFIDMNDQL
LCNRAVATAI KHDDDAHGVE PYTSAIAMRS GWSWKIPMLG RFGTGYVYSS
RFAEKDEATL DFCRMWGLDP ENTPLNQVAF RVGRNRRAWV KNCVSIGLAS
CFLEPLESTG IYFITAAIYQ LTQHFPDRTF ALALSDAFNH EIEAMFDDTR
DFIQAHFYVS PRTDTPFWKA NKDLHLPEQM REKIAMYKAG LPINAPVTDE
STYYGRFEAE FRNFWTNGSY YCIFAGLGLR PDNPLPMLRH RPEQVREAQA
LFAGVKDKQR ELVETLPSNL EFLRSLHGK
SEQ ID NO: 42 (KtzQ)
MDDNRIRSIL VLGGGTAGWM SACYLSKALG PGVEVTVLEA PSISRIRVGE
ATIPNLHKVF FDFLGIAEDE WMRECNASYK AAVRFVNWRT PGDGQATPRR
RPDGRPDHFD HLFGQLPEHE NLPLSQYWAH RRLNGLTDEP FDRSCYVQPE
LLDRKLSPRL MDGTKLASYA WHFDADLVAD FLCRFAVQKL NVTHVQDVFT
HADLDQRGHI TAVNTESGRT LAADLFIDCS GFRSVLMGKV MQEPFLDMSK
HLLNDRAVAL MLPHDDEKVG IEPYTSSLAM RSGWSWKIPL LGRFGSGYVY
SSQFTSQDEA AEELCRMWDV DPAEQTFNNV RFRVGRSRRA WVRNCVAIGV
SAMFVEPLES TGLYFSYASL YQLVKHFPDK RFRPILADRF NREVATMYDD
TRDFLQAHFS LSPRDDSEFW RACKELPFAD GFAEKVEMYR AGLPVELPVT
IDDGHYYGNF EAEFRNFWTN SNYYCIFAGL GFLPEHPLPV LEFRPEAVDR
AEPVFAAVRR RTEELVATAP TMQAYLRRLH QGT
SEQ ID NO: 43 (PmA)
MNKPIKNIVIVGGGTAGWMAASYLVRALQQQANITLIESAAIPRIGVGEATIPSLQKV
FFDFLGIPEREWMPQVNGAFKAAIKFVNWRKSPDPSRDDHFYHLFGNVPNCDGVPLT
HYWLRKREQGFQQPMEYACYPQPGALDGKLAPCLSDGTRQMSHAWHFDAHLVAD
FLKRWAVERGVNRVVDEVVDVRLNNRGYISNLLTKEGRTLEADLFIDCSGMRGLLI
NQALKEPFIDMSDYLLCDSAVASAVPNDDARDGVEPYTSSIAMNSGWTWKIPMLGR
FGSGYVFSSHFTSRDQATADFLKLWGLSDNQPLNQIKFRVGRNKRAWVNNCVSIGLS
SCFLEPLESTGIYFIYAALYQLVKHFPDTSFDPRLSDAFNAEIVHMFDDCRDFVQAHY
FTTSRDDTPFWLANRHDLRLSDAIKEKVQRYKAGLPLTTTSFDDSTYYETFDYEFKN
FWLNGNYYCIFAGLGMLPDRSLPLLQHRPESIEKAEAMFASIRREAERLRTSLPTNYD
YLRSLRDGDAGLSRGQRGPKLAAQESL
SEQ ID NO: 44 (Rebli)
MHHGFTTPSRAIAVLSTETIRGNITFTQVQDGKVHVQGGITGLPPGEYGFHVHEKGDL
SGGCLSTGSHFNPEHKDHGHPNDVNRHVGDLGNVVFDENHYSRIDLVDDQISLSGPH
GIIGRAVVLHEKADDYGKSDHPDSRKTGNAGGRVACGVIGIL
Primers used for in vitro in-frame deletion of the tar14 gene
SEQ ID NO: 45 (Tar14_KO-gRNA1-F)
GACTGACACTGATAATACGACTCACTATAGGATGCCGTCATCCACCCGGGTTTTA
GAGCTAGAAATAGCAAGTT
SEQ ID NO: 46 (Tar14_KO-gRNA2-F)
GACTGACACTGATAATACGACTCACTATAGGCGAGCTGTACAACCGGTGTTTTAG
AGCTAGAAATAGCAAGTT
SEQ ID NO: 47 (Tar14_KO-gRNA-R)
AAAAGCACCGACTCGGTGC
SEQ ID NO: 48 (Tar14_KO-conf-F)
GATAGCGCTGTACGAATACTG
SEQ ID NO: 49 (Tar14_KO-conf-R)
CATACTCACGCTGCACAATGC
Primers used for cloning genes for heterologous expression
SEQ ID NO: 50 (Tar14_pCJW93_F)
GGCCTGGTGCCGCGCGGCAGCCATATGTCTGTCAGTGGCTCCGAAAGATCGGCC
SEQ ID NO: 51 (Tar14_pCJW93_R)
GATCTGGGGAATTCGGATCCAAGCTTTTAGCGTCCGGCCCGCATGGCCGCGAAGT
A
SEQ ID NO: 52 (Tar13_F)
CCTGGTGCCGCGCGGCAGCCATATGACCGAGCGGACCGCCACCCGAACGG
SEQ ID NO: 53 (Tar13_R)
GTGGTGGTGGTGCTCGAGTGCGGCCGCCTATCCGCCTGTCCCGGATGCCCTCCG
SEQ ID NO: 54 (Tar15_F)
CCTGGTGCCGCGCGGCAGCCATATGTCGATCGGTCGAAGCACTGCCGAG
SEQ ID NO: 55 (Tar15_R)
GTGGTGGTGGTGCTCGAGTGCGGCCGCTCACCTCGCTTCGTCGAGCAGGCTC
SEQ ID NO: 56 (Tar16_F)
CCTGGTGCCGCGCGGCAGCCATATGGCTGCGGCGGTGTTCCGGTCGTAC
SEQ ID NO: 57 (Tar16_R)
GTGGTGGTGGTGCTCGAGTGCGGCCGCTCATTCGACAAGGCGGGCAACCGCCGC

What is claimed is:

1. A genetically engineered microbe, wherein the genetically engineered microbe comprises an exogenous Tar14 encoding nucleic acid, an exogenous Tar13 encoding nucleic acid, or an exogenous Tar16 encoding nucleic acid.

2. The genetically engineered microbe of claim 1, wherein the genetically engineered microbe comprises an exogenous Tar 14 enzyme, an exogenous Tar13 enzyme, or an exogenous Tar16 enzyme.

3. A genetically engineered microbe, wherein the genetically engineered microbe comprises one or more of an exogenous Tar14 encoding nucleic acid, an exogenous Tar13 encoding nucleic acid, or an exogenous Tar16 encoding nucleic acid.

4. The genetically engineered microbe of claim 3, wherein the genetically engineered microbe comprises one or more of an exogenous Tar 14 enzyme, an exogenous Tar13 enzyme, or an exogenous Tar16 enzyme.

5. The genetically engineered microbe of claim 1, wherein the genetically engineered microbe does not comprise an endogenous Tar14 encoding nucleic acid, an endogenous Tar13 encoding nucleic acid, or an endogenous Tar16 encoding nucleic acid

6. The genetically engineered microbe of claim 1, wherein the genetically engineered microbe does not comprise one or more of an endogenous Tar14 encoding nucleic acid, an endogenous Tar13 encoding nucleic acid, or an endogenous Tar16 encoding nucleic acid.

7. The genetically engineered microbe of claim 6, wherein the genetically engineered microbe does not comprise an endogenous Tar14 encoding nucleic acid.

8. The genetically engineered microbe of claim 6, wherein the genetically engineered microbe does not comprise an endogenous Tar13 encoding nucleic acid.

9. The genetically engineered microbe of claim 6, wherein the genetically engineered microbe does not comprise an endogenous Tar16 encoding nucleic acid.

10. The genetically engineered microbe of claim 6, wherein the genetically engineered microbe does not comprise an endogenous Tar13 encoding nucleic acid or an endogenous Tar14 encoding nucleic acid.

11. The genetically engineered microbe of claim 6, wherein the genetically engineered microbe does not comprise an endogenous Tar13 encoding nucleic acid or an endogenous Tar16 encoding nucleic acid.

12. The genetically engineered microbe of claim 6, wherein the genetically engineered microbe does not comprise an endogenous Tar14 encoding nucleic acid or an endogenous Tar16 encoding nucleic acid.

13. The genetically engineered microbe of claim 1, wherein the genetically engineered microbe comprises an exogenous nucleic acid that has at least 85% nucleotide identity to SEQ ID NO:11, SEQ ID NO:13, or SEQ ID NO:17.

14. The genetically engineered microbe of claim 3, wherein the genetically engineered microbe comprises one or more of an exogenous nucleic acid having at least 85% nucleotide identity to SEQ ID NO:11, SEQ ID NO:13, or SEQ ID NO:17.

15. The genetically engineered microbe of claim 13, wherein the exogenous nucleic acid has at least 85% nucleotide identity to SEQ ID NO:11.

16. The genetically engineered microbe of claim 13, wherein the exogenous nucleic acid has at least 85% nucleotide identity to SEQ ID NO:13.

17. The genetically engineered microbe of claim 13, wherein the exogenous nucleic acid has at least 85% nucleotide identity to SEQ ID NO:17.

18. The genetically engineered microbe of claim 1, wherein the genetically engineered microbe comprises an exogenous Flavin reductase encoding nucleic acid.

19. The genetically engineered microbe of claim 1, wherein the genetically engineered microbe comprises an exogenous Flavin reductase.

20. The genetically engineered microbe of claim 1, wherein the microbe comprises an exogenous Tar15 encoding nucleic acid.

21. The genetically engineered microbe of claim 1, wherein the genetically engineered microbe comprises an exogenous Tar15 enzyme.

22. The genetically engineered microbe of claim 20, wherein the exogenous Tar15 encoding nucleic acid has at least 85% nucleotide identity to SEQ ID NO:15.

23. The genetically engineered microbe of claim 1, wherein the encoding nucleic acid has at least 85% nucleotide identity to SEQ ID NO:10, SEQ ID NO:12, or SEQ ID NO:16.

24. The genetically engineered microbe of any of claim 1, wherein the exogenous Tar14 encoding nucleic acid, exogenous Tar13 encoding nucleic acid, or exogenous Tar16 encoding nucleic acid further comprises an exogenous promoter.

25. The genetically engineered microbe of claim 24, wherein the exogenous promoter is BG51, Pfer, Ptac, Pem7, arcB, aroF, glk, mqsR, recA, rpoS, rpsU, or sigX.

26. The genetically engineered microbe of claim 20, wherein the exogenous Tar15 encoding nucleic acid further comprises an exogenous promoter.

27. The genetically engineered microbe of claim 26, wherein the exogenous promoter is BG51, Pfer, Ptac, Pem7, arcB, aroF, glk, mqsR, recA, rpoS, rpsU, or sigX.

28. The genetically engineered microbe of claim 1, wherein the microbe is a gram negative bacterium.

29. The genetically engineered microbe of claim 28, wherein the gram negative bacterium is E. coli or P. putida.

30. The genetically engineered microbe of claim 1, wherein the microbe is a human gastrointestinal microbe.

31. A method of producing L-4-Cl-Kyn comprising contacting the genetically engineered microbe of any one of claim 1 with L-tryptophan.

32. The method of claim 31, comprising isolating L-4-Cl-Kyn from cells.

33. A genetically engineered microbe, wherein the genetically engineered microbe comprises a nucleic acid coding for an exogenous tryptophan halogenase.

34. The genetically engineered microbe of claim 33, wherein the genetically engineered microbe comprises an exogenous tryptophan halogenase.

35. The genetically engineered microbe of claim 34, wherein the exogenous tryptophan halogenase is Tar14, ClaH, AbeH, PyrH, ThdH, Th-Hal, SttH, KtzR, BorH, KtzQ, PrnA, RebH, or AtmH.

36. The genetically engineered microbe of claim 33, wherein the exogenous tryptophan halogenase has at least 85% identity to SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44.

37. The genetically engineered microbe of claim 33, wherein the encoded nucleic acid comprises at least on optimized codon.

38. The genetically engineered microbe of claim 33, wherein the nucleic acid encoding the exogenous tryptophan halogenase further comprises an exogenous promoter.

39. The genetically engineered microbe of claim 38, wherein the exogenous promoter is BG51, Pfer, Ptac, Pem7, arcB, aroF, glk, mqsR, recA, rpoS, rpsU, or sigX.

40. The genetically engineered microbe of claim 33, wherein the microbe is a gram negative bacterium.

41. The genetically engineered microbe of claim 40, wherein the gram negative bacterium is E. coli or P. putida.

42. The genetically engineered microbe of claim 33, wherein the microbe is a human gastrointestinal microbe.

43. A method of synthesizing L-4-Cl-Kyn, said method comprising contacting L-Trp with a Tar14 enzyme, a Tar13 enzyme, and a Tar16 enzyme.

44. The method of claim 43, further comprising a Flavin reductase.

45. The method of claim 44, wherein the Flavin reductase is Tar15 enzyme.

46. An isolated nucleic acid, said isolated nucleic acid comprising a Tar14 encoding nucleic acid, a Tar13 encoding nucleic acid, a Tar16 encoding nucleic acid, or a Tar15 nucleic acid.

47. An isolated nucleic acid, said isolated nucleic acid comprising one or more of a Tar14 encoding nucleic acid, a Tar13 encoding nucleic acid, a Tar16 encoding nucleic acid, or a Tar15 nucleic acid.

48. The isolated nucleic acid of claim 46, wherein said isolated nucleic acid has at least 85% nucleotide identity to SEQ ID NO:11, SEQ ID NO:13, SEQ ID NO:15, or SEQ ID NO:17.

49. The isolated nucleic acid of claim 47, comprising one or more sequences having at least 85% nucleotide identity to SEQ ID NO:11, SEQ ID NO:13, SEQ ID NO:15, or SEQ ID NO:17.

50. The isolated nucleic acid of claim 46, wherein the isolated nucleic acid comprises at least one optimized codon

51. An isolated enzyme, said isolated enzyme comprising Tar 14, Tar13, Tar16, or Tar15, or enzymatically active fragment or variant thereof.

52. The isolated enzyme of claim 51, wherein said enzyme has at least 85% identity to SEQ ID NO:1, SEQ ID NO:3, SEQ ID NO:6, or SEQ ID NO:8.


SEQUENCE LISTING (57 sequences)

SEQ ID NO: 1 - protein, 236 aa, Saccharomonospora sp. CNQ-490
MTERTATRTEPAYGEILRLDELLELACVNDEADRALFLSAHQACEIWFAVVLRHLEDVTDALSLDDGATAAELLERLPRIITVIIEHFEVLGTLKPEAFDRIRADLGSSSGFQSVQYREIEYLCGARDTRFLNTAGFRDRDRRRLRERLAKRSLSNVFLEYRGRAGDRDACRISDALHEFDDSVRALRLRHAGIAELFLGSIPGTAGTAGAAYLRRSASRTLFPELFDRRASGTGG

SEQ ID NO: 2 - protein, 256 aa, Artificial Sequence
  Synthetic Polypeptide
MGSSHHHHHHSSGLVPRGSHMTERTATRTEPAYGEILRLDELLELACVNDEADRALFLSAHQACEIWFAVVLRHLEDVTDALSLDDGATAAELLERLPRIITVIIEHFEVLGTLKPEAFDRIRADLGSSSGFQSVQYREIEYLCGARDTRFLNTAGFRDRDRRRLRERLAKRSLSNVFLEYRGRAGDRDACRISDALHEFDDSVRALRLRHAGIAELFLGSIPGTAGTAGAAYLRRSASRTLFPELFDRRASGTGG

SEQ ID NO: 3 - protein, 534 aa, Saccharomonospora sp. CNQ-490
MSVSGSERSAEGNRKKRVVIVGGGTAGWMTASYLTAAFGDRVDLTVVESAQIGTIGVGEATFSDIRHFFEFLRLEESDWMPECNATYKLAVRFENWREPGHHFYHPFEQMSSVDGFPLSDWWLRNPTTSRFDKDSFVMTSLCDAGVSPRYLDGSLIDQDFVEQERDDDSARSTIAEYQGAQFPYAYHFEAHLLAKYLTGYATRRGTRHIVDNVVDVALDERGWISHVRTEEHGDLEADLFVDCTGFRGLLLNKALGEPFVSYQDTLPNDSAVALQVPLDMEREPIRPCTTATAQEAGWIWTIPLISRVGTGYVYASDYTTPEQAERVLRDFVGPAAADVPANHIKMRIGRSRRSWVNNCVGVGLSSGFVEPLESTGIFFIHHAIEQIVKYFPSGGAGDDRLRELYNRSVGHVMDGVREFLVLHYRSAKRADNQYWKDTKTRTVPDSLAERIEFWKHKVPDAETVYPYYHGLPPYSYNCILLGMGGIDVNYSPALDWANEKAALAEFERIRVKAEKLVQELPTQNEYFAAMRAGR

SEQ ID NO: 4 - protein, 554 aa, Artificial Sequence
  Synthetic Polypeptide
MGSSHHHHHHSSGLVPRGSHMSVSGSERSAEGNRKKRVVIVGGGTAGWMTASYLTAAFGDRVDLTVVESAQIGTIGVGEATFSDIRHFFEFLRLEESDWMPECNATYKLAVRFENWREPGHHFYHPFEQMSSVDGFPLSDWWLRNPTTSRFDKDSFVMTSLCDAGVSPRYLDGSLIDQDFVEQERDDDSARSTIAEYQGAQFPYAYHFEAHLLAKYLTGYATRRGTRHIVDNVVDVALDERGWISHVRTEEHGDLEADLFVDCTGFRGLLLNKALGEPFVSYQDTLPNDSAVALQVPLDMEREPIRPCTTATAQEAGWIWTIPLISRVGTGYVYASDYTTPEQAERVLRDFVGPAAADVPANHIKMRIGRSRRSWVNNCVGVGLSSGFVEPLESTGIFFIHHAIEQIVKYFPSGGAGDDRLRELYNRSVGHVMDGVREFLVLHYRSAKRADNQYWKDTKTRTVPDSLAERIEFWKHKVPDAETVYPYYHGLPPYSYNCILLGMGGIDVNYSPALDWANEKAALAEFERIRVKAEKLVQELPTQNEYFAAMRAGR

SEQ ID NO: 5 - protein, 537 aa, Artificial Sequence
  Synthetic Polypeptide
GSHMSVSGSERSAEGNRKKRVVIVGGGTAGWMTASYLTAAFGDRVDLTVVESAQIGTIGVGEATFSDIRHFFEFLRLEESDWMPECNATYKLAVRFENWREPGHHFYHPFEQMSSVDGFPLSDWWLRNPTTSRFDKDSFVMTSLCDAGVSPRYLDGSLIDQDFVEQERDDDSARSTIAEYQGAQFPYAYHFEAHLLAKYLTGYATRRGTRHIVDNVVDVALDERGWISHVRTEEHGDLEADLFVDCTGFRGLLLNKALGEPFVSYQDTLPNDSAVALQVPLDMEREPIRPCTTATAQEAGWIWTIPLISRVGTGYVYASDYTTPEQAERVLRDFVGPAAADVPANHIKMRIGRSRRSWVNNCVGVGLSSGFVEPLESTGIFFIHHAIEQIVKYFPSGGAGDDRLRELYNRSVGHVMDGVREFLVLHYRSAKRADNQYWKDTKTRTVPDSLAERIEFWKHKVPDAETVYPYYHGLPPYSYNCILLGMGGIDVNYSPALDWANEKAALAEFERIRVKAEKLVQELPTQNEYFAAMRAGR

SEQ ID NO: 6 - protein, 178 aa, Saccharomonospora sp. CNQ-490
MSIGRSTAEAGAMASFRDAMASFPTGVSVVTTMHTDGAPRGMTCSALCSVSMEPPLLLVCLRTASPTLDAIRVRGGFVVNLLKYQARDTARLFASGDTGRFDQVAWRHHPGTAGPCLVDDAHAAVDCQVLRRDEAGDHVVVLGEVVGVRTLSGAAPLLYGLRRYARWPDASSLLDEAR

SEQ ID NO: 7 - protein, 198 aa, Artificial Sequence
  Synthetic Polypeptide
MGSSHHHHHHSSGLVPRGSHMSIGRSTAEAGAMASFRDAMASFPTGVSVVTTMHTDGAPRGMTCSALCSVSMEPPLLLVCLRTASPTLDAIRVRGGFVVNLLKYQARDTARLFASGDTGRFDQVAWRHHPGTAGPCLVDDAHAAVDCQVLRRDEAGDHVVVLGEVVGVRTLSGAAPLLYGLRRYARWPDASSLLDEAR

SEQ ID NO: 8 - protein, 288 aa, Saccharomonospora sp. CNQ-490
MAAAVFRSYDQHELDIQYSPSSRVDDVQSYLREYARLSARARTEIDGFVEIRYGEFPEQVVDYFPAGTSGGSLLVFVHGGYWQELSRRESAFMAADLIERGVSVAALGYGLAPRYTVPEIVTMVSEGVRWLCRNAAGLPGSPRRVVLSGSSAGAHLTTMSLLDEAGWRRDGWRPAEAVSGAVLLSGVYDLDPVRRTYVNAPLGLDADTALACSPQRRPLAGLPPLVVARGDNETGEFARQQREFVAAVRRAGGSVNDLVVRGRNHFDLAFDLGDPATSLGAAVARLVE

SEQ ID NO: 9 - protein, 308 aa, Artificial Sequence
  Synthetic Polypeptide
MGSSHHHHHHSSGLVPRGSHMAAAVFRSYDQHELDIQYSPSSRVDDVQSYLREYARLSARARTEIDGFVEIRYGEFPEQVVDYFPAGTSGGSLLVFVHGGYWQELSRRESAFMAADLIERGVSVAALGYGLAPRYTVPEIVTMVSEGVRWLCRNAAGLPGSPRRVVLSGSSAGAHLTTMSLLDEAGWRRDGWRPAEAVSGAVLLSGVYDLDPVRRTYVNAPLGLDADTALACSPQRRPLAGLPPLVVARGDNETGEFARQQREFVAAVRRAGGSVNDLVVRGRNHFDLAFDLGDPATSLGAAVARLVE

SEQ ID NO: 10 - DNA, 774 bp, Artificial Sequence
  Synthetic Polynucleotide
atgaccgagcgtacggccacccgcaccgaacctgcatatggcgagattttacgtcttgatgagctccttgagctcgcgtgtgtaaacgacgaagcagatcgcgcattattcttatctgctcaccaggcgtgtgagatttggttcgcggttgttttacgccacttagaagacgtgacggatgcgctgagtttagacgacggtgctacggctgcggagttgcttgagcgtctccctcgcatcatcaccgttatcattgagcacttcgaagtcctcggtacccttaagcccgaggcttttgatcgcatccgcgctgatctgggcagcagctccggctttcaatcggttcaataccgtgagatcgagtatctctgcggagcccgcgacacgcgcttccttaatacggcgggcttccgtgatcgcgatcgccgccgtctccgtgaacgtttagcgaagcgctcgctgagcaacgttttccttgaatatcgtggtcgcgccggtgaccgcgatgcatgccgtatttcggacgcgcttcatgaatttgacgactcagttcgtgccctgcgcttgcgccatgctggtatcgctgagctgttcttaggcagcatccctggcacggcaggcacggcgggagcggcgtatctccgtcgttctgcttcccgcaccctgtttccagaattgtttgaccgccgtgcgtccggaacaggtggcgcgctcgtcccgcgtggttctcaccatcaccatcaccacgactacaaggacgacgacgacaaatga

SEQ ID NO: 11 - DNA, 708 bp, Saccharomonospora sp. CNQ-490
atgaccgagcgtacggccacccgcaccgaacctgcatatggcgagattttacgtcttgatgagctccttgagctcgcgtgtgtaaacgacgaagcagatcgcgcattattcttatctgctcaccaggcgtgtgagatttggttcgcggttgttttacgccacttagaagacgtgacggatgcgctgagtttagacgacggtgctacggctgcggagttgcttgagcgtctccctcgcatcatcaccgttatcattgagcacttcgaagtcctcggtacccttaagcccgaggcttttgatcgcatccgcgctgatctgggcagcagctccggctttcaatcggttcaataccgtgagatcgagtatctctgcggagcccgcgacacgcgcttccttaatacggcgggcttccgtgatcgcgatcgccgccgtctccgtgaacgtttagcgaagcgctcgctgagcaacgttttccttgaatatcgtggtcgcgccggtgaccgcgatgcatgccgtatttcggacgcgcttcatgaatttgacgactcagttcgtgccctgcgcttgcgccatgctggtatcgctgagctgttcttaggcagcatccctggcacggcaggcacggcgggagcggcgtatctccgtcgttctgcttcccgcaccctgtttccagaattgtttgaccgccgtgcgtccggaacaggtggc

SEQ ID NO: 12 - DNA, 1668 bp, Artificial Sequence
  Synthetic Polynucleotide
atgtccgtgtctggtagcgagcgcagcgccgaaggaaatcgtaagaaacgtgtggtcatcgttggcggtggcaccgccgggtggatgactgcaagttatcttaccgcagcgtttggagatcgtgtagacttgaccgtcgtagaatcagcacaaattggaaccatcggtgttggagaggcgacattttcggacatccgccatttcttcgaatttctgcgcttagaggagagcgactggatgccggaatgtaatgcgacatacaaactggcagtacgttttgagaattggcgtgaaccagggcaccatttctatcatccttttgagcagatgtcctctgttgacggcttccctttaagtgactggtggttgcgtaatccaacaaccagccgcttcgataaagatagctttgttatgacctcgttatgtgatgcgggagtatctccacgctacttagacggctcattaattgatcaagatttcgtcgaacaagagcgcgatgacgactcggcgcgcagtacaatcgcggagtatcaaggcgcgcaatttccgtatgcatatcacttcgaggcacacctcttggcgaagtacttaacgggatatgccacccgtcgtggtacgcgtcacatcgtggacaatgtagtggacgtggcactcgatgagcgtggctggatcagccatgtacgcacagaggagcacggggatttagaagcagacttgttcgttgattgtactgggttccgtggccttttgctgaataaggccttaggcgagccttttgtgtcttatcaagacacgctcccgaatgacagcgcagtggccctgcaagttcctctggatatggaacgtgagccaatccgtccttgcactactgccaccgcccaagaggccggctggatttggacgattccactgatcagccgtgtgggaacgggctatgtttacgcgtcggattacacaacccccgagcaagctgaacgtgtgcttcgtgattttgtaggtccagcagctgcagacgtaccagcgaaccacatcaagatgcgtatcggccgcagtcgtcgcagctgggttaataattgtgtcggtgtcgggttatccagcggattcgtcgagccgttggagtcaacgggcatcttctttatccatcacgcaattgaacaaattgtgaagtattttccgtcgggcggcgcgggagatgaccgcttgcgtgagctttacaatcgcagcgttgggcacgtgatggacggagttcgtgaattcttggttttacattatcgttcagcaaagcgtgcggataaccaatattggaaggataccaagacacgtaccgtacctgactcgttggcggagcgtatcgaattctggaaacacaaggtacccgatgctgagacggtatatccgtactatcacggccttccgccctatagctacaattgtattcttctcggaatgggcggcattgatgttaactacagccccgcattggattgggcaaatgagaaggccgcgttggccgagttcgaacgcattcgcgttaaagcagagaaactcgtccaggagctgcctacacaaaatgagtacttcgcggccatgcgtgcgggccgcgcgctggttccgcgcggcagtcatcaccaccatcaccatgattacaaggacgacgacgataaatga

SEQ ID NO: 13 - DNA, 1602 bp, Saccharomonospora sp. CNQ-490
atgtccgtgtctggtagcgagcgcagcgccgaaggaaatcgtaagaaacgtgtggtcatcgttggcggtggcaccgccgggtggatgactgcaagttatcttaccgcagcgtttggagatcgtgtagacttgaccgtcgtagaatcagcacaaattggaaccatcggtgttggagaggcgacattttcggacatccgccatttcttcgaatttctgcgcttagaggagagcgactggatgccggaatgtaatgcgacatacaaactggcagtacgttttgagaattggcgtgaaccagggcaccatttctatcatccttttgagcagatgtcctctgttgacggcttccctttaagtgactggtggttgcgtaatccaacaaccagccgcttcgataaagatagctttgttatgacctcgttatgtgatgcgggagtatctccacgctacttagacggctcattaattgatcaagatttcgtcgaacaagagcgcgatgacgactcggcgcgcagtacaatcgcggagtatcaaggcgcgcaatttccgtatgcatatcacttcgaggcacacctcttggcgaagtacttaacgggatatgccacccgtcgtggtacgcgtcacatcgtggacaatgtagtggacgtggcactcgatgagcgtggctggatcagccatgtacgcacagaggagcacggggatttagaagcagacttgttcgttgattgtactgggttccgtggccttttgctgaataaggccttaggcgagccttttgtgtcttatcaagacacgctcccgaatgacagcgcagtggccctgcaagttcctctggatatggaacgtgagccaatccgtccttgcactactgccaccgcccaagaggccggctggatttggacgattccactgatcagccgtgtgggaacgggctatgtttacgcgtcggattacacaacccccgagcaagctgaacgtgtgcttcgtgattttgtaggtccagcagctgcagacgtaccagcgaaccacatcaagatgcgtatcggccgcagtcgtcgcagctgggttaataattgtgtcggtgtcgggttatccagcggattcgtcgagccgttggagtcaacgggcatcttctttatccatcacgcaattgaacaaattgtgaagtattttccgtcgggcggcgcgggagatgaccgcttgcgtgagctttacaatcgcagcgttgggcacgtgatggacggagttcgtgaattcttggttttacattatcgttcagcaaagcgtgcggataaccaatattggaaggataccaagacacgtaccgtacctgactcgttggcggagcgtatcgaattctggaaacacaaggtacccgatgctgagacggtatatccgtactatcacggccttccgccctatagctacaattgtattcttctcggaatgggcggcattgatgttaactacagccccgcattggattgggcaaatgagaaggccgcgttggccgagttcgaacgcattcgcgttaaagcagagaaactcgtccaggagctgcctacacaaaatgagtacttcgcggccatgcgtgcgggccgc

SEQ ID NO: 14 - DNA, 600 bp, Artificial Sequence
  Synthetic Polynucleotide
atgagtattgggcgttcgacagcagaggcgggtgctatggcgagttttcgcgacgccatggcaagcttcccaactggcgtaagtgttgtgacgactatgcacacggacggggcgccccgcgggatgacttgttccgcgttgtgtagcgtttcgatggagccgccgttacttctggtgtgtctccgtacggcaagcccaacattggacgcaatccgcgttcgtggcggcttcgtagttaacctgttaaagtatcaggcccgcgatacggcgcgtttattcgcctcgggtgacactggccgctttgaccaagtagcatggcgtcatcatcccggaactgcaggcccatgtcttgtggacgacgcacatgccgctgttgactgtcaagtactgcgtcgcgatgaagcaggggatcacgtggtggttttaggcgaggtcgtcggtgtacgcactctgagcggcgcagctcctcttctctatggactccgtcgctacgcccgttggccagatgcctcaagtcttttggacgaggcacgtgcacttgttccacgcggcagccatcaccaccatcaccacgactacaaggacgatgatgataagtga

SEQ ID NO: 15 - DNA, 534 bp, Saccharomonospora sp. CNQ-490
atgagtattgggcgttcgacagcagaggcgggtgctatggcgagttttcgcgacgccatggcaagcttcccaactggcgtaagtgttgtgacgactatgcacacggacggggcgccccgcgggatgacttgttccgcgttgtgtagcgtttcgatggagccgccgttacttctggtgtgtctccgtacggcaagcccaacattggacgcaatccgcgttcgtggcggcttcgtagttaacctgttaaagtatcaggcccgcgatacggcgcgtttattcgcctcgggtgacactggccgctttgaccaagtagcatggcgtcatcatcccggaactgcaggcccatgtcttgtggacgacgcacatgccgctgttgactgtcaagtactgcgtcgcgatgaagcaggggatcacgtggtggttttaggcgaggtcgtcggtgtacgcactctgagcggcgcagctcctcttctctatggactccgtcgctacgcccgttggccagatgcctcaagtcttttggacgaggcacgt

SEQ ID NO: 16 - DNA, 927 bp, Artificial Sequence
  Synthetic Polynucleotide
atggggagtagccaccaccatcaccaccattcctgtggcttggtcccgcgcggttcgcacatggcggccgccgtattccgcagctacgatcagcatgagttagacattcagtatagcccaagctcgcgtgtcgacgacgtgcaatcataccttcgcgagtacgctcgtcttagtgcacgtgcccgcaccgagatcgacggatttgtagagattcgttacggtgaattcccggaacaagttgtggattactttccagctggaacgtcgggcggctcgctcttagtgtttgtgcacggcggctactggcaagaattgtcccgccgtgagtccgcgttcatggcagcggacttaatcgagcgcggtgtttcagtggcagctctgggctatggtttagcacctcgttatactgtgccggaaatcgtgaccatggtgagcgaaggtgtccgttggttgtgtcgtaatgcggccggcctgccggggagcccacgtcgtgttgtactgtcgggtagctccgcaggcgctcatcttaccaccatgagcctgttagatgaagcggggtggcgtcgcgacggttggcgtcctgcagaggcggtgagcggtgcggttttgttaagcggcgtgtacgacttagacccggtccgtcgcacatacgtcaatgcaccattgggactggacgctgatacagctctggcctgttctcctcagcgtcgtccgttggccggcctgccccctcttgttgtggcccgcggcgacaatgaaaccggtgaatttgcacgtcaacaacgtgagttcgttgcggcagtgcgccgcgcgggtggaagtgtgaatgacctggtggtgcgcggtcgcaaccactttgacttagcattcgacttgggcgacccagccacgtcacttggcgctgcagtggcacgtctcgttgaatga

SEQ ID NO: 17 - DNA, 867 bp, Saccharomonospora sp. CNQ-490
atggcggccgccgtattccgcagctacgatcagcatgagttagacattcagtatagcccaagctcgcgtgtcgacgacgtgcaatcataccttcgcgagtacgctcgtcttagtgcacgtgcccgcaccgagatcgacggatttgtagagattcgttacggtgaattcccggaacaagttgtggattactttccagctggaacgtcgggcggctcgctcttagtgtttgtgcacggcggctactggcaagaattgtcccgccgtgagtccgcgttcatggcagcggacttaatcgagcgcggtgtttcagtggcagctctgggctatggtttagcacctcgttatactgtgccggaaatcgtgaccatggtgagcgaaggtgtccgttggttgtgtcgtaatgcggccggcctgccggggagcccacgtcgtgttgtactgtcgggtagctccgcaggcgctcatcttaccaccatgagcctgttagatgaagcggggtggcgtcgcgacggttggcgtcctgcagaggcggtgagcggtgcggttttgttaagcggcgtgtacgacttagacccggtccgtcgcacatacgtcaatgcaccattgggactggacgctgatacagctctggcctgttctcctcagcgtcgtccgttggccggcctgccccctcttgttgtggcccgcggcgacaatgaaaccggtgaatttgcacgtcaacaacgtgagttcgttgcggcagtgcgccgcgcgggtggaagtgtgaatgacctggtggtgcgcggtcgcaaccactttgacttagcattcgacttgggcgacccagccacgtcacttggcgctgcagtggcacgtctcgttgaatga

SEQ ID NO: 18 - DNA, 56 bp, Artificial Sequence
  Synthetic Polynucleotide
aggcctcgtggtctacttgacatccgacattcgcgactgtataataagttgagggc

SEQ ID NO: 19 - DNA, 104 bp, Artificial Sequence
  Synthetic Polynucleotide
ggcgagcggtagtaaaaaacttcaaaataaacgcttgacatgtcacgtcgcgtgattataattgcgcgtccgacatgatcatcagtacaataggagatatcgcc

SEQ ID NO: 20 - DNA, 128 bp, Artificial Sequence
  Synthetic Polynucleotide
ggcgctatggaggtcaggtatgattactattgacaattaatcatcggctcgtataatgtgatcagacctggaattgtgagcggataacaattcttaagattaactcacacacgagggtatcatgagcg

SEQ ID NO: 21 - DNA, 76 bp, Artificial Sequence
  Synthetic Polynucleotide
tgggcgttgttgacaattaatcatcggcatagtatatcggcatagtataatacgacaaggtgaggaactaaaccgg

SEQ ID NO: 22 - DNA, 161 bp, Artificial Sequence
  Synthetic Polynucleotide
gagcgcatgctcgagtacttcgcctggaccatgctcgccgtcgtcttcggcttcctgcacttcgtcaacctcgcctacgtccccctcggccactgggccgagacgttcgccggcttcttcaaattttcagggctgccgcaccccatcgactgggggctaag

SEQ ID NO: 23 - DNA, 67 bp, Artificial Sequence
  Synthetic Polynucleotide
attatcgctcgtgctcgccgcgaccttcctcttcatgcacctcttcggcatcgggctgcacaatcta

SEQ ID NO: 24 - DNA, 181 bp, Artificial Sequence
  Synthetic Polynucleotide
ggaatctccttctcgcctctttcgctgaacacgaaagcgaatccgttcagcacacactttacgatatggcgcaaaaagtcctcggctgcgtgccggaagtgaaagacatccacctcaccatgcccaacaagcattgcctgctcgtggacctgtcccgcttcggtcaggacaatcccaacga

SEQ ID NO: 25 - DNA, 141 bp, Artificial Sequence
  Synthetic Polynucleotide
ctcgctctatctccatcacccgaccgcgaccgacccaaaaatggcgccgccgggccattcgaccttctacgcgctcgcgccggtcccccatctcggcaaattccccgtcgactgggcgcgcgtcgggccaatagtatctta

SEQ ID NO: 26 - DNA, 95 bp, Escherichia coli
gtcgttgaggggaattccgcatttctcacacaatttataacgtaactgtcagaattgggtattattggggcaggttgtcgtgaaggaattcccta

SEQ ID NO: 27 - DNA, 192 bp, Escherichia coli
aagcatagcggattgttttcaaagggagtgtaaatttatctatacagaggtaagggttgaaagcgcgactaaattgcctgtgtaaataaaaatgtacgaaatatggattgaaaactttactttatgtgttatcgttacgtcatcctcgctgaggatcaactatcgcaaacgagcataaacaggatcgccatc

SEQ ID NO: 28 - DNA, 203 bp, Escherichia coli
gttctattccttatgcggggtcagatacttagtttgcccagcttgcaaaaaggcatcgctgcaattggtgctgaaacgataaagtaattgtgtgacccagatcgatatttacagggagcctgcctttccggcgttgttgttatgcccccaggtatttacagtgtgagaaagaattattttgactttagcggagcagttgaaga

SEQ ID NO: 29 - DNA, 204 bp, Escherichia coli
aacccccgcctccctgttactttagttataacctaaaaggttaattacagcaatgaaaaagcacctaaaaggttagttagatgtacggagatagtgaccacacaaaacgtattctttaaggaaagtgattgaccatataagaaagtggcgcattagtagcgccagtttgaagcaggaatttataagggaagctggagtcaggca

SEQ ID NO: 30 - DNA, 103 bp, Pseudomonas putida
atcgacgacaggggtttgcgcgggcgtctgcctgtggaataatactggctacttatacaggtattccggccgtcagggccaagtcgaacacgtgaggatttca

SEQ ID NO: 31 - DNA, 100 bp, Pseudomonas putida
cccagcctgttcctgtgatgtagaggggacaggctcaagcgctgccagggagaaaggtgccgctcgagtctgagttcgaactcagcaaaggattataaca

SEQ ID NO: 32 - DNA, 193 bp, Pseudomonas putida
ttgtgggggctgaattcgaagccgcgcatgatagtccccgtgtcgggtgccgaccagcggttttcgatcagaggctttgcattccggcttgctaagggttaacatccgcaacccttgaaaaccgacgttctccagcacacctttgttttgccaggagcacgtctaccccggtaatgaattaaggtagccctgg

SEQ ID NO: 33 - DNA, 113 bp, Pseudomonas putida
ccgcacaaaagctgttaatgtatgccgccgcgaaattcgacccacggggtcgcgcggcgacattgacctgactgtcggccagatccgttttgaataaagttcattcgccgccc

SEQ ID NO: 34 - protein, 513 aa, Streptomyces uncialis
MLESIVVVGGGTSGWMTASYLSAAFGERISVTVVESARVGTIGVGEATFSTVRHFFEYLGLSEETWMPACNATYKLGIRFENWRAPGHHFYHPFERQRVVDGFTLPDWWLADGGATERFDKECFLVGTLCDTMRSPRHMDGALFEGDLTDRPAGRSTLAEQGTQFPYAYHFDAALLADFLRDYAVARGVLHVVDDVVHVARDERGWISHVATRGSGDLAGDLFVDCTGFRGLLINDALDEPFESYQDTLPNDSAVALRVPVDMEREGLRPCTTSTAQAAGWIWTIPLFGRVGTGYVYARDYCTPEEAERTLRRFVGPAADDLEANHIRMRIGRSRRSWVNNCVAVGLSSGFVEPLESTGIFFIQHAIEQLVKHFPDADWDPALRSAYNTLVNRCMDGVREFLVLHYYGAARADNEYWRDTKTRKIPDSLAERVEQWRTKLPHPESVYPHYHGFEAYSYVCMVLGLGGIPLKPSPALRMLDPSAAQREFRLLATQAEDLRRTLPSQYAYFAQFR

SEQ ID NO: 35 - protein, 515 aa, Uncultered Bacterium AB1650
MLKNVVVVGGGTAGWMTASYLTAAFGDRIGVTLVESKRVGSIGVGEATFSTVRHFFEYLGLEEKEWMPACNATYKLAIRFENWREPGHHFYHPFERQRVVDGFPLTDWWLREPRSDRFDKDCFLVGTLCDDLKSPRQLNGELFEGGLGGRSAYRTTLAEQTTQFPYAYHFDATLVANYLRDYAVARGVKHVLDDVQDVALDDRGWISHVVTGESGNLTGDLFIDCTGFRSLLLGKALAEPFQSYQDSLPNDSAVALRVPQDMENRGLRPCTTATAQEAGWIWTIPLFDRIGTGYVYAGDYISPEEAERTLRAFVGPAAEHADANHIKMRIGRSNRHWVNNCVAVGLSSGFVEPLESTGIFFIQHAIEQLVKHFPDERWDDGLRTAYNKLVNNVMDGVREFLVVHYYAAKRQDNQYWKDAKTRPLPDGLAERLERWQTRLPDNESVFPHYHGFESYSYVCMLLGLGGLDLKSSPALGLMDAAPARHEFKLVGEQAAELARTLPTQYEYFAQLHRAR

SEQ ID NO: 36 - protein, 532 aa, Streptomyces rugosporus
MERRKRERLGSLGRPTKKELRMIRSVVIVGGGTAGWMTASYLKAAFDDRIDVTLVESGNVRRIGVGEATFSTVRHFFDYLGLDEREWLPRCAGGYKLGIRFENWSEPGEYFYHPFERLRVVDGFNMAEWWLAVGDRRTSFSEACYLTHRLCEAKRAPRMLDGSLFASQVDESLGRSTLAEQRAQFPYAYHFDADEVARYLSEYAIARGVRHVVDDVQHVGQDERGWISGVHTKQHGEISGDLFVDCTGFRGLLINQTLGGRFQSFSDVLPNNRAVALRVPRENDEDMRPYTTATAMSAGWMWTIPLFKRDGNGYVYSDEFISPEEAERELRSTVAPGRDDLEANHIQMRIGRNERTWINNCVAVGLSAAFVEPLESTGIFFIQHAIEQLVKHFPGERWDPVLISAYNERMAHMVDGVKEFLVLHYKGAQREDTPYWKAAKTRAMPDGLARKLELSASHLLDEQTIYPYYHGFETYSWITMNLGLGIVPERPRPALLHMDPAPALAEFERLRREGDELIAALPSCYEYLASIQ

SEQ ID NO: 37 - protein, 531 aa, Streptomyces albogriseolus
MDNRIKTVVILGGGTAGWMTAAYLGKALQNTVKIVVLEAPTIPRIGVGEATVPNLQRAFFDYLGIPEEEWMRECNASYKMAVKFINWRTPGEGSPDPRTLDDGHTDTFHHPFGLLPSADQIPLSHYWAAKRLQGETDENFDEACFADTAIMNAKKAPRFLDMRRATNYAWHFDASKVAAFLRNFAVTKQAVEHVEDEMTEVLTDERGFITALRTKSGRILQGDLFVDCSGFRGLLINKAMEEPFIDMSDHLLCNSAVATAVPHDDEKNGVEPYTSSIAMEAGWTWKIPMLGRFGSGHVYSDHFATQDEATLAFSKLWGLDPDNTEFNHVRFRVGRNRRAWVRNCVSVGLASCFVEPLESSGIYFIYAAIHMLAKHFPDKTFDKVLVDRFNREIEEMFDDTRDFLQAHYYFSPRVDTPFWRANKELKLADSIKDKVETYRAGLPVNLPVTDEGTYYGNFEAEFRNFWTNGSYYCIFAGLGLMPRNPLPALAYKPQSIAEAELLFADVKRKGDTLVESLPSTYDLLRQLHGAS

SEQ ID NO: 38 - protein, 513 aa, Streptomyces violaceusniger
LNNVVIVGGGTAGWMTASYLKAAFGDRIDITLVESGHIGAVGVGEATFSDIRHFFEFLGLKEKDWMPACNATYKLAVRFENWREKGHYFYHPFEQMRSVNGFPLTDWWLKQGPTDRFDKDCFVMASVIDAGLSPRHQDGTLIDQPFDEGADEMQGLTMSEHQGKTQFPYAYQFEAALLAKYLTKYSVERGVKHIVDDVREVSLDDRGWITGVRTGEHGDLTGDLFIDCTGFRGLLLNQALEEPFISYQDTLPNDSAVALQVPMDMERRGILPCTTATAQDAGWIWTIPLTGRVGTGYVYAKDYLSPEEAERTLREFVGPAAADVEANHIRMRIGRSRNSWVKNCVAIGLSSGFVEPLESTGIFFIHHAIEQLVKNFPAADWNSMHRDLYNSAVSHVMDGVREFLVLHYVAAKRNDTQYWRDTKTRKIPDSLAERIEKWKVQLPDSETVYPYYHGLPPYSYMCILLGMGGIELKPSPALALADGGAAQREFEQIRNKTQRLTEVLPKAYDYFTQ

SEQ ID NO: 39 - protein, 523 aa, Streptomyces toxytricini
MNTRNPDKVVIVGGGTAGWMTASYLKKAFGERVSVTLVESGTIGTVGVGEATFSDIRHFFEFLDLREEEWMPACNATYKLAVRFQDWQRPGHHFYHPFEQMRSVDGFPLTDWWLQNGPTDRFDRDCFVMASLCDAGRSPRYLNGSLLQQEFDERAEEPAGLTMSEHQGKTQFPYAYHFEAALLAEFLSGYSKDRGVKHVVDEVLEVKLDDRGWISHVVTKEHGDIGGDLFVDCTGFRGVLLNQALGVPFVSYQDTLPNDSAVALQVPLDMEARGIPPYTRATAKEAGWIWTIPLIGRIGTGYVYAKDYCSPEEAERTLREFVGPEAADVEANHIRMRIGRSEQSWKNNCVAIGLSSGFVEPLESTGIFFIHHAIEQLVKHFPAGDWHPQLRAGYNSAVANVMDGVREFLVLHYLGAARNDTRYWKDTKTRAVPDALAERIERWKVQLPDSENVFPYYHGLPPYSYMAILLGTGAIGLRPSPALALADPAAAEKEFTAIRDRARFLVDTLPSQYEYFAAMGQRV

SEQ ID NO: 40 - protein, 505 aa, Kutzneria sp. 744
MTAAYLKTAFGDRLSITVVESSRIGTIGVGEATFSDIQHFFQFLNLREQDWMPACNATYKLGIRFENWRHVGHHFYQPFEQIRPVYGFPLTDWWLHDAPTDRFDTDCFVMPNLCEAGRSPRHLDGTLADEDFVEEGDELANRTMSEHQGKSQFPYAYHFEAALLAKFLTGYAVDRGVEHVVDDVLDVRLDQRGWIEHVVTAEHGEIHGDLFVDCTGFRGLLLNKALGVPFVSYQDTLPNDSAVALQVPLDMQRRGIVPNTTATAREAGWIWTIPLFGRVGTGYVYAKDYLSPEEAERTLREFVGPAAADVEANHIRMRIGRSQESWRNNCVAIGLSSGFVEPLESTGIFFIHHAIEQLVKHFPAADWNPKSRDMYNSAVAHVMDGIREFLVIHYRGAARADNQYWRDTKTRPLPDGLAERIECWQTQLPDTETIYPYYHGLPPYSYMCILMGGGAIRTPASAALALTDQGAAQKEFAAVRDRAAQLRDTLPSHYEYLARMRGLDV

SEQ ID NO: 41 - protein, 529 aa, Uncultered Bacterium AB1091
MDNRINRIVILGGGTAGWMTASYLAKALGDTVTITLLEAPAIGRIGVGEATVPNLQRVFFDFLGLREEEWMPECNAAFKTAVKFINWRTPGPGEAKARTIDGRPDHFYHPFGLLPEHGQVPLSHYWAYNRAAGTTDEPFDYACFAETAAMDAVRAPKWLDGRPATRYAWHFDAHLVAEFLRRHATERLNVEHVQGEMQQVLRDERGFITALRTVEGRDLEGDLFIDCSGFRGLLINKAMEEPFIDMNDQLLCNRAVATAIKHDDDAHGVEPYTSAIAMRSGWSWKIPMLGRFGTGYVYSSRFAEKDEATLDFCRMWGLDPENTPLNQVAFRVGRNRRAWVKNCVSIGLASCFLEPLESTGIYFITAAIYQLTQHFPDRTFALALSDAFNHEIEAMFDDTRDFIQAHFYVSPRTDTPFWKANKDLHLPEQMREKIAMYKAGLPINAPVTDESTYYGRFEAEFRNFWTNGSYYCIFAGLGLRPDNPLPMLRHRPEQVREAQALFAGVKDKQRELVETLPSNLEFLRSLHGK

SEQ ID NO: 42 - protein, 533 aa, Kutzneria sp. 744
MDDNRIRSILVLGGGTAGWMSACYLSKALGPGVEVTVLEAPSISRIRVGEATIPNLHKVFFDFLGIAEDEWMRECNASYKAAVRFVNWRTPGDGQATPRRRPDGRPDHFDHLFGQLPEHENLPLSQYWAHRRLNGLTDEPFDRSCYVQPELLDRKLSPRLMDGTKLASYAWHFDADLVADFLCRFAVQKLNVTHVQDVFTHADLDQRGHITAVNTESGRTLAADLFIDCSGFRSVLMGKVMQEPFLDMSKHLLNDRAVALMLPHDDEKVGIEPYTSSLAMRSGWSWKIPLLGRFGSGYVYSSQFTSQDEAAEELCRMWDVDPAEQTFNNVRFRVGRSRRAWVRNCVAIGVSAMFVEPLESTGLYFSYASLYQLVKHFPDKRFRPILADRFNREVATMYDDTRDFLQAHFSLSPRDDSEFWRACKELPFADGFAEKVEMYRAGLPVELPVTIDDGHYYGNFEAEFRNFWTNSNYYCIFAGLGFLPEHPLPVLEFRPEAVDRAEPVFAAVRRRTEELVATAPTMQAYLRRLHQGT

SEQ ID NO: 43 - protein, 538 aa, Pseudomonas fluorescens
MNKPIKNIVIVGGGTAGWMAASYLVRALQQQANITLIESAAIPRIGVGEATIPSLQKVFFDFLGIPEREWMPQVNGAFKAAIKFVNWRKSPDPSRDDHFYHLFGNVPNCDGVPLTHYWLRKREQGFQQPMEYACYPQPGALDGKLAPCLSDGTRQMSHAWHFDAHLVADFLKRWAVERGVNRVVDEVVDVRLNNRGYISNLLTKEGRTLEADLFIDCSGMRGLLINQALKEPFIDMSDYLLCDSAVASAVPNDDARDGVEPYTSSIAMNSGWTWKIPMLGRFGSGYVFSSHFTSRDQATADFLKLWGLSDNQPLNQIKFRVGRNKRAWVNNCVSIGLSSCFLEPLESTGIYFIYAALYQLVKHFPDTSFDPRLSDAFNAEIVHMFDDCRDFVQAHYFTTSRDDTPFWLANRHDLRLSDAIKEKVQRYKAGLPLTTTSFDDSTYYETFDYEFKNFWLNGNYYCIFAGLGMLPDRSLPLLQHRPESIEKAEAMFASIRREAERLRTSLPTNYDYLRSLRDGDAGLSRGQRGPKLAAQESL

SEQ ID NO: 44 - protein, 157 aa, Lechevalieria aerocolonigenes
MHHGFTTPSRAIAVLSTETIRGNITFTQVQDGKVHVQGGITGLPPGEYGFHVHEKGDLSGGCLSTGSHFNPEHKDHGHPNDVNRHVGDLGNVVFDENHYSRIDLVDDQISLSGPHGIIGRAVVLHEKADDYGKSDHPDSRKTGNAGGRVACGVIGIL

SEQ ID NO: 45 - DNA, 74 bp, Artificial Sequence
  Synthetic Polynucleotide
gactgacactgataatacgactcactataggatgccgtcatccacccgggttttagagctagaaatagcaagtt

SEQ ID NO: 46 - DNA, 73 bp, Artificial Sequence
  Synthetic Polynucleotide
gactgacactgataatacgactcactataggcgagctgtacaaccggtgttttagagctagaaatagcaagtt

SEQ ID NO: 47 - DNA, 19 bp, Artificial Sequence
  Synthetic Polynucleotide
aaaagcaccgactcggtgc

SEQ ID NO: 48 - DNA, 21 bp, Artificial Sequence
  Synthetic Polynucleotide
gatagcgctgtacgaatactg

SEQ ID NO: 49 - DNA, 21 bp, Artificial Sequence
  Synthetic Polynucleotide
catactcacgctgcacaatgc

SEQ ID NO: 50 - DNA, 54 bp, Artificial Sequence
  Synthetic Polynucleotide
ggcctggtgccgcgcggcagccatatgtctgtcagtggctccgaaagatcggcc

SEQ ID NO: 51 - DNA, 56 bp, Artificial Sequence
  Synthetic Polynucleotide
gatctggggaattcggatccaagcttttagcgtccggcccgcatggccgcgaagta

SEQ ID NO: 52 - DNA, 50 bp, Artificial Sequence
  Synthetic Polynucleotide
cctggtgccgcgcggcagccatatgaccgagcggaccgccacccgaacgg

SEQ ID NO: 53 - DNA, 54 bp, Artificial Sequence
  Synthetic Polynucleotide
gtggtggtggtgctcgagtgcggccgcctatccgcctgtcccggatgccctccg

SEQ ID NO: 54 - DNA, 49 bp, Artificial Sequence
  Synthetic Polynucleotide
cctggtgccgcgcggcagccatatgtcgatcggtcgaagcactgccgag

SEQ ID NO: 55 - DNA, 52 bp, Artificial Sequence
  Synthetic Polynucleotide
gtggtggtggtgctcgagtgcggccgctcacctcgcttcgtcgagcaggctc

SEQ ID NO: 56 - DNA, 49 bp, Artificial Sequence
  Synthetic Polynucleotide
cctggtgccgcgcggcagccatatggctgcggcggtgttccggtcgtac

SEQ ID NO: 57 - DNA, 54 bp, Artificial Sequence
  Synthetic Polynucleotide
gtggtggtggtgctcgagtgcggccgctcattcgacaaggcgggcaaccgccgc